(19) United States
(12) Patent Application Publication
(10) Pub. No.: US 2009/0087845 A1
(43) Pub. Date: Apr. 2, 2009

(54) Genetic Markers Of True Low Birth Weight

(76) Inventors: Michael Morgan Myers, Elmsford, NY (US); Morris Cohen, Bronx, NJ (US)

Correspondence Address: BAKER BOTTS L.L.P., 30 ROCKEFELLER PLAZA, 44TH FLOOR, NEW YORK, NY, 10112-4498, US

(21) Appl. No.: 12/117,453

(22) Filed: May 8, 2008

Related U.S. Application Data
(63) Continuation of application No. PCT/US06/43385, filed on Nov. 7, 2006.
(60) Provisional application No. 60/735,087, filed on Nov. 8, 2005.
(60) Provisional application No. 60/814,672, filed on Jun. 16, 2006.
(60) Provisional application No. 60/834,285, filed on Jul. 28, 2006.
(60) Provisional application No. 60/854,394, filed on Oct. 25, 2006.

(51) Int. Cl. C12Q 1/68

(52) U.S. Cl. 435/6

(57) ABSTRACT

The present application provides for a method of diagnosing TLBW via measurement of expression of various TLBW-related genes. The present application also provides kits for the diagnosis of TLBW in a subject. The present application also provides a method for determining the basis for appropriate therapy for a subject suffering from TLBW.

### CROSS-REFERENCE TO RELATED APPLICATIONS

This application claims the benefit of U.S. provisional application Ser. Nos. 60/735,087 filed Nov. 8, 2005, 60/814,672 filed Jun. 16, 2006, 60/834,285 filed Jul. 28, 2006 and Ser. No. ______ not yet assigned filed Oct. 25, 2006, each of which is incorporated by reference in its entirety herein.

### GRANT INFORMATION

The subject matter of this application was developed at least in part under National Institutes of Health (NIEHS) Grant No. ES0110596, so that the United States government holds certain rights herein.

### FIELD OF THE INVENTION

The present invention relates to a method of diagnosing true low birth weight (TLBW) in a subject, kits for the diagnosis of TLBW in a subject, and a method for determining the basis for appropriate therapy for a subject diagnosed with TLBW.

### BACKGROUND OF THE INVENTION

Converging evidence demonstrates that the propensity to acquire certain adult diseases is influenced by factors that impinge upon the individual early in life, as well as by genetic and environmental interactions in adulthood. Epidemiological studies indicate that various risk factors, such as sub-optimal nutrition during gestation, resulting in reduced rates of fetal growth and birth weight, is linked to increased risk for cardiovascular (CV) disease, diabetes, obesity, and mental illness later in life. A parallel body of literature using animal models confirms that risk factors resulting in reduced rates of fetal growth and birth weight also lead to disease susceptibility. Examples of these risk factors include restricted uterine blood flow, alterations in the proportions of protein and carbohydrate in the maternal diet, and overall reductions in maternal food availability. As adults, offspring born in these experiments express a variety of phenotypes including increased insulin resistance, increased fat deposition, and hyperphagia, and elevated blood pressure (BP). It has also been found that compromised fetal growth leads to changes in autonomic nervous system development and that these effects can be seen soon after birth.

### Low Birth Weight

During the earlier part of the twentieth century, low birth weight (LBW) was presumed to be caused by preterm delivery, sometimes referred to as premature birth; the terms “LBW” and “premature” were often used interchangeably. However, epidemiologic data accumulated during the 1950s and 1960s established a distinction between infants with low birth weight due to preterm delivery, and babies which were simply constitutionally small but otherwise healthy.

It is now recognized that low birth weight is not necessarily the result of preterm birth. Low birth weight (LBW) is defined by the World Health Organization as a weight at birth of less than 5.5 pounds (2,500 grams), measured within the first hour following birth. This weight cut-off is based upon epidemiological studies and observations, and is used primarily for comparative health statistics. A multitude of factors are implicated as causes for low birth weight. These factors include, but are not limited to, the mother's nutrition (e.g., malnourishment, unbalanced diet, such as excessive carbohydrate diet or a poor protein diet), lifestyle (e.g., alcohol, tobacco, or drug abuse), and health (e.g., infections, illness, genetic factors). LBW is particularly prevalent in un-industrialized countries and impoverished areas, where the typical cause is malnourishment. While these factors may have an adverse affect on the infant's fetal growth and birth weight and result in the adverse health effects associated with LBW, other factors exist which may result in a smaller baby, but without associated adverse health effects. These include, but are not limited to, the baby's sex, gestation time, and the mother's physical characteristics (e.g., height, weight). It is noted that because birth weight may vary based upon one or more of these factors which are not associated with adverse health effects, a clinical weight cut-off value for use in diagnosing low birth weight may vary between regions, and possibly between individuals, to account for these factors. Thus, the WHO definition of LBW as less than 5.5 pounds may miscategorize infants, excluding heavier infants who are smaller than they should be, yet including healthy babies who weigh less than 5.5 pounds, for example, for genetic reasons.

Failure to identify infants whose birth weight is outside the WHO definition but who are smaller than they should be can have serious consequences, as true low birth weight (“TLBW”) is considered an indicator and predictor of health. TLBW has been shown to increase the likelihood of child mortality and disability, including higher incidence of morbidity and mortality from infectious disease and sudden infant death syndrome (SIDS), and has also been associated with an increased incidence of adult diseases such as diabetes, hypertension, coronary heart disease and other cardiovascular diseases, and stroke. From studies of the effects of famine, TLBW is also known to be associated with an increased risk for a number of psychiatric disorders including antisocial behavior, depression, and schizophrenia.

Intrauterine growth restriction (IUGR), sometimes referred to as intrauterine growth retardation, is a subtype of low birth weight characterized by constrained growth in the womb, and is typically defined as birth weight falling under the 10th percentile of gestational age. IUGR infants may have normal gestational periods, and although they fall within the lower range for their gestational age, their birth weight may exceed 5.5 pounds and therefore are not diagnosed as low birth weight. Causes of IUGR include, but are not limited to, abnormal or retarded growth of the uterus, abnormal or retarded formation of the placenta, maternal malnutrition, illness and infection, multiple gestation, and various behavioral risk factors such as tobacco, alcohol, and drug abuse. Differential expression of maternally and paternally imprinted genes has also been implicated in IUGR. McMinn et al., Unbalanced Placental Expression of Imprinted Genes in Human Intrauterine Growth Restriction, Placenta, 2006, (6-7):540-9 (electronic publication on Aug. 24, 2005).

Fetal alcohol syndrome (FAS) is a subtype of low birth weight characterized by exposure to alcohol during gestation. FAS encompasses a number of alcohol-related conditions which include, but are not limited to, fetal alcohol effects (FAE), partial fetal alcohol syndrome (PFAS), alcohol related neurodevelopmental disorder (ARND), and alcohol related birth defects (ARBD). A diagnosis of FAS typically consists of three criteria: (1) characteristic facial features including a flattened midface, thin upper lip, indistinct/absent philtrum, and short eye slits; (2) growth retardation characterized by low birth weight, sometimes characterized by height and/or weight below the 5th percentile; and (3) central nervous system neurodevelopmental abnormalities. A child suffering from FAS may display impaired fine motor skills, learning disabilities, and/or behavior disorders.

### Current Diagnostic Methods

Presently, low birth weight is determined by measuring the newborn infant's weight within the first hour following birth. As noted above, although the generally accepted birth weight cut-off is 5.5 pounds, it is sometimes appropriate to utilize a different cut-off for different populations or regions. However, regardless of the specific weight value utilized, an absolute cut-off weight fails to distinguish between infants who are pathologically low birth weight and normal, healthy infants which are simply small. Similarly, it fails to identify infants which exceed 5.5 pounds but may still have reduced fetal growth and birth weight, and may be at risk for the associated health problems.

### Current Therapies

Currently, there are no specialized therapies and interventions for infants diagnosed with TLBW. The standard procedure is to simply feed the infant and make certain the infant is kept warm, and to administer antibiotics if necessary. Current therapies thus do not account for the specific cause of the true low birth weight (e.g., insufficient calories, low protein), and do not necessarily correct the underlying defects which may contribute to future health risks.

Thus, there is a need for a method which accurately and objectively diagnoses true LBW which does not utilize an absolute cut-off. Accordingly, the present application provides for a method of diagnosing TLBW via measurement of expression of various TLBW-related genes, for example, in the placenta. The present application also provides kits for the diagnosis of TLBW in a subject. The present application also provides a method for determining the appropriate therapy for a subject suffering from TLBW.

### SUMMARY OF THE INVENTION

The present invention relates to a method of diagnosing true low birth weight (“TLBW”) in a subject, kits for the diagnosis of TLBW in a subject, and methods of determining the basis for treatment for a subject diagnosed with TLBW. It is based, at least in part, on the discovery that the level of insulin-like growth factor-1 (“IGF1”) expression (as measured in placental RNA) was decreased in TLBW infants, based on measurements of increased blood pressure and heart rate responses to feeding and head-up tilting; and, on the discovery that babies with birth weights on the low end of the normal distribution, but not classified as LBW or small for gestational age (SGA) by current criteria, and which had low levels of expression of both IGF1 and IGF2 in their placentas, were thinner, had lower baseline heart rates, and had increased heart rate responses to feeding, all potentially indicative of TLBW. It is further based, in part, on the discovery of a number of “TLBW related genes,” the expression levels of which significantly differ (are increased or decreased) from control levels in an animal model of TLBW.

The present invention provides a method of diagnosing TLBW comprising: (a) measuring the level of gene expression of one or more TLBW related genes in a subject sample (preferably a placental tissue sample); (b) comparing measured expression of the TLBW related genes to a standard or control; wherein a change in gene expression characterized by a p-value, relative to gene expression in a healthy subject, which is less than or equal to 0.1, less than or equal to 0.05, less than or equal to 0.01, or less than or equal to 0.001 indicates a diagnosis of true low birth weight. The standard or control may be one or more samples derived from one or more healthy, non-TLBW infant. Although the sample is preferably placenta, the subject sample may be derived from any source which may provide fetal or infant tissue or fluid, and may include maternal tissue and/or blood samples. In a preferred embodiment, gene expression is measured by a DNA chip or quantitative real-time polymerase chain reaction (PCR).

The method of the present invention may be used to evaluate the status of a subject believed to be at risk for true low birth weight. Risk factors for true low birth weight include, but are not limited to, a mother's poor nutrition (e.g., malnourishment, caloric restriction, unbalanced diet, such as excessive carbohydrate diet or a poor protein diet), lifestyle (e.g., alcohol, tobacco, or drug abuse), and/or health (e.g., infections, illness, genetic factors).

In another embodiment, the TLBW related genes are selected from the group consisting of genes encoding an insulin-like growth factor (“IGF,” e.g., IGF1, IGF2), and/or genes encoding IGF binding proteins (e.g., ALS, CTGF/CCN2, Endocan, IGFBP-1, IGFBP-2, IGFBP-3, IGFBP-4, IGFBP-5, IGFBP-6, IGFBP-7, Nov/CCN3), and genes encoding IGF receptors. In a preferred embodiment, the gene expression of more than one gene is evaluated. Certain embodiments include determining the level of IGF gene expression levels; other embodiments do not include determining the level of IGF gene expression levels. Genes which may be used in the present invention may be selected from the genes represented in Tables 1, 2, 6, 7, 10, 11, 12, and/or 13. It will be known to a person of ordinary skill in the art that sequence homology between the rat genes and the human equivalents will allow extrapolation of this data for use in humans and other species. Based upon this information, a person of ordinary skill in the art will be capable of selecting genes which display an increase or decrease in gene expression.

In another non-limiting embodiment, the present invention provides a method of diagnosing fetal alcohol syndrome (FAS) comprising: (a) measuring the level of gene expression of one or more FAS related genes in a subject sample (preferably a placental tissue sample); (b) comparing measured expression of the FAS related genes to a standard or control; wherein a change in gene expression characterized by a p-value, relative to gene expression in a healthy subject, which is less than or equal to 0.1, less than or equal to 0.05, less than or equal to 0.01, or less than or equal to 0.001 indicates a diagnosis of fetal alcohol syndrome. Genes which may be used in the present invention to diagnose fetal alcohol syndrome may be selected from the genes represented in Tables 1, 2, 6, 7, 10, 11, 12, and/or 13, and are preferably selected from Tables 11, 12, and/or 13. The standard or control may be one or more samples derived from one or more healthy, non-FAS infant. Although the sample is preferably placenta, the subject sample may be derived from any source which may provide fetal or infant tissue or fluid, and may include maternal tissue and/or blood samples. In a preferred embodiment, gene expression is measured by a DNA chip or quantitative real-time polymerase chain reaction (PCR).

The present invention provides for a kit for diagnosis of true low birth weight comprising: (1) one or more oligonucleotide probes directed to true low birth weight related genes; (2) reagents and equipment for measuring gene expression; and (3) control reagents. The kit may optionally include reagents and equipment for the collection of the tissues and fluids required for these determinations. In one embodiment, the oligonucleotide probes may bind to genes selected from the group consisting of genes encoding an insulin-like growth factor (“IGF,” e.g., IGF1, IGF2), and/or genes encoding IGF binding proteins (e.g., ALS, CTGF/CCN2, Endocan, IGFBP-1, IGFBP-2, IGFBP-3, IGFBP-4, IGFBP-5, IGFBP-6, IGFBP-7, Nov/CCN3), and/or genes encoding IGF receptors. In another embodiment, the oligonucleotide probes may bind to genes selected from the genes represented in Tables 1, 2, 6, 7, 10, 11, 12, and/or 13.

The present invention provides for a kit for diagnosis of fetal alcohol syndrome (FAS) comprising: (1) one or more oligonucleotide probes directed to fetal alcohol syndrome related genes; (2) reagents and equipment for measuring gene expression; and (3) control reagents. The kit may optionally include reagents and equipment for the collection of the tissues and fluids required for these determinations. In one embodiment, the oligonucleotide probes may bind to genes selected from the genes represented in Tables 1, 2, 6, 7, 10, 11, 12, and/or 13, and are preferably selected from Tables 11, 12, and/or 13.

The present invention also provides for a method for determining the basis for appropriate therapy for a subject diagnosed with true low birth weight, comprising: a) identifying one or more true low birth weight associated genes which display differential expression in a subject and are associated with a risk factor; and b) selecting a treatment which offsets the risk factor; wherein offsetting the risk factor means counteracting the exposures, for example, by providing a factor to which the subject was underexposed, or limiting a factor to which the subject was overexposed. Optionally, the method may also include the step of: c) administering the selected (offsetting) treatment.

### DEFINITIONS

As used herein, the term “true low birth weight” or “TLBW” refers to a condition where a newborn infant has been identified to have appropriately increased or decreased expression of a TLBW related gene and/or displays one or more of the following clinical findings, in particular:

A TLBW infant may exhibit an increase in baseline systolic blood pressure wherein the increase is characterized by a p-value relative to the systolic blood pressure in a healthy control infant which is less than or equal to 0.1, less than or equal to 0.05, less than or equal to 0.01, or less than or equal to 0.001; and/or

A TLBW infant may exhibit an increase in heart rate response during feeding that is characterized by a p-value relative to the heart rate response during feeding in a healthy control infant which is less than or equal to 0.1, less than or equal to 0.05, less than or equal to 0.01, or less than or equal to 0.001; and/or

A TLBW infant may exhibit a heart rate increase following a 30° head-up tilt that is characterized by a p-value relative to the heart rate increase in a healthy control infant following a 30° head-up tilt which is less than or equal to 0.1, less than or equal to 0.05, less than or equal to 0.01, or less than or equal to 0.001; and/or

A TLBW infant may exhibit a baseline heart rate that is lower than in healthy control infants wherein the difference in baseline heart rate is characterized by a p-value relative to the baseline heart rate in a healthy subject which is less than or equal to 0.1, less than or equal to 0.05, less than or equal to 0.01, or less than or equal to 0.001; and/or

A TLBW infant may exhibit a decrease in ponderal index, a marker of thinness in newborn infants, that is characterized by a p-value relative to the ponderal index in a healthy control infant which is less than or equal to 0.1, less than or equal to 0.05, less than or equal to 0.01, or less than or equal to 0.001.

As a non-limiting example, a TLBW infant may exhibit an increase in baseline systolic blood pressure of at least 10 mmHG and/or 5 mmHG in diastolic pressure above that of non-TLBW infants.

As a non-limiting example, a TLBW infant may exhibit an increase in heart rate response during feeding that is at least 10 BPM greater than in non-TLBW infants.

As a non-limiting example, a TLBW infant may exhibit a heart rate increase that is at least 2 BPM greater than in non-TLBW infants following a 30° head-up tilt.

As a non-limiting example, a TLBW infant may exhibit a baseline heart rate that is 10 BPM lower than in non-TLBW infants.

As a non-limiting example, a TLBW infant may exhibit a decrease in ponderal index, a marker of thinness in newborn infants, that is at least 5% relative to a healthy control infant.

As used herein, the term “cDNA” can refer to a single-stranded or double-stranded DNA molecule. For a single-stranded cDNA molecule, the DNA strand is complementary to the messenger RNA (“mRNA”) transcribed from a gene. For a double-stranded cDNA molecule, one DNA strand is complementary to the mRNA and the other is complementary to the first DNA strand.

As used herein, the term “gene” refers to a DNA molecule that either directly or indirectly encodes a nucleic acid or protein product that has a defined biological activity. Such genes may also be referred to as “biologically active” genes.

As used herein, two nucleic acid molecules are “functionally equivalent” when they share one or more quantifiable biological function. For example, nucleic acid molecules of different primary sequence may encode identical polypeptides; such molecules, while distinct, are functionally equivalent. In this example, these molecules will also share a high degree of sequence homology. Similarly, nucleic acid molecules of different primary sequence may share activity as a promoter of RNA transcription, wherein said RNA transcription occurs in a specific subpopulation of cells, and responds to a unique group of regulatory substances; such nucleic acid molecules are also functionally equivalent.

As used herein, two nucleic acid molecules are “homologous” when at least about 60% to 75%, at least about 80%, at least about 90%, at least about 95%, or at least about 100% of the corresponding nucleotides comprising the nucleic acid molecule are identical over a defined length of the molecule, as determined using standard sequence analysis software such as Vector NTI, GCG, or BLAST. DNA sequences that are homologous may be identified by hybridization under stringent conditions, as defined for the particular system. Defining appropriate hybridization conditions is within the skill of the art. See e.g. Current Protocols in Molecular Biology, Volume I, Ausubel et al., eds. John Wiley:New York N.Y., first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised of 40 mM NaPO4, pH 7.2, 1-2% SDS and 1 mM EDTA. Again, a washing temperature of at least 65-68° C. is recommended, but the optimal temperature required for a truly stringent wash will depend on the length of the nucleic acid probe, its GC content, the concentration of monovalent cations and the percentage of formamide, if any, that was contained in the hybridization solution (Ausubel et al., supra).

As used herein, the term “nucleic acid molecule” includes both DNA and RNA and, unless otherwise specified, includes both double-stranded and single-stranded nucleic acids. Also included are molecules comprising both DNA and RNA, either DNA/RNA heteroduplexes, also known as DNA/RNA hybrids, or chimeric molecules containing both DNA and RNA in the same strand. Nucleic acid molecules of the invention may contain modified bases. The present invention provides for nucleic acid molecules in both the “sense” orientation (i.e. in the same orientation as the coding strand of the gene) and in the “antisense” orientation (i.e. in an orientation complementary to the coding strand of the gene).

As used in this application, the term “purifying” refers to separation of the target nucleic acid from one or more components of the biological sample (e.g., other nucleic acids, proteins, carbohydrates or lipids). Preferably, a purifying step removes at least about 50%, more preferably about 70% or more, and even more preferably about 90% or more of the other sample components.

As used herein, the term “sequence” refers to a nucleic acid molecule having a particular arrangement of nucleotides, or a particular function, e.g. a termination sequence.

As used herein, the term “subject” refers to an animal, e.g., a bird or mammal. In one embodiment, the subject is a human. In another embodiment, the subject is a newborn infant human or a pregnant human female at term.

As used herein, the term “derived” means “obtained from,” “descending from,” or “produced by.” In the context of tissue or fluid samples derived from a particular parent source, the term derived refers to obtaining the tissue or fluid samples from the parent source. In the context of nucleic acids or polypeptides derived from a particular parent source, the term derived refers to the use of the parent source as a template for the nucleic acid sequence or the amino acid sequence. The nucleic acid or polypeptide derived from the parent source may possess all or part of the nucleic acid or amino acid sequence of the parent source, in the presence or absence of deletions, substitutions, or modification.

As used herein, the term “probe” refers to a nucleic acid oligomer that hybridizes specifically to a nucleic acid target sequence, under conditions that promote hybridization, thereby allowing detection of the target sequence. Detection may either be direct (i.e., resulting from a probe hybridizing directly to the target sequence) or indirect (i.e., resulting from a probe hybridizing to an intermediate molecular structure that links the probe and target sequences). The “target sequence” of a probe refers to a sequence within a nucleic acid, preferably in an amplified nucleic acid, which hybridizes specifically to at least a portion of a probe oligomer. A probe may hybridize under appropriate hybridization conditions even if not completely complementary to the target sequence, if the probe is sufficiently homologous to the target sequence.

The probe may be labeled, i.e., joined directly or indirectly to a detectable molecular moiety or a compound that leads to a detectable signal. Direct labeling can occur through bonds or interactions that link the label to the probe, including covalent bonds and non-covalent interactions (e.g. hydrogen bonding, hydrophobic and ionic interactions), or formation of chelates or coordination complexes. Indirect labeling occurs through use of a bridging moiety (a “linker”), that joins a label to the probe, and which can amplify a detectable signal (e.g., see PCT No. WO 95/16055 (Urdea et al.)). Labels are well known and include, for example, radionuclides, ligands (e.g., biotin, avidin), enzymes and/or enzyme substrates, reactive groups, redox active moieties such as transition metals (e.g., Ruthenium), chromophores (e.g., a moiety that imparts a detectable color), luminescent compounds (e.g., bioluminescent, phosphorescent or chemiluminescent labels) and fluorescent compounds. Those skilled in the art will appreciate that a labeled probe may be a mixture of labeled and unlabeled oligonucleotides that hybridize specifically to the target sequence, to optimize the specific activity of the probe reagent for detection.

### BRIEF DESCRIPTION OF THE FIGURES

FIG. 1. A multiple regression analysis of the use of placental IGF1 expression and body length as predictors of heart rate (HR) response to feeding (x-axis) is compared to the actual measured heart rate (HR) response to feeding (y-axis). Infants were measured for length and placental IGF1 gene expression, and this information was used to predict the HR response to feeding (data not shown). The predicted HR response was then compared to the actual measured HR response. Length and placental IGF1 gene expression are both negatively correlated with HR response to feeding (rs=0.32, 0.34; ps<0.0-5) and together provide an even better prediction of HR responses. The predicted HR response to feeding is an accurate predictor of actual HR response to feeding (Multiple R=0.42, N=59, p=0.004, p<0.005). As IGF1 expression and body length both decrease, the HR response is predicted to increase.

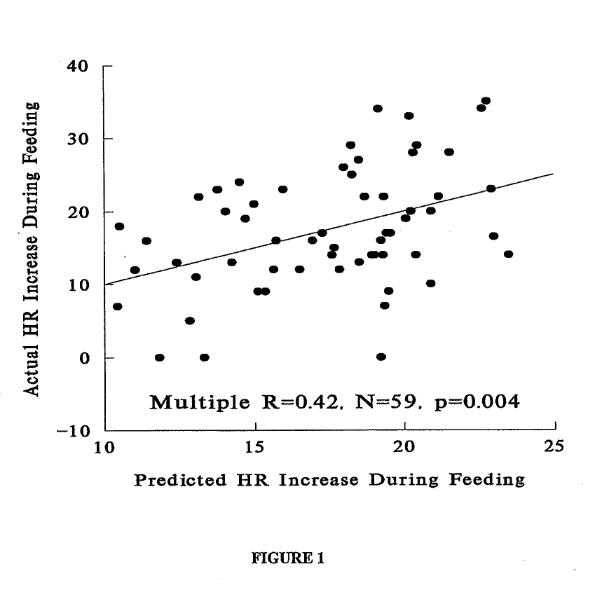

FIG. 2. IGF1 expression differs greatly with birth weight although this relationship is not linear. IGF1 expression was determined (measured with real time PCR as level of fluorescent intensity relative to actin) in babies classified as low birth weight (L), medium birth weight (M), or High birth weight (H). Expression differed greatly between the groups with L and M having expressing lower levels of IGF1 than H, and M expressing IGF1 at levels lower than L.

FIG. 3. True low BW babies with IGF1 expression have larger placentas, but were not thicker nor did they weigh more. Placental diameter was measured in babies classified as having low birth weight. Those babies with lower levels of IGF1 expression (1) had placental diameters that were larger than those babies with higher IGF1 expression levels.

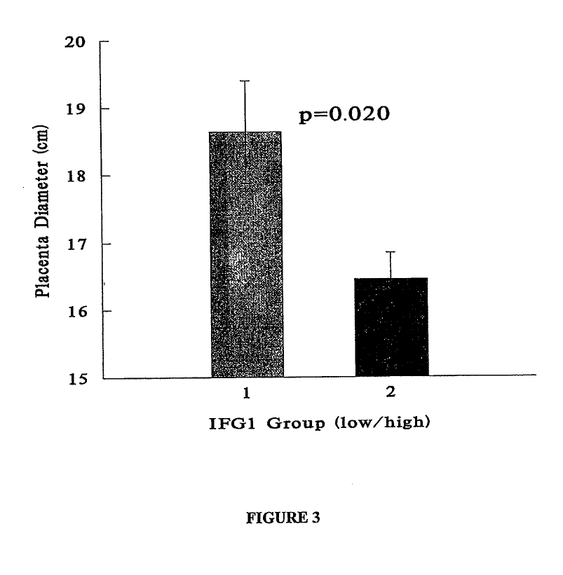

FIG. 4. True low BW babies with low IGF1 expression have higher blood pressures during the period before feeding. Systolic (gray column) and Diastolic (black column) blood pressure was measured in low birth weight babies with low IGF1 expression (1) and high IGF1 expression (2) during the period before feeding. In both Systolic and Diastolic BP, the low birth weight babies with low IGF1 expression had higher levels.

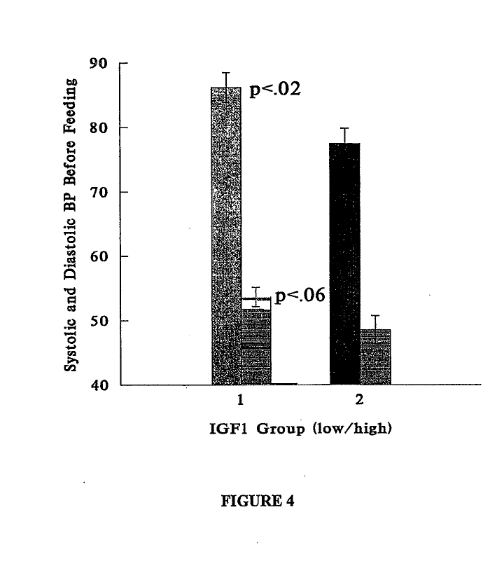

FIG. 5. True low BW babies with low IGF1 expression have greater heart rate (HR) responses to feeding. HR was measured in low birth weight babies with low IGF1 expression (1) and high IGF1 expression (2) in response to feeding. The low birth weight babies with low IGF1 expression had HR response to feeding that low birth weight babies with higher IGF1 expression.

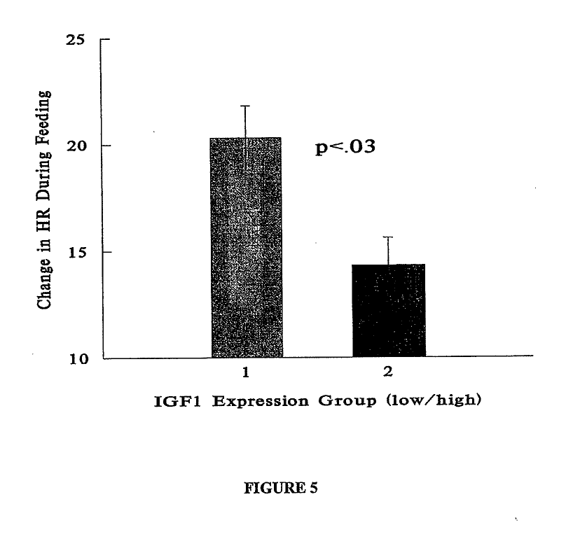

FIG. 6. True low BW babies with low IGF1 expression have greater heart rate (HR) responses to head-up tilting. HR was measured in low birth weight babies with low IGF1 expression (1) and high IGF1 expression (2) in response to head-up tilting. The low birth weight babies with low IGF1 expression had HR response to feeding that low birth weight babies with higher IGF1 expression.

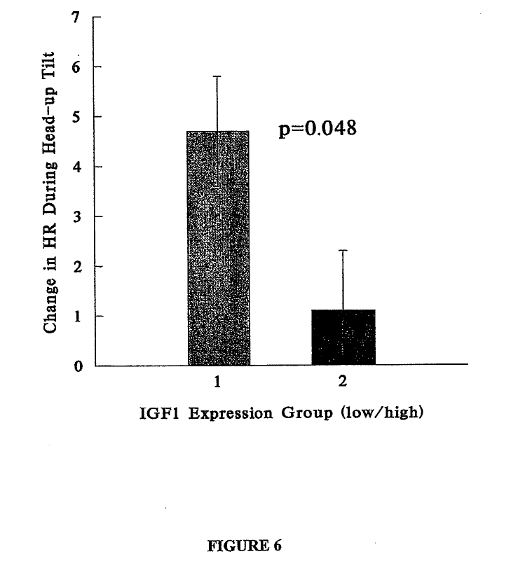

FIG. 7. Expression levels in log units of apolipoprotein C-1 (designated “G3”) in the placentas of control rats and those fed high fat diets.

FIG. 8. Expression levels in rat placentas (in log units) of vascular adhesion molecule 1 (designated “G7”) for animals in a 70% caloric reduction during pregnancy group, 50% caloric reduction during pregnancy group, and a control group.

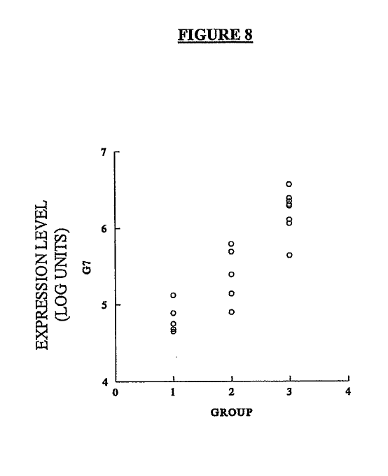

FIG. 9. Results from rat studies with nutrition manipulations during pregnancy. Predicted caloric restriction scores (CRS) for the control group, 50% caloric restriction group, 70% caloric restriction group, high fat group, and SSRI treated group. The CRS was calculated assigning a value of 1, 2, or 3 to the gene data from the control, 50% caloric restriction group, and 70% caloric restriction group, respectively.

FIG. 10. Predicted caloric restriction scores (CRS) for the control group, 50% caloric restriction group, 70% caloric restriction group, high fat group, and SSRI treated group. The CRS was calculated assigning a value of 100, 50, or 30 to the gene data from the control, 50% caloric restriction group, and 70% caloric restriction group, respectively.

FIG. 11. The relationship between body weight and the prediction score for percent normal nutrition is shown. Fetal weight was measured at day 21 of gestation, i.e., one day prior to the expected delivery date.

FIG. 12. The mean (±SE) expression values for each of these 6 genes. This figure shows a clear dose-response change in expression associated with alcohol exposure during pregnancy.

FIG. 13. The mean estimates of alcohol exposure for each group are compared to the actual alcohol exposure for each group. Expression levels of these genes can be used to produce very accurate estimates of exposure level.

### DETAILED DESCRIPTION OF THE INVENTION

### Method of Diagnosing True Low Birth Weight

The present invention relates to a method of diagnosing true low birth weight (TLBW) in newborn infants. This method may comprise: (1) measuring the expression of one or more genes associated with true low birth weight in an infant subject in a placental sample or a tissue sample collected from the infant; (2) comparing the measured expression of the TLBW related gene in the sample to the expression level of the same gene or genes in one or more control samples and/or against a standard value. Gene expression may be measured by any method known in the art, by measurement of mRNA levels (for example, via microarray or rtPCR) or by measurement of protein levels. Not by way of limitation, the control samples may be from TLBW subjects or normal subjects, and may be a placental sample or samples derived from another source. The sample or samples may be analyzed essentially immediately or may be preserved for later analysis (e.g., frozen, or the RNA may be collected and stored under standard laboratory conditions). The control samples may be derived from subjects with comparable birth weight, e.g., in the same quintile for birth weight, where a subject is not a TLBW infant. The method may further comprise measurement of a standardized, internal control having constant gene expression, to which relative expression of the TLBW related gene may be compared. Appropriate standardized internal controls are discussed in more detail below. Alternatively, or in addition, the characteristic patterns of gene expression in infants diagnosed with TLBW associated with various types of fetal exposures may be embodied in a database or otherwise categorized to provide standard values. Such a database may comprise gene expression patterns for both TLBW infants, and healthy, non-TLBW infants. Standardized values, which may be derived from the database, may be displayed in the form of charts providing characteristic levels of gene expression. The database, and outputs from the database, such as charts, may be further categorized by various factors, including but not limited to, the subject's birth weight, the subject's gestation time, exposures, as well as other medical data.

For genes which are downregulated in TLBW infants, a decrease in gene expression characterized by a p-value relative to gene expression in a healthy subject which is less than or equal to 0.1, less than or equal to 0.05, less than or equal to 0.01, or less than or equal to 0.001 indicates a diagnosis of true low birth weight. As a non-limiting example, the downregulation may be characterized by a decrease in gene expression of at least 5 percent, at least 10 percent, at least 20 percent, at least 50 percent, or at least 90 percent. Alternatively, for genes which are upregulated in TLBW infants, an increase in gene expression characterized by a p-value relative to gene expression in a healthy subject which is less than or equal to 0.1, less than or equal to 0.05, less than or equal to 0.01, or less than or equal to 0.001 indicates a diagnosis of true low birth weight. As a non-limiting example, the upregulation may be characterized by an increase in gene expression of at least 5 percent, at least 10 percent, at least 20 percent, at least 50 percent, or at least 90 percent.

Threshold values for gene expression for TLBW related genes may be determined by measuring the gene expression in healthy, normal non-TLBW infants. In one embodiment, gene expression in subjects (e.g., laboratory animals) which have been subjected to restricted nutritional intake to induce TLBW, for example, caloric restriction (CR), may be compared to the gene expression in comparable subjects with normal nutritional intake, which will be healthy and of normal birth weight, and will not have TLBW. Other nutritional or metabolic factors which may be varied include, but are not limited to: calories, oxygen, vitamins, minerals, and the appropriate combinations of protein, carbohydrates, and fats. Increases or decreases in gene expression in the TLBW group, relative to the non-TLBW group, indicates a change in gene expression induced by TLBW. The level of gene expression in the TLBW group may be used as the threshold value for use in the method of diagnosing TLBW. It is noted that under- or over-exposure to different nutritional metabolic factors may result in increase or decrease in different TLBW related genes.

In another embodiment, determination of a threshold value of gene expression of TLBW related genes may be performed by measuring gene expression in newborn infants and correlating increases or decreases in gene expression with other TLBW-associated factors, such as changes heart rate, blood pressure, and patterns of postnatal growth. The level of gene expression for a given gene in an infant which clinically manifests TLBW may be used as the threshold value in the method of diagnosing TLBW. Based upon the methods described herein, and the examples provided below, a person of ordinary skill in the art will be enabled to determine threshold values for use in the method of the present invention.

In one embodiment, the threshold value of gene expression of a TLBW related gene in a healthy subject is the same or similar regardless of the birth weight of the subject. The threshold value of gene expression for many TLBW related genes does not vary significantly among infants, regardless of birth weight. For these TLBW related genes, the same threshold value of gene expression may be utilized to measure potential changes in gene expression in a subject. A change in gene expression of a TLBW related gene in a subject which is indicative of TLBW may be characterized by a p-value relative to the gene expression of the TLBW related gene in a healthy subject, which is less than or equal to 0.1, less than or equal to 0.05, less than or equal to 0.01, or less than or equal to 0.001.

In another non-limiting embodiment, the threshold value of gene expression of a TLBW related gene may vary based upon the birth weight of the subject. Healthy infants born in different quintiles of birth weight may have different threshold levels of expression of TLBW related genes. For example, the threshold gene expression of a TLBW related gene in healthy subjects of the lowest quintile (1-20%) may differ from the threshold gene expression of the same TLBW related gene in healthy subjects in the middle quintile (41-60%) and/or the highest quintile (81-20%). Thus, while the gene expression of a TLBW related gene may increase or decrease in infants from one quintile relative to infants in a different quintile, the relative change in TLBW related gene expression is not necessarily an indication of TLBW. Accordingly, in preferred but-non-limiting embodiments of the invention, for genes in which the level of gene expression may vary by birth weight quintile, the threshold values of TLBW related gene expression which is used may be determined from healthy subjects of the same birth weight quintile.

The threshold value for a given birth weight quintile may be determined by measuring the gene expression of a TLBW related gene in infants from that birth weight quintile. In one non-limiting embodiment, gene expression may be measured relative to a standardized, internal control which displays constant gene expression, such as 18S mRNA, GAPDH, or actin. Measurement of gene expression may thus be expressed in logarithmic (base-10) values relative to the standardized, internal control. A value of log(1) indicates a ten-fold increase in expression relative to the standardized, internal control, a value of log(2) indicates a one-hundred-fold increase in expression relative to the standardized, internal control, and so on. Where the threshold value of gene expression is expressed as a logarithmic (base-10) value, relative to a standardized, internal control, the measurement of gene expression of a particular TLBW related gene must be measured relative to the same standardized, internal control as well. Because the standardized, internal control is the same, the measured level of gene expression may be compared as an absolute value against the threshold value of gene expression.

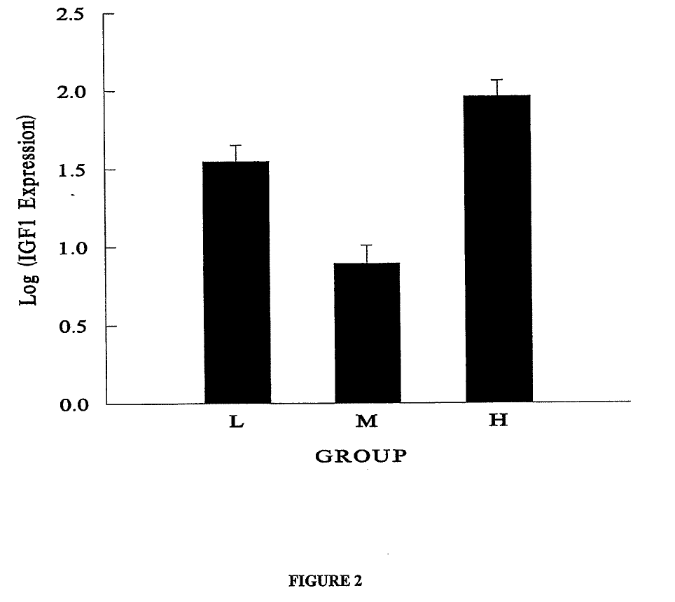

In one non-limiting embodiment, the threshold level of gene expression of IGF1, relative to a standardized internal control comprising actin, differs for infants in the lowest birth weight quintile, the medium birth weight quintile, and the highest birth weight quintile. FIG. 2 shows the baseline gene expression of IGF1 in infants in the three quintiles. As shown in FIG. 2, the threshold gene expression of IGF1 relative to actin is about log(1.5) for infants in the lower quintile (1-20 percent) of birth weight. The threshold gene expression of IGF1 relative to actin is about log(1) for infants in the middle quintile (41-60 percent) of birth weight. The threshold gene expression of IGF1 relative to actin is about log(2) for infants in the upper quintile (81-100 percent) of birth weight. See FIG. 2. These threshold levels of IGF1 gene expression relative to actin may be used to determine whether changes in gene expression in a subject are indicative of TLBW. As one illustrative example, an IGF1 gene expression value, relative to actin, of log(1.2) would be indicative of TLBW in a subject in the lowest quintile and the highest quintile of birth weight, but would not be indicative of TLBW in a subject in the middle quintile of birth weight. In another example, based upon the threshold values provided above, an IGF1 gene expression value of log(0.1), relative to actin, would be indicative of TLBW in a subject in the lowest, middle, and highest quintiles, as it falls below the threshold values in each category.

Increases or decreases in gene expression are indicative of TLBW if they are statistically significant, as indicated by the p-values discussed above. To confirm a diagnosis of TLBW, at least one TLBW-related gene is evaluated, and preferably, the gene expression of additional TLBW-related genes are concurrently evaluated. The TLBW-related genes evaluated may be utilized as a standard set of genes, wherein the same set of genes are evaluated between the test subject(s) and the control subject(s). The set of genes evaluated may include at least 1 TLBW-related gene, at least 2 TLBW-related genes, at least 3 TLBW-related genes, at least 4 TLBW-related genes, at least 5 TLBW-related genes, at least 6 TLBW-related genes, at least 7 TLBW-related genes, at least 8 TLBW-related genes, at least 9 TLBW-related genes, at least 10 TLBW-related genes, at least 15 TLBW-related genes, at least 20 TLBW-related genes, or at least 25 TLBW-related genes.

In a preferred embodiment, the TLBW related genes evaluated include: insulin-like growth factors (“IGF,” e.g., IGF1, IGF2), genes encoding IGF binding proteins (e.g., ALS, CTGF/CCN2, Endocan, IGFBP-1, IGFBP-2, IGFBP-3, IGFBP-4, IGFBP-5, IGFBP-6, IGFBP-7, Nov/CCN3), and genes encoding IGF receptors. TLBW related genes which may be evaluated may also be selected from the genes represented in Tables 1, 2, 6, 7, and/or 10. Gene expression of a TLBW related gene is decreased in a subject sample if the decrease in gene expression is characterized by a p-value relative to gene expression in a healthy subject which is less than or equal to 0.1, less than or equal to 0.05, less than or equal to 0.01, or less than or equal to 0.001. Gene expression a TLBW related gene is increased in a subject sample if the increase in gene expression is characterized by a p-value relative to gene expression in a healthy subject which is less than or equal to 0.1, less than or equal to 0.05, less than or equal to 0.01, or less than or equal to 0.001.

In a non-limiting embodiment, the TLBW related genes are FAS related genes, and the method of diagnosing TLBW is utilized to diagnose fetal alcohol syndrome. Increases or decreases in gene expression of FAS related genes are indicative of FAS if they are statistically significant, as indicated by the p-values discussed above. To confirm a diagnosis of FAS, at least one FAS-related gene is evaluated, and preferably, the gene expression of additional FAS-related genes are concurrently evaluated. The FAS-related genes evaluated may be utilized as a standard set of genes, wherein the same set of genes are evaluated between the test subject(s) and the control subject(s). The set of genes evaluated may include at least one FAS-related gene, at least 2 FAS-related genes, at least 3 FAS-related genes, at least 4 FAS-related genes, at least 5 FAS-related genes, at least 6 FAS-related genes, at least 7 FAS-related genes, at least 8 FAS-related genes, at least 9 FAS-related genes, at least 10 FAS-related genes, at least 15 FAS-related genes, at least 20 FAS-related genes, or at least 25 FAS-related genes.

In a preferred embodiment, the FAS-related genes evaluated include: Gusb, Pex6, Arts1, Centa2, Hadhsc, Vps35 (mapped), Tpar1, RGD1305243 (predicted), Cdca7, RGD1565289 (predicted), Crsp9 (predicted), Ggps1, Cul2 (predicted), Trp53rk (predicted), Prdm2 (mapped), Ddx59, Mcpt6, Ndufb5 (predicted), Mcts1, Enpp5, MGC72992, Sumf1 (predicted), Plek, Plag1, Rnd3, Inpp1, Zfp207, Akr7a2, Cp, St3gal3, LOC287533, Slc39a4 (predicted), Polr3e (predicted), Pcmtd2 (predicted), Kbtbd4 (predicted), Asah1, RGD1564011 (predicted), Mss4, RT1-Aw2, LOC303515, Wig1, Phyh, RGD1308959, Csnk1d, Zfp365, and Lama5. FAS related genes which may be evaluated may also be selected from the genes represented in Tables 1, 2, 6, 7, 10, 11, 12, and/or 13, and are preferably selected from Tables 11, 12, and 13. Gene expression of a FAS related gene is decreased in a subject sample if the decrease in gene expression is characterized by a p-value relative to gene expression in a healthy subject which is less than or equal to 0.1, less than or equal to 0.05, less than or equal to 0.01, or less than or equal to 0.001. Gene expression a FAS related gene is increased in a subject sample if the increase in gene expression is characterized by a p-value relative to gene expression in a healthy subject which is less than or equal to 0.1, less than or equal to 0.05, less than or equal to 0.01, or less than or equal to 0.001.

Gene expression may be measured from nucleic acids derived from fetal tissue or fluid samples. A preferred tissue sample is placental tissue, which is composed largely of fetal tissue. Preferably, a suitable cross section is obtained which includes all cell layers, i.e., amnion, chorion, decidua parietalis, endometrial veins and arteries, and myometrium. Any fetal tissue or fluid sample from a subject may be used. Tissue and fluid samples may be derived from the newborn infant, or from maternal sources which may contain fetal tissue or fluid. Tissue or fluid samples which may be useful for assaying the expression level of genes of interest are any tissues or fluids which may exhibit differential gene expression of the target genes. Examples of tissue samples that may be used include, but are not limited to: placental tissue, fetal blood, cord tissue, cord blood, amniotic fluid, maternal blood, and endometrium. Tissue and fluid samples may be acquired via any method known in the art, including, but not limited to surgical excision, aspiration, or biopsy. The tissue and fluid samples may be fresh, frozen, or otherwise preserved. In a preferred embodiment, placental tissue is sampled within 48 hours following birth, and placed in a preservative or stabilizer. Examples of preservatives and stabilizers include TRIZOL™ (Invitrogen, Carlsbad, Calif.) and RNALATER™ (Ambion, Austin, Tex.).

### True Low Birth Weight Associated Genes

As used herein, a “true low birth weight related gene,” “true low birth weight associated gene,” “TLBW-related gene,” or “TLBW-associated gene” refer to a gene which is expressed at a level, in a TLBW infant, which is different from (increased or decreased) its expression level in a normal, healthy infant. TLBW-related genes may be useful for the diagnosis, treatment, or prevention of true low birth weight, and may serve as a guide for treatment of the TLBW subject. Genes identified to be TLBW-related may be used as diagnostic targets for the early detection and diagnosis of TLBW, and may be used to differentiate between a subject with true low birth weight due to exposure risk factors such as malnutrition, or a subject which is simply small due to other factors, such as small parents. Genes identified to be TLBW-related may be used to identify exposure to various risk factors, including but not limited to, alcohol abuse, drug abuse, and malnutrition. For example, the methods of the present invention may provide information about nutritional and metabolic deficits and surfeits during gestation, including but not limited to, calories, oxygen, vitamins, minerals, and the appropriate combinations of protein, carbohydrates, and fats. TLBW-related genes may be targets for genetic therapy or for agents which modulate their expression, for the treatment or prevention of TLBW.

Differential expression of genes is defined herein as either an increase or decrease in gene expression, depending on the gene, as compared to expression in a healthy control subject. Gene expression is considered increased when the increase in gene expression is characterized by a p-value which is less than or equal to 0.1, less than or equal to 0.05, less than or equal to 0.01, or less than or equal to 0.001, relative to a comparable sample of a healthy control subject. In non-limiting embodiments, gene expression may be considered increased when the gene expression is increased by at least 5 percent, at least 20 percent, at least 50 percent, or at least 90 percent, as compared to the gene expression in a comparable sample of a healthy control subject. Gene expression is considered decreased when the decrease in gene expression is characterized by a p-value which is less than or equal to 0.1, less than or equal to 0.05, less than or equal to 0.01, or less than or equal to 0.001, relative to a comparable sample of a healthy control subject. In non-limiting embodiments, the decrease in gene expression may be characterized by a decrease in gene expression of at least 5 percent, at least 20 percent, at least 50 percent, or at least 90 percent, as compared to the gene expression in a comparable sample of a healthy control subject.

TLBW-related genes which may be measured according to the methods of the present invention may be selected from the genes represented by the probe sets listed in Table 1, and the human homologs thereof, where the probe sets are representative of data generated using an Affymetrix Rat Genome Chip™ (230.2) (Affymetrix, Santa Clara, Calif.). The genes represented by the probe sets listed may be deduced via the NETAFFX™ Analysis Center software (Affymetrix, Santa Clara, Calif.) (available at www.affymetrix.com/analysis/index.affx under the terms and conditions set forth therein). Table 1 includes data derived from rat experiments involving caloric restriction (CR) of pregnant rats. Table 1 lists probe sets which have been identified as having increased expression in newborn rats diagnosed with TLBW (designated by “up”) due to CR, as well as probe sets which have decreased expression in newborn rats diagnosed with TLBW (designated “down”) due to CR. Accordingly, measurement of the genes represented by the probe sets listed in Table 1 will be useful for the diagnosis of TLBW. Sequence homology between the rat genes and their human equivalents will allow extrapolation of the data of Table 1 for use in humans and, by analogy, other species.

The present invention also encompasses oligonucleotide sequences which are complementary to TLBW-related genes. The oligonucleotide sequences may be complementary to the genes represented by the probe sets identified in Table 1. The oligonucleotides may be utilized as primers for amplification of the TLBW-related genes, for example, by polymerase chain reaction (PCR). The oligonucleotides may also be utilized as probes for the detection of TLBW-related genes or the measurement of TLBW-related gene expression. Preparation of primers or probes using well-known methods based upon the identified TLBW-related genes will be readily apparent to those of ordinary skill in the art.

Differential expression of particular TLBW-related genes may be associated with prenatal exposure to particular risk factors. Deficits or surfeits of metabolic and nutritional factors are risk factors for TLBW and may cause differential expression of TLBW related genes. Metabolic and nutritional factors include, but are not limited to, calories, oxygen, vitamins, minerals, and the appropriate combinations of protein, carbohydrates, and fats. Different risk factors may therefore result in different changes in gene expression for a given TLBW related gene. Genes which display differential expression which may be measured according to the methods of the present invention may be selected from the genes represented in Tables 1, 2, 6, 7, 10, 11, 12, and/or 13.

Table 2, based on rat data, sets forth rat genes which show that exposure to caloric restriction during gestation may cause an increase in a particular TLBW related gene, whereas IUGR may cause a decrease in the same TLBW related gene. Table 6 sets forth probe sets identifiers for rat genes (based on an Affymetrix Rat Genome Chip™ (230.2) (see supra)) which display differential expression due to exposure to a high fat diet during gestation. Table 7 sets forth probe sets identifiers for rat genes (based on an Affymetrix Rat Genome Chip™ (230.2) (see supra)) which demonstrate a rank ordered change in expression due to various levels of caloric restriction. Table 10 sets forth probe sets identifiers for rat genes (based on an Affymetrix Rat Genome Chip™ (230.2) (see supra)), which display differential expression due to exposure to selective serotonin reuptake inhibitors (SSRIs). The differences in gene expression arising from different exposures during gestation, such as the differences in gene expression set forth in the foregoing tables, may be utilized to distinguish between exposure to different risk factors. The human homologs of the TLBW-related rat genes represented by the foregoing tables are encompassed by the invention. Accordingly, the multiple TLBW related genes may be selected and simultaneously screened to assist in the differentiation of potential exposure to various risk factors during gestation.

Caloric restriction during gestation may cause differential expression of a particular set of genes, whereas IUGR caused by other risk factors may cause differential expression of different genes. Table 2 identifies genes with differential levels of expression in instances of caloric restriction (CR) and instances of IUGR. This data indicates that differential expression of certain genes may vary based upon caloric restriction, but do not necessarily correlate with differential gene expression due to IUGR. As shown in Table 2, various genes are up regulated in both IUGR and CR. These genes include but are not limited to: proviral integration site 1; homocysteine-inducible, endoplasmic reticulum stress-inducible, ubiquitin-like domain member 1; Protein C receptor, endothelial (predicted); Solute carrier family 2 (facilitated glucose transporter), member 2; glycoprotein hormones, alpha subunit; similar to hypothetical protein FLJ13511 (predicted); phospholipase C-like 2 (predicted); similar to Hypothetical WD-repeat protein CGI-48 (predicted); peroxiredoxin 6; Sorting nexin 10 (predicted); leptin; Similar to IER7; Small fragment nuclease (predicted); Tissue factor pathway inhibitor; Similar to IER6; syndecan 1; Jun D proto-oncogene; huntingtin interacting protein 2 (predicted); Ferredoxin 1; solute carrier family 11 (proton-coupled divalent metal ion transporters), member 2; neuron specific gene family member 1; and Hydroxysteroid dehydrogenase-1, delta<5>-3-beta (predicted).

Table 2 also shows that various genes are down regulated in both IUGR and CR. These genes include but are not limited to: Catalase; colony stimulating factor 1 (macrophage); Zinc finger protein 262 (predicted); immunoglobulin (CD79A) binding protein 1; Hepatocyte growth factor; bone morphogenetic protein 5 (predicted); adaptor-related protein complex 3, mu 2 subunit; Similar to hypothetical protein FLJ22344 (predicted); Glycine amidinotransferase (L-arginine:glycine amidinotransferase); similar to map kinase interacting kinase; SMC6 structural maintenance of chromosomes 6-like 1 (yeast) (predicted); aquarius (predicted); sprouty homolog 2 (Drosophila) (predicted); cytoplasmic FMR1 interacting protein 1 (predicted); similar to TBC1 domain family member 4; folate receptor 2 (fetal) (predicted); mitogen-activated protein kinase kinase kinase kinase 4 (predicted); Adducin 3 (gamma); FERM domain containing 4B; dystonin (predicted); O-linked N-acetylglucosamine (GlcNAc) transferase (UDP-N-acetylglucosamine:polypeptide-N-acetylglucosaminyl transferase); platelet derived growth factor receptor, beta polypeptide; lysosomal-associated protein transmembrane 4B (predicted); myosin X (predicted); laminin, alpha 2 (predicted); low density lipoprotein receptor-related protein 6 (predicted); ubiquitin conjugation factor E4 A; transforming growth factor beta 1 induced transcript 1; RT1 class I, CE16; RT1 class Ia, locus A2; procollagen, type XV (predicted); protocadherin gamma subfamily C, 3; CD4 antigen; stabilin 1 (predicted); guanylate cyclase 1, soluble, beta 3; Ras homolog gene family, member E; procollagen C-proteinase enhancer protein; peripheral myelin protein 22; fibromodulin; wingless-related MMTV integration site 2; CD44 antigen; intercellular adhesion molecule 2; myeloid cell nuclear differentiation antigen (predicted); complement component 1, r subcomponent (predicted); dihydropyrimidinase-like 3; RT1 class I, CE15; RT1 class Ib, locus Aw2; RT1-149 protein; amine oxidase, copper containing 3; procollagen, type VI, alpha 3 (predicted); Cytochrome b-245, beta polypeptide; Neuropilin 1; caspase 1; Down syndrome critical region homolog 1 (human); Similar to E430002G05Rik protein (predicted); Collagen, type V, alpha 2; Nuclear receptor subfamily 3, group C, member 1; similar to hypothetical protein FLJ10652 (predicted); guanylate cyclase 1, soluble, alpha 3; potassium voltage-gated channel, delayed-rectifier, subfamily S, member 3; procollagen, type I, alpha 3; insulin-like growth factor 1; allograft inflammatory factor 1; allograft inflammatory factor 2; procollagen, type I, alpha 2; and plasma glutamate carboxypeptidase.

Table 2 also shows that various genes are down regulated in IUGR but are up regulated in CR. These genes include but are not limited to: CUG triplet repeat, RNA-binding protein 2; aldehyde dehydrogenase family 3, subfamily A2; core-binding factor, runt domain, alpha subunit 2; translocated to, 1; cyclin D-related (predicted); collagen, type V, alpha 3; Nuclear receptor subfamily 2, group F, member 2; ATPase, Ca++ transporting, cardiac muscle, slow twitch 2; similar to dJ202D23.2 (novel protein similar to C21ORF5 (KIAA0933)) (predicted); Transducin-like enhancer of split 4, E(spl) homolog (Drosophila); Potassium channel, subfamily K, member 3; apurinic/apyrimidinic endonuclease 1; gap junction membrane channel protein alpha 1; ATPase, Ca++transporting, plasma membrane 4; Transducin-like enhancer of split 4, E(spl) homolog (Drosophila); Solute carrier family 5 (inositol transporters), member 3; Klotho; tripartite motif protein 27 (predicted); myosin Ib; chromosome condensation 1-like; thymus cell antigen 1, theta; CTD (carboxy-terminal domain, RNA polymerase II, polypeptide A) small phosphatase-like (predicted); cAMP responsive element binding protein 1; Ectonucleotide pyrophosphatase/phosphodiesterase 2; LRRC36 homolog (human); Growth arrest specific 6; CDC16 cell division cycle 16 homolog (S. cerevisiae) (predicted); and matrix metallopeptidase 2.

Table 2 also shows that various genes are up regulated in IUGR but are down regulated in CR. These genes include but are not limited to: Similar to RIKEN cDNA 1500006O09 (predicted); growth differentiation factor 15; Basic leucine zipper and W2 domains 4; colony stimulating factor 2 receptor, beta 1, low-affinity (granulocyte-macrophage); eukaryotic translation termination factor 1 (predicted); similar to RIKEN cDNA 1110012L19; Similar to RIKEN cDNA 3930401K13 (predicted); ubiquitin-conjugating enzyme E2 variant 2; Tankyrase, TRF1-interacting ankyrin-related ADP-ribose polymerase 2 (predicted); RAS guanyl releasing protein 1; ornithine decarboxylase antizyme inhibitor; Thymine-DNA glycosylase; microfibrillar-associated protein 3-like (predicted); actin related protein 2/3 complex, subunit 5-like (predicted); polymerase (RNA) II (DNA directed) polypeptide H (predicted); ectonucleoside triphosphate diphosphohydrolase 1; microtubule-associated protein 7 (predicted); Tankyrase, TRF1-interacting ankyrin-related ADP-ribose polymerase 2 (predicted); Similar to methyl-CpG binding protein MBD2; cytochrome P450, family 19, subfamily a, polypeptide 1; hexosaminidase B (predicted); Similar to testis specific protein, Ddc8; Serine/threonine kinase 3; inhibin beta-A; microfibrillar associated protein 5 (predicted); GULP, engulfment adaptor PTB domain containing 1 (predicted); solute carrier family 11 (proton-coupled divalent metal ion transporters), member 3; calpain 6; and Transferrin receptor.

Accordingly, identification of gene expression in various genes may allow for the diagnosis of CR and IUGR, and may further allow for the differentiation of CR from IUGR. In a non-limiting example, increased expression of certain genes, such as myosin 1b, or decreased expression of certain genes, such as growth differentiation factor 15, may be indicative of TLBW due to caloric restriction (CR), but not due to IUGR. See Table 2; see also McMinn et al., supra. Gene expression of certain genes may allow for the diagnosis of CR or IUGR, but may not be useful to differentiate between a diagnosis of CR or IUGR. In a non-limiting example, increases in some genes may be indicative of both IUGR and CR, such as an increase in expression of peroxiredoxin 6. Similarly, decreases in some genes may be indicative of both IUGR and CR, such as an decrease in expression of catalase. To differentiate between IUGR and CR, the gene expression of genes which are up regulated in IUGR but are down regulated in CR may be measured. Similarly, the gene expression of genes which are down regulated in IUGR but are up regulated in CR may be measured. In a non-limiting example, a change in expression of myosin 1b may be used to distinguish between IUGR and CR. Up regulation of myosin 1b is indicative of CR, whereas down regulation is indicative of IUGR. This data therefore allows a person of ordinary skill in the art to differentiate between true low birth weight due to CR and IUGR, and in some cases, may allow identification of the cause of TLBW, such as CR. This further allows selection of an appropriate treatment, for example, increased caloric intake, which is discussed in more detail below. It is understood that the human homologs of the TLBW genes represented in Table 2 are encompassed by the present invention.

It will be apparent to a person of ordinary skill in the art that gene expression data correlated to other risk factors may be utilized to determine exposure to one or more risk factors. For example, Tables 5 and 6 identify probe sets for genes with differential gene expression due to exposure to a high fat diet during gestation. Tables 8 and 10 identify probe sets for genes with differential expression due to exposure to an SSRI during gestation. Measurement of gene expression of the genes represented in Tables 5, 6, 8, and 10 may allow a person of ordinary skill in the art to determine whether a subject has been exposed to a high fat diet or an SSRI during gestation, based upon the appropriate increase or decrease in gene expression in a gene associated with exposure to a high fat diet or an SSRI. This further allows selection of an appropriate treatment based upon the exposure identified, as discussed in more detail below. It is understood that the human homologs of the TLBW genes listed in Table 5, 6, 8, and 10 are encompassed by the present invention.

### Fetal Alcohol Syndrome Related Genes

As used herein, a “fetal alcohol syndrome related gene,” “FAS related gene,” “fetal alcohol syndrome associated gene,” and “FAS associated gene” is a TLBW related gene which is expressed at a level, in an infant with fetal alcohol syndrome, which is different from (increased or decreased) its expression level in a normal, healthy infant. FAS related genes may also refer to a TLBW related gene which is expressed at a level, in an infant with fetal alcohol syndrome, which is different from (increased or decreased) its expression level in an infant with TLBW caused by exposure to risk factors other than alcohol. FAS-related genes may be useful for the diagnosis, treatment, or prevention of fetal alcohol syndrome, and may serve as a guide for treatment of the FAS subject. Genes identified to be FAS-related may be used as diagnostic targets for the early detection and diagnosis of FAS, and may be used to differentiate between a subject with fetal alcohol syndrome or a subject with true low birth rate due to other risk factors, such as malnutrition, or a subject which is simply small due to other factors, such as small parents. For example, the methods of the present invention may provide information regarding exposure to alcohol during gestation, including but not limited to, the amount of alcohol exposure and the time of alcohol exposure. FAS-related genes may be targets for genetic therapy or for agents which modulate their expression, for the treatment or prevention of FAS.

FAS-related genes which may be measured according to the methods of the present invention may be selected from the genes represented by the probe sets listed in Tables 1, 2, 6, 7, 10, 11, 12, and/or 13, preferably selected from Tables 11, 12, and/or 13, and the human homologs thereof, where the probe sets are representative of data generated using an Affymetrix Rat Genome Chip™ (230.2) (Affymetrix, Santa Clara, Calif.). The genes represented by the probe sets listed may be deduced via the NETAFFX™ Analysis Center software (Affymetrix, Santa Clara, Calif.) (available at www.affymetrix.com/analysis/index.affx under the terms and conditions set forth therein). Table 11 includes data derived from rat experiments involving pregnant rats exposed to alcohol during gestation, and indicates genes identified as down-regulated due to exposure to alcohol. The genes in the shaded rows indicate FAS related genes which do not show significant down-regulation due to exposure during gestation to an SSRI, caloric restriction, or exposure to a high fat diet. Table 12 includes data derived from rat experiments involving pregnant rats exposed to alcohol during gestation, and indicates genes identified as up-regulated due to exposure to alcohol. The genes in the shaded rows indicate FAS related genes which do not show significant up-regulation due to exposure during gestation to an SSRI, caloric restriction, or exposure to a high fat diet. Accordingly, measurement of the genes represented by the probe sets listed in Tables 11 and 12 will be useful for the diagnosis of FAS, and to distinguish FAS related genes from other TLBW related genes which may show differential expression due to exposure to risk factors other than alcohol. Sequence homology between the rat genes and their human equivalents will allow extrapolation of the data of Tables 11, 12, and 13 for use in humans and, by analogy, other species.

The present invention also encompasses oligonucleotide sequences which are complementary to FAS-related genes. The oligonucleotide sequences may be complementary to the genes represented by the probe sets identified in Tables 1, 2, 6, 7, 10, 11, 12, and/or 13, and are preferably complementary to the genes represented by the probe sets identified in Tables 11, 12, and/or 13. The oligonucleotides may be utilized as primers for amplification of the FAS-related genes, for example, by polymerase chain reaction (PCR). The oligonucleotides may also be utilized as probes for the detection of FAS-related genes or the measurement of FAS-related gene expression. Preparation of primers or probes using well-known methods based upon the identified FAS-related genes will be readily apparent to those of ordinary skill in the art.

Differential expression of particular FAS-related genes may be associated with prenatal exposure to alcohol. As discussed above, different risk factors may result in different changes in gene expression for a given TLBW related gene. Accordingly, identification of differential expression in an FAS related gene but not in an TLBW related gene provides a useful method of differentiating prenatal exposure to alcohol from prenatal exposure to other risk factors. Similarly, identification of differential expression of an FAS gene in addition to the identification of differential expression of a different TLBW related gene provides information regarding specific prenatal exposure to alcohol, in addition to potential exposures to other risk factors. Genes which display differential expression which may be measured according to the methods of the present invention may be selected from the genes represented in Tables 1, 2, 6, 7, 10, 11, 12, and/or 13.

As discussed in more detail above, Tables 1, 2, 6, 7, 10, 11, 12, and 13 set forth probe sets identifiers for rat genes (based on an Affymetrix Rat Genome Chip™ (230.2) (see supra)), which display differential expression due to exposure to caloric restriction, exposure to a high fat diet, exposure to selective serotonin reuptake inhibitors (SSRIs), and exposure to alcohol. The differences in gene expression arising from different exposures during gestation, such as the differences in gene expression set forth in the foregoing tables, may be utilized to distinguish between exposure to different risk factors. It will be apparent to a person of ordinary skill in the art that gene expression data correlated to other risk factors may be utilized to determine exposure to one or more risk factors. The human homologs of the TLBW-related or FAS related rat genes represented by the foregoing tables are encompassed by the invention. Accordingly, the multiple TLBW related and/or FAS related genes may be selected and simultaneously screened to assist in the differentiation of potential exposure to various risk factors during gestation.

### Measurement of TLBW Related Gene Expression

The following description of the measurement of TLBW related gene expression is intended as an example, and is non-limiting. The described methods of measuring TLBW related genes may be utilized to measure TLBW related gene expression due to a variety of exposures, including but not limited exposure to alcohol. A tissue sample is obtained from the subject, preferably immediately following birth, within one hour following birth, within 24 hours following birth, or within 48 hours following birth. The present invention further encompasses a tissue sample obtained in utero, for example, a placental biopsy performed pre-delivery. Preferably, the tissue sample is placental tissue (which has been maintained in a condition to protect the integrity of the placental RNA), which is excised from the portion of the placenta from which the umbilical cord protrudes. RNA is extracted from the tissue sample immediately where possible. If RNA cannot be extracted until a later time, the tissue sample is placed in a suitable stabilizer or preservative. An example of a suitable stabilizer is RNALATER™. RNA is preferably extracted by the guanidine thiocyanate method. An example of a reagent which may be utilized in the guanidine thiocyanate method is TRIZOL™.

RNA which has been purified utilizing the methods described above may be amplified using any nucleic acid amplification assay utilized for detection of low numbers of RNA molecules. RNA may be amplified utilizing primers directed to the TLBW related genes, which allows for measurement of the relative expression of the TLBW related gene. Preferably, methods of RNA amplification which preserve the relative composition of the RNA are utilized. In a preferred embodiment, nucleic acids may be detected by utilizing quantitative polymerase chain reaction (PCR). Quantitative PCR allows for the accurate measurement of the relative amount of RNA transcripts present in a sample, which correlates with the relative level of gene expression. For example, quantitative PCR utilizing primers directed to IGF1 may be used. Amplification and detection of the expression of the IGF1 gene may be performed in both a test sample (a sample derived from the subject being tested for TLBW) and a control sample (a sample derived from a subject of known gene expression, such as a healthy, non-TLBW infant). A standardized, internal control sample which has constant gene expression may also be used as a reference for normalization of gene expression. Suitable standardized, internal controls will be known to those of ordinary skill in the art, and include, but are not limited to, 18S mRNA, GAPDH, and actin. Gene expression may be expressed as a base-10 logarithmic increase relative to the standardized, internal control sample. For example, a value of log(1) indicates a 10-fold greater expression than the internal control sample. Any methods known in the art for amplifying RNA may be utilized, and include, but are not limited to: reverse transcriptase polymerase chain reaction, ligase chain reaction, branched DNA signal amplification, amplifiable RNA reporters, Q-beta replication, transcription-based amplification, boomerang DNA amplification, strand displacement activation, cycling probe technology, isothermal nucleic acid sequence based amplification, and other self-sustained sequence replication assays. See Sambrook, supra.

In another embodiment, nucleic acids may be detected by hybridization with a complementary sequence, such as an oligonucleotide probe. See U.S. Pat. No. 5,503,980 (Cantor), U.S. Pat. No. 5,202,231 (Drmanac et al.), U.S. Pat. No. 5,149,625 (Church et al.), U.S. Pat. No. 5,112,736 (Caldwell et al.), U.S. Pat. No. 5,068,176 (Vijg et al.), and U.S. Pat. No. 5,002,867 (Macevicz). Methods of detecting gene expression via hybridization with oligonucleotide probes include northern blots, phosphorimaging, southern blots, and dot blots. See Sambrook, supra. In a non-limiting example, detection may be performed utilizing an array of oligonucleotide probes assembled on a chip, referred to as a DNA chip or a DNA microarray, may be used to detect nucleic acids by hybridization. See U.S. Pat. Nos. 5,837,832 and 5,861,242 (Chee et al.). An example of a DNA chip is the GENECHIP™, available from Affymetrix (Santa Clara, Calif.). The DNA chip may contain oligonucleotide probes which are homologous to known genetic sequences, and are used to identify specific genes. Nucleic acids isolated from the tissue and fluid samples will hybridize to complementary sequences on the DNA chip, and the resulting DNA chip may be analyzed to determine which oligonucleotide probes have been hybridized. Analysis of the DNA chip may be performed by biotinylating the nucleic acids isolated from the tissue and fluid samples; once the nucleic acids are hybridized to the DNA chip, streptavidin coupled to a fluorescent dye may be added. Alternatively, streptavidin may be added, followed by staining with an anti-streptavidin antibody. The anti-streptavidin antibody may be conjugated to a fluorescent dye, or may be bound by an additional antibody which is conjugated to a fluorescent dye. The resulting fluorescence-stained DNA chip may be scanned with a confocal laser, which causes the fluorescent dye to fluoresce. The resulting fluorescence pattern may be used to determine which oligonucleotide probes have been hybridized. In a preferred embodiment, GENECHIPs™ may be used to determine expression.

Labeled probes may be utilized to detect a nucleic acid. If a labeled probe hybridizes to the isolated nucleic acid, the label is preferably one that can be detected in a homogeneous system (i.e., one that does not require unbound probe to be separated from the isolated nucleic acid hybridized to probe for detection of bound probes). Alternatively, isolated nucleic acids or fragments thereof may be hybridized to an array of probes as on a DNA chip and those probes that specifically hybridize to the isolated nucleic acids are detected to provide sequence information about the isolated nucleic acids. Those skilled in the art will appreciate that more than one procedure may be used to detect the isolated nucleic acids.

### Kits for Diagnosing True Low Birth Weight

The present invention further provides kits for diagnosing true low birth weight in a subject. The methods, PCR primers, and nucleotide sequences described herein may be efficiently utilized in the assembly of a diagnostic kit, which may be used to diagnose TLBW in a subject. The kit is useful in distinguishing between newborn infants suffering from TLBW due to the presence of the risk factors identified above and normal, healthy newborn infants which are simply small. Such a diagnostic kit contains the components necessary to practice the methods as described above.

Thus, the kit may contain a sufficient amount of at least one probe complementary to an TLBW-related gene. The kit may also contain a sufficient amount of at least one PCR primer pair for an TLBW-related gene, for the amplification of the TLBW-related gene or detection of the TLBW-related gene. In a preferred embodiment, the primer pair is used for the detection of the TLBW-related gene utilizing RT-PCR. The kit may optionally comprise reagents and instruments necessary for the collection of samples. The kit may optionally comprise components of a detectable labeling system, vials for containing the tissue or fluid samples, substrates for the preservation of tissue or fluid samples, control tissue or fluid samples (e.g., dried or frozen tissue or fluid from a healthy fetus, newborn infant, or mother), protein samples, and the like. Control reagents may comprise healthy tissue samples, or tissue or fluid samples which have known expression levels for particular genes. Control samples may be included for one or more birth weight quintiles. A reference standard, comprising nucleic acid at a concentration that would be found in a control sample, may also be provided. An internal control sample, for example, actin, may also be included. The control reagents may be fresh, frozen, or otherwise preserved. The kit may also include a means for extracting the tissue or fluid samples. The kit may also provide reagents and materials for preserving the tissue or fluid samples. Other conventional components of such diagnostic kits may also be included. In a preferred embodiment, the oligonucleotide probes comprise sequences complementary to portions of genes encoding an insulin-like growth factor (“IGF,” e.g., IGF1, IGF2), or a gene related to IGF, including but not limited to, genes encoding IGF binding proteins (e.g., ALS, CTGF/CCN2, Endocan, IGFBP-1, IGFBP-2, IGFBP-3, IGFBP-4, IGFBP-5, IGFBP-6, IGFBP-7, Nov/CCN3), and genes encoding IGF receptors. The kits may also comprise oligonucleotide probes comprising sequences complementary to portions of the genes represented in Tables 1, 2, 6, 7, 10, 11, 12, and 13. In specific non-limiting embodiments, the oligonucleotides may be a primer pair for use in PCR. In a preferred embodiment, the kit contains oligonucleotide probes directed to more than one TLBW-related genes. Oligonucleotide probes representing TLBW-related genes may be mounted on a substrate, such as a gene chip.

The kit may contain a sufficient amount of at least one probe complementary to an FAS-related gene. The kit may also contain a sufficient amount of at least one PCR primer pair for an FAS-related gene, for the amplification of the FAS-related gene or detection of the FAS-related gene. In a preferred embodiment, the primer pair is used for the detection of the FAS-related gene utilizing RT-PCR. The kit may optionally comprise reagents and instruments necessary for the collection of samples. The kit may optionally comprise components of a detectable labeling system, vials for containing the tissue or fluid samples, substrates for the preservation of tissue or fluid samples, control tissue or fluid samples (e.g., dried or frozen tissue or fluid from a healthy fetus, newborn infant, or mother), protein samples, and the like. Control reagents may comprise healthy tissue samples, or tissue or fluid samples which have known expression levels for particular genes. Control samples may be included for one or more birth weight quintiles. A reference standard, comprising nucleic acid at a concentration that would be found in a control sample, may also be provided. An internal control sample, for example, actin, may also be included. The control reagents may be fresh, frozen, or otherwise preserved. The kit may also include a means for extracting the tissue or fluid samples. The kit may also provide reagents and materials for preserving the tissue or fluid samples. Other conventional components of such diagnostic kits may also be included. In a preferred embodiment, the oligonucleotide probes comprise sequences complementary to portions of the following genes: Gusb, Pex6, Arts1, Centa2, Hadhsc, Vps35 (mapped), Tpar1, RGD1305243 (predicted), Cdca7, RGD1565289 (predicted), Crsp9 (predicted), Ggps1, Cul2 (predicted), Trp53rk (predicted), Prdm2 (mapped), Ddx59, Mcpt6, Ndufb5 (predicted), Mcts1, Enpp5, MGC72992, Sumf1 (predicted), Plek, Plag1, Rnd3, Inpp1, Zfp207, Akr7a2, Cp, St3gal3, LOC287533, Slc39a4 (predicted), Polr3e (predicted), Pcmtd2 (predicted), Kbtbd4 (predicted), Asah1, RGD1564011 (predicted), Mss4, RT1-Aw2, LOC303515, Wig1, Phyh, RGD1308959, Csnk1d, Zfp365, and Lama5. The kits may also comprise oligonucleotide probes comprising sequences complementary to portions of the genes represented in Tables 1, 2, 6, 7, 10, 11, 12, and 13. In specific non-limiting embodiments, the oligonucleotides may be a primer pair for use in PCR. In a preferred embodiment, the kit contains oligonucleotide probes directed to more than one FAS-related genes. Oligonucleotide probes representing FAS-related genes may be mounted on a substrate, such as a gene chip.

The diagnostic kits may also include instructions for using the included components. The kit may also include computer software to aid in the measurement of gene expression or the calculation of expression ratios. The kit may include or provide access to a database comprising characteristic gene expression information, allowing for the levels of measured gene expression to be compared to a set of standardized data. The kit may include information regarding characteristic gene expression ratios, for example, in the form of charts.

The kits may include a means for extracting tissue or fluid samples. Preferably, the means will be capable of extracting a cross-section of the tissue which captures all cell layers in the target sample. The kits may optionally comprise reagents for preserving RNA or DNA in a tissue or fluid sample. The kits may additionally comprise reagents and equipment for purifying nucleic acids from tissue or fluid samples, which may include any reagents or equipment known to persons of ordinary skill in the art for purification of nucleic acids. Reagents and equipment for measuring gene expression may include any reagents or equipment known to persons of ordinary skill in the art for detecting gene expression.

### Intervention and Treatment of True Low Birth Weight

The present invention further relates to methods for treating infants diagnosed with true low birth weight utilizing the methods of the present invention. As noted above, differential expression of particular genes may be associated with exposure to particular risk factors. The present invention thus provides for a method of identifying the exposures of the infant subject, and further provides for identification of appropriate treatments to offset the deleterious effects of the exposures. As used herein, the term “exposures” refers to exposure of fetuses to the risk factors detailed above, and may refer to over-exposure or under-exposure to the risk factors. For example, exposures may refer to deficits and surfeits during gestation of calories, oxygen, vitamins, minerals, and the appropriate combinations of protein, carbohydrates, and fats.

Exposure to risk factors may be correlated to gene expression by measuring the changes in gene expression in genes that are associated with the risk factors. Determination of which genes are associated with a particular risk factor may be performed by measuring the gene expression in subjects which have been exposed to the risk factor, and comparing the measured gene expression to the gene expression of the same gene in a healthy subject. Where gene expression of a particular gene in the subject exposed to a risk factor exhibits a statistically significant change relative to gene expression in the healthy subject, then changes in expression of the gene may be correlated to the risk factor. Examples of genes correlated to various risk factors are provided below. Measurement of gene expression may be conducted in laboratory animals, such as mice or rats, which have been exposed to a risk factor. It will be known to a person of ordinary skill in the art that sequence homology between the rat genes and the human equivalents will allow extrapolation of this data for use in other species, such as humans. The measurement of gene expression may also be made in human subjects at the time of birth, and exposure to risk factors during gestation may be identified via examination of the mother's relevant medical records.

In non-limiting embodiments, gene expression may be measured in subjects which have been exposed to caloric restriction, a high fat diet, or SSRIs. In a non-limiting example, Table 1 provides a list of genes which exhibit changes in expression where the fetus was exposed to caloric restriction (CR). Table 2 also provides a list of genes which exhibit changes in expression where the fetus was exposed to CR during gestation, and which also suffers from IUGR. Accordingly, measurement of changes in gene expression of the genes represented in Table 1 or Table 2 may allow a person of ordinary skill in the art to determine if a fetus has been exposed to CR during gestation and/or suffers from IUGR. Distinguishing between CR and IUGR is discussed in greater detail above.

In another non-limiting example, Table 6 provides a list of probe sets representative of rat genes which exhibit changes in expression when the fetus is exposed to a high fat diet during gestation. Accordingly, measurement of changes in gene expression of the genes represented in Table 6 and their human homologs may allow a person of ordinary skill in the art to determine if a fetus has been exposed to a high fat diet during gestation. The genes represented by Table 6 indicate a decrease in gene expression where the gene expression in subjects with a high fat diet (designated by “HF”) is decreased relative to the gene expression of the control subjects (designated “Cont”). The genes represented by Table 6 indicate an increase in gene expression where the gene expression in subjects with a high fat diet (designated by “HF”) is increased relative to the gene expression of the control subjects (designated “Cont”). Based upon this information, a person of ordinary skill in the art will be capable of identifying exposure to a high fat diet based upon an increase or decrease in expression of one or more genes represented by Table 6.

In another non-limiting example Table 10 provides a list of probe sets representing genes which exhibit changes in expression when the fetus is exposed to a selective serotonin reuptake inhibitor (SSRI) during gestation. Accordingly, measurement of changes in gene expression of the genes represented in Table 10 may allow a person of ordinary skill in the art to determine if a fetus has been exposed to SSRIs during gestation. The genes represented by Table 10 indicate a decrease in gene expression where the gene expression in subjects exposed to an SSRI (designated by “SSRI”) is decreased relative to the gene expression of the control subjects (designated “C”). The genes represented by Table 10 indicate an increase in gene expression where the gene expression in subjects exposed to an SSRI (designated by “SSRI”) is increased relative to the gene expression of the control subjects (designated “C”). Based upon this information, a person of ordinary skill in the art will be capable of identifying exposure to an SSRI based upon an increase or decrease in expression of one or more genes represented by Table 10.

In another non-limiting example Tables 11, 12, and 13 provide lists of probe sets representing genes which exhibit changes in expression when the fetus is exposed to alcohol during gestation. Accordingly, measurement of changes in gene expression of the genes represented in Tables 11, 12, and/or 13 may allow a person of ordinary skill in the art to determine if a fetus has been exposed to alcohol during gestation. The genes represented by Tables 11 indicate a decrease in gene expression where the gene expression in subjects exposed to alcohol is decreased relative to the gene expression of control subjects. The genes represented by Table 12 indicate an increase in gene expression where the gene expression in subjects exposed to alcohol is increased relative to the gene expression of control. Based upon this information, a person of ordinary skill in the art will be capable of identifying exposure to alcohol based upon an increase or decrease in expression of one or more genes represented by Tables 11, 12, and/or 13.

It is envisioned that methods of treatment of infants diagnosed with true low birth weight or which have been diagnosed to have been exposed to one or more risk factors will be tailored to the infant depending upon the TLBW-related gene identified. The treatment is optimally selected to offset the exposures experienced by the newborn infant. As used herein, the term “offset” means counteracting the exposures. It will be within the ability of those skilled in the art to identify and select an appropriate treatment, such as a modified diet or administration of a dietary supplement, which will offset exposure to the risk factor. In a non-limiting example, expression of an TLBW-related gene associated with a deficit or surfeit of one or more nutrients, including but not limited to vitamins, minerals, protein, carbohydrates, and fats, may indicate the necessity to administer a nutrient-enriched formula to offset the exposure. For example, poor protein consumption during gestation may indicate the necessity for administration of a protein-enriched formula, or a change in expression of an TLBW-related gene associated with carbohydrate overfeeding may also indicate the necessity for administration of a carbohydrate-reduced formula. Selection and administration of the appropriate treatment to offset exposure to a risk factor will be apparent to those of ordinary skill in the art. Such treatment may include administration of diets with increased or reduced nutrient intake, based upon the exposure identified. The treatment may also include administration of diets with appropriately balanced level of nutrients, such as vitamins, minerals, proteins, carbohydrates, and fats. The treatment may also include administration of agents drugs or other supplements, which may assist in the uptake of nutrients or may counteract the effects of the exposures.

In a non-limiting example, an identification of differential expression of an TLBW-related gene associated with caloric restriction may indicate the necessity for administration of an enriched formula and/or an increase in caloric intake. Examples of genes which may be measured to determine if the subject has been exposed to caloric restriction can be found in the genes represented by the probe sets listed in Table 1 and the genes listed in Table 2, and their human homologs. Furthermore, as discussed in more detail above, the list of genes represented in Table 2 may be used to determine whether a subject has been exposed to caloric restriction and/or which suffers from IUGR. Where a TLBW gene represented in Tables 1 or 2 (including human homologs) has been identified as appropriately increased or decreased in a subject, the method of treatment will be apparent to a person of ordinary skill in the art. For example, the subject may be administered an diet with increased caloric intake, or the subject may be administered dietary supplements.

In another non-limiting example, an identification of differential expression of an TLBW-related gene associated with exposure to a high fat diet during gestation may indicate the necessity for administration of a nutrient-enriched formula and/or reduced fat diet. Examples of genes which may be measured to determine if the subject has been exposed to a high fat diet during gestation can be selected from the genes represented by probe sets listed in Table 6. Where a TLBW gene from Table 6 has been identified as appropriately increased or decreased in a subject, the method of treatment will be apparent to a person of ordinary skill in the art. For example, the subject may be administered a diet with reduced fat, or the subject may be administered a diet containing the appropriately balanced level of nutrients.

The methods described herein may be used to identify exposure to non-dietary risk factors such as tobacco, alcohol, and drug abuse. For example, Table 10 provides a list of probe sets representing genes which exhibit a change in expression where the subject is exposed to a selective serotonin reuptake inhibitor (SSRI). Where a TLBW gene represented by Table 10 has been identified as appropriately increased or decreased in a subject, the method of treatment will be apparent to a person of ordinary skill in the art. For example, the subject may be administered an enriched-diet, or administered agents which may counteract the effects of the SSRIs.

### Statistical Analysis

Genetic screening data obtained utilizing the methods described above may be analyzed via well known statistical methods. Any method of statistical analysis which is well known in the art may be utilized in the present invention. Computer software may be utilized to perform the statistical analysis. An example of computer software which may be used in the present invention is SYSTAT™ (Systat Software, Inc., Point Richmond, Calif.).

As a non-limiting example, ANOVA (analysis of variations) may be utilized to analyze data sets. The analysis may be corrected for multiple comparisons, utilizing, for example, the Benjamini-Hochberg correction. Utilizing statistical analysis techniques to compare data, a t-test may be performed to compare the subject sample to the control sample and to determine if differences in value are due to random fluctuations or are due to other contributing factors. Differences in value represent increases or decreases in gene expression, and whether they are due to random fluctuations or other contributing factors will depend upon the p-value derived. A p-value may be derived from the t-test results, and is a measure of the probability that increases or decreases in gene expression are due to random variations. Thus, a larger p-value indicates a greater likelihood that increases or decreases are most likely due to random variation. Conversely, a smaller p-value indicates that increases or decreases are less likely to be random, and are caused by another contributing factor, such as genetic dysregulation due to dietary restrictions. As used herein, an increase or decrease in gene expression is “statistically significant” if the p-value for the increase or decrease, relative to gene expression in a healthy subject, is less than or equal to 0.1, less than or equal to 0.05, less than or equal to 0.01, or less than or equal to 0.001.

As another non-limiting example, discriminant analysis may also be utilized to analyze the data sets. Discriminant analysis may be used to determine if any identified variables discriminate between two or more naturally occurring groups. For example, expression of a particular gene in a given group may be reduced relative to the control group, may be the same as the control group, or may be increased relative to the control group. Therefore, discriminant analysis may be used to determine which group a particular gene falls into, based upon its measured level of expression. Discriminant analysis may be performed to determine what variables may be used as a predictor of gene expression. Methods of performing discriminant analysis are well known in the art, and will be within the knowledge and capabilities of persons of ordinary skill in the art.

In another non-limiting example, linear regression may also be utilized to analyze the data sets. Linear regression may be used to determine whether two variables are related, and to what degree, by fitting a linear equation to the observed data. Methods of performing linear regression are well known in the art, and will be within the knowledge and capabilities of persons of ordinary skill in the art.

Other methods of statistical analysis are well known in the art, and may also be performed by those of ordinary skill in the art.

The following nonlimiting examples serve to further illustrate the present invention.

### EXAMPLES

### Example 1

Epidemiological studies indicate that babies born in the lower extreme of birth weight (BW) are at risk for physical and mental diseases in adulthood. Heart rate (HR) and blood pressure (BP) responses to feeding were measured within the first days of life in order to determine whether signs or markers of this increased vulnerability could be detected early in life. Preliminary studies indicate that term babies with low BWs had the greatest increases in HR during feeding. Placenta gene expression markers associated with fetal growth were examined to determine if they may be related to these physiological responses.

To briefly summarize, 33 term infants, enrolled to include a broad range of BWs, were bottle-fed a sweetened solution of 5% dextrose for 5 minutes. HR and BP changes were measured as values during feeding minus values during baseline just before feeding. Placenta samples were taken shortly after delivery and expression of Insulin-like Growth Factor I (IGF1) was measured using quantitative real-time PCR.

Gene expression in newborn human infants was tested in conjunction with identification of physiologic correlates of birth weight. Heart rate and blood pressure differences was measured between term infants with birth weights on the low end of the normal distribution versus those with average or high birth weights. Placental gene expression of IGF1 was assessed to determine if infants could be identified which might be at greatest risk.

### Subjects

Healthy, full-term infants that were in the low, middle, or high quintiles of birth weight were enrolled for these studies. Infants were excluded from the study if one of the following factors was present: evidence of drug abuse, congenital anomalies, APGAR (Activity Pulse Grimace Appearance Respiration) scores less than 7 at one or five minutes, admission to the neonatal intensive care unit, and gestational ages less than 38 or greater than 41 weeks.

Newborn infants were tested in the hospital prior to discharge at between 12 and 98 hours of age. Testing was done during regularly scheduled feedings between 09:00 and 15:00. Bottle feeding infants were fed either sweetened water (D5W) or formula by a research assistant. Prior to feeding, a cuff of appropriate size for newborns (4-5 cm) was placed on the infant's leg below the knee for measurement of blood pressure. Blood pressure and heart rate measurements were made using a Dinamap clinical monitor.

Physiological measurements were made a few minutes before feeding while being held in the supine position by the feeder, during the first 5 minutes of feeding, and again after feeding was completed. During each period, a series of five blood pressure measurements was made, each separated by about one minute. At the end of the first five minutes of feeding the volume of nutrient consumed is noted, and the babies were then allowed to complete the feeding.

After feeding, babies underwent a series of tilt challenges involving four tilts, twice to a 30° head-up position and twice to a 30° head-down position. Babies were held in each position for 90 seconds.

### IGF1 Gene Expression

Placentas samples were collected 24-36 hours after delivery. Samples were dissected in a consistent manner so that each sample had the same part of the placenta. Dissections were done at a quick pace on top of a petri-dish containing cold RNAlater. Using a dissecting razor, center sections of the placenta were taken (‘center’ meaning the portion of the placenta from which the umbilical vein protruded). The portion was cut through all cell layers (Amnion, Chorion, Decidua Parietalis, Endometrial veins and arteries and Myometrium) so as to include all sections of the placenta and ensure a complete look into the genetic makeup of the tissue. Dissected tissue were stored in 200 μl of TRIZOL™. Total RNA was extracted using the guanidine thiocyanate method (using the TRIZOL™ reagent; Invitrogen, Carlsbad, Calif.).

Small PCR products (<100 base-pairs) were amplified in quadruplets on an Opticon real-time PCR machine (MJ Research, Waltham, Mass.), using universal PCR conditions (65° C. to 59° C. touch-down, followed by 35 cycles [15 minutes at 95 C, 10 minutes at 59 C and 10 minutes at 72 C]), as described previously (Galfalvy et al. BMC Bioinformatics, 2003, 4:37). 150 pg of cDNA was amplified in 20 μl reactions [0.3× Sybr-green, 3 mM MgCl₂, 200 μM dNTPs, 200 μM primers, 0.5 unit Platinum Taq DNA polymerase (Invitrogen, Carlsbad, Calif.)]. Primer-dimers were assessed by amplifying primers without cDNA. Primers were retained if they produced no primer-dimers or non-specific signal only after 35 cycles. Results were calculated as relative intensity compared to actin. The last cycle was retained as baseline for comparison with “absent” genes. To limit the effect of putative RNA degradation on PCR amplification, primers were designed in the extreme 3′ end of the gene transcript and gene-specific cDNAs were produced for each samples using gene-specific reverse primers during the reverse transcription reaction (Sibille et al. J. Neuroscience, 2000, 20(8)2758-65).

### Results

Multiple regression analyses that included IGF1 expression, gestational age, birth weight, length, and head, abdomen and chest circumferences showed that length and IGF1 expression were the best predictors of HR responses to feeding. Each measure showed a negative correlation with HR reactivity and together had a multiple R of 0.623, p<0.002. Using these two variables, we could identify 10 of the 11 infants with HR responses that were greater than 20 BPM. These results suggest that physical and gene expression markers of fetal growth provide good predictors of infant physiology and, perhaps markers of later cardiovascular disease vulnerability.

Measurement of placental IGF1 expression levels in these babies shows that IGF1 varies across body weight classifications, with the heaviest baby class having the highest level of IGF1 expression (FIG. 2). In addition to IGF1 expression levels, TLBW babies also show variation in cardiovascular function such as HR and blood pressure (BP) response to feeding. This data therefore shows that IGF1 expression may be used as a reliable and objective method for diagnosing TLBW.

### Example 2

### Test of Expression Profile Sensitivity and Specificity

### Methods

Gene expression data from pregnant rats under different forms of dietary restriction was gathered. Five groups of rats were fed different diets or administered fluoxetine as follows:
Group 1 (Controls): Rats with ad libitum food through out pregnancy. (N=8)
Group 2 (CR50): Rats Given 50% of their Normal Daily Intake of Food. (N=5)
Group 3 (CR70): Rats Given 70% of their Normal Daily Intake of Food. (N=5)
Group 4 (HF): Rats Given a High Fat (45% Animal Fat)/High Energy (4.73 kcal/g) diet throughout gestation. (N=6)
Group 5 (SSRI): Rats give Fluoxetine (˜10 mg/kg/day) throughout gestation. (N=6)
   Placentas from the rats of each Group were harvested on Day 21 of gestation. RNA was extracted and gene expression was measured based on hybridization of RNA to Affymetrix Rat Genome Chips™ (230.2) (Affymetrix, Santa Clara, Calif.).

### Results

### Discrimination of High Fat Diet

Probe sets which showed a significant difference between Control and HF groups were first identified. When utilizing Benjamini adjusted p values where p<0.05 was used as the cut-off, 6,939 probe sets were identified which exhibited altered expression based on the high fat diet. When utilizing Benjamini adjusted p values where p<0.001 was the cutoff, 386 probe sets were identified. The entire list of the 386 probe sets identified are attached as Table 6. From the 386 probe sets identified, 5 genes were chosen to include in the analysis. The 5 genes are shown below in Table 5. There was no systematic basis for this selection other than the probe set had an identified gene name. Any set of genes which exhibit a significant change in gene expression due to dietary restriction may be used.

TABLE 5

| PROBE SET | NAME | FUNCTION |
|---|---|---|
| 1369179 | peroxisome proliferation | adipocyte differentiation |
| | activated receptor (gamma) | |
| 1368271 | fatty acid binding protein 4 | lipid binding |
| 1368587 | apolipoprotein C-1 | lipid transport activity |
| 1387027 | lectin, galactose binding | ion transport, sugar |
| | binding, soluble 9 | binding |
| 1387663 | glia maturation factor beta | cell growth |

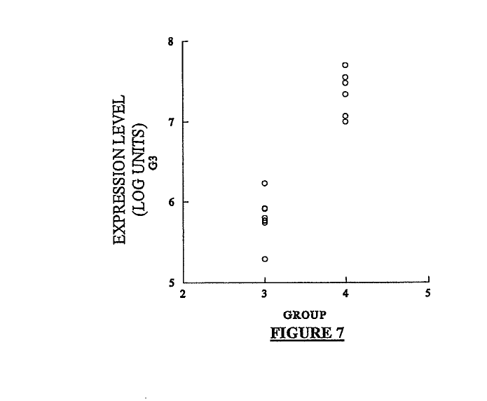

FIG. 7 shows the expression levels (log units) for the control group (group 3) and the HF group (group 4) for apolipoprotein C-1 (probe set 1368587) (designated “G3”).

### Discrimination of Caloric Restriction

Utilizing ANOVA analysis, Affymetrix probe sets were identified which showed a significant difference between Control and CR50 or Control and CR70 groups. 4,729 probe sets were identified which exhibited a Benjamini adjusted p value of p<0.05. From the 4,729 probe sets, 208 genes were identified which demonstrated rank ordered expression with regard to level of deprivation (Control<CR50<CR70, N=64; Control>CR50>CR70, N=144). These 208 probe sets showing rank ordered expression are provided in Table 7. Rank ordered expression of the genes indicates that the gene expression in rats at 50% caloric restriction (CR50) was increased relative to the control rats (CR50>CONT) and where the gene expression in rats at 70% caloric restriction (CR70) was increased relative to the CR50 rats (CR70>CR50). These rats are designated “TRUE” in the “upup” column. Alternatively, rank ordered expression of the genes may indicate that the gene expression in rats at 50% caloric restriction (CR50) was decreased relative to the control rats (CR50<CONT) and where the gene expression in rats at 70% caloric restriction (CR&)) was decreased relative to the CR50 rats (CR70<CR50). These rats are designated “TRUE” in the “downdown” column. From the latter set of 208 genes, 5 exemplar genes were selected to include in the analysis. As with the HF gene selection, there was no systematic basis for this selection other than the probe set had an identified gene name. The 5 genes selected are shown below in Table 8. Table 8 shows the individual expression (log units) for the CR70 group (group 1), CR50 group (group 2), and the control group (group 3) for vascular adhesion molecule 1 (probe set 1368474) (designated “G7”).

TABLE 8

| PROBE SET | NAME | FUNCTION |
|---|---|---|
| 1369132 | solute carrier 18, member 2 | neurotransmitter |
| | | transport |
| 1368474 | vascular adhesion | cell adhesion |
| | molecule 1 | |
| 1388116 | collagen type 1 alpha | collagen |
| 1368558 | allograft inflammatory | macrophage activation |
| | factor 1 | |
| 1387796 | arachidonate 12 | electron transport |
| | lipoxygenase | |

### Discriminant Analysis of Four Nutritional Groups

The data for the 10 genes identified above in Tables 5 and 8 was analyzed for each of the 24 animals (8 Control, 5 CR50, 5 CR70, 6 HF) utilizing a Discriminant Analysis, which allows for the classification of a set of observations into predefined classes. From the expression level data for all 10 genes, Discriminant Analysis produces probabilities for group membership, which in turn allows for a projected group classification. The results from this analysis are shown in Table 9. Based upon the Discriminant Analysis, the expression levels of these 10 genes was used to predict the membership of the each animal to a particular group, i.e., the control group, CR50 group, CR70 group, or the HF group. Based upon the discriminant analysis, each animal was correctly classified into the correct group with 100% accuracy. These results support the hypothesis that prospectively defined sets of genes, given appropriate weighting values can be used to classify individual infants with regard to at least some important characteristics of there their prenatal nutritional experiences. This provides a valuable tool for determining if prenatal growth was optimal, and as a potential guide for future treatment of the newborn infant.

### Discrimination and Specificity of SSRI Exposure

In order to determine whether placental gene expression profiling can be used to identify other prenatal exposures such as toxicants and drugs, a group of rats with drug exposure was monitored as described above. The SSRI group described above were fed a serotonin selective reuptake inhibitor (SSRI, Fluoxetine) in their drinking water throughout pregnancy at a dose of approximately 10 mg/kg/day. This dosage was estimated based upon the average water intake per day per animal, average size of the animals, and the dosage of fluoxetine provided in the water. As with control, CR50, CR70, and HF groups, placentas from the mothers were sampled after 21 days of gestation and gene expression monitored utilizing an Affymetrix Rat Genome Chip. Although many fewer genes were affected by this treatment than for either caloric restriction or high fat diets, expression of 281 probe sets was altered by exposure to fluoxetine. These probe sets are shown in Table 10. Of the 10 genes showing the greatest increase or decrease in expression, relative to the control group, none were among the genes affected by the HF diet (at the p<0.001 level), and none were among the genes that exhibited dose response effects of caloric restriction. This data supports the hypothesis that placental gene expression profiling can provide specific markers of drug exposure.

### Development of a Caloric Restriction Score

Utilizing statistical analysis methods, a caloric restriction score can be generated based upon gene expression data. The caloric restriction score may be used to determine dietary conditions during pregnancy.

To generate a caloric restriction score, linear regression formulas are first generated for each gene being examined. To generate linear regression formulas, genes which have been identified to be differentially expressed based upon dietary restrictions during gestation are selected. The data samples are collected for genes from subjects which experience varying levels of dietary restriction during pregnancy. The data sample for each gene is assigned an arbitrary value depending upon the dietary restrictions during gestation. It will be recognized that the arbitrary value is useful for the purposes of statistical analysis to provide a comparative value between data samples, and the selection of appropriate values will be within the capabilities of a person of ordinary skill in the art. For example, data samples from control subjects which are under no dietary restriction may be assigned a value of 1 (one), data samples from subjects with a 50% dietary restriction (caloric intake reduced by 50%) may be assigned a value of 2 (two), and data samples with a 70% dietary restriction (caloric intake reduced by 70%) may be assigned a value of 3 (three). Alternatively, values corresponding to the level of dietary restriction may be assigned. For example, data samples from control subjects which are under no dietary restriction may be assigned a value of 100 (representing 100% caloric intake, i.e., subjects fed ad libitum), data samples from subjects with a 50% dietary restriction (caloric intake reduced by 50%) may be assigned a value of 50 (representing 50% caloric intake, relative to subjects fed ad libitum), and data samples with a 70% dietary restriction (caloric intake reduced by 70%) may be assigned a value of 30 (representing 30% caloric intake, relative to subjects fed ad libitum). The data samples for each gene are analyzed by a linear regression model, and a weighting factor (i.e., slope or beta weight) is generated for the gene. Based upon the weighting factor and linear regression model, a linear regression formula is deduced which may be used to determine intercept values for the expression of the gene in a given sample, by inputting the gene expression data.

A caloric restriction score can be computed as a composite score of the gene expression for each gene being examined. An example of linear regression data and a formula for calculating the caloric restriction score can be seen in Table 3 and Table 4, utilizing the genes represented by the probe sets listed in Table 8. By way of example, a caloric restriction score can be computed for five genes (G6, G7, G8, G9, and G10) utilizing the following formula:

(Caloric Restriction Score)=(Constant)+(G6 coefficient)×(G6 expression)+(G7 coefficient)×(G7 expression)+(G8 coefficient)×(G8 expression)+(G9 coefficient)×(G9 expression)+(G10 coefficient)×(G10 expression)

The (Constant) variable may be calculated as the intercept value from a linear regression model based on the expression of all genes being examined, i.e., G6, G7, G8, G9, and G10. In the exemplary formula above, the “G6 coefficient” represents the weighting factor determined via the linear regression analysis for gene G6, the “G7 coefficient” represents the weighting factor determined via the linear regression analysis for gene G7, the “G8 coefficient” represents the weighting factor determined via the linear regression analysis for gene G8, the “G9 coefficient” represents the weighting factor determined via the linear regression analysis for gene G9, the “G10 coefficient” represents the weighting factor determined via the linear regression analysis for gene G10. It will be apparent to a person of ordinary skill in the art that more or fewer genes may be utilized to calculate the caloric restriction score by adding or removing terms to the above equation.

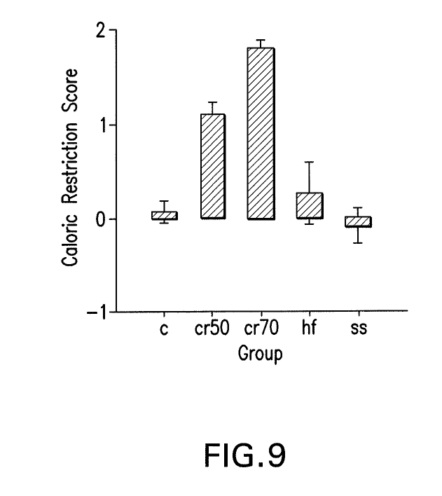

The results from the Control group, the CR50 group (50% dietary restriction), and the CR70 group (70% dietary restriction) were used to construct a Caloric Restriction Score (CRS). Statistical analysis of the data was run utilizing Systat™ software (available from Systat Software, Inc., Point Richmand, Calif.). The caloric restriction formula was first produced for each of the 5 genes that were significantly related to caloric restriction by running a linear regression model, wherein the Control group was assigned a value of 1, the CR50 group was assigned a value of 2, and the CR70 group was assigned a value of 3. From these formulas, a weighting factor for each gene was derived and intercepts calculated for the gene expression of each gene for each animal. These values were then used to produce a CRS for each animal. FIG. 9 shows the means and standard errors for these predicted CRSs for each group. As would be expected, the CRS increases above Control levels with increasing levels of caloric restriction. In addition, this analysis indicates that the profile of gene expression for the high fat group (HF) and the SSRI exposed group (SS) do not fit a pattern consistent with overall caloric restriction.

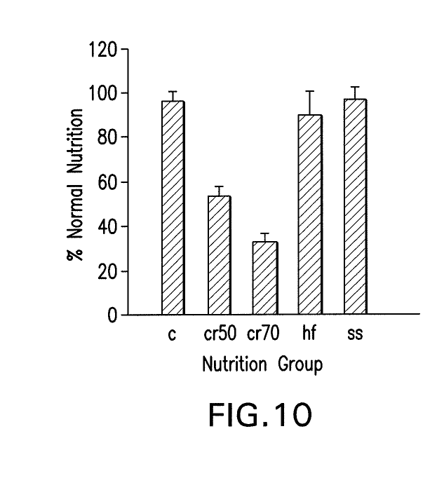

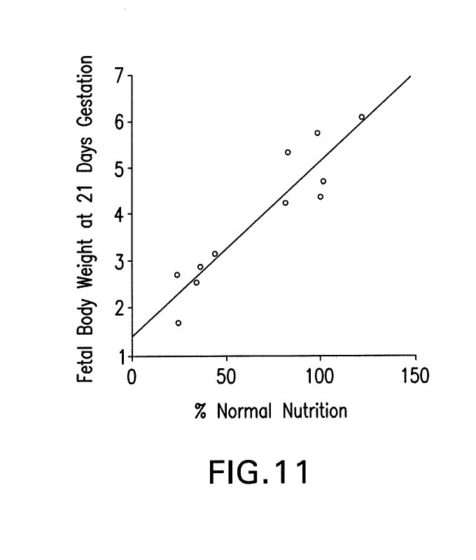

Alternatively, the linear regression model may be calculated wherein the Control group is assigned a value of 100, the CR50 group is assigned a value of 50, and the CR70 group is assigned a value of 30. The remaining steps are performed as described above. The complete data resulting from this analysis can be found in Tables 3 and 4. FIG. 10 shows the means and standard errors for these predicted CRSs for each group. The caloric restriction score resulting from this analysis indicates the predicted percent of normal nutrition. Based upon this analysis, the body weights of rat fetuses were measured at day 21, i.e., the day before expected delivery, and the body weights compared to the caloric restriction score generated for each fetus. FIG. 11 shows the relationship between body weight and the caloric restriction score representing the predicted percent of normal nutrition. This data demonstrates that the caloric restriction score can be used to track group mean differences, but also can provide a good marker for individual level of caloric restriction and fetal growth.

### Example 3

### Effect of Caloric Restriction During Pregnancy on Placental Gene Expression in Rats

Gene expression from placentas of 6 control and 5 food restricted (70%) pregnancies were measured in rats. Placentas from 6 pups from each litter were pooled. RNA was extracted and over 30,000 genes and expressed sequences were analyzed using Affymetrix chips. Of that total, 6143 (˜20%) showed no overlap in expression between control and IUGR placentas. Of the 6143, 2819 (˜46%) were down-regulated, and 3324 (˜54%) were up-regulated.

Within the group of down-regulated genes there are five fibroblast growth factor genes and 16 solute carrier genes including the facilitated glucose transporter (Slc2a5) that are significantly altered the caloric restriction. In addition, several insulin-related genes were down regulated including, insulin itself (Ins 2), insulin degrading enzyme, insulin receptor-related receptor, insulin-like growth factor I, insulin-like growth factor binding protein 6, and insulin-like growth factor binding protein 5. Within the up-regulated gene group there are 6 genes associated with calcium channels, corticotrophin receptor hormone (CRH) binding protein and CRH receptor, and 6 fibroblast growth factors. There were also 11 solute carrier genes that were up-regulated including 2 amino acid transporters (1-proline and a cationic AA transporter). The insulin-like protein 6 and insulin-like growth factor 2 were also among this group.

Data analysis is being conducted to characterize the patterns of change and functional clusters of genes that are altered by prenatal malnutrition. Assays based upon expression of the identified genes will afford new strategies for determining if individual fetuses have been subjected to deviations from normal patterns of nutrient delivery. Gene expression in rats also provides candidate genes to be assayed in human placental samples.

### Placental Gene Expression in Growth Restriction: Comparison of Rat and Human Microarray Studies.

In the 70% restriction model in rats discussed above, several hundred genes were found that show no overlap between control and under-grown groups. To determine if at least some of these changes might also be related to under-growth of the human fetus, the results were compared with recently published work by McMinn and colleagues. McMinn et al., supra. McMinn examined mRNA expression in 14 IUGR placentas with maternal vascular under-perfusion compared to 15 non-IUGR placentas using Affymetrix microarrays. As was the case in the rat study above, McMinn found numerous differences in expression in IUGR placentas. Id. For example, increased expression of PHLDA2 and decreased expression of MEST, MEG3, GATM, GNAS and PLAGL1 in IUGR placentas was found. Id. In addition to imprinted genes, differences were detected in endocrine signaling (LEP, CRH, HPGD, INHBA), tissue growth (IGF1), immune modulation (INDO, PSG-family genes), oxidative metabolism (GLRX), vascular function (AGTR1, DSCR1) and metabolite transport (SLC-family solute carriers). Id.

Comparison of the data from McMinn and the present rat studies resulted in several lists of genes, shown in Table 2. McMinn et al., supra. Table 2 contains genes that were observed to have increased expression in both the caloric restriction rat model and in McMinn's IUGR human analyses. Table 2 also provides a list of genes with reduced expression in both data sets. Lastly, Table 2 provides lists of genes with reduced expression in one data set, and increased expression in the other data set. This comparative data supports the hypothesis that there will be distinct patterns of placental gene expression associated with fetal over-growth. The specificity of these patterns are tested by enriching groups of infants with those exhibiting catch-down growth during the early postnatal period and by convergent results obtained from experimental manipulation of weight gain in an animal model.

### Example 4

### Placental Gene Expression Results from Prenatal Alcohol Exposure Study

Microarray placental gene expression results were obtained from a total of 18 pregnancies; 9 animals with no exposure to alcohol, 5 given 5% alcohol in their drinking water throughout pregnancy, and 4 given 10% alcohol in their drinking water throughout pregnancy. The Affymetrix chip for the rat genome quantifies expression of 31,101 probe sets, and identifies sequences of mRNA known to be expressed in the rat. Because a large number of comparisons between exposed and unexposed animals are possible, a statistical strategy was devised to reduce the likelihood of finding false positives. For the first phase of analysis three criteria were combined to reduce the problem of false discovery. First, t-tests were performed for all probe sets, testing for differences between control and 5% samples. This is shown in Tables 11 and 12, in the columns marked “T-val contr.vs.5.” Second, t-tests were performed for all probes sets, testing for differences between 5% and 10% samples. This is shown in Tables 11 and 12, in the columns marked “T-val 5 vs. 10.” To be included in the list of candidate genes, it was required that the probability of a significant difference between groups (identified utilizing the p-value) was less than 0.05 for both tests. The combined probability of false detection was thus 0.05×0.05, or 0.0025. In addition, the genes were included only if group differences were ordered in 2 of the four possible ways; 0<5%<10% or 0>5%>10%. This is shown in Tables 11 and 12 in the columns marked “Contr<5,” “5<10,” “c<5<10,” “Contr>5,” “5>10,” and “c>5>10.” Thus, for each of the two possible patterns of group differences, the overall expected rate of false identification was 0.05×0.05×0.25(one quarter of the possible patterns)×31,101=19 genes.

Using the above criteria 57 probe sets were identified with reduced expression in the alcohol exposed animals (that is, the 0>5%>10% pattern of group differences), or approximately 3 times more than would be expected by chance alone. The probe sets with reduced expression in the alcohol exposed animals selected under the criteria described above are shown in Table 11. In contrast, only 8 probe sets were identified as being up-regulated by alcohol, thus giving less confidence that this type of change was due to chance alone. The probe sets with increased expression in the alcohol exposed animals selected under the criteria described above are shown in Table 12.

Following this screening procedure it was next determined which of the 65 potential alcohol responsive genes were found in prior data sets that had tested for effects of caloric restriction, high fat diets, and SSRI (such as fluoxetine) exposure during pregnancy. The prior data for the effects of caloric restriction, high fat diets, and SSRI exposure during pregnancy are reflected in Tables 11 and 12 in the columns marked “sig SSRI,” “Sig HF,” “Sig MMMCR,” “sig up,” and “sig down.” After excluding all overlapping genes, 35 placental genes remained that were down-regulated by exposure to alcohol and 7 placental genes remained that were up-regulated by exposure to alcohol. The 35 genes remaining that were down regulated by alcohol are shown by the shaded rows in Table 11. The 7 remaining genes that were up regulated by alcohol are shown by the shaded rows in Table 12.

From this list of 42 candidate genes 6 probes were chosen for further analysis that were identified as genes that showed the most pronounced group differences. Expressed sequences which have not been characterized as known genes were not included. The 5 down-regulated genes were: peroxisomal biogenesis factor 6 (Pex6), cell division cycle associated 7 (Cdca7), TP53 regulating kinase (Trp53rk), pleckstrin (Plek), and inositol polyphosphate-1-phosphatase (Inpp1). The one up-regulated gene included was Zinc finger protein 365 (Zfp365).

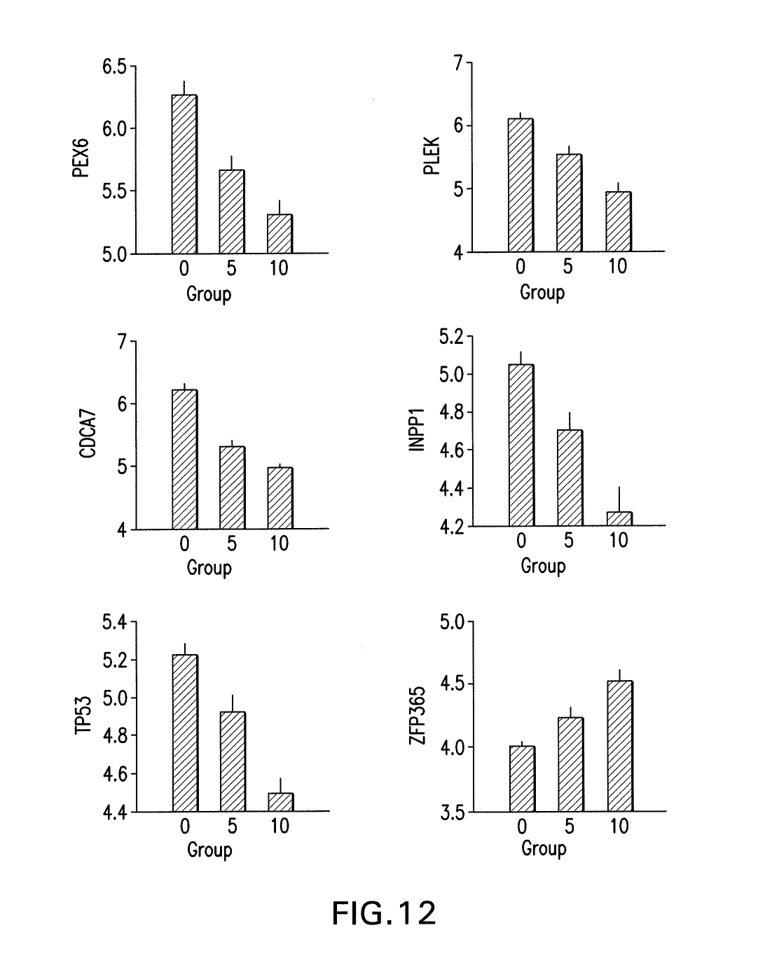

FIG. 12 shows the mean (±SE) expression values for each of these 6 genes. As can be seen in this figure, there is a clear dose-response change in expression associated with alcohol exposure during pregnancy. A multivariate regression analysis was performed to determine how well expression levels of these 6 genes predicated alcohol exposure. As can be seen in Table 13, together these genes were highly predicted of level of alcohol exposure, accounting for 96.4% of the variance in exposure.

TABLE 13
Results from multivariate analysis of variance using expression of 6
candidate genes to predict level of alcohol exposure.
DEP VAR: ALCAMT N: 18 R = 0.982 R² = 0.964 P < .001

| VARIABLE | COEFFICIENT | STD ERROR | STD COEF | TOLERANCE | T | P(2 TAIL) |
|---|---|---|---|---|---|---|
| INTERCEPT | 19.668 | 11.979 | 0.000 | — | 1.642 | 0.129 |
| PEX6 | −1.168 | 1.377 | −0.130 | 0.138 | −0.849 | 0.414 |
| CDCA7 | −2.038 | 0.928 | −0.291 | 0.184 | −2.197 | 0.050 |
| TP53 | −0.775 | 2.014 | −0.063 | 0.121 | −0.385 | 0.708 |
| PLEK | −0.290 | 1.187 | −0.037 | 0.137 | −0.244 | 0.812 |
| INPP1 | −2.925 | 1.034 | −0.268 | 0.361 | −2.827 | 0.016 |
| ZFP365 | 5.229 | 1.864 | 0.304 | 0.274 | 2.805 | 0.017 |

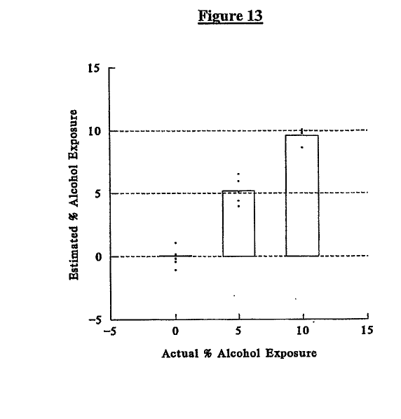

Next, to determine how accurately expression levels of these genes could estimate level of alcohol exposure, the coefficients from the regression model were used to calculate estimated values. FIG. 13 shows the mean estimates for alcohol exposure for each group compared to the actual exposure. As can be seen in this figure, expression levels of these genes can be used to produce very accurate estimates of exposure level.

Finally, discriminate analysis was performed which used the gene expression values to predict exposure group membership. As can be seen in Table 14, by combining expression values from these six genes, which placentas were in which exposure group could be predicted with 100% accuracy.

TABLE 14

| | | Actual Treatment | | |
|---|---|---|---|---|
| | | Group | | |
| | | Control | 5% | 10% |
| Predicted | Control | 9 | 0 | 0 |
| Group | 5% | 0 | 5 | 0 |
| | 10% | 0 | 0 | 4 |

Together, these results clearly indicate that quantifying profiles of gene expression in the placenta affords a novel strategy for assessment of level and specificity of alcohol exposure during fetal development.

Various references are cited herein, which are hereby incorporated by reference in their entireties.

TABLE 1

| | Change in | | Change in | | Change in |
|---|---|---|---|---|---|
| Probe Set | expression | Probe Set | expression | Probe Set | expression |
| 1371198_at | down | 1392413_at | down | 1377863_at | up |
| 1383551_at | down | 1392416_at | down | 1391156_at | up |
| 1388544_at | down | 1392526_at | down | 1379272_at | up |
| 1369124_at | down | 1392571_at | down | 1376008_at | up |
| 1387493_at | down | 1392584_at | down | 1389247_at | up |
| 1390662_at | down | 1392600_a_at | down | 1371958_at | up |
| 1387909_at | down | 1392607_at | down | 1374175_at | up |
| 1383740_at | down | 1392608_at | down | 1390473_at | up |
| 1385123_at | down | 1392609_at | down | 1390250_x_at | up |
| 1380504_at | down | 1392637_at | down | 1390816_at | up |
| 1370856_at | down | 1392641_at | down | 1375301_at | up |
| 1388128_at | down | 1392675_at | down | 1393692_at | up |
| 1369341_a_at | down | 1392680_at | down | 1392436_at | up |
| 1387276_at | down | 1392683_at | down | 1393274_at | up |
| 1369526_at | down | 1392686_at | down | 1382504_at | up |
| 1369734_at | down | 1392762_at | down | 1376279_at | up |
| 1376022_at | down | 1392846_at | down | 1375332_at | up |
| 1368933_at | down | 1392962_at | down | 1390044_at | up |
| 1368370_at | down | 1393041_at | down | 1378196_at | up |
| 1380643_at | down | 1393077_at | down | 1383685_at | up |
| 1376268_at | down | 1393154_at | down | 1374828_at | up |
| 1374178_at | down | 1393187_at | down | 1393642_at | up |
| 1389967_at | down | 1393291_at | down | 1380873_at | up |
| 1368021_at | down | 1393298_at | down | 1372603_at | up |
| 1391657_at | down | 1393432_a_at | down | 1382812_at | up |
| 1368558_s_at | down | 1393434_at | down | 1381536_at | up |
| 1377700_at | down | 1393446_at | down | 1388519_at | up |
| 1393596_at | down | 1393479_at | down | 1386422_at | up |
| 1378955_at | down | 1393492_at | down | 1389653_at | up |
| 1388007_x_at | down | 1393512_at | down | 1385435_at | up |
| 1368465_at | down | 1393514_at | down | 1377079_a_at | up |
| 1392135_at | down | 1393515_at | down | 1373741_at | up |
| 1372615_at | down | 1393529_at | down | 1385298_at | up |
| 1393000_at | down | 1393530_at | down | 1391231_at | up |
| 1387289_at | down | 1393547_at | down | 1391392_at | up |
| 1370846_at | down | 1393577_at | down | 1393300_at | up |
| 1387100_at | down | 1393580_at | down | 1375326_at | up |
| 1368621_at | down | 1393597_at | down | 1393307_at | up |
| 1387796_at | down | 1393607_at | down | 1374332_at | up |
| 1367988_at | down | 1393615_at | down | 1395401_at | up |
| 1369026_at | down | 1393653_at | down | 1392246_at | up |
| 1369873_at | down | 1393734_at | down | 1398422_at | up |
| 1393958_at | down | 1393820_at | down | 1391340_at | up |
| 1397415_at | down | 1393927_at | down | 1397312_at | up |
| 1370350_x_at | down | 1394079_at | down | 1397747_at | up |
| 1397224_at | down | 1394107_at | down | 1378704_at | up |
| 1369342_at | down | 1394129_at | down | 1373631_at | up |
| 1368769_at | down | 1394259_at | down | 1375156_at | up |
| 1370465_at | down | 1394283_at | down | 1392092_at | up |
| 1398265_at | down | 1394284_at | down | 1397314_at | up |
| 1368561_at | down | 1394434_at | down | 1379311_at | up |
| 1387184_at | down | 1394447_at | down | 1394037_at | up |
| 1390429_at | down | 1394455_at | down | 1376855_at | up |
| 1369248_a_at | down | 1394504_at | down | 1374962_at | up |
| 1369084_a_at | down | 1394528_at | down | 1396087_at | up |
| 1373733_at | down | 1394549_at | down | 1373593_at | up |
| 1368482_at | down | 1394551_at | down | 1383500_at | up |
| 1388144_at | down | 1394577_at | down | 1371445_at | up |
| 1371440_at | down | 1394578_at | down | 1375560_at | up |
| 1375284_at | down | 1394584_at | down | 1377083_at | up |
| 1369426_at | down | 1394630_at | down | 1398372_at | up |
| 1378427_at | down | 1394694_at | down | 1374114_at | up |
| 1388075_at | down | 1394699_at | down | 1394523_at | up |
| 1387938_at | down | 1394717_at | down | 1377448_at | up |
| 1370849_at | down | 1394756_at | down | 1389171_at | up |
| 1389821_at | down | 1394801_at | down | 1376603_at | up |
| 1387540_at | down | 1394809_at | down | 1388665_at | up |
| 1377817_at | down | 1394820_at | down | 1376192_at | up |
| 1371199_at | down | 1394861_at | down | 1386194_at | up |
| 1368523_at | down | 1394890_at | down | 1376239_at | up |
| 1368642_at | down | 1394891_at | down | 1379809_at | up |
| 1370657_at | down | 1394919_at | down | 1394519_at | up |
| 1397177_at | down | 1394933_at | down | 1384318_at | up |
| 1369647_at | down | 1394971_at | down | 1377252_at | up |
| 1377640_at | down | 1395010_at | down | 1377268_at | up |
| 1378073_at | down | 1395028_at | down | 1375351_at | up |
| 1370133_at | down | 1395056_at | down | 1371534_at | up |
| 1377518_at | down | 1395057_at | down | 1379254_at | up |
| 1389824_at | down | 1395096_at | down | 1396059_at | up |
| 1368156_at | down | 1395151_at | down | 1375076_at | up |
| 1368955_at | down | 1395194_at | down | 1391865_at | up |
| 1376345_at | down | 1395203_at | down | 1384884_at | up |
| 1368823_at | down | 1395207_at | down | 1384670_at | up |
| 1384532_at | down | 1395218_at | down | 1391840_at | up |
| 1368131_at | down | 1395260_at | down | 1373728_at | up |
| 1387991_at | down | 1395359_at | down | 1381407_at | up |
| 1367785_at | down | 1395368_at | down | 1385001_at | up |
| 1368905_at | down | 1395373_at | down | 1373071_at | up |
| 1370363_at | down | 1395383_at | down | 1375208_at | up |
| 1368913_at | down | 1395389_at | down | 1381991_at | up |
| 1369186_at | down | 1395390_at | down | 1385885_at | up |
| 1387690_at | down | 1395432_at | down | 1395913_at | up |
| 1368637_at | down | 1395433_at | down | 1373023_at | up |
| 1387005_at | down | 1395438_at | down | 1393203_at | up |
| 1369865_at | down | 1395499_at | down | 1376500_at | up |
| 1368555_at | down | 1395575_at | down | 1386614_at | up |
| 1368975_at | down | 1395635_at | down | 1395647_at | up |
| 1369483_at | down | 1395656_at | down | 1371452_at | up |
| 1370891_at | down | 1395726_at | down | 1377945_at | up |
| 1367679_at | down | 1395747_at | down | 1395190_at | up |
| 1395116_at | down | 1395762_at | down | 1377837_at | up |
| 1388013_at | down | 1395820_at | down | 1397884_at | up |
| 1388053_at | down | 1395834_at | down | 1390418_at | up |
| 1384085_at | down | 1395902_at | down | 1375876_at | up |
| 1382113_at | down | 1395912_at | down | 1379228_at | up |
| 1378832_at | down | 1396057_at | down | 1392671_at | up |
| 1370391_at | down | 1396071_at | down | 1397683_at | up |
| 1393680_at | down | 1396119_at | down | 1397882_at | up |
| 1375977_at | down | 1396157_at | down | 1383911_at | up |
| 1383121_at | down | 1396162_at | down | 1383779_at | up |
| 1387709_at | down | 1396281_at | down | 1389777_at | up |
| 1369983_at | down | 1396343_at | down | 1382187_at | up |
| 1387969_at | down | 1396377_at | down | 1374484_at | up |
| 1379365_at | down | 1396378_at | down | 1391626_at | up |
| 1369633_at | down | 1396520_at | down | 1376866_at | up |
| 1387956_s_at | down | 1396577_at | down | 1389888_at | up |
| 1397076_at | down | 1396602_at | down | 1380265_at | up |
| 1376800_at | down | 1396607_at | down | 1374634_at | up |
| 1394833_at | down | 1396613_at | down | 1380152_at | up |
| 1387032_at | down | 1396619_at | down | 1390481_a_at | up |
| 1369112_at | down | 1396669_at | down | 1398374_at | up |
| 1368734_at | down | 1396670_at | down | 1394216_at | up |
| 1388054_a_at | down | 1396725_at | down | 1383902_at | up |
| 1388142_at | down | 1396796_at | down | 1379759_at | up |
| 1388265_x_at | down | 1396807_at | down | 1383891_a_at | up |
| 1371672_at | down | 1396815_at | down | 1394509_at | up |
| 1369951_at | down | 1396837_at | down | 1383128_at | up |
| 1368658_at | down | 1396854_at | down | 1379981_at | up |
| 1376711_at | down | 1396863_at | down | 1376501_at | up |
| 1389944_at | down | 1396947_at | down | 1377848_at | up |
| 1378431_at | down | 1396979_at | down | 1393773_at | up |
| 1369800_at | down | 1396980_at | down | 1394960_at | up |
| 1382194_at | down | 1396997_at | down | 1381755_x_at | up |
| 1374779_at | down | 1397034_at | down | 1372712_at | up |
| 1369724_at | down | 1397048_at | down | 1391501_at | up |
| 1384063_at | down | 1397081_at | down | 1391749_a_at | up |
| 1376099_at | down | 1397109_at | down | 1385650_at | up |
| 1369529_at | down | 1397122_at | down | 1374980_at | up |
| 1387893_at | down | 1397125_at | down | 1385154_at | up |
| 1368695_at | down | 1397135_at | down | 1378312_at | up |
| 1368742_at | down | 1397158_at | down | 1378788_at | up |
| 1387446_at | down | 1397195_at | down | 1394458_at | up |
| 1376051_at | down | 1397346_at | down | 1373177_x_at | up |
| 1393008_at | down | 1397369_at | down | 1380032_at | up |
| 1387496_a_at | down | 1397374_at | down | 1389472_at | up |
| 1387897_at | down | 1397376_at | down | 1389140_at | up |
| 1383075_at | down | 1397404_at | down | 1398177_at | up |
| 1368083_at | down | 1397428_at | down | 1389641_at | up |
| 1387296_at | down | 1397431_at | down | 1391609_at | up |
| 1371274_at | down | 1397560_at | down | 1395638_at | up |
| 1369113_at | down | 1397585_at | down | 1393332_at | up |
| 1367782_at | down | 1397591_at | down | 1377016_at | up |
| 1387913_at | down | 1397610_at | down | 1393315_at | up |
| 1368934_at | down | 1397654_at | down | 1398485_at | up |
| 1369444_at | down | 1397655_at | down | 1376461_at | up |
| 1368990_at | down | 1397676_at | down | 1375066_at | up |
| 1393155_at | down | 1397734_at | down | 1395174_at | up |
| 1367924_at | down | 1397751_at | down | 1390162_at | up |
| 1370774_at | down | 1397759_at | down | 1384776_x_at | up |
| 1392520_at | down | 1397760_at | down | 1382502_at | up |
| 1388257_at | down | 1397783_at | down | 1379084_at | up |
| 1369434_at | down | 1397790_at | down | 1397856_at | up |
| 1368631_at | down | 1397879_at | down | 1392541_at | up |
| 1388194_at | down | 1397881_at | down | 1379802_at | up |
| 1397218_at | down | 1397889_at | down | 1373906_at | up |
| 1369390_a_at | down | 1397912_at | down | 1394911_at | up |
| 1368673_at | down | 1397926_at | down | 1391134_at | up |
| 1395925_s_at | down | 1397937_at | down | 1390705_at | up |
| 1370507_at | down | 1397968_at | down | 1384345_at | up |
| 1387795_at | down | 1397979_at | down | 1397171_at | up |
| 1369421_at | down | 1397987_at | down | 1391482_a_at | up |
| 1368064_a_at | down | 1398002_at | down | 1385897_at | up |
| 1378789_at | down | 1398003_at | down | 1376516_at | up |
| 1380232_at | down | 1398026_at | down | 1378523_at | up |
| 1380233_x_at | down | 1398099_at | down | 1377895_at | up |
| 1387514_at | down | 1398109_at | down | 1383634_at | up |
| 1387490_at | down | 1398115_at | down | 1396319_at | up |
| 1369686_at | down | 1398132_at | down | 1381386_at | up |
| 1368903_at | down | 1398138_at | down | 1393939_at | up |
| 1380763_at | down | 1398156_at | down | 1372639_at | up |
| 1368699_at | down | 1398202_at | down | 1396593_at | up |
| 1387457_at | down | 1398238_at | down | 1378639_at | up |
| 1370576_at | down | 1398368_at | down | 1395326_at | up |
| 1380160_at | down | 1398392_at | down | 1388363_at | up |
| 1384866_at | down | 1398509_at | down | 1398993_at | up |
| 1373932_at | down | 1398530_at | down | 1389666_at | up |
| 1379344_at | down | 1398560_at | down | 1381722_at | up |
| 1393415_at | down | 1398692_at | down | 1386685_at | up |
| 1372745_at | down | 1398710_at | down | 1397964_at | up |
| 1378728_at | down | 1398741_at | down | 1393069_at | up |
| 1382766_at | down | 1398743_at | down | 1376255_at | up |
| 1387272_at | down | 1398990_at | down | 1393422_at | up |
| 1374224_at | down | 1399044_at | down | 1374748_at | up |
| 1384159_at | down | 1367508_at | down | 1375477_at | up |
| 1370033_at | down | 1374783_at | down | 1389198_at | up |
| 1381732_at | down | 1374829_at | down | 1389275_at | up |
| 1382887_at | down | 1375242_at | down | 1391800_at | up |
| 1388108_at | down | 1375404_at | down | 1383903_at | up |
| 1387351_at | down | 1375823_at | down | 1393801_at | up |
| 1370037_at | down | 1376550_at | down | 1381475_at | up |
| 1369604_at | down | 1377050_at | down | 1382350_at | up |
| 1369608_at | down | 1377076_at | down | 1372925_at | up |
| 1387606_at | down | 1377366_at | down | 1374080_at | up |
| 1387643_at | down | 1377511_at | down | 1374424_at | up |
| 1392865_at | down | 1378114_at | down | 1397479_at | up |
| 1367700_at | down | 1378152_at | down | 1396250_at | up |
| 1370132_at | down | 1378438_at | down | 1377467_at | up |
| 1395026_at | down | 1378623_at | down | 1377438_at | up |
| 1375744_at | down | 1379437_at | down | 1383083_at | up |
| 1371048_at | down | 1380070_at | down | 1385781_at | up |
| 1387386_at | down | 1380146_at | down | 1378731_at | up |
| 1369018_at | down | 1380193_at | down | 1376088_at | up |
| 1385372_at | down | 1380207_at | down | 1389747_at | up |
| 1387685_at | down | 1380413_at | down | 1377933_at | up |
| 1369916_at | down | 1380489_at | down | 1392811_at | up |
| 1371216_s_at | down | 1380587_at | down | 1375361_at | up |
| 1398818_at | down | 1380806_at | down | 1375257_at | up |
| 1381738_at | down | 1380864_at | down | 1379839_at | up |
| 1370744_at | down | 1380874_at | down | 1385225_at | up |
| 1387526_at | down | 1381121_at | down | 1374063_at | up |
| 1368299_at | down | 1381235_at | down | 1391858_at | up |
| 1387666_at | down | 1381287_at | down | 1379822_at | up |
| 1387241_at | down | 1381380_at | down | 1397254_at | up |
| 1369755_at | down | 1381408_at | down | 1376396_a_at | up |
| 1369844_at | down | 1381820_at | down | 1395699_at | up |
| 1387729_at | down | 1382090_at | down | 1394363_at | up |
| 1387207_at | down | 1382485_at | down | 1374691_at | up |
| 1369167_at | down | 1382520_at | down | 1374326_at | up |
| 1397587_at | down | 1382601_at | down | 1390043_at | up |
| 1386944_a_at | down | 1382788_at | down | 1392561_at | up |
| 1370725_a_at | down | 1382870_at | down | 1397777_at | up |
| 1387954_a_at | down | 1382950_at | down | 1384908_at | up |
| 1396696_at | down | 1383120_at | down | 1379237_at | up |
| 1387309_a_at | down | 1384374_at | down | 1377490_at | up |
| 1370998_at | down | 1384415_at | down | 1377548_at | up |
| 1368690_a_at | down | 1384599_at | down | 1384885_at | up |
| 1367806_at | down | 1384650_at | down | 1393850_at | up |
| 1382616_at | down | 1384745_at | down | 1391228_at | up |
| 1398378_at | down | 1384785_at | down | 1378941_at | up |
| 1387491_at | down | 1385098_at | down | 1385044_at | up |
| 1387696_a_at | down | 1385821_at | down | 1397182_at | up |
| 1373773_at | down | 1386257_at | down | 1378567_at | up |
| 1370389_at | down | 1389213_at | down | 1375920_at | up |
| 1380833_at | down | 1390713_at | down | 1381958_at | up |
| 1368395_at | down | 1390969_at | down | 1376485_at | up |
| 1370963_at | down | 1391106_at | down | 1380755_at | up |
| 1367930_at | down | 1391174_at | down | 1396430_at | up |
| 1398293_a_at | down | 1391276_at | down | 1390077_at | up |
| 1391599_at | down | 1391378_at | down | 1384049_at | up |
| 1372011_at | down | 1391596_at | down | 1378919_at | up |
| 1387505_at | down | 1391659_at | down | 1385443_at | up |
| 1368332_at | down | 1392093_at | down | 1377119_at | up |
| 1368154_at | down | 1392183_at | down | 1398068_at | up |
| 1391627_at | down | 1392364_at | down | 1373973_at | up |
| 1374046_at | down | 1392809_at | down | 1375596_at | up |
| 1369834_at | down | 1393406_at | down | 1373960_at | up |
| 1379482_at | down | 1393482_at | down | 1382513_at | up |
| 1383980_at | down | 1393589_at | down | 1377290_at | up |
| 1369855_at | down | 1393718_at | down | 1374356_at | up |
| 1390100_s_at | down | 1394472_at | down | 1374464_at | up |
| 1391731_at | down | 1394709_at | down | 1371917_at | up |
| 1370969_at | down | 1395240_at | down | 1372407_at | up |
| 1368302_at | down | 1396283_at | down | 1397499_at | up |
| 1375274_at | down | 1396442_at | down | 1393241_at | up |
| 1368319_a_at | down | 1396502_at | down | 1379842_at | up |
| 1370454_at | down | 1396534_at | down | 1393377_at | up |
| 1370656_a_at | down | 1396846_at | down | 1398472_at | up |
| 1389384_at | down | 1397202_at | down | 1384064_at | up |
| 1387548_at | down | 1397380_at | down | 1379123_at | up |
| 1394857_at | down | 1397724_at | down | 1395379_at | up |
| 1372922_at | down | 1397823_at | down | 1384404_at | up |
| 1377940_at | down | 1398059_at | down | 1393985_at | up |
| 1381391_at | down | 1398064_at | down | 1377985_at | up |
| 1384684_at | down | 1398166_at | down | 1388772_at | up |
| 1380037_at | down | 1398518_at | down | 1376851_at | up |
| 1389766_at | down | 1377086_at | down | 1386277_at | up |
| 1374294_at | down | 1377514_at | down | 1384421_at | up |
| 1382694_at | down | 1378709_at | down | 1375919_at | up |
| 1383726_at | down | 1379417_at | down | 1385609_at | up |
| 1393778_at | down | 1382757_at | down | 1393784_at | up |
| 1394720_at | down | 1385388_at | down | 1376528_at | up |
| 1372278_at | down | 1391235_at | down | 1385041_at | up |
| 1398651_at | down | 1394637_at | down | 1390245_at | up |
| 1377803_at | down | 1395795_at | down | 1386122_at | up |
| 1393851_at | down | 1397173_at | down | 1376063_at | up |
| 1375435_at | down | 1397256_at | down | 1381034_at | up |
| 1396668_at | down | 1397536_at | down | 1372232_at | up |
| 1367716_at | down | 1376530_at | down | 1380108_at | up |
| 1370594_at | down | 1377443_at | down | 1393453_at | up |
| 1375183_at | down | 1391030_at | down | 1389119_at | up |
| 1385923_at | down | 1394517_at | down | 1388912_at | up |
| 1394022_at | down | 1371448_at | down | 1379289_at | up |
| 1387078_at | down | 1372425_at | down | 1386454_at | up |
| 1388048_a_at | down | 1373326_at | down | 1375903_a_at | up |
| 1370077_at | down | 1375449_at | down | 1384800_at | up |
| 1387815_at | down | 1375923_at | down | 1380514_at | up |
| 1387652_at | down | 1376096_a_at | down | 1393105_at | up |
| 1369514_at | down | 1376186_at | down | 1379800_at | up |
| 1370333_a_at | down | 1376266_at | down | 1390361_at | up |
| 1372168_s_at | down | 1376380_at | down | 1378953_at | up |
| 1370960_at | down | 1376458_at | down | 1391189_a_at | up |
| 1369038_at | down | 1376823_at | down | 1397284_at | up |
| 1368707_at | down | 1376829_at | down | 1398413_at | up |
| 1367696_at | down | 1377475_at | down | 1382237_at | up |
| 1369266_at | down | 1378105_at | down | 1386831_at | up |
| 1387758_at | down | 1378111_at | down | 1390725_at | up |
| 1387440_at | down | 1378382_at | down | 1383762_at | up |
| 1398214_at | down | 1378429_at | down | 1373256_at | up |
| 1391796_at | down | 1378515_at | down | 1377241_at | up |
| 1378449_at | down | 1378526_at | down | 1394404_at | up |
| 1370436_at | down | 1378738_at | down | 1368483_a_at | up |
| 1369306_at | down | 1378791_at | down | 1371248_at | up |
| 1388068_at | down | 1378818_at | down | 1368013_at | up |
| 1374794_at | down | 1378849_at | down | 1369419_at | up |
| 1396214_at | down | 1378862_at | down | 1389475_at | up |
| 1395515_at | down | 1378951_at | down | 1370655_a_at | up |
| 1391619_at | down | 1379012_at | down | 1383597_at | up |
| 1378332_at | down | 1379017_at | down | 1380424_at | up |
| 1368363_at | down | 1379418_at | down | 1367959_a_at | up |
| 1398159_at | down | 1379922_at | down | 1369693_a_at | up |
| 1387882_at | down | 1380155_at | down | 1370467_at | up |
| 1373987_at | down | 1380202_at | down | 1371205_at | up |
| 1389922_at | down | 1380209_at | down | 1387550_a_at | up |
| 1389157_at | down | 1380278_at | down | 1397803_at | up |
| 1387067_at | down | 1380292_at | down | 1380467_at | up |
| 1368493_at | down | 1380914_at | down | 1387567_at | up |
| 1371222_at | down | 1380962_at | down | 1398267_at | up |
| 1386023_at | down | 1381210_at | down | 1369386_at | up |
| 1387149_at | down | 1381237_at | down | 1369160_a_at | up |
| 1370550_at | down | 1381260_at | down | 1379066_at | up |
| 1370733_at | down | 1381472_at | down | 1398457_at | up |
| 1398612_at | down | 1381781_at | down | 1369237_at | up |
| 1378089_at | down | 1382296_at | down | 1368582_at | up |
| 1398467_at | down | 1382700_at | down | 1371201_at | up |
| 1393811_at | down | 1382830_at | down | 1369770_at | up |
| 1382292_at | down | 1383494_at | down | 1368782_at | up |
| 1391629_at | down | 1383637_at | down | 1395985_at | up |
| 1380343_at | down | 1383814_at | down | 1370999_at | up |
| 1397297_at | down | 1384047_at | down | 1392428_at | up |
| 1384852_at | down | 1384592_at | down | 1367692_at | up |
| 1391265_at | down | 1384814_at | down | 1369709_at | up |
| 1367749_at | down | 1386754_at | down | 1372954_at | up |
| 1369120_a_at | down | 1389802_at | down | 1387012_at | up |
| 1370056_at | down | 1389949_at | down | 1370041_at | up |
| 1370369_at | down | 1390364_at | down | 1377489_at | up |
| 1383785_at | down | 1390935_at | down | 1368797_at | up |
| 1378110_at | down | 1391428_at | down | 1385473_x_at | up |
| 1368006_at | down | 1391480_at | down | 1390895_at | up |
| 1389543_at | down | 1391782_at | down | 1398877_at | up |
| 1390585_at | down | 1391922_at | down | 1371296_at | up |
| 1371116_at | down | 1392152_at | down | 1367997_at | up |
| 1369572_at | down | 1392225_at | down | 1370255_at | up |
| 1387162_at | down | 1392317_at | down | 1384664_at | up |
| 1387229_at | down | 1392853_at | down | 1369022_at | up |
| 1378225_at | down | 1393619_at | down | 1381276_at | up |
| 1368595_at | down | 1393792_at | down | 1369482_a_at | up |
| 1368604_at | down | 1394399_at | down | 1391565_at | up |
| 1387439_at | down | 1394425_at | down | 1387073_at | up |
| 1370622_at | down | 1394623_at | down | 1369058_at | up |
| 1395741_at | down | 1394739_at | down | 1368417_at | up |
| 1387158_at | down | 1394945_at | down | 1370514_a_at | up |
| 1369391_at | down | 1394998_at | down | 1394802_at | up |
| 1371117_at | down | 1395759_at | down | 1370515_x_at | up |
| 1380177_at | down | 1395964_at | down | 1370518_a_at | up |
| 1375401_at | down | 1396660_at | down | 1391698_at | up |
| 1388164_at | down | 1396852_at | down | 1369924_at | up |
| 1371003_at | down | 1397004_at | down | 1368752_at | up |
| 1368137_at | down | 1397149_at | down | 1387138_at | up |
| 1367682_at | down | 1397524_at | down | 1387474_at | up |
| 1387160_at | down | 1397559_at | down | 1368484_at | up |
| 1376058_at | down | 1397710_at | down | 1370754_at | up |
| 1380321_at | down | 1398052_at | down | 1393786_at | up |
| 1369102_at | down | 1398098_at | down | 1370626_at | up |
| 1377831_at | down | 1398126_at | down | 1388209_at | up |
| 1393189_at | down | 1374700_at | down | 1388029_at | up |
| 1370831_at | down | 1379091_at | down | 1370593_at | up |
| 1373544_at | down | 1380994_at | down | 1368309_at | up |
| 1371702_at | down | 1389808_at | down | 1370764_a_at | up |
| 1395554_at | down | 1391208_at | down | 1387983_at | up |
| 1369786_at | down | 1391488_at | down | 1387852_at | up |
| 1375476_at | down | 1392021_at | down | 1369750_at | up |
| 1387040_at | down | 1394469_at | down | 1387449_at | up |
| 1368810_a_at | down | 1395769_at | down | 1369919_at | up |
| 1370434_a_at | down | 1396961_at | down | 1397797_at | up |
| 1387181_at | down | 1397422_at | down | 1395337_at | up |
| 1368093_at | down | 1397602_at | down | 1369243_at | up |
| 1398248_s_at | down | 1397624_at | down | 1383613_at | up |
| 1396178_at | down | 1398050_at | down | 1385264_at | up |
| 1368415_at | down | 1398736_at | down | 1385351_at | up |
| 1388157_at | down | 1397667_at | down | 1385490_at | up |
| 1371015_at | down | 1369682_at | down | 1390917_at | up |
| 1369202_at | down | 1380200_at | down | 1395235_at | up |
| 1387283_at | down | 1369853_at | down | 1385012_at | up |
| 1376590_at | down | 1388011_a_at | down | 1394990_at | up |
| 1370827_at | down | 1367570_at | down | 1372979_at | up |
| 1368153_a_at | down | 1370685_at | down | 1373236_at | up |
| 1396249_at | down | 1369059_at | down | 1373315_at | up |
| 1392770_at | down | 1370139_a_at | down | 1373756_at | up |
| 1386964_at | down | 1369535_at | down | 1375012_at | up |
| 1384583_at | down | 1390292_at | down | 1375999_at | up |
| 1369420_at | down | 1373345_at | down | 1376093_at | up |
| 1371212_at | down | 1388149_at | down | 1376422_at | up |
| 1388258_at | down | 1370935_at | down | 1377197_at | up |
| 1369404_a_at | down | 1370198_at | down | 1377687_at | up |
| 1386697_at | down | 1387486_at | down | 1379021_a_at | up |
| 1367845_at | down | 1375615_at | down | 1381234_at | up |
| 1387288_at | down | 1368724_a_at | down | 1382319_at | up |
| 1387208_at | down | 1371339_at | down | 1382557_at | up |
| 1369717_at | down | 1370412_at | down | 1383014_at | up |
| 1387332_at | down | 1368995_at | down | 1383056_a_at | up |
| 1397246_at | down | 1369901_at | down | 1390770_at | up |
| 1393172_at | down | 1387691_at | down | 1391674_at | up |
| 1371483_at | down | 1384842_s_at | down | 1393133_at | up |
| 1387589_at | down | 1368762_at | down | 1394557_at | up |
| 1371052_at | down | 1399143_at | down | 1394926_at | up |
| 1369522_a_at | down | 1387206_at | down | 1395527_at | up |
| 1390426_at | down | 1396536_at | down | 1395562_at | up |
| 1388340_at | down | 1370393_at | down | 1397801_at | up |
| 1369047_at | down | 1383005_at | down | 1398706_at | up |
| 1369679_a_at | down | 1375178_at | down | 1380976_at | up |
| 1388167_at | down | 1368474_at | down | 1395312_at | up |
| 1371202_a_at | down | 1373807_at | down | 1377773_at | up |
| 1385273_at | down | 1377146_at | down | 1379838_at | up |
| 1371162_at | down | 1369098_at | down | 1384398_at | up |
| 1368221_at | down | 1370549_at | down | 1392137_at | up |
| 1368222_at | down | 1368854_at | down | 1396787_at | up |
| 1369007_at | down | 1381446_at | down | 1397470_at | up |
| 1369067_at | down | 1370712_at | down | 1397822_at | up |
| 1387573_a_at | down | 1389560_at | down | 1392185_at | up |
| 1384859_at | down | 1391383_at | down | 1392841_at | up |
| 1388725_at | down | 1387751_at | down | 1380004_at | up |
| 1370632_at | down | 1393829_at | down | 1396857_at | up |
| 1387981_at | down | 1368908_at | down | 1394515_at | up |
| 1397492_at | down | 1394306_at | down | 1396651_at | up |
| 1369109_at | down | 1380416_at | down | 1372337_at | up |
| 1387661_a_at | down | 1368877_at | down | 1381364_at | up |
| 1388025_at | down | 1390003_at | down | 1381916_at | up |
| 1370575_a_at | down | 1370705_at | down | 1398517_at | up |
| 1379003_at | down | 1369464_at | down | 1378123_x_at | up |
| 1369671_at | down | 1371269_at | down | 1382654_at | up |
| 1370572_at | down | 1373776_at | down | 1390564_at | up |
| 1387968_at | down | 1378006_at | down | 1381202_at | up |
| 1378002_at | down | 1378514_at | down | 1395768_at | up |
| 1387197_at | down | 1378771_at | down | 1396220_at | up |
| 1383830_a_at | down | 1379087_at | down | 1396422_at | up |
| 1390923_a_at | down | 1380995_at | down | 1389946_at | up |
| 1368728_at | down | 1381381_at | down | 1378712_at | up |
| 1392837_at | down | 1381563_at | down | 1372236_at | up |
| 1390582_s_at | down | 1381977_at | down | 1396845_at | up |
| 1390772_at | down | 1382095_at | down | 1379621_at | up |
| 1388012_at | down | 1382871_at | down | 1393715_s_at | up |
| 1368554_at | down | 1382951_at | down | 1380950_at | up |
| 1371050_at | down | 1382987_at | down | 1375890_at | up |
| 1371284_at | down | 1384565_at | down | 1371928_at | up |
| 1397789_at | down | 1385112_at | down | 1382409_at | up |
| 1387298_at | down | 1385205_at | down | 1377492_at | up |
| 1370516_at | down | 1385217_at | down | 1396390_at | up |
| 1392715_at | down | 1385481_at | down | 1389398_at | up |
| 1370583_s_at | down | 1385734_at | down | 1385705_at | up |
| 1398309_at | down | 1386114_at | down | 1378302_at | up |
| 1369042_at | down | 1391614_at | down | 1376890_at | up |
| 1387680_at | down | 1391738_at | down | 1373323_at | up |
| 1371219_a_at | down | 1391867_at | down | 1383289_at | up |
| 1369365_at | down | 1392230_at | down | 1396501_at | up |
| 1369157_at | down | 1392354_at | down | 1385276_a_at | up |
| 1371014_at | down | 1392358_at | down | 1382930_at | up |
| 1368165_at | down | 1393662_at | down | 1379051_at | up |
| 1387085_at | down | 1393814_at | down | 1394936_at | up |
| 1368399_a_at | down | 1393867_at | down | 1391028_at | up |
| 1380205_at | down | 1393948_at | down | 1376554_at | up |
| 1391394_s_at | down | 1394989_at | down | 1391318_x_at | up |
| 1387616_at | down | 1396408_at | down | 1378318_at | up |
| 1393782_at | down | 1396427_at | down | 1376717_at | up |
| 1369968_at | down | 1396507_at | down | 1392620_at | up |
| 1370354_at | down | 1396962_at | down | 1393497_at | up |
| 1369269_at | down | 1397210_at | down | 1396922_at | up |
| 1376646_at | down | 1397435_at | down | 1383275_at | up |
| 1388005_at | down | 1397496_at | down | 1385056_at | up |
| 1395102_at | down | 1397835_at | down | 1377122_at | up |
| 1369232_at | down | 1398191_at | down | 1372335_at | up |
| 1369280_at | down | 1385261_s_at | down | 1375924_at | up |
| 1370110_at | down | 1391286_at | down | 1376199_at | up |
| 1369261_at | down | 1396506_at | down | 1379404_at | up |
| 1372929_at | down | 1397776_at | down | 1380359_at | up |
| 1396664_at | down | 1371531_at | down | 1382695_at | up |
| 1369144_a_at | down | 1372060_at | down | 1382791_at | up |
| 1369769_at | down | 1372996_at | down | 1389049_at | up |
| 1387845_at | down | 1373194_at | down | 1391521_at | up |
| 1368343_at | down | 1373368_at | down | 1391874_at | up |
| 1370995_at | down | 1375082_at | down | 1393769_at | up |
| 1387291_at | down | 1375175_at | down | 1397847_at | up |
| 1387281_a_at | down | 1375577_at | down | 1377084_at | up |
| 1387854_at | down | 1377538_at | down | 1393898_at | up |
| 1370944_at | down | 1377578_at | down | 1395459_at | up |
| 1369882_at | down | 1377705_at | down | 1377976_at | up |
| 1387886_at | down | 1377716_at | down | 1378199_at | up |
| 1370636_at | down | 1377991_at | down | 1391641_at | up |
| 1367886_at | down | 1378308_at | down | 1396911_at | up |
| 1375328_at | down | 1378350_at | down | 1391477_at | up |
| 1391320_at | down | 1378484_at | down | 1377984_at | up |
| 1387546_at | down | 1378580_at | down | 1385528_at | up |
| 1387398_at | down | 1378822_at | down | 1383558_at | up |
| 1371600_at | down | 1378882_at | down | 1375068_at | up |
| 1383122_at | down | 1379018_at | down | 1394620_at | up |
| 1393082_at | down | 1379118_at | down | 1391464_at | up |
| 1386968_at | down | 1379233_at | down | 1386554_at | up |
| 1377870_at | down | 1379357_at | down | 1375074_at | up |
| 1367881_at | down | 1379454_at | down | 1378339_at | up |
| 1368589_at | down | 1379602_at | down | 1378206_a_at | up |
| 1388015_at | down | 1379818_at | down | 1392991_at | up |
| 1398449_at | down | 1379946_at | down | 1384547_at | up |
| 1387112_at | down | 1379952_at | down | 1385567_at | up |
| 1367786_at | down | 1380279_at | down | 1394439_at | up |
| 1370186_at | down | 1380581_at | down | 1385704_at | up |
| 1391013_at | down | 1380662_at | down | 1391938_at | up |
| 1390821_at | down | 1380664_at | down | 1377068_at | up |
| 1388551_at | down | 1380669_at | down | 1397534_at | up |
| 1370606_at | down | 1380717_at | down | 1394587_at | up |
| 1369812_at | down | 1380880_at | down | 1372141_at | up |
| 1388889_at | down | 1380916_at | down | 1375345_at | up |
| 1379932_at | down | 1380923_at | down | 1378358_at | up |
| 1369429_at | down | 1381008_at | down | 1396738_at | up |
| 1371058_at | down | 1381066_at | down | 1384518_at | up |
| 1388235_at | down | 1381072_at | down | 1393873_s_at | up |
| 1369004_at | down | 1381115_at | down | 1380993_at | up |
| 1369054_at | down | 1381130_at | down | 1379777_at | up |
| 1387902_a_at | down | 1381188_at | down | 1393565_at | up |
| 1388293_at | down | 1381261_at | down | 1389118_s_at | up |
| 1388277_at | down | 1381299_at | down | 1375403_at | up |
| 1388166_at | down | 1381338_at | down | 1391046_at | up |
| 1388469_at | down | 1381489_at | down | 1381379_at | up |
| 1392569_at | down | 1381699_at | down | 1382044_at | up |
| 1375523_at | down | 1381729_at | down | 1392755_at | up |
| 1371196_at | down | 1381745_at | down | 1396765_at | up |
| 1369353_at | down | 1381791_at | down | 1395976_at | up |
| 1373028_at | down | 1381812_at | down | 1374857_at | up |
| 1368627_at | down | 1381927_at | down | 1375114_at | up |
| 1369957_at | down | 1381950_at | down | 1377193_at | up |
| 1388217_a_at | down | 1382038_at | down | 1374123_at | up |
| 1388185_at | down | 1382277_at | down | 1375738_at | up |
| 1387575_at | down | 1382675_at | down | 1376681_at | up |
| 1372315_at | down | 1382738_at | down | 1376815_at | up |
| 1393026_at | down | 1382761_at | down | 1382484_at | up |
| 1385905_at | down | 1382953_at | down | 1383190_at | up |
| 1396812_at | down | 1382956_at | down | 1390852_x_at | up |
| 1386517_at | down | 1383093_at | down | 1390924_at | up |
| 1392232_at | down | 1383189_at | down | 1395208_at | up |
| 1383768_at | down | 1383195_at | down | 1396569_at | up |
| 1389921_at | down | 1383394_at | down | 1396906_at | up |
| 1369826_at | down | 1383589_at | down | 1397335_at | up |
| 1380746_at | down | 1383642_at | down | 1397580_at | up |
| 1370972_x_at | down | 1383669_at | down | 1397942_at | up |
| 1388254_a_at | down | 1383774_at | down | 1398498_at | up |
| 1381029_at | down | 1383864_at | down | 1392395_at | up |
| 1388283_at | down | 1383955_at | down | 1392976_at | up |
| 1370822_at | down | 1384104_at | down | 1382755_at | up |
| 1377334_at | down | 1384174_at | down | 1396198_at | up |
| 1370883_at | down | 1384231_at | down | 1381830_x_at | up |
| 1370383_s_at | down | 1384276_at | down | 1396230_at | up |
| 1370261_at | down | 1384438_at | down | 1397442_at | up |
| 1387327_at | down | 1384440_at | down | 1373331_at | up |
| 1368252_at | down | 1384534_at | down | 1396929_at | up |
| 1392068_at | down | 1384543_at | down | 1386101_at | up |
| 1390119_at | down | 1384692_at | down | 1383704_at | up |
| 1388021_at | down | 1384738_at | down | 1393724_at | up |
| 1367566_at | down | 1384815_at | down | 1380731_at | up |
| 1370978_at | down | 1384826_a_at | down | 1389499_at | up |
| 1387723_at | down | 1384947_at | down | 1392256_at | up |
| 1368926_at | down | 1384984_at | down | 1391159_at | up |
| 1371143_at | down | 1385054_at | down | 1395212_at | up |
| 1372440_at | down | 1385192_at | down | 1378812_at | up |
| 1381012_at | down | 1385229_at | down | 1392515_at | up |
| 1387131_at | down | 1385231_at | down | 1393599_at | up |
| 1393936_at | down | 1385257_at | down | 1397791_at | up |
| 1369712_at | down | 1385274_at | down | 1374511_at | up |
| 1368751_at | down | 1385291_a_at | down | 1391660_at | up |
| 1369539_at | down | 1385292_at | down | 1379436_a_at | up |
| 1380616_at | down | 1385293_at | down | 1390950_at | up |
| 1368835_at | down | 1385304_at | down | 1397102_at | up |
| 1372757_at | down | 1385363_at | down | 1391624_at | up |
| 1375806_at | down | 1385369_at | down | 1391904_at | up |
| 1374094_at | down | 1385390_at | down | 1381286_at | up |
| 1376013_at | down | 1385416_at | down | 1375774_at | up |
| 1393913_at | down | 1385501_at | down | 1375831_at | up |
| 1377497_at | down | 1385557_at | down | 1381257_at | up |
| 1384021_at | down | 1385593_at | down | 1380351_at | up |
| 1379951_at | down | 1385661_at | down | 1389707_at | up |
| 1380565_at | down | 1385674_at | down | 1393371_at | up |
| 1393573_at | down | 1385731_at | down | 1394422_at | up |
| 1396021_at | down | 1385757_at | down | 1391931_at | up |
| 1388439_at | down | 1385786_at | down | 1393250_at | up |
| 1380391_at | down | 1385836_at | down | 1390623_at | up |
| 1374445_at | down | 1385841_at | down | 1389847_at | up |
| 1394012_at | down | 1385861_x_at | down | 1380472_at | up |
| 1393581_at | down | 1386043_at | down | 1397273_at | up |
| 1396163_at | down | 1386168_at | down | 1392724_at | up |
| 1398206_at | down | 1386183_at | down | 1385722_at | up |
| 1372595_at | down | 1386237_at | down | 1396591_at | up |
| 1373024_at | down | 1386289_at | down | 1391382_at | up |
| 1375587_at | down | 1386331_at | down | 1389960_at | up |
| 1397607_at | down | 1386350_at | down | 1386090_at | up |
| 1394895_at | down | 1386511_x_at | down | 1386584_x_at | up |
| 1383472_at | down | 1386608_at | down | 1378423_at | up |
| 1381599_at | down | 1386621_at | down | 1376847_at | up |
| 1382063_at | down | 1389020_at | down | 1381204_at | up |
| 1375110_at | down | 1389186_at | down | 1391400_at | up |
| 1391907_at | down | 1389977_at | down | 1378403_at | up |
| 1374989_at | down | 1390014_at | down | 1376291_at | up |
| 1384928_at | down | 1390270_at | down | 1395860_at | up |
| 1381223_at | down | 1390309_a_at | down | 1376670_at | up |
| 1395466_at | down | 1390369_at | down | 1397819_at | up |
| 1390956_at | down | 1390509_a_at | down | 1380894_at | up |
| 1373493_at | down | 1391024_at | down | 1395581_at | up |
| 1381504_at | down | 1391145_at | down | 1374908_at | up |
| 1380726_at | down | 1391327_at | down | 1377044_at | up |
| 1392161_at | down | 1391492_at | down | 1389356_at | up |
| 1384623_at | down | 1391551_at | down | 1390663_at | up |
| 1394093_at | down | 1391655_a_at | down | 1378330_at | up |
| 1399042_at | down | 1391809_at | down | 1394660_at | up |
| 1371614_at | down | 1391854_at | down | 1375092_at | up |
| 1382735_at | down | 1392025_x_at | down | 1384849_at | up |
| 1397670_at | down | 1392110_at | down | 1393318_at | up |
| 1383943_at | down | 1392121_at | down | 1375036_at | up |
| 1382130_at | down | 1392172_at | down | 1389095_at | up |
| 1383007_at | down | 1392296_at | down | 1376992_a_at | up |
| 1380572_at | down | 1392336_at | down | 1378272_at | up |
| 1374173_at | down | 1392721_at | down | 1394604_at | up |
| 1382648_at | down | 1392753_at | down | 1377071_at | up |
| 1380463_at | down | 1392954_at | down | 1380289_at | up |
| 1378448_at | down | 1393211_at | down | 1381051_at | up |
| 1378566_at | down | 1393469_at | down | 1391351_at | up |
| 1384320_at | down | 1393502_at | down | 1384522_at | up |
| 1395479_at | down | 1393765_at | down | 1382534_at | up |
| 1380377_at | down | 1393766_at | down | 1384753_at | up |
| 1385086_at | down | 1393840_at | down | 1376869_at | up |
| 1385122_at | down | 1394121_at | down | 1397232_at | up |
| 1391043_at | down | 1394141_at | down | 1391616_at | up |
| 1393632_at | down | 1394167_at | down | 1394460_at | up |
| 1392576_at | down | 1394235_at | down | 1376825_at | up |
| 1374039_at | down | 1394277_at | down | 1390256_at | up |
| 1390822_at | down | 1394349_at | down | 1391207_at | up |
| 1374872_at | down | 1394354_at | down | 1385568_at | up |
| 1389166_at | down | 1394542_at | down | 1385544_at | up |
| 1376287_at | down | 1394611_at | down | 1393978_at | up |
| 1384772_at | down | 1394612_at | down | 1377381_at | up |
| 1371835_at | down | 1394627_at | down | 1394174_at | up |
| 1374942_at | down | 1394757_at | down | 1397321_at | up |
| 1381443_at | down | 1394759_at | down | 1373646_at | up |
| 1375725_at | down | 1394863_at | down | 1389565_at | up |
| 1375060_at | down | 1394901_at | down | 1390227_at | up |
| 1392082_a_at | down | 1394950_at | down | 1384686_at | up |
| 1381972_at | down | 1395025_at | down | 1380903_at | up |
| 1376436_at | down | 1395076_at | down | 1378706_at | up |
| 1397696_at | down | 1395134_at | down | 1393408_at | up |
| 1384879_at | down | 1395179_at | down | 1372502_at | up |
| 1373246_at | down | 1395293_at | down | 1375031_at | up |
| 1379538_at | down | 1395498_at | down | 1379615_at | up |
| 1383717_at | down | 1395597_at | down | 1392202_at | up |
| 1374358_at | down | 1395624_at | down | 1381139_at | up |
| 1390887_at | down | 1395777_at | down | 1393462_at | up |
| 1392838_at | down | 1395800_at | down | 1390979_at | up |
| 1390321_at | down | 1395803_at | down | 1373359_at | up |
| 1384844_at | down | 1395893_at | down | 1373761_at | up |
| 1378468_at | down | 1395899_at | down | 1374269_at | up |
| 1394498_at | down | 1395924_at | down | 1375007_at | up |
| 1379764_at | down | 1396006_at | down | 1375487_at | up |
| 1391980_at | down | 1396208_at | down | 1375509_at | up |
| 1381448_at | down | 1396209_at | down | 1375822_at | up |
| 1382290_at | down | 1396235_at | down | 1376388_at | up |
| 1398553_at | down | 1396239_at | down | 1376455_at | up |
| 1393443_a_at | down | 1396260_at | down | 1376527_at | up |
| 1384777_at | down | 1396269_at | down | 1376719_at | up |
| 1383543_at | down | 1396679_at | down | 1377372_at | up |
| 1381993_at | down | 1396892_at | down | 1378215_at | up |
| 1385128_at | down | 1396950_at | down | 1378314_at | up |
| 1385157_at | down | 1397041_at | down | 1378432_at | up |
| 1397950_at | down | 1397065_at | down | 1378463_at | up |
| 1392646_at | down | 1397123_at | down | 1378785_at | up |
| 1377828_at | down | 1397244_at | down | 1378799_at | up |
| 1382588_at | down | 1397408_at | down | 1378877_at | up |
| 1376090_at | down | 1397544_at | down | 1379009_at | up |
| 1396255_at | down | 1397550_at | down | 1379415_at | up |
| 1388982_at | down | 1397657_at | down | 1379438_at | up |
| 1375026_at | down | 1397772_at | down | 1379534_at | up |
| 1393891_at | down | 1397897_x_at | down | 1379562_at | up |
| 1381328_at | down | 1398014_at | down | 1379983_at | up |
| 1391830_at | down | 1398083_at | down | 1380069_at | up |
| 1393099_at | down | 1398119_at | down | 1380734_at | up |
| 1392836_at | down | 1398494_at | down | 1380851_at | up |
| 1371753_at | down | 1398539_at | down | 1381013_at | up |
| 1384068_at | down | 1398563_at | down | 1381096_at | up |
| 1388299_at | down | 1398567_at | down | 1381425_at | up |
| 1378269_at | down | 1398624_x_at | down | 1381900_at | up |
| 1392273_at | down | 1398628_at | down | 1382665_at | up |
| 1390130_at | down | 1398748_at | down | 1384217_at | up |
| 1388893_at | down | 1399119_at | down | 1384347_at | up |
| 1372847_at | down | AFFX-LysX-5 | down | 1384688_at | up |
| 1396661_at | down | 1378613_at | down | 1384746_at | up |
| 1382153_at | down | 1379496_at | down | 1384870_at | up |
| 1392258_at | down | 1380150_at | down | 1385978_at | up |
| 1397684_at | down | 1380153_at | down | 1386639_at | up |
| 1395981_at | down | 1380912_at | down | 1386749_at | up |
| 1375096_at | down | 1381097_at | down | 1390665_at | up |
| 1372584_at | down | 1381803_at | down | 1390746_at | up |
| 1393611_at | down | 1382505_at | down | 1391009_a_at | up |
| 1393982_at | down | 1382846_at | down | 1391209_at | up |
| 1382976_at | down | 1383039_at | down | 1391233_at | up |
| 1380532_at | down | 1384994_at | down | 1391291_at | up |
| 1396113_at | down | 1385299_at | down | 1391842_at | up |
| 1383067_a_at | down | 1385334_at | down | 1391870_at | up |
| 1377369_at | down | 1385444_at | down | 1392365_at | up |
| 1380922_at | down | 1385630_at | down | 1392757_at | up |
| 1389737_at | down | 1385725_at | down | 1392822_at | up |
| 1392259_at | down | 1391329_at | down | 1393483_at | up |
| 1398420_at | down | 1393484_at | down | 1394559_at | up |
| 1388926_at | down | 1393813_at | down | 1394635_at | up |
| 1375966_at | down | 1394175_at | down | 1394840_at | up |
| 1385945_at | down | 1394930_at | down | 1394938_at | up |
| 1385104_at | down | 1395785_at | down | 1395569_at | up |
| 1396259_at | down | 1395932_at | down | 1395583_at | up |
| 1391462_at | down | 1396065_at | down | 1395660_at | up |
| 1372587_at | down | 1396127_at | down | 1395708_at | up |
| 1377470_at | down | 1396540_at | down | 1395793_at | up |
| 1398689_at | down | 1396559_at | down | 1395845_at | up |
| 1385309_at | down | 1396756_at | down | 1395987_at | up |
| 1391374_at | down | 1397002_at | down | 1396186_at | up |
| 1373590_at | down | 1397434_at | down | 1396296_at | up |
| 1371677_at | down | 1397483_at | down | 1396312_at | up |
| 1389482_at | down | 1398022_at | down | 1396720_at | up |
| 1379954_at | down | 1398089_at | down | 1396778_at | up |
| 1395702_at | down | 1398170_at | down | 1396826_at | up |
| 1391986_at | down | 1398178_at | down | 1396882_at | up |
| 1379354_at | down | 1375070_at | down | 1396908_at | up |
| 1393210_at | down | 1393791_at | down | 1397009_at | up |
| 1393910_at | down | 1397722_at | down | 1397093_at | up |
| 1383657_at | down | 1397922_at | down | 1397616_at | up |
| 1392693_at | down | 1398376_at | down | 1397625_at | up |
| 1378992_at | down | 1372235_at | down | 1397661_at | up |
| 1374283_at | down | 1375086_at | down | 1397682_at | up |
| 1376070_at | down | 1379152_at | down | 1397720_at | up |
| 1379162_at | down | 1380268_at | down | 1376529_at | up |
| 1395578_at | down | 1380953_at | down | 1377242_at | up |
| 1384525_at | down | 1381944_at | down | 1377442_at | up |
| 1377552_at | down | 1382961_at | down | 1377491_at | up |
| 1390348_at | down | 1385023_at | down | 1382697_at | up |
| 1376229_at | down | 1385048_at | down | 1382966_at | up |
| 1375961_at | down | 1385380_at | down | 1385186_at | up |
| 1393914_at | down | 1386158_at | down | 1391029_at | up |
| 1397877_at | down | 1390708_at | down | 1392305_at | up |
| 1388395_at | down | 1391150_at | down | 1394516_at | up |
| 1374683_at | down | 1393976_at | down | 1371459_at | up |
| 1386226_at | down | 1394768_at | down | 1371532_at | up |
| 1385051_at | down | 1395080_at | down | 1371831_at | up |
| 1374449_at | down | 1395606_at | down | 1371927_at | up |
| 1391309_at | down | 1395934_at | down | 1372005_at | up |
| 1391601_at | down | 1396440_at | down | 1372024_at | up |
| 1378100_at | down | 1396550_at | down | 1372268_at | up |
| 1374024_at | down | 1396834_at | down | 1372272_at | up |
| 1380600_at | down | 1397119_at | down | 1372382_at | up |
| 1393121_at | down | 1397457_at | down | 1372616_at | up |
| 1385876_at | down | 1397686_at | down | 1372838_at | up |
| 1373567_at | down | 1397742_at | down | 1372915_at | up |
| 1396407_at | down | 1397917_at | down | 1372992_at | up |
| 1382351_at | down | 1398507_at | down | 1373148_at | up |
| 1383699_at | down | 1377247_at | down | 1373218_at | up |
| 1394746_at | down | 1379216_at | down | 1373522_at | up |
| 1396135_at | down | 1381109_at | down | 1373587_at | up |
| 1395012_at | down | 1384564_at | down | 1373726_at | up |
| 1382826_at | down | 1392429_at | down | 1373789_at | up |
| 1393672_at | down | 1396927_at | up | 1374020_at | up |
| 1380235_at | down | 1369809_at | up | 1374029_at | up |
| 1389015_at | down | 1387741_at | up | 1374091_at | up |
| 1396404_at | down | 1370093_at | up | 1374102_at | up |
| 1377267_at | down | 1387688_at | up | 1374148_at | up |
| 1398630_at | down | 1388070_a_at | up | 1374262_at | up |
| 1384367_at | down | 1368798_at | up | 1374371_at | up |
| 1391311_at | down | 1398036_at | up | 1374546_at | up |
| 1383453_at | down | 1370134_at | up | 1374623_at | up |
| 1392203_at | down | 1369328_at | up | 1374681_at | up |
| 1394851_at | down | 1369765_at | up | 1374688_at | up |
| 1386143_at | down | 1370291_at | up | 1374699_at | up |
| 1382182_at | down | 1369238_at | up | 1374720_at | up |
| 1390667_at | down | 1370534_at | up | 1374755_at | up |
| 1394550_at | down | 1368891_at | up | 1374777_at | up |
| 1385216_at | down | 1379835_at | up | 1374782_at | up |
| 1386057_at | down | 1370584_a_at | up | 1374818_at | up |
| 1380523_at | down | 1380107_a_at | up | 1374881_at | up |
| 1397952_at | down | 1368892_at | up | 1374937_at | up |
| 1372957_at | down | 1367978_at | up | 1374950_at | up |
| 1378904_at | down | 1389492_at | up | 1374955_at | up |
| 1392510_at | down | 1379014_at | up | 1374967_at | up |
| 1379494_at | down | 1391268_at | up | 1374994_at | up |
| 1383490_at | down | 1368534_at | up | 1375001_at | up |
| 1373346_at | down | 1387708_at | up | 1375005_at | up |
| 1384989_at | down | 1387159_at | up | 1375083_at | up |
| 1383900_at | down | 1368718_at | up | 1375265_at | up |
| 1381032_at | down | 1368130_at | up | 1375308_at | up |
| 1395297_at | down | 1368365_at | up | 1375319_at | up |
| 1378072_at | down | 1398310_at | up | 1375392_at | up |
| 1393800_at | down | 1368686_at | up | 1375507_at | up |
| 1380667_at | down | 1369509_a_at | up | 1375588_at | up |
| 1395448_at | down | 1379390_at | up | 1375618_at | up |
| 1382530_at | down | 1371140_a_at | up | 1375624_at | up |
| 1373294_at | down | 1387555_at | up | 1375698_at | up |
| 1379342_at | down | 1388078_a_at | up | 1375733_at | up |
| 1382902_at | down | 1394838_at | up | 1375798_at | up |
| 1397799_at | down | 1378845_at | up | 1375820_at | up |
| 1389779_at | down | 1387425_at | up | 1375957_at | up |
| 1394626_at | down | 1371364_a_at | up | 1376048_at | up |
| 1367510_at | down | 1368569_at | up | 1376141_at | up |
| 1396478_at | down | 1369100_at | up | 1376154_at | up |
| 1385748_at | down | 1368143_at | up | 1376172_at | up |
| 1382507_at | down | 1370435_a_at | up | 1376188_at | up |
| 1389514_at | down | 1389873_at | up | 1376232_at | up |
| 1390870_at | down | 1369683_at | up | 1376361_at | up |
| 1378914_a_at | down | 1386910_a_at | up | 1376377_at | up |
| 1380314_at | down | 1387397_at | up | 1376386_at | up |
| 1380419_at | down | 1368469_at | up | 1376409_at | up |
| 1390142_at | down | 1368776_at | up | 1376453_at | up |
| 1383883_at | down | 1387018_at | up | 1376480_at | up |
| 1375004_at | down | 1369146_a_at | up | 1376541_at | up |
| 1395966_at | down | 1387255_at | up | 1376549_at | up |
| 1374605_at | down | 1375458_at | up | 1376599_at | up |
| 1384263_at | down | 1387925_at | up | 1376710_at | up |
| 1376106_at | down | 1397920_at | up | 1376778_at | up |
| 1396499_at | down | 1370396_x_at | up | 1376820_at | up |
| 1386750_at | down | 1389270_x_at | up | 1376902_x_at | up |
| 1390330_at | down | 1368108_at | up | 1376922_at | up |
| 1374249_at | down | 1368698_at | up | 1376952_at | up |
| 1372657_at | down | 1370602_at | up | 1377075_at | up |
| 1395923_at | down | 1371134_at | up | 1377155_at | up |
| 1385702_at | down | 1383229_at | up | 1377201_at | up |
| 1372709_at | down | 1389886_at | up | 1377226_at | up |
| 1372456_at | down | 1370418_s_at | up | 1377231_at | up |
| 1385043_at | down | 1380092_at | up | 1377240_at | up |
| 1375597_at | down | 1387338_s_at | up | 1377260_at | up |
| 1389176_at | down | 1369611_at | up | 1377266_at | up |
| 1372013_at | down | 1398272_at | up | 1377313_at | up |
| 1373992_at | down | 1388385_at | up | 1377361_at | up |
| 1385842_at | down | 1369430_at | up | 1377370_at | up |
| 1395725_at | down | 1368535_at | up | 1377378_at | up |
| 1394362_at | down | 1374152_at | up | 1377435_at | up |
| 1397184_at | down | 1386774_at | up | 1377472_at | up |
| 1391897_at | down | 1371127_at | up | 1377481_at | up |
| 1393230_s_at | down | 1370649_at | up | 1377510_at | up |
| 1393060_at | down | 1368678_at | up | 1377521_at | up |
| 1398185_at | down | 1387444_at | up | 1377529_at | up |
| 1392505_at | down | 1387934_at | up | 1377533_at | up |
| 1384494_at | down | 1383926_at | up | 1377544_at | up |
| 1372400_at | down | 1385184_at | up | 1377584_at | up |
| 1390897_at | down | 1380317_at | up | 1377629_at | up |
| 1395233_at | down | 1371018_at | up | 1377636_at | up |
| 1377877_at | down | 1387488_a_at | up | 1377658_at | up |
| 1398633_at | down | 1390358_at | up | 1377764_at | up |
| 1394964_at | down | 1367822_at | up | 1377800_at | up |
| 1382265_at | down | 1387846_at | up | 1377853_at | up |
| 1375791_at | down | 1369706_at | up | 1377861_at | up |
| 1385547_at | down | 1369086_a_at | up | 1377973_at | up |
| 1390927_at | down | 1371000_at | up | 1378004_at | up |
| 1396048_at | down | 1370641_s_at | up | 1378060_at | up |
| 1380385_at | down | 1368904_at | up | 1378068_at | up |
| 1393909_at | down | 1369441_at | up | 1378108_at | up |
| 1374373_at | down | 1388176_at | up | 1378113_at | up |
| 1374428_at | down | 1370853_at | up | 1378142_at | up |
| 1377689_at | down | 1368660_at | up | 1378212_at | up |
| 1393092_at | down | 1371129_at | up | 1378265_at | up |
| 1395553_at | down | 1369849_at | up | 1378281_at | up |
| 1396078_at | down | 1369869_at | up | 1378329_at | up |
| 1394068_x_at | down | 1369512_at | up | 1378393_at | up |
| 1384497_at | down | 1391132_at | up | 1378430_at | up |
| 1372443_at | down | 1374275_at | up | 1378450_at | up |
| 1373847_at | down | 1391970_at | up | 1378470_at | up |
| 1396066_at | down | 1368396_at | up | 1378512_at | up |
| 1378676_at | down | 1370352_at | up | 1378537_at | up |
| 1390404_at | down | 1368293_at | up | 1378557_at | up |
| 1373199_at | down | 1368257_at | up | 1378562_at | up |
| 1377283_at | down | 1370125_at | up | 1378589_at | up |
| 1380181_at | down | 1387137_at | up | 1378612_at | up |
| 1383335_at | down | 1371027_at | up | 1378635_at | up |
| 1394561_at | down | 1370528_at | up | 1378678_at | up |
| 1389154_at | down | 1370900_at | up | 1378696_at | up |
| 1383184_at | down | 1371277_at | up | 1378718_at | up |
| 1389059_at | down | 1393323_at | up | 1378725_at | up |
| 1375728_at | down | 1377407_at | up | 1378735_at | up |
| 1381789_at | down | 1381879_x_at | up | 1378763_at | up |
| 1391728_at | down | 1370448_at | up | 1378783_at | up |
| 1381403_at | down | 1370620_at | up | 1378805_at | up |
| 1371875_at | down | 1387655_at | up | 1378833_at | up |
| 1392133_at | down | 1379039_at | up | 1378884_at | up |
| 1382000_at | down | 1368765_at | up | 1378903_at | up |
| 1389433_at | down | 1388175_at | up | 1378946_at | up |
| 1393280_at | down | 1370800_at | up | 1378961_at | up |
| 1385712_at | down | 1387700_at | up | 1378987_at | up |
| 1391857_at | down | 1388191_at | up | 1379038_at | up |
| 1378418_at | down | 1387736_at | up | 1379090_at | up |
| 1375063_at | down | 1369091_at | up | 1379096_at | up |
| 1376014_at | down | 1369143_at | up | 1379110_at | up |
| 1382760_at | down | 1369137_at | up | 1379149_at | up |
| 1393164_at | down | 1387727_at | up | 1379153_at | up |
| 1395982_at | down | 1371233_at | up | 1379160_at | up |
| 1384675_at | down | 1387541_at | up | 1379164_at | up |
| 1381627_at | down | 1393419_at | up | 1379319_at | up |
| 1379780_at | down | 1396150_at | up | 1379379_at | up |
| 1381859_at | down | 1387470_at | up | 1379426_at | up |
| 1374422_at | down | 1369609_at | up | 1379495_at | up |
| 1396256_at | down | 1398878_at | up | 1379503_at | up |
| 1395126_at | down | 1375708_at | up | 1379507_at | up |
| 1376390_at | down | 1368475_at | up | 1379518_at | up |
| 1371541_at | down | 1387199_a_at | up | 1379532_at | up |
| 1382239_at | down | 1371227_at | up | 1379557_at | up |
| 1376789_at | down | 1371109_at | up | 1379586_at | up |
| 1378708_at | down | 1387731_at | up | 1379697_at | up |
| 1377422_at | down | 1391455_at | up | 1379707_at | up |
| 1392330_at | down | 1370185_at | up | 1379711_at | up |
| 1396096_at | down | 1369908_at | up | 1379722_at | up |
| 1381388_at | down | 1387683_at | up | 1379738_at | up |
| 1378264_at | down | 1398289_a_at | up | 1379758_at | up |
| 1384748_at | down | 1369985_at | up | 1379768_at | up |
| 1380008_at | down | 1370279_at | up | 1379789_at | up |
| 1397632_at | down | 1367684_at | up | 1379808_at | up |
| 1396492_at | down | 1374384_at | up | 1379813_at | up |
| 1388943_at | down | 1387978_at | up | 1379852_at | up |
| 1372035_at | down | 1387879_a_at | up | 1379869_at | up |
| 1390454_at | down | 1370693_a_at | up | 1379873_at | up |
| 1395462_at | down | 1370722_at | up | 1379915_at | up |
| 1393144_at | down | 1370345_at | up | 1380051_at | up |
| 1393078_at | down | 1378622_at | up | 1380056_at | up |
| 1373278_at | down | 1370404_at | up | 1380074_at | up |
| 1376112_a_at | down | 1378451_at | up | 1380104_at | up |
| 1392732_at | down | 1375127_at | up | 1380166_at | up |
| 1385280_at | down | 1388055_at | up | 1380176_at | up |
| 1383940_at | down | 1368265_at | up | 1380199_at | up |
| 1374575_at | down | 1370497_at | up | 1380206_at | up |
| 1393764_at | down | 1387305_s_at | up | 1380222_at | up |
| 1390793_at | down | 1391328_at | up | 1380258_at | up |
| 1373911_at | down | 1370633_at | up | 1380299_at | up |
| 1383263_at | down | 1370160_at | up | 1380307_at | up |
| 1378994_at | down | 1398881_at | up | 1380325_at | up |
| 1380631_at | down | 1380438_at | up | 1380342_at | up |
| 1379938_at | down | 1372659_at | up | 1380348_at | up |
| 1376924_a_at | down | 1368619_at | up | 1380397_at | up |
| 1380062_at | down | 1387943_at | up | 1380412_at | up |
| 1391348_at | down | 1370791_at | up | 1380454_at | up |
| 1388895_at | down | 1388238_at | up | 1380481_at | up |
| 1384544_at | down | 1381384_at | up | 1380507_at | up |
| 1395059_at | down | 1387626_at | up | 1380543_at | up |
| 1393646_at | down | 1370443_at | up | 1380554_at | up |
| 1373528_at | down | 1377424_at | up | 1380567_at | up |
| 1382762_at | down | 1369817_at | up | 1380601_at | up |
| 1374616_at | down | 1368282_at | up | 1380612_at | up |
| 1390687_at | down | 1376183_at | up | 1380645_at | up |
| 1379335_at | down | 1385097_at | up | 1380673_at | up |
| 1395100_at | down | 1398276_at | up | 1380686_at | up |
| 1381004_at | down | 1369997_at | up | 1380762_at | up |
| 1375844_at | down | 1370127_at | up | 1380773_at | up |
| 1397965_at | down | 1368664_at | up | 1380801_at | up |
| 1376019_at | down | 1367751_at | up | 1380805_at | up |
| 1385361_at | down | 1368478_at | up | 1380849_at | up |
| 1382470_at | down | 1387597_at | up | 1380863_at | up |
| 1388950_at | down | 1382696_at | up | 1380907_at | up |
| 1379232_at | down | 1368676_at | up | 1380911_at | up |
| 1389447_at | down | 1368649_at | up | 1380918_at | up |
| 1383887_at | down | 1369017_at | up | 1380928_at | up |
| 1377840_at | down | 1382169_x_at | up | 1380946_at | up |
| 1377935_at | down | 1378247_at | up | 1380959_at | up |
| 1397476_at | down | 1369511_at | up | 1380963_at | up |
| 1393055_at | down | 1371158_at | up | 1380975_at | up |
| 1394431_at | down | 1370903_a_at | up | 1380986_at | up |
| 1397948_s_at | down | 1369594_at | up | 1381044_at | up |
| 1384729_at | down | 1368325_at | up | 1381067_at | up |
| 1391056_at | down | 1371100_at | up | 1381094_at | up |
| 1379551_at | down | 1369760_a_at | up | 1381101_at | up |
| 1376619_at | down | 1393427_s_at | up | 1381116_at | up |
| 1384594_at | down | 1388042_at | up | 1381133_at | up |
| 1380333_at | down | 1374402_at | up | 1381147_at | up |
| 1392002_at | down | 1397466_at | up | 1381181_at | up |
| 1380604_at | down | 1377081_at | up | 1381245_at | up |
| 1377550_at | down | 1386997_at | up | 1381250_at | up |
| 1381005_at | down | 1387998_at | up | 1381267_at | up |
| 1394735_at | down | 1373144_at | up | 1381397_at | up |
| 1395421_at | down | 1368182_at | up | 1381422_at | up |
| 1382866_at | down | 1369188_at | up | 1381523_at | up |
| 1372296_at | down | 1389827_at | up | 1381529_at | up |
| 1394682_at | down | 1392820_at | up | 1381540_at | up |
| 1395418_at | down | 1369600_at | up | 1381552_at | up |
| 1385240_at | down | 1370581_at | up | 1381601_at | up |
| 1382028_at | down | 1369606_at | up | 1381617_at | up |
| 1376657_at | down | 1369616_at | up | 1381777_at | up |
| 1379252_at | down | 1391665_at | up | 1381785_at | up |
| 1384259_at | down | 1369314_at | up | 1381792_at | up |
| 1379833_at | down | 1370630_a_at | up | 1381858_at | up |
| 1396231_at | down | 1371179_a_at | up | 1381898_at | up |
| 1378684_at | down | 1388168_a_at | up | 1381912_at | up |
| 1395596_at | down | 1383904_at | up | 1382011_at | up |
| 1382767_at | down | 1389844_at | up | 1382057_at | up |
| 1393249_at | down | 1389759_at | up | 1382089_at | up |
| 1395358_at | down | 1388289_s_at | up | 1382246_at | up |
| 1398582_at | down | 1375607_at | up | 1382256_at | up |
| 1381828_at | down | 1376995_at | up | 1382304_at | up |
| 1375297_at | down | 1397723_s_at | up | 1382383_at | up |
| 1388358_at | down | 1369318_at | up | 1382428_at | up |
| 1376083_at | down | 1374103_at | up | 1382524_at | up |
| 1379206_at | down | 1377796_at | up | 1382529_at | up |
| 1395063_at | down | 1368622_at | up | 1382538_at | up |
| 1371700_at | down | 1392351_at | up | 1382600_at | up |
| 1376161_at | down | 1390936_at | up | 1382645_at | up |
| 1384407_at | down | 1368207_at | up | 1382651_at | up |
| 1392200_a_at | down | 1385607_at | up | 1382681_at | up |
| 1393975_a_at | down | 1369763_at | up | 1382717_at | up |
| 1372780_at | down | 1381904_at | up | 1382799_at | up |
| 1391950_at | down | 1387088_at | up | 1382827_at | up |
| 1375712_at | down | 1370701_at | up | 1382845_at | up |
| 1394155_at | down | 1369904_at | up | 1382899_at | up |
| 1372646_at | down | 1369796_at | up | 1382941_at | up |
| 1398746_at | down | 1368481_at | up | 1382949_at | up |
| 1374291_at | down | 1387595_at | up | 1382985_at | up |
| 1396128_at | down | 1368611_at | up | 1383032_at | up |
| 1377900_at | down | 1369548_at | up | 1383244_at | up |
| 1377928_at | down | 1385517_at | up | 1383293_at | up |
| 1391352_at | down | 1395970_at | up | 1383308_a_at | up |
| 1393737_at | down | 1369699_at | up | 1383318_at | up |
| 1389976_at | down | 1369075_a_at | up | 1383343_at | up |
| 1381572_at | down | 1394514_at | up | 1383409_at | up |
| 1382111_at | down | 1388016_a_at | up | 1383456_at | up |
| 1392958_at | down | 1368401_at | up | 1383476_at | up |
| 1385448_at | down | 1371013_at | up | 1383491_at | up |
| 1392506_at | down | 1369128_at | up | 1383521_at | up |
| 1389191_at | down | 1369745_at | up | 1383526_at | up |
| 1372282_at | down | 1368951_at | up | 1383683_at | up |
| 1394414_at | down | 1371236_at | up | 1383783_at | up |
| 1375679_at | down | 1396468_at | up | 1383811_at | up |
| 1397730_at | down | 1369510_at | up | 1383988_at | up |
| 1379709_at | down | 1369560_at | up | 1384003_at | up |
| 1384627_at | down | 1371363_at | up | 1384025_at | up |
| 1384710_at | down | 1379334_at | up | 1384080_at | up |
| 1373026_at | down | 1391785_at | up | 1384215_at | up |
| 1372553_at | down | 1369659_at | up | 1384226_at | up |
| 1379124_at | down | 1387535_at | up | 1384284_at | up |
| 1377620_at | down | 1369118_a_at | up | 1384322_at | up |
| 1373891_at | down | 1387465_a_at | up | 1384393_at | up |
| 1386254_at | down | 1372159_at | up | 1384410_at | up |
| 1374906_at | down | 1391038_at | up | 1384459_at | up |
| 1380118_at | down | 1369411_at | up | 1384491_at | up |
| 1372682_at | down | 1380421_s_at | up | 1384516_at | up |
| 1383807_at | down | 1369494_a_at | up | 1384546_at | up |
| 1378541_at | down | 1370423_at | up | 1384569_at | up |
| 1381084_at | down | 1388057_a_at | up | 1384593_at | up |
| 1389553_at | down | 1379552_s_at | up | 1384598_at | up |
| 1388996_at | down | 1370148_at | up | 1384630_at | up |
| 1384765_at | down | 1370018_at | up | 1384649_at | up |
| 1395372_at | down | 1387047_at | up | 1384663_at | up |
| 1389295_at | down | 1368247_at | up | 1384744_at | up |
| 1388625_at | down | 1370912_at | up | 1384770_at | up |
| 1377053_at | down | 1385541_at | up | 1384868_at | up |
| 1380460_at | down | 1367903_at | up | 1384892_at | up |
| 1396325_at | down | 1387506_at | up | 1385169_at | up |
| 1380175_at | down | 1369289_at | up | 1385196_at | up |
| 1386811_at | down | 1387096_at | up | 1385221_at | up |
| 1381973_at | down | 1387996_a_at | up | 1385281_at | up |
| 1393198_at | down | 1386897_at | up | 1385333_at | up |
| 1380754_at | down | 1398725_at | up | 1385345_at | up |
| 1395701_at | down | 1387307_at | up | 1385364_at | up |
| 1375590_at | down | 1377499_a_at | up | 1385377_at | up |
| 1374874_at | down | 1391301_at | up | 1385381_at | up |
| 1391637_at | down | 1382224_at | up | 1385549_at | up |
| 1391787_at | down | 1368873_at | up | 1385560_at | up |
| 1394934_at | down | 1369028_at | up | 1385732_at | up |
| 1396932_at | down | 1399163_a_at | up | 1385749_at | up |
| 1394494_at | down | 1368872_a_at | up | 1385892_at | up |
| 1373161_at | down | 1369622_at | up | 1385910_at | up |
| 1386378_at | down | 1389132_at | up | 1385940_at | up |
| 1397227_at | down | 1370643_a_at | up | 1385972_at | up |
| 1372507_at | down | 1370487_a_at | up | 1385997_at | up |
| 1373970_at | down | 1370395_at | up | 1386008_at | up |
| 1389581_at | down | 1370710_at | up | 1386053_at | up |
| 1393463_at | down | 1381955_at | up | 1386551_at | up |
| 1378174_at | down | 1393386_at | up | 1386658_at | up |
| 1378193_at | down | 1382200_at | up | 1386709_at | up |
| 1397333_at | down | 1389807_at | up | 1388606_at | up |
| 1383794_at | down | 1380605_at | up | 1388671_at | up |
| 1395435_at | down | 1392303_at | up | 1388736_at | up |
| 1372741_at | down | 1381941_at | up | 1388811_at | up |
| 1395585_at | down | 1374167_at | up | 1388987_at | up |
| 1373931_at | down | 1375327_at | up | 1389161_at | up |
| 1371805_at | down | 1382929_at | up | 1389212_at | up |
| 1390550_at | down | 1392932_at | up | 1389281_at | up |
| 1395739_at | down | 1376044_at | up | 1389376_at | up |
| 1395133_at | down | 1395603_at | up | 1389417_at | up |
| 1384858_a_at | down | 1373006_at | up | 1389471_at | up |
| 1388984_at | down | 1392160_at | up | 1389512_at | up |
| 1385427_at | down | 1397513_at | up | 1389550_at | up |
| 1380498_at | down | 1384880_at | up | 1389584_at | up |
| 1396035_at | down | 1382293_at | up | 1389593_at | up |
| 1390271_at | down | 1392227_at | up | 1389751_at | up |
| 1391277_at | down | 1397860_at | up | 1389798_at | up |
| 1372852_at | down | 1376369_at | up | 1389842_at | up |
| 1389437_at | down | 1380519_at | up | 1389959_at | up |
| 1397334_at | down | 1375014_at | up | 1390018_at | up |
| 1397946_at | down | 1377186_at | up | 1390072_at | up |
| 1397765_at | down | 1384221_at | up | 1390094_at | up |
| 1385511_at | down | 1383481_at | up | 1390216_a_at | up |
| 1383701_at | down | 1376269_at | up | 1390222_at | up |
| 1391566_at | down | 1383958_at | up | 1390240_at | up |
| 1375818_at | down | 1382858_at | up | 1390317_at | up |
| 1375364_at | down | 1397735_at | up | 1390349_at | up |
| 1386817_at | down | 1374944_at | up | 1390370_at | up |
| 1376486_at | down | 1384066_at | up | 1390402_at | up |
| 1389575_at | down | 1380149_at | up | 1390428_at | up |
| 1367476_at | down | 1392563_at | up | 1390496_at | up |
| 1389420_at | down | 1376355_at | up | 1390581_at | up |
| 1392957_at | down | 1378875_a_at | up | 1390610_at | up |
| 1392642_at | down | 1370486_a_at | up | 1390644_at | up |
| 1395990_at | down | 1367943_at | up | 1390675_at | up |
| 1389344_at | down | 1382328_at | up | 1390686_at | up |
| 1380105_at | down | 1369016_at | up | 1390703_at | up |
| 1393875_at | down | 1397216_at | up | 1390716_at | up |
| 1383319_at | down | 1368803_at | up | 1390792_a_at | up |
| 1389173_at | down | 1388252_at | up | 1390872_at | up |
| 1371779_at | down | 1371206_a_at | up | 1390932_at | up |
| 1382451_at | down | 1398322_at | up | 1390974_a_at | up |
| 1384843_at | down | 1396152_s_at | up | 1391027_at | up |
| 1374892_at | down | 1370937_a_at | up | 1391103_at | up |
| 1397828_at | down | 1370750_a_at | up | 1391118_at | up |
| 1377070_at | down | 1369315_at | up | 1391122_at | up |
| 1390919_at | down | 1376445_at | up | 1391147_at | up |
| 1381073_at | down | 1368763_at | up | 1391153_at | up |
| 1384973_at | down | 1369191_at | up | 1391173_at | up |
| 1398440_at | down | 1369208_at | up | 1391197_at | up |
| 1392813_at | down | 1369697_at | up | 1391219_at | up |
| 1392231_at | down | 1369433_at | up | 1391275_at | up |
| 1381671_at | down | 1385386_at | up | 1391298_at | up |
| 1373017_at | down | 1377778_at | up | 1391308_at | up |
| 1373000_at | down | 1369681_at | up | 1391334_at | up |
| 1384437_at | down | 1394886_at | up | 1391445_at | up |
| 1381691_at | down | 1388872_at | up | 1391473_at | up |
| 1380630_at | down | 1395311_at | up | 1391496_at | up |
| 1379791_at | down | 1388593_at | up | 1391513_at | up |
| 1377874_at | down | 1368749_at | up | 1391556_at | up |
| 1381070_at | down | 1370289_x_at | up | 1391586_at | up |
| 1384787_at | down | 1381156_at | up | 1391620_at | up |
| 1383140_at | down | 1368521_at | up | 1391638_at | up |
| 1384526_at | down | 1370499_at | up | 1391647_at | up |
| 1383276_at | down | 1370694_at | up | 1391730_at | up |
| 1390948_at | down | 1388034_at | up | 1391760_at | up |
| 1375057_at | down | 1378891_at | up | 1391778_at | up |
| 1390878_at | down | 1382787_at | up | 1391825_at | up |
| 1372426_at | down | 1369380_at | up | 1391831_at | up |
| 1397692_at | down | 1371095_at | up | 1391992_at | up |
| 1393866_at | down | 1369361_at | up | 1392004_at | up |
| 1382531_at | down | 1388971_s_at | up | 1392034_at | up |
| 1397609_at | down | 1390076_at | up | 1392050_at | up |
| 1393809_at | down | 1368755_at | up | 1392097_at | up |
| 1398987_at | down | 1370781_a_at | up | 1392106_at | up |
| 1383996_at | down | 1369603_at | up | 1392156_at | up |
| 1380066_at | down | 1370773_a_at | up | 1392167_at | up |
| 1382146_at | down | 1368915_at | up | 1392194_at | up |
| 1376647_at | down | 1387838_at | up | 1392222_at | up |
| 1389609_at | down | 1384395_at | up | 1392270_at | up |
| 1378052_at | down | 1368811_at | up | 1392288_at | up |
| 1392496_at | down | 1368812_at | up | 1392314_at | up |
| 1379135_at | down | 1395354_at | up | 1392318_at | up |
| 1374887_at | down | 1367887_at | up | 1392338_at | up |
| 1385835_at | down | 1368848_at | up | 1392363_at | up |
| 1377353_a_at | down | 1381374_at | up | 1392373_at | up |
| 1379677_at | down | 1389982_at | up | 1392434_at | up |
| 1393591_at | down | 1370466_at | up | 1392442_at | up |
| 1390487_at | down | 1370698_at | up | 1392489_at | up |
| 1388502_at | down | 1395683_at | up | 1392596_at | up |
| 1390345_at | down | 1393892_at | up | 1392649_at | up |
| 1375397_at | down | 1378273_at | up | 1392657_at | up |
| 1381310_at | down | 1393812_at | up | 1392661_at | up |
| 1377941_at | down | 1374520_at | up | 1392713_a_at | up |
| 1389216_at | down | 1379655_at | up | 1392782_at | up |
| 1390099_at | down | 1374576_at | up | 1392794_at | up |
| 1397274_at | down | 1381104_at | up | 1392808_at | up |
| 1395054_at | down | 1376064_at | up | 1392850_at | up |
| 1395943_at | down | 1383218_at | up | 1392921_at | up |
| 1398373_at | down | 1380629_at | up | 1392968_at | up |
| 1390775_at | down | 1382729_at | up | 1393013_at | up |
| 1372078_at | down | 1375167_at | up | 1393024_at | up |
| 1392168_at | down | 1384918_at | up | 1393030_at | up |
| 1379173_at | down | 1396982_at | up | 1393058_at | up |
| 1380499_at | down | 1380990_at | up | 1393083_at | up |
| 1384964_at | down | 1385770_at | up | 1393124_at | up |
| 1396897_at | down | 1393931_at | up | 1393140_at | up |
| 1375122_at | down | 1378136_at | up | 1393169_at | up |
| 1391472_at | down | 1371145_at | up | 1393220_at | up |
| 1379200_at | down | 1384766_a_at | up | 1393283_at | up |
| 1390427_at | down | 1383364_at | up | 1393355_at | up |
| 1393564_at | down | 1369300_at | up | 1393405_at | up |
| 1391307_at | down | 1383320_at | up | 1393531_at | up |
| 1379589_at | down | 1379004_at | up | 1393542_at | up |
| 1393395_at | down | 1392872_at | up | 1393608_at | up |
| 1394074_at | down | 1375128_at | up | 1393616_at | up |
| 1379578_at | down | 1398816_at | up | 1393626_at | up |
| 1371520_at | down | 1390230_at | up | 1393816_at | up |
| 1383053_x_at | down | 1381821_at | up | 1393965_at | up |
| 1380529_at | down | 1373681_at | up | 1393971_at | up |
| 1397132_at | down | 1369170_at | up | 1394028_at | up |
| 1391255_at | down | 1376525_at | up | 1394057_at | up |
| 1371638_at | down | 1370405_at | up | 1394156_at | up |
| 1375984_at | down | 1372422_at | up | 1394202_x_at | up |
| 1381902_at | down | 1370416_at | up | 1394313_at | up |
| 1381373_at | down | 1368756_at | up | 1394359_at | up |
| 1382649_at | down | 1371168_at | up | 1394396_at | up |
| 1387319_at | down | 1370668_a_at | up | 1394402_at | up |
| 1370165_at | down | 1369218_at | up | 1394410_at | up |
| 1368051_at | down | 1388267_a_at | up | 1394443_at | up |
| 1394163_at | down | 1370124_at | up | 1394457_at | up |
| 1383600_at | down | 1376713_at | up | 1394468_at | up |
| 1394983_at | down | 1394704_at | up | 1394475_at | up |
| 1371040_at | down | 1376289_at | up | 1394507_at | up |
| 1387932_at | down | 1369277_at | up | 1394532_at | up |
| 1371130_at | down | 1398174_at | up | 1394632_at | up |
| 1368745_at | down | 1368020_at | up | 1394655_at | up |
| 1370468_at | down | 1371087_a_at | up | 1394669_at | up |
| 1370494_at | down | 1368364_at | up | 1394765_at | up |
| 1369132_at | down | 1394722_at | up | 1394769_at | up |
| 1368460_at | down | 1378751_at | up | 1394776_at | up |
| 1387093_at | down | 1398297_at | up | 1394821_at | up |
| 1368296_at | down | 1370985_at | up | 1394915_at | up |
| 1391516_at | down | 1375355_at | up | 1394942_at | up |
| 1369099_at | down | 1391612_at | up | 1394967_at | up |
| 1383632_at | down | 1390881_at | up | 1394986_at | up |
| 1370610_at | down | 1370926_at | up | 1394994_at | up |
| 1369756_a_at | down | 1369062_at | up | 1395017_at | up |
| 1387372_at | down | 1369330_at | up | 1395031_at | up |
| 1387542_at | down | 1369245_at | up | 1395048_at | up |
| 1370667_at | down | 1398257_at | up | 1395085_at | up |
| 1387584_at | down | 1368861_a_at | up | 1395091_at | up |
| 1369469_s_at | down | 1368263_a_at | up | 1395121_at | up |
| 1386596_at | down | 1393752_at | up | 1395140_at | up |
| 1368991_at | down | 1368310_at | up | 1395161_at | up |
| 1382691_at | down | 1381219_at | up | 1395168_at | up |
| 1368992_a_at | down | 1388040_a_at | up | 1395239_at | up |
| 1384817_at | down | 1367928_at | up | 1395247_at | up |
| 1369551_at | down | 1395485_s_at | up | 1395253_at | up |
| 1387623_at | down | 1387744_at | up | 1395288_at | up |
| 1368157_at | down | 1371259_at | up | 1395304_at | up |
| 1378237_at | down | 1398499_at | up | 1395309_at | up |
| 1369721_at | down | 1386948_at | up | 1395321_at | up |
| 1392986_at | down | 1398269_at | up | 1395351_at | up |
| 1370036_at | down | 1386263_at | up | 1395445_at | up |
| 1369531_at | down | 1368939_a_at | up | 1395472_at | up |
| 1368733_at | down | 1375765_at | up | 1395599_at | up |
| 1383399_at | down | 1371192_at | up | 1395608_at | up |
| 1392903_at | down | 1369205_at | up | 1395626_at | up |
| 1370070_at | down | 1387515_at | up | 1395631_at | up |
| 1388586_at | down | 1369436_at | up | 1395657_at | up |
| 1368499_at | down | 1368255_at | up | 1395676_at | up |
| 1387614_at | down | 1369730_a_at | up | 1395688_at | up |
| 1368276_at | down | 1368972_at | up | 1395731_at | up |
| 1368865_at | down | 1387267_at | up | 1395740_at | up |
| 1368864_at | down | 1387735_at | up | 1395763_at | up |
| 1370766_at | down | 1387413_at | up | 1395784_at | up |
| 1384716_at | down | 1398049_at | up | 1395812_at | up |
| 1369046_at | down | 1369987_at | up | 1395843_at | up |
| 1391036_at | down | 1375522_at | up | 1395907_at | up |
| 1368352_at | down | 1384389_at | up | 1395931_at | up |
| 1398245_at | down | 1387635_at | up | 1395942_at | up |
| 1368429_at | down | 1388132_at | up | 1395952_at | up |
| 1387628_at | down | 1368966_at | up | 1395961_at | up |
| 1381464_at | down | 1387480_at | up | 1396064_at | up |
| 1390627_a_at | down | 1377049_at | up | 1396073_at | up |
| 1393160_at | down | 1384303_s_at | up | 1396121_at | up |
| 1377591_at | down | 1389908_at | up | 1396126_at | up |
| 1388275_at | down | 1381213_at | up | 1396134_at | up |
| 1384045_at | down | 1368488_at | up | 1396184_at | up |
| 1387069_a_at | down | 1380989_at | up | 1396213_at | up |
| 1369499_at | down | 1367919_at | up | 1396241_at | up |
| 1369784_at | down | 1395863_at | up | 1396271_at | up |
| 1371084_at | down | 1368476_at | up | 1396294_at | up |
| 1388650_at | down | 1369217_at | up | 1396305_at | up |
| 1368840_at | down | 1387048_at | up | 1396310_at | up |
| 1371122_at | down | 1369442_at | up | 1396318_at | up |
| 1393663_at | down | 1380691_at | up | 1396330_at | up |
| 1384820_at | down | 1370711_a_at | up | 1396387_at | up |
| 1385191_at | down | 1394832_at | up | 1396514_at | up |
| 1395325_s_at | down | 1374951_at | up | 1396523_at | up |
| 1397205_at | down | 1380494_at | up | 1396533_at | up |
| 1395230_at | down | 1388014_at | up | 1396539_at | up |
| 1395690_at | down | 1387927_a_at | up | 1396547_at | up |
| 1397396_at | down | 1387724_at | up | 1396558_at | up |
| 1372114_at | down | 1370763_at | up | 1396573_at | up |
| 1372331_at | down | 1388092_at | up | 1396579_at | up |
| 1373912_at | down | 1370761_at | up | 1396590_at | up |
| 1374286_at | down | 1388226_at | up | 1396609_at | up |
| 1374666_at | down | 1371034_at | up | 1396640_at | up |
| 1374862_at | down | 1387102_at | up | 1396647_at | up |
| 1375555_at | down | 1375557_at | up | 1396657_at | up |
| 1375797_at | down | 1397739_at | up | 1396693_at | up |
| 1377736_at | down | 1368496_at | up | 1396698_at | up |
| 1378351_at | down | 1381509_at | up | 1396705_at | up |
| 1378668_at | down | 1368312_at | up | 1396712_at | up |
| 1381057_at | down | 1371220_at | up | 1396718_at | up |
| 1382511_at | down | 1387992_at | up | 1396763_at | up |
| 1383112_at | down | 1375485_at | up | 1396776_at | up |
| 1383139_at | down | 1384234_at | up | 1396784_at | up |
| 1389726_at | down | 1374477_at | up | 1396808_at | up |
| 1394130_at | down | 1384325_at | up | 1396831_at | up |
| 1395027_at | down | 1392112_at | up | 1396841_at | up |
| 1397977_at | down | 1387516_at | up | 1396849_at | up |
| 1374054_at | down | 1369196_at | up | 1396864_at | up |
| 1374721_at | down | 1369030_at | up | 1396874_at | up |
| 1392431_at | down | 1392342_at | up | 1396880_at | up |
| 1393716_at | down | 1369138_a_at | up | 1396888_at | up |
| 1395953_at | down | 1381221_at | up | 1396935_at | up |
| 1381915_at | down | 1398614_at | up | 1396939_at | up |
| 1382391_at | down | 1373045_at | up | 1396955_at | up |
| 1392053_at | down | 1375727_at | up | 1396967_at | up |
| 1376749_at | down | 1370502_at | up | 1396971_at | up |
| 1385248_a_at | down | 1378745_at | up | 1396988_at | up |
| 1388843_at | down | 1367969_at | up | 1397001_at | up |
| 1384460_at | down | 1367885_at | up | 1397006_at | up |
| 1392783_at | down | 1379361_at | up | 1397016_at | up |
| 1385143_at | down | 1387740_at | up | 1397028_at | up |
| 1380287_at | down | 1370647_at | up | 1397035_at | up |
| 1396652_at | down | 1369719_at | up | 1397056_at | up |
| 1382683_a_at | down | 1378893_at | up | 1397074_at | up |
| 1394732_at | down | 1397572_at | up | 1397087_at | up |
| 1393273_at | down | 1368750_a_at | up | 1397116_at | up |
| 1394453_at | down | 1391902_at | up | 1397126_at | up |
| 1386310_at | down | 1395839_at | up | 1397143_at | up |
| 1393108_at | down | 1387483_at | up | 1397169_at | up |
| 1373695_at | down | 1370679_at | up | 1397208_at | up |
| 1395336_at | down | 1387384_at | up | 1397252_at | up |
| 1397832_at | down | 1381490_at | up | 1397271_at | up |
| 1386258_at | down | 1374399_at | up | 1397280_at | up |
| 1372381_at | down | 1387063_at | up | 1397294_at | up |
| 1382748_at | down | 1395617_at | up | 1397298_at | up |
| 1376595_at | down | 1379374_at | up | 1397318_at | up |
| 1390520_at | down | 1371099_at | up | 1397348_at | up |
| 1378748_at | down | 1394736_at | up | 1397388_at | up |
| 1374912_at | down | 1399021_at | up | 1397421_at | up |
| 1392243_at | down | 1387441_at | up | 1397433_at | up |
| 1382279_at | down | 1398313_a_at | up | 1397489_at | up |
| 1375715_at | down | 1387264_at | up | 1397593_at | up |
| 1381002_at | down | 1369782_a_at | up | 1397597_at | up |
| 1376169_at | down | 1391007_s_at | up | 1397605_at | up |
| 1381355_at | down | 1370970_at | up | 1397614_at | up |
| 1394270_at | down | 1387467_at | up | 1397650_at | up |
| 1394408_at | down | 1369795_at | up | 1397680_at | up |
| 1376258_at | down | 1368793_at | up | 1397687_at | up |
| 1388447_at | down | 1369741_at | up | 1397707_at | up |
| 1398363_at | down | 1369742_at | up | 1397718_at | up |
| 1383792_at | down | 1369334_at | up | 1397774_at | up |
| 1378857_at | down | 1370472_a_at | up | 1397837_at | up |
| 1374768_at | down | 1369767_a_at | up | 1397914_at | up |
| 1376382_at | down | 1369687_at | up | 1397980_at | up |
| 1398127_at | down | 1369802_at | up | 1397994_at | up |
| 1375302_at | down | 1388173_at | up | 1398015_at | up |
| 1379806_at | down | 1387476_at | up | 1398021_at | up |
| 1372147_at | down | 1369145_a_at | up | 1398029_at | up |
| 1386106_at | down | 1369133_a_at | up | 1398034_at | up |
| 1391061_at | down | 1369134_x_at | up | 1398040_at | up |
| 1397329_at | down | 1369350_at | up | 1398123_at | up |
| 1373895_at | down | 1369762_at | up | 1398128_at | up |
| 1377611_at | down | 1369761_at | up | 1398140_at | up |
| 1377734_at | down | 1387677_at | up | 1398149_at | up |
| 1382630_at | down | 1369236_at | up | 1398215_at | up |
| 1390181_at | down | 1396262_at | up | 1398222_at | up |
| 1391026_at | down | 1370336_at | up | 1398227_at | up |
| 1393751_at | down | 1387836_at | up | 1398232_at | up |
| 1394862_at | down | 1391059_at | up | 1398239_at | up |
| 1397261_at | down | 1371092_at | up | 1398403_at | up |
| 1397498_at | down | 1398271_at | up | 1398428_at | up |
| 1394707_at | down | 1395805_at | up | 1398504_at | up |
| 1396891_at | down | 1398321_a_at | up | 1398532_at | up |
| 1391878_at | down | 1397059_x_at | up | 1398544_at | up |
| 1373725_at | down | 1370227_at | up | 1398556_at | up |
| 1393324_at | down | 1387939_at | up | 1398658_at | up |
| 1385914_at | down | 1390855_at | up | 1398711_at | up |
| 1375239_at | down | 1370187_at | up | 1372025_at | up |
| 1379882_a_at | down | 1367835_at | up | 1373357_at | up |
| 1398112_at | down | 1388214_at | up | 1374655_at | up |
| 1393373_at | down | 1373057_at | up | 1374927_at | up |
| 1395587_at | down | 1367851_at | up | 1376318_at | up |
| 1376225_at | down | 1381806_at | up | 1376923_at | up |
| 1371450_at | down | 1367554_at | up | 1376993_at | up |
| 1375661_at | down | 1388123_at | up | 1377017_at | up |
| 1390453_at | down | 1381794_at | up | 1377028_at | up |
| 1396277_at | down | 1391768_at | up | 1377057_at | up |
| 1376582_at | down | 1398869_at | up | 1377419_at | up |
| 1393622_at | down | 1368184_at | up | 1377441_at | up |
| 1394493_at | down | 1386930_at | up | 1377454_at | up |
| 1397888_at | down | 1369105_a_at | up | 1377946_at | up |
| 1379637_at | down | 1387459_at | up | 1378213_at | up |
| 1395405_at | down | 1390720_at | up | 1378376_at | up |
| 1390095_at | down | 1378769_at | up | 1378502_at | up |
| 1397367_at | down | 1383421_at | up | 1378707_at | up |
| 1374635_at | down | 1368982_at | up | 1378774_at | up |
| 1376393_at | down | 1373952_at | up | 1378846_at | up |
| 1389874_at | down | 1389662_at | up | 1378920_at | up |
| 1392026_at | down | 1373257_at | up | 1378966_at | up |
| 1384962_at | down | 1389855_at | up | 1378976_x_at | up |
| 1391280_at | down | 1369297_at | up | 1379085_at | up |
| 1380387_at | down | 1398469_at | up | 1379097_at | up |
| 1376034_at | down | 1394681_at | up | 1379129_at | up |
| 1383917_at | down | 1368421_at | up | 1379508_at | up |
| 1378369_at | down | 1368516_at | up | 1379739_at | up |
| 1394906_at | down | 1371115_at | up | 1379984_at | up |
| 1391663_at | down | 1387537_at | up | 1380007_at | up |
| 1396761_at | down | 1384485_at | up | 1380352_at | up |
| 1379268_at | down | 1368956_at | up | 1380508_at | up |
| 1377733_at | down | 1393671_at | up | 1380756_at | up |
| 1383858_at | down | 1377171_at | up | 1381045_at | up |
| 1375614_at | down | 1385148_at | up | 1381105_at | up |
| 1373820_at | down | 1368226_at | up | 1381182_at | up |
| 1383129_at | down | 1370659_at | up | 1381491_at | up |
| 1393996_at | down | 1388050_at | up | 1381510_at | up |
| 1384661_at | down | 1370653_at | up | 1381533_at | up |
| 1375799_at | down | 1370664_a_at | up | 1381574_at | up |
| 1392855_x_at | down | 1369543_s_at | up | 1381635_at | up |
| 1382185_at | down | 1370565_at | up | 1381761_at | up |
| 1390812_a_at | down | 1397620_at | up | 1381778_at | up |
| 1384160_at | down | 1386917_at | up | 1381887_x_at | up |
| 1398645_at | down | 1379447_at | up | 1382587_at | up |
| 1382426_at | down | 1387263_at | up | 1382900_at | up |
| 1378227_at | down | 1375344_at | up | 1382965_at | up |
| 1379428_x_at | down | 1395727_at | up | 1383457_at | up |
| 1390242_at | down | 1387176_at | up | 1383598_at | up |
| 1393539_at | down | 1369564_at | up | 1385826_at | up |
| 1376660_at | down | 1369471_at | up | 1389799_at | up |
| 1378421_at | down | 1375406_s_at | up | 1389887_at | up |
| 1379548_at | down | 1382623_at | up | 1389909_at | up |
| 1381342_at | down | 1370394_at | up | 1389937_at | up |
| 1389601_at | down | 1396672_at | up | 1390086_at | up |
| 1389797_at | down | 1372290_at | up | 1390645_at | up |
| 1393192_at | down | 1387411_at | up | 1390882_at | up |
| 1398513_at | down | 1387209_at | up | 1391299_at | up |
| 1375237_at | down | 1378461_at | up | 1391358_at | up |
| 1378205_at | down | 1367904_at | up | 1391717_at | up |
| 1377702_at | down | 1387403_at | up | 1391832_at | up |
| 1393232_at | down | 1381561_at | up | 1391843_at | up |
| 1391633_at | down | 1396867_at | up | 1392005_at | up |
| 1376779_at | down | 1386486_at | up | 1392304_at | up |
| 1376515_at | down | 1370989_at | up | 1392795_at | up |
| 1377762_at | down | 1378217_at | up | 1392977_at | up |
| 1377632_at | down | 1368112_at | up | 1393537_at | up |
| 1390766_at | down | 1381892_at | up | 1393761_at | up |
| 1385571_at | down | 1390079_at | up | 1393835_s_at | up |
| 1378533_at | down | 1370038_at | up | 1394058_at | up |
| 1375601_at | down | 1368638_at | up | 1394533_at | up |
| 1380576_at | down | 1376487_at | up | 1394564_at | up |
| 1389892_at | down | 1387379_at | up | 1394777_at | up |
| 1378228_at | down | 1370021_at | up | 1394961_at | up |
| 1382857_at | down | 1375375_at | up | 1394987_at | up |
| 1374314_at | down | 1398764_at | up | 1394995_at | up |
| 1384914_at | down | 1367597_at | up | 1395423_at | up |
| 1395443_at | down | 1389882_at | up | 1395648_at | up |
| 1393279_at | down | 1375202_at | up | 1395764_at | up |
| 1396244_at | down | 1370356_at | up | 1395836_at | up |
| 1382493_at | down | 1377486_at | up | 1396088_at | up |
| 1375729_at | down | 1385599_at | up | 1396251_at | up |
| 1393109_at | down | 1370742_at | up | 1396580_at | up |
| 1393528_at | down | 1369110_x_at | up | 1396648_at | up |
| 1395073_at | down | 1387839_at | up | 1396673_at | up |
| 1395494_at | down | 1371267_at | up | 1397007_at | up |
| 1375672_at | down | 1390946_at | up | 1397021_at | up |
| 1391569_at | down | 1375479_at | up | 1397094_at | up |
| 1392663_at | down | 1387252_at | up | 1397175_at | up |
| 1374675_at | down | 1383266_at | up | 1397389_at | up |
| 1375902_at | down | 1387257_at | up | 1397606_at | up |
| 1384359_at | down | 1368656_at | up | 1397775_at | up |
| 1383428_at | down | 1370551_a_at | up | 1397804_at | up |
| 1384057_at | down | 1385759_at | up | 1397827_at | up |
| 1384356_at | down | 1369547_at | up | 1398731_at | up |
| 1385717_at | down | 1368256_at | up | 1378465_at | up |
| 1388818_at | down | 1387140_at | up | 1378965_at | up |
| 1395484_at | down | 1369040_at | up | 1381763_at | up |
| 1398516_at | down | 1375441_at | up | 1391815_at | up |
| 1392440_at | down | 1388279_at | up | 1394179_s_at | up |
| 1383344_at | down | 1370596_a_at | up | 1394712_at | up |
| 1391855_at | down | 1371063_at | up | 1396200_at | up |
| 1398390_at | down | 1367658_at | up | 1396584_at | up |
| 1375303_at | down | 1368445_at | up | 1391894_at | up |
| 1375982_at | down | 1385451_at | up | 1373759_at | up |
| 1373762_a_at | down | 1387615_at | up | 1374308_at | up |
| 1381829_at | down | 1383285_at | up | 1376399_at | up |
| 1398663_at | down | 1396449_at | up | 1376781_at | up |
| 1385525_at | down | 1376476_at | up | 1377547_at | up |
| 1376920_at | down | 1392688_at | up | 1377682_at | up |
| 1392322_at | down | 1376263_at | up | 1378071_at | up |
| 1393424_at | down | 1397491_at | up | 1378074_at | up |
| 1396798_at | down | 1375022_at | up | 1378202_at | up |
| 1383716_at | down | 1391198_at | up | 1378268_at | up |
| 1388680_at | down | 1390141_at | up | 1378284_at | up |
| 1378609_at | down | 1378426_at | up | 1378615_at | up |
| 1375861_at | down | 1374375_at | up | 1379007_at | up |
| 1393511_at | down | 1380344_at | up | 1379024_at | up |
| 1392965_a_at | down | 1377827_at | up | 1379156_at | up |
| 1377091_at | down | 1379075_at | up | 1379771_at | up |
| 1393003_at | down | 1393016_s_at | up | 1380111_at | up |
| 1396799_at | down | 1386645_at | up | 1380225_at | up |
| 1378436_at | down | 1379537_at | up | 1380384_at | up |
| 1395609_at | down | 1376136_at | up | 1380491_at | up |
| 1382576_at | down | 1385269_s_at | up | 1380738_at | up |
| 1390510_at | down | 1377948_at | up | 1380776_at | up |
| 1380981_at | down | 1389425_at | up | 1380779_at | up |
| 1384468_at | down | 1394710_at | up | 1380897_at | up |
| 1398659_at | down | 1384370_at | up | 1381513_at | up |
| 1376233_at | down | 1391163_at | up | 1381532_at | up |
| 1399130_at | down | 1385326_at | up | 1381577_at | up |
| 1378494_at | down | 1376884_a_at | up | 1381664_at | up |
| 1392667_at | down | 1384714_x_at | up | 1381736_at | up |
| 1375493_at | down | 1373976_at | up | 1381758_at | up |
| 1397338_at | down | 1396726_at | up | 1382582_at | up |
| 1383079_at | down | 1393865_at | up | 1382794_at | up |
| 1396842_at | down | 1374849_at | up | 1382883_at | up |
| 1398444_at | down | 1372194_at | up | 1383367_at | up |
| 1378065_at | down | 1385632_at | up | 1383688_at | up |
| 1380375_at | down | 1394617_at | up | 1383991_at | up |
| 1383800_at | down | 1373012_at | up | 1384291_at | up |
| 1396901_at | down | 1391595_at | up | 1384294_at | up |
| 1389513_at | down | 1385315_at | up | 1384425_at | up |
| 1374345_at | down | 1392406_at | up | 1384749_at | up |
| 1391368_at | down | 1391896_at | up | 1384803_at | up |
| 1391558_at | down | 1380866_at | up | 1384996_at | up |
| 1395042_at | down | 1379392_at | up | 1385384_at | up |
| 1390961_at | down | 1383395_at | up | 1385602_at | up |
| 1381080_at | down | 1386309_at | up | 1385849_at | up |
| 1393776_at | down | 1384073_at | up | 1385975_at | up |
| 1372817_at | down | 1377813_at | up | 1385983_at | up |
| 1384604_at | down | 1376955_at | up | 1386003_at | up |
| 1384253_at | down | 1379308_at | up | 1386109_at | up |
| 1375776_at | down | 1396196_at | up | 1386565_at | up |
| 1376194_at | down | 1382855_at | up | 1389379_at | up |
| 1376281_at | down | 1397598_at | up | 1389401_at | up |
| 1376463_at | down | 1378859_at | up | 1389495_at | up |
| 1376551_at | down | 1377498_at | up | 1389669_at | up |
| 1376686_at | down | 1381748_at | up | 1389770_at | up |
| 1377421_at | down | 1393278_at | up | 1391108_at | up |
| 1377426_at | down | 1383018_at | up | 1391354_at | up |
| 1377460_at | down | 1391813_at | up | 1391357_at | up |
| 1377551_at | down | 1372301_at | up | 1391598_at | up |
| 1378062_at | down | 1397327_at | up | 1391615_at | up |
| 1378115_at | down | 1378552_at | up | 1391716_at | up |
| 1378208_at | down | 1398944_at | up | 1391763_at | up |
| 1378472_at | down | 1391658_at | up | 1391868_at | up |
| 1378525_at | down | 1389916_at | up | 1391934_at | up |
| 1378539_at | down | 1372352_at | up | 1391937_at | up |
| 1378943_at | down | 1373096_at | up | 1391973_at | up |
| 1379143_at | down | 1380488_at | up | 1392145_at | up |
| 1379155_at | down | 1390045_at | up | 1392148_at | up |
| 1379274_at | down | 1379192_at | up | 1392163_at | up |
| 1379510_at | down | 1376207_at | up | 1392376_at | up |
| 1379798_at | down | 1391893_at | up | 1392380_at | up |
| 1379841_a_at | down | 1373660_at | up | 1394471_at | up |
| 1380034_at | down | 1372149_at | up | 1394684_at | up |
| 1380142_at | down | 1386081_at | up | 1394733_at | up |
| 1380273_at | down | 1374455_at | up | 1394835_at | up |
| 1380350_at | down | 1393379_a_at | up | 1394929_at | up |
| 1380423_at | down | 1381994_at | up | 1395143_at | up |
| 1380588_at | down | 1390552_at | up | 1395315_at | up |
| 1380758_at | down | 1382663_at | up | 1395866_at | up |
| 1380896_at | down | 1381804_at | up | 1396017_at | up |
| 1380965_at | down | 1391437_at | up | 1396367_at | up |
| 1381036_at | down | 1377189_at | up | 1396394_at | up |
| 1381047_at | down | 1398482_at | up | 1396425_at | up |
| 1381145_at | down | 1371633_at | up | 1396457_at | up |
| 1381209_at | down | 1379134_at | up | 1396504_at | up |
| 1381302_at | down | 1384044_at | up | 1396665_at | up |
| 1381413_at | down | 1383888_at | up | 1396758_at | up |
| 1382258_at | down | 1397454_at | up | 1396811_at | up |
| 1382390_at | down | 1375241_at | up | 1396821_at | up |
| 1382814_at | down | 1389457_at | up | 1396824_at | up |
| 1382926_s_at | down | 1388274_at | up | 1396974_at | up |
| 1382960_at | down | 1394299_at | up | 1397174_at | up |
| 1383483_at | down | 1376519_at | up | 1397301_at | up |
| 1383786_at | down | 1385606_at | up | 1397324_at | up |
| 1384051_at | down | 1375314_at | up | 1397330_at | up |
| 1384375_at | down | 1375671_at | up | 1397482_at | up |
| 1384380_at | down | 1393567_at | up | 1397601_at | up |
| 1384756_at | down | 1398005_at | up | 1397619_at | up |
| 1384969_at | down | 1385459_at | up | 1397623_at | up |
| 1385147_at | down | 1372981_at | up | 1398058_at | up |
| 1385249_at | down | 1379751_at | up | 1398163_at | up |
| 1385747_at | down | 1382579_at | up | 1375925_at | up |
| 1389761_at | down | 1377993_at | up | 1379193_at | up |
| 1389809_at | down | 1393897_at | up | 1380112_at | up |
| 1390322_at | down | 1393147_at | up | 1380687_at | up |
| 1390677_at | down | 1384784_at | up | 1380735_at | up |
| 1391142_at | down | 1379592_at | up | 1384557_at | up |
| 1391175_at | down | 1386121_at | up | 1384671_at | up |
| 1391588_at | down | 1390807_at | up | 1391289_at | up |
| 1392099_at | down | 1393397_at | up | 1391621_at | up |
| 1392144_at | down | 1376437_at | up | 1392195_at | up |
| 1392290_at | down | 1382378_at | up | 1394461_at | up |
| 1392703_at | down | 1382145_at | up | 1394588_at | up |
| 1392923_at | down | 1371641_at | up | 1395446_at | up |
| 1393018_at | down | 1398160_at | up | 1395468_at | up |
| 1394767_at | down | 1396323_at | up | 1396681_at | up |
| 1394804_at | down | 1392602_at | up | 1396694_at | up |
| 1395214_at | down | 1378102_at | up | 1396699_at | up |
| 1395529_at | down | 1391483_at | up | 1398041_at | up |
| 1395729_at | down | 1393518_at | up | 1369148_at | up |
| 1395742_at | down | 1393560_at | up | 1387374_at | up |
| 1396136_at | down | 1398605_at | up | 1369684_at | up |
| 1396439_at | down | 1373716_at | up | 1368591_at | up |
| 1396667_at | down | 1378027_at | up | 1387495_at | up |
| 1396833_at | down | 1397363_at | up | 1391323_at | up |
| 1397018_at | down | 1375987_at | up | 1378963_at | up |
| 1397128_at | down | 1378300_at | up | 1369504_at | up |
| 1397152_at | down | 1381654_at | up | 1369368_at | up |
| 1397481_at | down | 1376270_at | up | 1370022_at | up |
| 1397689_at | down | 1381300_at | up | 1391940_at | up |
| 1397704_at | down | 1376897_at | up | 1369394_at | up |
| 1397829_at | down | 1395850_at | up | 1377027_at | up |
| 1397944_at | down | 1374328_at | up | 1375379_at | up |
| 1398176_at | down | 1391069_at | up | 1367976_at | up |
| 1398230_at | down | 1393758_at | up | 1370450_at | up |
| 1398474_at | down | 1385100_at | up | 1368838_at | up |
| 1398558_at | down | 1382261_at | up | 1389846_at | up |
| 1398733_at | down | 1373964_at | up | 1369283_at | up |
| 1377947_at | down | 1372397_at | up | 1369362_at | up |
| 1379130_at | down | 1395835_at | up | 1390311_at | up |
| 1379740_at | down | 1375907_at | up | 1369900_at | up |
| 1392229_at | down | 1392797_at | up | 1385575_at | up |
| 1397058_at | down | 1381331_at | up | 1373443_a_at | up |
| 1397145_at | down | 1390982_at | up | 1387352_at | up |
| 1371472_at | down | 1373489_at | up | 1396074_at | up |
| 1371755_at | down | 1397532_at | up | 1368346_at | up |
| 1371766_at | down | 1390483_at | up | 1375755_at | up |
| 1371816_at | down | 1372206_at | up | 1387955_at | up |
| 1371906_at | down | 1375674_at | up | 1387290_at | up |
| 1371922_at | down | 1392228_at | up | 1384506_at | up |
| 1371986_at | down | 1389739_at | up | 1375169_at | up |
| 1372568_at | down | 1396960_at | up | 1367507_at | up |
| 1372585_at | down | 1390186_at | up | 1387944_at | up |
| 1372669_at | down | 1375514_at | up | 1396477_at | up |
| 1372748_at | down | 1382208_at | up | 1397961_x_at | up |
| 1372816_at | down | 1391713_at | up | 1387253_at | up |
| 1372820_at | down | 1376555_at | up | 1382156_at | up |
| 1372901_at | down | 1375325_at | up | 1396462_at | up |
| 1372998_at | down | 1388839_at | up | 1380854_at | up |
| 1373078_at | down | 1394008_x_at | up | 1368556_at | up |
| 1373100_at | down | 1384615_at | up | 1369582_at | up |
| 1373114_at | down | 1381573_at | up | 1368388_at | up |
| 1373264_at | down | 1393588_at | up | 1379796_at | up |
| 1373320_at | down | 1382833_at | up | 1388103_at | up |
| 1373410_at | down | 1374711_at | up | 1370784_a_at | up |
| 1373511_at | down | 1378583_at | up | 1388079_at | up |
| 1373607_at | down | 1395117_at | up | 1388087_at | up |
| 1373614_at | down | 1382474_at | up | 1369344_at | up |
| 1373627_at | down | 1385700_at | up | 1393955_at | up |
| 1373628_at | down | 1377168_at | up | 1369723_at | up |
| 1373659_at | down | 1377549_at | up | 1376444_at | up |
| 1373703_at | down | 1376826_at | up | 1380428_at | up |
| 1373944_at | down | 1378379_at | up | 1381886_at | up |
| 1374017_at | down | 1390640_at | up | 1369524_at | up |
| 1374096_at | down | 1397360_at | up | 1387293_at | up |
| 1374143_at | down | 1395658_at | up | 1391487_at | up |
| 1374158_at | down | 1382164_at | up | 1374657_at | up |
| 1374161_at | down | 1398063_x_at | up | 1375116_at | up |
| 1374164_at | down | 1377176_at | up | 1375294_at | up |
| 1374174_at | down | 1394676_at | up | 1377440_at | up |
| 1374242_at | down | 1391453_at | up | 1377512_at | up |
| 1374274_at | down | 1381851_at | up | 1379092_at | up |
| 1374365_at | down | 1391413_at | up | 1380006_at | up |
| 1374493_at | down | 1390338_at | up | 1380603_at | up |
| 1374543_at | down | 1377219_at | up | 1380647_at | up |
| 1374563_at | down | 1391249_at | up | 1380978_at | up |
| 1374607_at | down | 1374506_at | up | 1381053_at | up |
| 1374640_at | down | 1375599_at | up | 1381273_at | up |
| 1374643_at | down | 1374470_at | up | 1381760_at | up |
| 1374645_at | down | 1385210_at | up | 1382773_at | up |
| 1374648_at | down | 1384677_at | up | 1383928_a_at | up |
| 1374709_at | down | 1397826_at | up | 1383968_at | up |
| 1374718_at | down | 1379215_at | up | 1383975_at | up |
| 1374724_at | down | 1393725_at | up | 1385317_s_at | up |
| 1374781_at | down | 1371806_at | up | 1385631_at | up |
| 1374817_at | down | 1384954_at | up | 1385652_at | up |
| 1374868_at | down | 1376684_at | up | 1386425_at | up |
| 1374988_at | down | 1392422_at | up | 1386458_x_at | up |
| 1375000_at | down | 1382103_at | up | 1388281_at | up |
| 1375053_at | down | 1396080_at | up | 1391105_at | up |
| 1375101_at | down | 1381197_at | up | 1392184_at | up |
| 1375191_at | down | 1381460_at | up | 1392255_at | up |
| 1375192_at | down | 1382093_at | up | 1392430_at | up |
| 1375287_at | down | 1398646_at | up | 1393717_at | up |
| 1375350_at | down | 1388532_at | up | 1393760_at | up |
| 1375383_at | down | 1391128_at | up | 1393852_at | up |
| 1375583_at | down | 1398478_at | up | 1393973_at | up |
| 1375586_at | down | 1377770_at | up | 1394114_at | up |
| 1375731_at | down | 1375416_at | up | 1394872_x_at | up |
| 1375887_at | down | 1374439_at | up | 1395050_at | up |
| 1375888_at | down | 1385020_at | up | 1395227_at | up |
| 1375992_at | down | 1372942_at | up | 1395770_at | up |
| 1376071_at | down | 1385199_a_at | up | 1396104_at | up |
| 1376111_at | down | 1371822_at | up | 1396389_at | up |
| 1376167_at | down | 1389487_at | up | 1396433_at | up |
| 1376224_at | down | 1380476_at | up | 1396516_at | up |
| 1376283_at | down | 1377023_at | up | 1396582_at | up |
| 1376285_at | down | 1383853_at | up | 1397146_at | up |
| 1376295_at | down | 1376130_a_at | up | 1398165_at | up |
| 1376327_at | down | 1374926_at | up | 1377120_at | up |
| 1376330_at | down | 1378364_at | up | 1393859_at | up |
| 1376395_at | down | 1390544_at | up | 1396391_at | up |
| 1376469_at | down | 1382710_at | up | 1396459_at | up |
| 1376534_at | down | 1385063_at | up | 1396700_at | up |
| 1376575_at | down | 1372729_at | up | 1398323_at | up |
| 1376624_at | down | 1373290_at | up | 1371276_at | up |
| 1376699_at | down | 1374367_at | up | 1371554_at | up |
| 1376706_at | down | 1389115_at | up | 1371990_at | up |
| 1376708_at | down | 1396299_at | up | 1372573_at | up |
| 1376731_at | down | 1398466_at | up | 1372631_at | up |
| 1376741_at | down | 1394633_at | up | 1373356_at | up |
| 1376743_at | down | 1388270_at | up | 1373774_at | up |
| 1376744_at | down | 1378353_at | up | 1373783_at | up |
| 1376798_at | down | 1382618_at | up | 1374267_at | up |
| 1376840_at | down | 1378921_at | up | 1374316_at | up |
| 1376881_at | down | 1382122_at | up | 1374336_at | up |
| 1376914_at | down | 1392149_at | up | 1375064_at | up |
| 1376938_at | down | 1397234_at | up | 1375275_at | up |
| 1376978_at | down | 1392665_at | up | 1375292_at | up |
| 1376991_at | down | 1382119_at | up | 1376367_at | up |
| 1377002_at | down | 1396956_at | up | 1376505_at | up |
| 1377043_at | down | 1392007_at | up | 1377056_at | up |
| 1377066_at | down | 1379341_at | up | 1377107_at | up |
| 1377129_at | down | 1392186_at | up | 1377217_at | up |
| 1377133_at | down | 1381675_at | up | 1377295_at | up |
| 1377161_at | down | 1389950_at | up | 1377418_at | up |
| 1377181_at | down | 1380192_at | up | 1377652_at | up |
| 1377183_at | down | 1380379_at | up | 1378120_at | up |
| 1377215_at | down | 1374654_at | up | 1378151_at | up |
| 1377308_a_at | down | 1384988_at | up | 1378437_at | up |
| 1377317_at | down | 1389783_s_at | up | 1378593_at | up |
| 1377562_at | down | 1389762_at | up | 1378643_at | up |
| 1377565_at | down | 1372496_at | up | 1378663_at | up |
| 1377626_at | down | 1392356_at | up | 1378686_at | up |
| 1377672_at | down | 1397669_at | up | 1378773_at | up |
| 1377786_at | down | 1398006_at | up | 1378797_at | up |
| 1377823_at | down | 1388528_at | up | 1379128_at | up |
| 1377825_at | down | 1391434_at | up | 1379133_at | up |
| 1377841_at | down | 1389096_at | up | 1379141_at | up |
| 1377917_at | down | 1390286_at | up | 1379172_at | up |
| 1377923_at | down | 1378853_at | up | 1379413_at | up |
| 1377927_at | down | 1398414_at | up | 1379860_at | up |
| 1377962_at | down | 1377679_at | up | 1379919_at | up |
| 1378010_at | down | 1375826_at | up | 1380011_at | up |
| 1378021_at | down | 1385605_at | up | 1380140_at | up |
| 1378025_at | down | 1393929_at | up | 1380145_at | up |
| 1378044_at | down | 1375858_at | up | 1380284_at | up |
| 1378053_at | down | 1391191_at | up | 1380303_at | up |
| 1378055_at | down | 1374305_at | up | 1380575_at | up |
| 1378064_at | down | 1396085_at | up | 1380705_at | up |
| 1378095_at | down | 1384255_at | up | 1380769_at | up |
| 1378132_at | down | 1372660_at | up | 1380786_at | up |
| 1378182_at | down | 1377506_at | up | 1381011_at | up |
| 1378184_at | down | 1392701_at | up | 1381083_at | up |
| 1378328_at | down | 1378501_at | up | 1381143_at | up |
| 1378457_at | down | 1382030_at | up | 1381176_at | up |
| 1378474_at | down | 1380586_at | up | 1381192_at | up |
| 1378486_at | down | 1381423_at | up | 1381392_at | up |
| 1378498_at | down | 1384978_at | up | 1381411_at | up |
| 1378536_at | down | 1374690_at | up | 1381441_at | up |
| 1378654_at | down | 1393357_at | up | 1381518_at | up |
| 1378673_at | down | 1390519_at | up | 1381634_at | up |
| 1378691_at | down | 1371942_at | up | 1381648_at | up |
| 1378778_a_at | down | 1394730_at | up | 1381679_at | up |
| 1378824_at | down | 1388641_at | up | 1381692_x_at | up |
| 1378839_at | down | 1377458_at | up | 1381706_at | up |
| 1378851_at | down | 1390710_x_at | up | 1381710_at | up |
| 1378858_at | down | 1386269_x_at | up | 1381733_at | up |
| 1379043_at | down | 1383035_at | up | 1381747_at | up |
| 1379055_x_at | down | 1398581_at | up | 1381819_at | up |
| 1379089_at | down | 1395937_at | up | 1381870_at | up |
| 1379117_at | down | 1380271_at | up | 1382002_at | up |
| 1379182_at | down | 1377485_at | up | 1382078_at | up |
| 1379205_at | down | 1388898_at | up | 1382286_at | up |
| 1379302_at | down | 1385620_at | up | 1382437_at | up |
| 1379324_at | down | 1375336_at | up | 1382586_at | up |
| 1379370_at | down | 1372144_at | up | 1382606_at | up |
| 1379387_at | down | 1393372_at | up | 1382753_at | up |
| 1379460_at | down | 1380217_at | up | 1382880_at | up |
| 1379487_at | down | 1378872_at | up | 1382958_at | up |
| 1379492_at | down | 1397955_at | up | 1382964_at | up |
| 1379544_at | down | 1380521_at | up | 1383725_at | up |
| 1379576_at | down | 1371294_at | up | 1383820_at | up |
| 1379599_at | down | 1384811_at | up | 1383836_at | up |
| 1379604_at | down | 1395533_at | up | 1384096_at | up |
| 1379609_at | down | 1398314_at | up | 1384151_at | up |
| 1379659_at | down | 1389728_at | up | 1384288_at | up |
| 1379663_at | down | 1385215_at | up | 1384378_at | up |
| 1379667_at | down | 1393901_at | up | 1384414_at | up |
| 1379683_at | down | 1383125_at | up | 1384556_at | up |
| 1379684_at | down | 1377297_at | up | 1384563_at | up |
| 1379695_at | down | 1385259_at | up | 1384576_at | up |
| 1379720_at | down | 1381168_at | up | 1384637_at | up |
| 1379767_at | down | 1388741_at | up | 1384902_at | up |
| 1379820_at | down | 1390236_at | up | 1384961_at | up |
| 1379834_at | down | 1390006_at | up | 1384967_at | up |
| 1379878_at | down | 1384754_at | up | 1385039_at | up |
| 1379880_at | down | 1379188_at | up | 1385110_at | up |
| 1379908_at | down | 1397659_at | up | 1385114_at | up |
| 1379927_at | down | 1374995_at | up | 1385133_at | up |
| 1379929_at | down | 1376447_at | up | 1385140_at | up |
| 1379943_at | down | 1383246_at | up | 1385145_at | up |
| 1379967_at | down | 1390350_at | up | 1385180_at | up |
| 1379971_at | down | 1380093_at | up | 1385203_at | up |
| 1379990_at | down | 1372699_at | up | 1385247_at | up |
| 1379999_at | down | 1384771_at | up | 1385310_at | up |
| 1380042_at | down | 1382147_at | up | 1385324_x_at | up |
| 1380043_at | down | 1390712_at | up | 1385406_at | up |
| 1380079_at | down | 1395331_at | up | 1385439_x_at | up |
| 1380097_at | down | 1390943_at | up | 1385464_at | up |
| 1380100_at | down | 1385073_at | up | 1385479_at | up |
| 1380120_at | down | 1377365_at | up | 1385588_at | up |
| 1380127_at | down | 1397191_at | up | 1385617_at | up |
| 1380129_at | down | 1383973_at | up | 1385641_at | up |
| 1380130_at | down | 1375210_at | up | 1385745_at | up |
| 1380131_at | down | 1395516_at | up | 1385780_at | up |
| 1380144_at | down | 1395520_at | up | 1385802_at | up |
| 1380161_at | down | 1382589_at | up | 1385812_at | up |
| 1380215_at | down | 1394500_at | up | 1385820_at | up |
| 1380293_at | down | 1376449_at | up | 1385844_at | up |
| 1380322_at | down | 1384373_at | up | 1385855_at | up |
| 1380442_at | down | 1383446_at | up | 1385916_at | up |
| 1380527_at | down | 1397556_at | up | 1386149_at | up |
| 1380574_at | down | 1382062_at | up | 1386172_at | up |
| 1380641_at | down | 1394847_at | up | 1386185_at | up |
| 1380659_at | down | 1379304_at | up | 1386256_at | up |
| 1380672_at | down | 1384606_at | up | 1386342_at | up |
| 1380715_at | down | 1383442_at | up | 1386361_at | up |
| 1380744_at | down | 1372370_at | up | 1386383_at | up |
| 1380745_at | down | 1380381_at | up | 1386414_at | up |
| 1380748_at | down | 1374459_at | up | 1386441_at | up |
| 1380799_at | down | 1382869_at | up | 1386472_at | up |
| 1380809_at | down | 1395225_at | up | 1386526_at | up |
| 1380821_a_at | down | 1375147_at | up | 1386635_at | up |
| 1380822_at | down | 1381108_at | up | 1386798_at | up |
| 1380845_at | down | 1390332_at | up | 1389516_at | up |
| 1380858_at | down | 1381256_at | up | 1389905_at | up |
| 1380861_at | down | 1392454_at | up | 1389936_at | up |
| 1380883_at | down | 1385009_at | up | 1390085_at | up |
| 1380884_at | down | 1381161_a_at | up | 1390324_at | up |
| 1380886_at | down | 1376695_at | up | 1390560_at | up |
| 1380891_at | down | 1393844_at | up | 1390635_at | up |
| 1380901_at | down | 1375543_at | up | 1390652_at | up |
| 1380940_at | down | 1377453_at | up | 1390744_at | up |
| 1380974_at | down | 1390167_at | up | 1391140_at | up |
| 1381028_at | down | 1389767_at | up | 1391339_at | up |
| 1381031_at | down | 1385830_at | up | 1391367_at | up |
| 1381077_at | down | 1389366_at | up | 1391377_at | up |
| 1381079_at | down | 1395706_at | up | 1391469_at | up |
| 1381087_at | down | 1384725_at | up | 1391696_at | up |
| 1381112_at | down | 1375930_a_at | up | 1391811_at | up |
| 1381172_at | down | 1375446_at | up | 1392066_at | up |
| 1381240_at | down | 1378895_at | up | 1392072_at | up |
| 1381316_at | down | 1395879_at | up | 1392142_at | up |
| 1381317_at | down | 1380929_at | up | 1392182_at | up |
| 1381321_at | down | 1395422_at | up | 1392253_at | up |
| 1381337_at | down | 1397394_at | up | 1392389_at | up |
| 1381344_at | down | 1377397_at | up | 1392394_at | up |
| 1381359_at | down | 1378948_at | up | 1392420_at | up |
| 1381418_at | down | 1381347_at | up | 1392459_x_at | up |
| 1381445_at | down | 1385825_at | up | 1392877_at | up |
| 1381507_at | down | 1382552_at | up | 1393111_at | up |
| 1381556_at | down | 1391919_at | up | 1393258_at | up |
| 1381595_at | down | 1375567_at | up | 1393413_at | up |
| 1381615_at | down | 1374258_at | up | 1393481_at | up |
| 1381631_at | down | 1383763_at | up | 1393549_at | up |
| 1381639_at | down | 1382388_at | up | 1393635_x_at | up |
| 1381669_at | down | 1386818_at | up | 1393660_at | up |
| 1381670_at | down | 1377203_at | up | 1393793_at | up |
| 1381720_at | down | 1375234_at | up | 1393834_at | up |
| 1381731_at | down | 1391419_at | up | 1393857_at | up |
| 1381800_at | down | 1385629_at | up | 1393877_at | up |
| 1381863_at | down | 1390035_at | up | 1393905_at | up |
| 1381865_at | down | 1396708_at | up | 1393951_at | up |
| 1381989_at | down | 1393536_at | up | 1393999_at | up |
| 1382023_at | down | 1377246_at | up | 1394112_at | up |
| 1382107_at | down | 1376507_a_at | up | 1394132_at | up |
| 1382271_at | down | 1385136_at | up | 1394144_at | up |
| 1382299_at | down | 1383732_at | up | 1394169_at | up |
| 1382324_at | down | 1390824_at | up | 1394186_at | up |
| 1382336_at | down | 1378975_at | up | 1394479_at | up |
| 1382339_a_at | down | 1389853_at | up | 1394563_at | up |
| 1382371_at | down | 1380036_at | up | 1394675_at | up |
| 1382404_at | down | 1395735_at | up | 1394708_at | up |
| 1382463_at | down | 1381827_at | up | 1394856_at | up |
| 1382483_at | down | 1382282_at | up | 1394866_at | up |
| 1382542_at | down | 1391531_at | up | 1394870_at | up |
| 1382631_at | down | 1383233_at | up | 1394958_at | up |
| 1382659_at | down | 1394327_at | up | 1395038_at | up |
| 1382660_at | down | 1393197_at | up | 1395067_at | up |
| 1382662_at | down | 1388760_at | up | 1395077_at | up |
| 1382692_at | down | 1393043_at | up | 1395101_at | up |
| 1382702_at | down | 1398712_at | up | 1395220_at | up |
| 1382706_at | down | 1373889_at | up | 1395277_at | up |
| 1382780_at | down | 1397521_at | up | 1395300_at | up |
| 1382806_at | down | 1376758_at | up | 1395450_at | up |
| 1382863_at | down | 1397539_at | up | 1395500_at | up |
| 1382867_at | down | 1391312_at | up | 1395567_at | up |
| 1382897_at | down | 1398055_at | up | 1395682_at | up |
| 1382898_at | down | 1371560_at | up | 1395714_at | up |
| 1382910_at | down | 1379563_at | up | 1395756_at | up |
| 1382936_at | down | 1389880_at | up | 1395849_at | up |
| 1382980_at | down | 1375503_at | up | 1395858_at | up |
| 1382994_at | down | 1393215_at | up | 1395935_at | up |
| 1383063_a_at | down | 1397973_x_at | up | 1396014_at | up |
| 1383078_at | down | 1384208_at | up | 1396032_at | up |
| 1383113_at | down | 1393321_at | up | 1396098_at | up |
| 1383194_a_at | down | 1380628_at | up | 1396102_at | up |
| 1383211_at | down | 1373900_at | up | 1396337_at | up |
| 1383351_at | down | 1371540_at | up | 1396364_at | up |
| 1383372_at | down | 1377291_at | up | 1396406_at | up |
| 1383385_at | down | 1381975_at | up | 1396410_at | up |
| 1383390_at | down | 1375609_at | up | 1396437_at | up |
| 1383434_at | down | 1381814_at | up | 1396441_at | up |
| 1383463_at | down | 1381126_at | up | 1396454_at | up |
| 1383468_at | down | 1391284_at | up | 1396475_at | up |
| 1383532_at | down | 1392134_x_at | up | 1396553_at | up |
| 1383552_at | down | 1378286_at | up | 1396562_at | up |
| 1383579_at | down | 1375564_at | up | 1396620_at | up |
| 1383621_at | down | 1377580_at | up | 1396632_at | up |
| 1383638_at | down | 1389636_at | up | 1396662_at | up |
| 1383758_at | down | 1391570_at | up | 1396680_at | up |
| 1383776_at | down | 1393796_at | up | 1396755_at | up |
| 1383795_at | down | 1383396_at | up | 1396769_at | up |
| 1383849_at | down | 1392727_at | up | 1396792_at | up |
| 1383869_at | down | 1392020_at | up | 1396818_at | up |
| 1383873_at | down | 1397201_at | up | 1397020_at | up |
| 1383882_at | down | 1385417_at | up | 1397069_at | up |
| 1383935_at | down | 1390261_at | up | 1397111_at | up |
| 1383982_at | down | 1373985_at | up | 1397139_at | up |
| 1384058_at | down | 1395271_at | up | 1397150_at | up |
| 1384062_at | down | 1379955_at | up | 1397379_at | up |
| 1384071_at | down | 1377090_at | up | 1397393_at | up |
| 1384109_at | down | 1397702_at | up | 1397459_at | up |
| 1384146_at | down | 1392421_at | up | 1397833_at | up |
| 1384184_at | down | 1380930_at | up | 1397864_at | up |
| 1384192_at | down | 1377118_at | up | 1397869_at | up |
| 1384245_at | down | 1378226_at | up | 1397903_at | up |
| 1384247_at | down | 1391065_at | up | 1397918_at | up |
| 1384250_a_at | down | 1375914_at | up | 1397933_at | up |
| 1384272_at | down | 1396129_at | up | 1398088_at | up |
| 1384310_at | down | 1372516_at | up | 1398095_at | up |
| 1384484_at | down | 1395828_at | up | 1398169_at | up |
| 1384514_at | down | 1397505_at | up | 1398189_at | up |
| 1384515_at | down | 1377748_at | up | 1398207_at | up |
| 1384527_at | down | 1385153_at | up | 1398665_at | up |
| 1384542_at | down | 1385064_at | up | 1398702_at | up |
| 1384551_at | down | 1384601_at | up | 1398730_at | up |
| 1384582_at | down | 1371317_at | up | 1398735_at | up |
| 1384587_at | down | 1383003_at | up | 1377373_at | up |
| 1384621_at | down | 1374237_at | up | 1378954_at | up |
| 1384681_at | down | 1383459_at | up | 1379189_at | up |
| 1384682_at | down | 1381466_at | up | 1380613_at | up |
| 1384696_at | down | 1374626_at | up | 1380674_at | up |
| 1384807_at | down | 1398578_at | up | 1380732_at | up |
| 1384808_at | down | 1381802_at | up | 1380787_at | up |
| 1384823_at | down | 1379803_at | up | 1381222_at | up |
| 1384840_at | down | 1396398_at | up | 1381271_at | up |
| 1384845_at | down | 1391736_at | up | 1381661_at | up |
| 1384874_at | down | 1384099_at | up | 1382247_a_at | up |
| 1384891_at | down | 1391667_at | up | 1382624_at | up |
| 1384897_at | down | 1375766_at | up | 1383784_at | up |
| 1384898_at | down | 1390968_at | up | 1385000_at | up |
| 1384922_at | down | 1392207_at | up | 1385045_at | up |
| 1384923_at | down | 1382519_at | up | 1385185_at | up |
| 1384940_at | down | 1394828_at | up | 1385260_at | up |
| 1384949_at | down | 1393917_at | up | 1385365_at | up |
| 1385005_at | down | 1391557_at | up | 1385603_at | up |
| 1385029_at | down | 1391425_at | up | 1385771_at | up |
| 1385070_at | down | 1381502_at | up | 1386265_at | up |
| 1385399_at | down | 1393229_at | up | 1386687_at | up |
| 1385401_at | down | 1389992_at | up | 1388290_at | up |
| 1385659_at | down | 1376009_at | up | 1391779_at | up |
| 1385667_x_at | down | 1395697_at | up | 1391892_at | up |
| 1385930_at | down | 1389634_at | up | 1392352_at | up |
| 1386026_at | down | 1377302_a_at | up | 1392377_at | up |
| 1386119_at | down | 1389914_at | up | 1392823_at | up |
| 1386224_at | down | 1374053_at | up | 1393787_at | up |
| 1386483_at | down | 1373069_at | up | 1393797_at | up |
| 1386671_at | down | 1378729_at | up | 1393858_at | up |
| 1388383_at | down | 1375260_at | up | 1393979_at | up |
| 1388652_at | down | 1374632_at | up | 1394000_at | up |
| 1388727_at | down | 1381270_at | up | 1395938_at | up |
| 1388771_at | down | 1374507_at | up | 1396338_at | up |
| 1388783_at | down | 1373814_at | up | 1396431_at | up |
| 1388930_at | down | 1376961_at | up | 1396458_at | up |
| 1388949_at | down | 1395791_at | up | 1396505_at | up |
| 1388999_at | down | 1389889_at | up | 1396759_at | up |
| 1389113_at | down | 1381660_at | up | 1396779_at | up |
| 1389193_at | down | 1375734_at | up | 1397057_at | up |
| 1389256_at | down | 1374974_at | up | 1397088_at | up |
| 1389310_at | down | 1386800_at | up | 1397144_at | up |
| 1389335_at | down | 1391285_at | up | 1397467_at | up |
| 1389462_at | down | 1372927_at | up | 1398035_at | up |
| 1389529_at | down | 1397105_at | up | 1398208_s_at | up |
| 1389583_at | down | 1375976_a_at | up | 1398228_at | up |
| 1389718_at | down | 1394905_at | up | 1398675_at | up |
| 1389790_at | down | 1380067_at | up | 1381224_at | up |
| 1390028_at | down | 1384298_at | up | 1385319_at | up |
| 1390083_at | down | 1378970_at | up | 1393789_at | up |
| 1390156_a_at | down | 1382025_at | up | 1396314_at | up |
| 1390204_at | down | 1395344_at | up | 1396479_at | up |
| 1390205_at | down | 1372402_at | up | 1377222_at | up |
| 1390396_at | down | 1396282_at | up | 1378815_at | up |
| 1390397_at | down | 1396146_at | up | 1379078_at | up |
| 1390621_at | down | 1383966_at | up | 1380014_at | up |
| 1390631_at | down | 1381120_at | up | 1380782_at | up |
| 1390737_at | down | 1395467_at | up | 1381195_at | up |
| 1390759_at | down | 1395458_at | up | 1381207_at | up |
| 1390828_at | down | 1392868_at | up | 1381766_at | up |
| 1390866_at | down | 1393946_at | up | 1382560_at | up |
| 1390886_at | down | 1381226_at | up | 1383038_at | up |
| 1390907_at | down | 1384207_at | up | 1384970_at | up |
| 1390913_at | down | 1396192_at | up | 1384999_at | up |
| 1390958_at | down | 1379560_at | up | 1385284_at | up |
| 1390989_at | down | 1371908_at | up | 1385446_at | up |
| 1391088_at | down | 1383759_at | up | 1385703_at | up |
| 1391092_at | down | 1391891_at | up | 1386189_at | up |
| 1391139_at | down | 1379742_at | up | 1386433_at | up |
| 1391179_at | down | 1380133_at | up | 1386463_at | up |
| 1391217_at | down | 1383898_at | up | 1386493_at | up |
| 1391349_at | down | 1392828_at | up | 1386744_x_at | up |
| 1391424_at | down | 1383119_at | up | 1386805_at | up |
| 1391459_at | down | 1376317_at | up | 1390320_at | up |
| 1391500_at | down | 1375467_at | up | 1390920_at | up |
| 1391563_at | down | 1381469_a_at | up | 1391111_at | up |
| 1391594_at | down | 1378375_at | up | 1391288_at | up |
| 1391605_at | down | 1380549_at | up | 1391995_at | up |
| 1391724_at | down | 1374939_at | up | 1393570_at | up |
| 1391742_at | down | 1380275_at | up | 1393674_at | up |
| 1391759_at | down | 1382924_at | up | 1393868_at | up |
| 1391774_at | down | 1392008_at | up | 1393934_at | up |
| 1391823_at | down | 1385736_at | up | 1393988_at | up |
| 1391824_at | down | 1385724_at | up | 1393991_x_at | up |
| 1391916_at | down | 1386739_at | up | 1394336_at | up |
| 1391947_at | down | 1384993_at | up | 1395815_at | up |
| 1391958_at | down | 1375440_at | up | 1396526_at | up |
| 1391978_at | down | 1374916_at | up | 1396772_at | up |
| 1392013_at | down | 1378169_at | up | 1396991_at | up |
| 1392024_at | down | 1385808_at | up | 1397091_at | up |
| 1392088_at | down | 1376043_at | up | 1397153_at | up |
| 1392118_at | down | 1375600_at | up | 1397462_at | up |
| 1392141_at | down | 1374969_at | up | 1397494_at | up |
| 1392155_at | down | 1382771_at | up | 1397859_x_at | up |
| 1392179_at | down | 1384589_at | up | 1398152_at | up |
| 1392201_at | down | 1377534_at | up | 1398661_at | up |
| 1392212_at | down | 1374590_at | up | 1398671_at | up |
| 1392236_at | down | 1384912_at | up | 1398715_at | up |
| 1392245_at | down | | | 1385197_at | up |
| 1392250_at | down | | | 1386152_at | up |
| 1392252_at | down | | | 1392829_at | up |
| 1392274_at | down | | | 1396395_at | up |
| 1392293_at | down | | | 1398216_at | up |

TABLE 2
Up regulated in IUGR and CR
proviral integration site 1
homocysteine-inducible, endoplasmic reticulum stress-inducible, ubiquitin-
like domain member 1
Protein C receptor, endothelial (predicted)
Solute carrier family 2 (facilitated glucose transporter), member 2
glycoprotein hormones, alpha subunit
similar to hypothetical protein FLJ13511 (predicted)
phospholipase C-like 2 (predicted)
similar to Hypothetical WD-repeat protein CGI-48 (predicted)
peroxiredoxin 6
Sorting nexin 10 (predicted)
leptin
Similar to IER7
Small fragment nuclease (predicted)
Tissue factor pathway inhibitor
Similar to IER6
syndecan 1
Jun D proto-oncogene
huntingtin interacting protein 2 (predicted)
Ferredoxin 1
solute carrier family 11 (proton-coupled divalent metal ion transporters),
member 2
neuron specific gene family member 1
Hydroxysteroid dehydrogenase-1, delta<5>-3-beta (predicted)
Down regulated in both iugr and cr
Catalase
colony stimulating factor 1 (macrophage)
Zinc finger protein 262 (predicted)
immunoglobulin (CD79A) binding protein 1
Hepatocyte growth factor
bone morphogenetic protein 5 (predicted)
adaptor-related protein complex 3, mu 2 subunit
Similar to hypothetical protein FLJ22344 (predicted)
Glycine amidinotransferase (L-arginine: glycine amidinotransferase)
similar to map kinase interacting kinase
SMC6 structural maintenance of chromosomes 6-like 1 (yeast) (predicted)
aquarius (predicted)
sprouty homolog 2 (Drosophila) (predicted)
cytoplasmic FMR1 interacting protein 1 (predicted)
similar to TBC1 domain family member 4
folate receptor 2 (fetal) (predicted)
mitogen-activated protein kinase kinase kinase kinase 4 (predicted)
Adducin 3 (gamma)
FERM domain containing 4B
dystonin (predicted)
O-linked N-acetylglucosamine (GlcNAc) transferase (UDP-N-
acetylglucosamine: polypeptide-N-acetylglucosaminyl transferase)
platelet derived growth factor receptor, beta polypeptide
lysosomal-associated protein transmembrane 4B (predicted)
myosin X (predicted)
laminin, alpha 2 (predicted)
low density lipoprotein receptor-related protein 6 (predicted)
ubiquitin conjugation factor E4 A
transforming growth factor beta 1 induced transcript 1
RT1 class I, CE16
RT1 class Ia, locus A2
procollagen, type XV (predicted)
protocadherin gamma subfamily C, 3
CD4 antigen
stabilin 1 (predicted)
guanylate cyclase 1, soluble, beta 3
Ras homolog gene family, member E
procollagen C-proteinase enhancer protein
peripheral myelin protein 22
fibromodulin
wingless-related MMTV integration site 2
CD44 antigen
intercellular adhesion molecule 2
myeloid cell nuclear differentiation antigen (predicted)
complement component 1, r subcomponent (predicted)
dihydropyrimidinase-like 3
RT1 class I, CE15
RT1 class Ib, locus Aw2
RT1-149 protein
amine oxidase, copper containing 3
procollagen, type VI, alpha 3 (predicted)
Cytochrome b-245, beta polypeptide
Neuropilin 1
caspase 1
Down syndrome critical region homolog 1 (human)
Similar to E430002G05Rik protein (predicted)
Collagen, type V, alpha 2
Nuclear receptor subfamily 3, group C, member 1
similar to hypothetical protein FLJ10652 (predicted)
guanylate cyclase 1, soluble, alpha 3
potassium voltage-gated channel, delayed-rectifier, subfamily S, member 3
procollagen, type I, alpha 3
insulin-like growth factor 1
allograft inflammatory factor 1
allograft inflammatory factor 2
procollagen, type I, alpha 2
plasma glutamate carboxypeptidase
Down in IUGR and Up in CR
CUG triplet repeat, RNA-binding protein 2
aldehyde dehydrogenase family 3, subfamily A2
core-binding factor, runt domain, alpha subunit 2; translocated to, 1; cyclin
D-related (predicted)
collagen, type V, alpha 3
Nuclear receptor subfamily 2, group F, member 2
ATPase, Ca++ transporting, cardiac muscle, slow twitch 2
similar to dJ202D23.2 (novel protein similar to C21ORF5 (KIAA0933))
(predicted)
Transducin-like enhancer of split 4, E(spl) homolog (Drosophila)
Potassium channel, subfamily K, member 3
apurinic/apyrimidinic endonuclease 1
gap junction membrane channel protein alpha 1
ATPase, Ca++ transporting, plasma membrane 4
Transducin-like enhancer of split 4, E(spl) homolog (Drosophila)
Solute carrier family 5 (inositol transporters), member 3
Klotho
tripartite motif protein 27 (predicted)
myosin Ib
chromosome condensation 1-like
thymus cell antigen 1, theta
CTD (carboxy-terminal domain, RNA polymerase II, polypeptide A) small
phosphatase-like (predicted)
cAMP responsive element binding protein 1
Ectonucleotide pyrophosphatase/phosphodiesterase 2
LRRC36 homolog (human)
Growth arrest specific 6
CDC16 cell division cycle 16 homolog (S. cerevisiae) (predicted)
matrix metallopeptidase 2
UP in IUGR and Down in CR
Similar to RIKEN cDNA 1500006O09 (predicted)
growth differentiation factor 15
Basic leucine zipper and W2 domains 4
colony stimulating factor 2 receptor, beta 1, low-affinity (granulocyte-
macrophage)
eukaryotic translation termination factor 1 (predicted)
similar to RIKEN cDNA 1110012L19
Similar to RIKEN cDNA 3930401K13 (predicted)
ubiquitin-conjugating enzyme E2 variant 2
Tankyrase, TRF1-interacting ankyrin-related ADP-ribose polymerase 2
(predicted)
RAS guanyl releasing protein 1
ornithine decarboxylase antizyme inhibitor
Thymine-DNA glycosylase
microfibrillar-associated protein 3-like (predicted)
actin related protein ⅔ complex, subunit 5-like (predicted)
polymerase (RNA) II (DNA directed) polypeptide H (predicted)
ectonucleoside triphosphate diphosphohydrolase 1
microtubule-associated protein 7 (predicted)
Tankyrase, TRF1-interacting ankyrin-related ADP-ribose polymerase 2
(predicted)
Similar to methyl-CpG binding protein MBD2
cytochrome P450, family 19, subfamily a, polypeptide 1
hexosaminidase B (predicted)
Similar to testis specific protein, Ddc8
Serine/threonine kinase 3
inhibin beta-A
microfibrillar associated protein 5 (predicted)
GULP, engulfment adaptor PTB domain containing 1 (predicted)
solute carrier family 11 (proton-coupled divalent metal ion transporters),
member 3
calpain 6
Transferrin receptor

TABLE 3

| | | | Body | | | | | Log | | | | | | Predicted |
|---|---|---|---|---|---|---|---|---|---|---|---|---|---|---|
| | | | wt @ | placenta | | | | base 2 | | | | | | percent |
| | | | 21days | wt at | | | | expression | | | | | placenta/ | of normal |
| SUB | SUBNUM | NUMPUPS | gest | 21 days | GROUP | GROUP$ | NUTR | for G6 | G7 | G8 | G9 | G10 | body wt | nutrition |
| 431 | 1 | | | | 1 | cont | 100 | 9.44 | 6.39 | 8.64 | 5.26 | 8.03 | | 93.23607 |
| 434 | 2 | | | | 1 | cont | 100 | 9.49 | 6.29 | 8.35 | 5.71 | 7.52 | | 92.90031 |
| 6063 | 3 | 14 | 5.35 | 0.59 | 1 | cont | 100 | 9.08 | 5.64 | 8.32 | 5.66 | 7.8 | 0.110280374 | 81.99082 |
| 6078 | 4 | 12 | 4.74 | 0.61 | 1 | cont | 100 | 9.4 | 6.31 | 9.52 | 5.55 | 8.23 | 0.128691983 | 100.7257 |
| 6081 | 5 | 16 | 4.27 | 0.602 | 1 | cont | 100 | 9.32 | 6.11 | 8.75 | 5.28 | 7.47 | 0.140983607 | 80.80017 |
| 6082 | 6 | 15 | 5.78 | 0.568 | 1 | cont | 100 | 9.56 | 6.35 | 9.27 | 5.44 | 8.3 | 0.098269896 | 97.44234 |
| 6089 | 7 | 16 | 6.12 | 0.578 | 1 | coot | 100 | 9.26 | 6.57 | 9.2 | 5.93 | 8.38 | 0.094444444 | 120.72533 |
| 7000 | 8 | 15 | 4.4 | 0.585 | 1 | cont | 100 | 9.26 | 6.06 | 9.24 | 5.32 | 9.03 | 0.132954545 | 99.36833 |
| 430 | 9 | | | | 2 | cr50 | 50 | 9.36 | 5.79 | 8.02 | 5.1 | 7.29 | | 63.71409 |
| 461 | 10 | | | | 2 | cr50 | 50 | 8.35 | 5.14 | 8.18 | 4.49 | 7.09 | | 45.71904 |
| 480 | 11 | | | | 2 | cr50 | 50 | 8.98 | 5.69 | 7.73 | 4.97 | 7.19 | | 62.94884 |
| 520 | 12 | | | | 2 | cr50 | 50 | 9.09 | 5.39 | 6.85 | 4.77 | 7.52 | | 50.54269 |
| 522 | 13 | | | | 2 | cr50 | 50 | 8.78 | 4.9 | 7.21 | 5.06 | 7.45 | | 46.49589 |
| 6052 | 14 | 15 | 2.56 | 0.362 | 3 | cr70 | 30 | 8.54 | 4.89 | 7.83 | 4.74 | 6.58 | 0.14140625 | 34.02256 |
| 6070 | 15 | 16 | 2.71 | 0.322 | 3 | cr70 | 30 | 8.22 | 4.75 | 7.11 | 4.24 | 6.63 | 0.118819188 | 24.39289 |
| 6073 | 16 | 15 | 1.69 | 0.349 | 3 | cr70 | 30 | 8.36 | 4.65 | 7.4 | 4.47 | 6.72 | 0.206508876 | 25.13745 |
| 6081 | 17 | 11 | 2.89 | 0.4 | 3 | cr70 | 30 | 8.67 | 4.68 | 7.19 | 4.93 | 7.22 | 0.138408304 | 36.16791 |
| 6088 | 18 | 14 | 3.16 | 0.378 | 3 | cr70 | 30 | 8.86 | 5.12 | 8.34 | 4.82 | 7.1 | 0.119620253 | 43.72432 |
| 447 | 19 | | | | 4 | high fat | | 8.08 | 5.36 | 8.44 | 5.31 | 8.11 | | 86.58862 |
| 448 | 20 | | | | 4 | high fat | | 8.57 | 5.35 | 9.12 | 5.69 | 8.39 | | 89.8461 |
| 475 | 21 | | | | 4 | high fat | | 8.22 | 5.79 | 7.61 | 5.46 | 8.03 | | 99.03123 |
| 476 | 22 | | | | 4 | high fat | | 8.46 | 5.54 | 8.4 | 5.33 | 7.74 | | 81.80713 |
| 477 | 23 | | | | 4 | high fat | | 8.08 | 4.59 | 7.73 | 4.47 | 8.35 | | 46.9611 |
| 490 | 24 | | | | 4 | high fat | | 8.77 | 6.9 | 7.47 | 6.17 | 7.19 | | 129.35374 |
| 423 | 25 | | | | 5 | ssri | | 8.97 | 5.83 | 8.34 | 5.07 | 7.19 | | 70.1036 |
| 424 | 26 | | | | 5 | ssri | | 9.76 | 6.83 | 8.25 | 5.61 | 7.79 | | 105.7582 |
| 462 | 27 | | | | 5 | ssri | | 8.56 | 5.81 | 7.56 | 6.11 | 8.37 | | 111.63444 |
| 463 | 28 | | | | 5 | ssri | | 8.71 | 5.78 | 7.7 | 5.44 | 8.23 | | 92.27523 |
| 486 | 29 | | | | 5 | ssri | | 8.41 | 6.62 | 7.57 | 5.3 | 6.76 | | 103.37632 |
| 493 | 30 | | | | 5 | ssri | | 9.1 | 6.05 | 7.69 | 5.39 | 8.03 | | 90.5559 |

TABLE 4
Results from regression model (output from Systat).
DEP VAR: NUTR N: 18 MULTIPLE R: 0.942 SQUARED MULTIPLE R: 0.888
ADJUSTED SQUARED MULTIPLE R: .841 STANDARD ERROR OF ESTIMATE: 12.618

| VARIABLE | COEFFICIENT | STD ERROR | STD COEF | TOLERANCE | T | P(2 TAIL) |
|---|---|---|---|---|---|---|
| CONSTANT | −154.181 | 112.434 | 0.000 | . | −1.371 | 0.195 |
| G6 | −17.098 | 19.339 | −0.232 | 0.137 | −0.884 | 0.394 |
| G7 | 30.792 | 14.584 | 0.650 | 0.099 | 2.111 | 0.056 |
| G8 | 0.860 | 7.287 | 0.022 | 0.260 | 0.118 | 0.908 |
| G9 | 21.483 | 13.291 | 0.316 | 0.245 | 1.616 | 0.132 |
| G10 | 11.411 | 8.265 | 0.238 | 0.315 | 1.381 | 0.193 |

ANALYSIS OF VARIANCE

| SOURCE | SUM-OF-SQUARES | DF | MEAN-SQUARE | F-RATIO | P |
|---|---|---|---|---|---|
| REGRESSION | 15089.559 | 5 | 3017.912 | 18.956 | 0.000 |
| RESIDUAL | 1910.441 | 12 | 159.203 | | |

percent normal nutrition = −154.181 + (−17.098 * G6) + (30.792 * G7) + (.860 * G8) + (21.483 * G9) + (11.411 * G10)

TABLE 6

| ProbeSet | HF | HF | HF | HF | HF | HF | Cont |
|---|---|---|---|---|---|---|---|
| 1368220_at | 11.00438 | 10.94605 | 10.97849 | 10.82925 | 10.77598 | 10.84353 | 9.758575 |
| 1374716_at | 8.504949 | 8.337197 | 8.454606 | 8.369065 | 8.498878 | 8.321859 | 7.382849 |
| 1389323_at | 11.19787 | 11.19813 | 11.25418 | 10.92503 | 10.84815 | 11.09756 | 9.759944 |
| 1372137_at | 9.386279 | 9.075376 | 9.245911 | 9.20572 | 9.138822 | 9.133685 | 8.340476 |
| 1372829_at | 10.74478 | 10.70918 | 10.75531 | 10.48642 | 10.55559 | 10.54746 | 9.530819 |
| 1382415_at | 9.290794 | 9.198184 | 9.485319 | 9.122929 | 9.070975 | 9.284197 | 8.184726 |
| 1398572_at | 10.39127 | 10.49282 | 10.33323 | 10.08627 | 10.2705 | 10.15836 | 9.456452 |
| 1372269_at | 10.01691 | 10.23635 | 10.16678 | 9.785023 | 9.890261 | 10.07748 | 9.077942 |
| 1373711_at | 7.725819 | 7.36752 | 7.445315 | 7.538301 | 7.501101 | 7.496567 | 6.459201 |
| 1379623_at | 8.773002 | 8.615665 | 8.393821 | 8.511766 | 8.50896 | 8.510308 | 7.49858 |
| 1387663_at | 9.887677 | 9.71076 | 9.643336 | 9.547209 | 9.627646 | 9.905763 | 8.806636 |
| 1371745_at | 10.3637 | 10.57099 | 10.49923 | 10.27754 | 10.19192 | 10.20092 | 9.326018 |
| 1372977_at | 9.093507 | 8.851905 | 8.944857 | 8.923531 | 8.608058 | 8.767383 | 7.736954 |
| 1373490_at | 9.322329 | 9.468861 | 9.402854 | 9.064308 | 9.233554 | 9.229498 | 8.489771 |
| 1372209_at | 10.91862 | 10.90722 | 10.74615 | 10.68275 | 10.70574 | 10.82427 | 10.16373 |
| 1374790_at | 8.410971 | 8.406079 | 8.159171 | 8.185976 | 8.120542 | 8.348803 | 7.194501 |
| 1373632_at | 9.313249 | 9.591816 | 9.705995 | 9.044372 | 9.573916 | 9.369567 | 8.038482 |
| 1377025_at | 8.935143 | 9.215185 | 8.939571 | 8.704502 | 9.110838 | 8.819142 | 7.718635 |
| 1373660_at | 8.476786 | 8.543437 | 8.318441 | 8.449807 | 8.369802 | 8.328536 | 7.856901 |
| 1388171_at | 10.46938 | 10.06451 | 10.12315 | 10.00891 | 10.1982 | 10.23223 | 9.094078 |
| 1375345_at | 10.67223 | 10.38428 | 10.26475 | 10.56421 | 10.10392 | 10.48604 | 9.266286 |
| 1368164_at | 7.515856 | 7.450585 | 7.259456 | 7.111779 | 7.292723 | 7.24609 | 5.862738 |
| 1367929_at | 12.40889 | 12.08841 | 12.32236 | 12.47241 | 12.43106 | 12.48097 | 11.21979 |
| 1367945_at | 10.58849 | 10.4998 | 10.55867 | 10.4129 | 10.27551 | 10.28025 | 9.601717 |
| 1389970_at | 11.06215 | 10.34947 | 10.71797 | 10.51968 | 10.99669 | 10.86433 | 9.398279 |
| 1371484_at | 9.404498 | 9.10155 | 9.194271 | 9.135844 | 9.475554 | 9.397057 | 8.50146 |
| 1371435_at | 12.24045 | 12.02909 | 12.13144 | 12.04379 | 11.99163 | 12.15008 | 10.95189 |
| 1398770_at | 12.01959 | 12.10871 | 11.86145 | 11.84417 | 11.91311 | 11.98752 | 10.97417 |
| 1371421_at | 9.908343 | 9.688636 | 9.710867 | 9.752025 | 9.729484 | 9.911593 | 9.06891 |
| 1386930_at | 10.33022 | 10.22295 | 9.972137 | 10.17075 | 10.26647 | 9.970412 | 9.107647 |
| 1369588_a_at | 11.80843 | 11.65836 | 11.66923 | 11.5056 | 11.77628 | 11.65619 | 10.67902 |
| 1370321_at | 9.636169 | 9.502361 | 9.331273 | 9.340487 | 9.392303 | 9.397918 | 8.611631 |
| 1382825_at | 5.313913 | 5.767877 | 5.476568 | 5.596724 | 5.597936 | 5.320471 | 6.251075 |
| 1397956_at | 4.551754 | 4.662863 | 5.069054 | 4.781731 | 4.740965 | 4.520352 | 6.007783 |
| 1373982_at | 7.754149 | 7.974967 | 7.922373 | 7.654488 | 7.899826 | 7.620515 | 6.885341 |
| 1372436_at | 9.427578 | 9.591035 | 9.237899 | 9.347602 | 9.017403 | 9.365184 | 8.594843 |
| 1389009_at | 8.404825 | 8.449807 | 8.314396 | 8.106933 | 8.161294 | 8.371829 | 7.761211 |
| 1388328_at | 10.71814 | 10.32473 | 10.382 | 10.36786 | 10.17849 | 10.57385 | 9.56632 |
| 1389053_at | 9.34087 | 9.22469 | 8.879764 | 8.591748 | 8.750491 | 8.874844 | 7.657208 |
| 1373184_at | 9.090719 | 8.945809 | 8.94398 | 9.218229 | 9.124262 | 9.104634 | 8.218808 |
| 1389409_at | 10.44495 | 9.58989 | 9.712718 | 10.05422 | 9.713592 | 9.709694 | 8.351042 |
| 1374291_at | 7.171646 | 7.282665 | 7.383197 | 7.107001 | 7.205973 | 6.897464 | 6.352909 |
| 1392517_at | 8.955887 | 8.481499 | 8.526306 | 8.346758 | 8.292787 | 8.334693 | 7.579317 |
| 1370931_at | 7.774107 | 7.737186 | 8.387272 | 7.980927 | 8.107859 | 8.131368 | 6.964884 |
| 1367454_at | 11.35181 | 10.96007 | 11.30247 | 10.84985 | 11.04809 | 11.0709 | 10.13024 |
| 1375329_at | 3.412013 | 3.422034 | 3.392105 | 3.509135 | 3.419963 | 3.579827 | 3.189142 |
| 1375788_at | 7.343741 | 7.247983 | 7.105453 | 6.678797 | 7.549122 | 7.605156 | 5.672048 |
| 1367492_at | 10.92063 | 10.69177 | 10.70298 | 10.80301 | 10.85659 | 10.9212 | 10.24124 |
| 1373346_at | 11.36842 | 11.36714 | 11.31991 | 11.22142 | 11.35562 | 11.11352 | 10.54271 |
| 1392613_at | 5.481221 | 5.455458 | 5.248688 | 5.717384 | 5.61351 | 5.370742 | 6.588736 |
| 1373868_at | 8.665543 | 8.52902 | 8.657764 | 8.287104 | 8.183376 | 8.143034 | 7.262546 |
| 1388974_at | 11.2481 | 11.39397 | 10.92157 | 11.00218 | 10.7927 | 11.09298 | 10.16239 |
| 1373155_at | 8.923241 | 8.496463 | 8.39192 | 8.292827 | 8.333815 | 8.479348 | 7.61178 |
| 1375423_at | 11.23431 | 10.86617 | 10.63329 | 10.62934 | 10.99127 | 10.72646 | 9.390361 |
| 1377983_at | 5.938665 | 5.831154 | 5.97904 | 5.707722 | 5.958846 | 5.929467 | 5.086642 |
| 1374703_at | 5.596724 | 5.999135 | 5.649075 | 5.739903 | 5.774045 | 5.916106 | 6.929027 |
| 1382288_at | 8.30569 | 8.032848 | 8.033499 | 8.053241 | 8.19287 | 8.373211 | 7.415974 |
| 1374411_at | 8.758145 | 8.850084 | 8.902061 | 8.798115 | 8.748045 | 8.861299 | 7.78839 |
| 1377748_at | 6.027023 | 6.001341 | 5.954138 | 5.824864 | 5.693348 | 5.679235 | 4.874882 |
| 1377257_at | 6.476609 | 6.330833 | 6.449109 | 6.550034 | 6.257298 | 6.576049 | 5.625702 |
| 1396280_at | 7.282989 | 7.204092 | 7.030595 | 6.855897 | 7.193504 | 6.984796 | 6.456774 |
| 1395832_at | 4.7224 | 4.692362 | 4.768027 | 4.507789 | 4.721272 | 4.78133 | 4.259237 |
| 1379759_at | 9.030967 | 9.0014 | 8.848308 | 8.930095 | 9.041847 | 9.064055 | 8.353512 |
| 1387002_at | 10.68916 | 10.80265 | 10.8981 | 10.36402 | 10.67421 | 10.80987 | 9.690964 |
| 1373824_at | 11.32534 | 10.97234 | 11.27261 | 11.10521 | 10.69277 | 11.0942 | 10.11758 |
| 1392258_at | 5.766108 | 5.97203 | 6.222736 | 5.786221 | 6.184369 | 5.8555 | 5.008726 |
| 1392514_at | 8.58175 | 8.427594 | 8.617246 | 8.023539 | 7.859771 | 8.294312 | 7.150292 |
| 1390789_at | 9.424225 | 9.321047 | 9.365505 | 9.471624 | 9.448804 | 9.518779 | 8.717967 |
| 1387258_a_at | 10.92697 | 10.57541 | 10.69772 | 10.70767 | 10.38302 | 10.63779 | 9.760903 |
| 1398107_at | 4.337737 | 4.310229 | 4.222336 | 4.304347 | 4.35013 | 4.448268 | 3.957803 |
| 1398919_at | 11.01715 | 11.24309 | 11.05123 | 10.79939 | 10.83436 | 10.96189 | 9.989695 |
| 1369975_at | 10.12799 | 9.711004 | 10.07836 | 9.865083 | 9.787584 | 9.856238 | 9.243368 |
| 1388798_at | 10.52856 | 10.18319 | 10.42785 | 10.31944 | 10.9574 | 10.33247 | 9.063936 |
| 1373475_at | 9.346588 | 9.791472 | 9.552687 | 9.289251 | 9.274326 | 9.508961 | 8.316184 |
| 1372653_at | 10.25368 | 10.65953 | 10.21312 | 10.16066 | 9.608513 | 9.755047 | 8.476985 |
| 1376144_at | 8.857995 | 8.46322 | 8.659594 | 8.425791 | 8.828238 | 8.793244 | 7.739981 |
| 1375416_at | 9.894835 | 9.457933 | 9.5208 | 9.613246 | 9.94061 | 9.521029 | 8.696179 |
| 1371480_at | 10.28274 | 10.31081 | 10.22345 | 9.669149 | 10.28217 | 9.914357 | 9.121711 |
| 1368266_at | 6.763295 | 6.734196 | 7.146474 | 6.800329 | 6.929252 | 7.29395 | 5.900055 |
| 1371783_at | 10.87707 | 10.74704 | 10.87561 | 10.6618 | 10.45152 | 10.78217 | 9.677347 |
| 1387151_at | 8.951893 | 9.263478 | 8.921948 | 8.555851 | 8.498266 | 8.821349 | 7.955859 |
| 1386818_at | 6.699419 | 6.44565 | 6.04898 | 5.982313 | 6.401855 | 6.625793 | 5.372614 |
| 1374502_at | 9.427487 | 9.325881 | 9.220198 | 9.355511 | 9.674848 | 9.35446 | 8.188889 |
| 1388217_a_at | 11.26729 | 10.92615 | 11.06716 | 11.24955 | 11.32316 | 11.26769 | 10.21776 |
| 1376576_at | 9.620916 | 9.512235 | 9.76146 | 9.354869 | 9.774289 | 9.815722 | 9.046168 |
| 1370000_at | 11.33644 | 11.27049 | 10.91856 | 11.14028 | 11.74913 | 10.78381 | 9.470313 |
| 1372141_at | 10.24416 | 10.16173 | 9.862221 | 9.851954 | 9.969874 | 10.19664 | 9.266925 |
| 1370393_at | 8.989789 | 9.140623 | 8.60826 | 8.125942 | 8.175069 | 8.3846 | 7.357737 |
| 1389576_at | 10.12005 | 10.00802 | 10.25134 | 10.04863 | 9.885474 | 10.07426 | 9.457385 |
| 1382830_at | 4.969063 | 4.957268 | 5.202344 | 4.922326 | 4.876946 | 5.055368 | 5.370588 |
| 1374033_at | 7.711859 | 7.601099 | 7.685368 | 7.637296 | 7.383688 | 7.697378 | 6.834644 |
| 1376671_at | 12.22296 | 11.69939 | 11.60081 | 11.97408 | 11.76723 | 11.75523 | 10.7084 |
| 1387358_at | 11.74066 | 11.18677 | 11.28169 | 11.62427 | 11.25416 | 11.59928 | 10.33291 |
| 1388770_at | 11.15406 | 11.13059 | 11.0718 | 10.80027 | 10.56684 | 11.06096 | 9.817394 |
| 1371423_at | 9.826571 | 10.12291 | 9.900524 | 9.679627 | 9.372558 | 9.840867 | 8.956264 |
| 1368271_a_at | 7.22645 | 7.158662 | 7.382331 | 7.077927 | 7.005694 | 6.791975 | 5.153542 |
| 1395460_at | 8.253933 | 8.568113 | 8.555215 | 8.54373 | 8.015136 | 8.453797 | 9.064542 |
| 1390825_at | 9.079012 | 8.66244 | 8.833469 | 8.703624 | 8.561205 | 8.90307 | 7.850392 |
| 1372445_at | 9.558017 | 9.656089 | 9.885727 | 9.489672 | 8.838308 | 9.56834 | 8.368073 |
| 1372697_at | 8.465547 | 8.258319 | 8.321157 | 8.126768 | 8.040684 | 8.284172 | 7.364254 |
| 1374327_at | 5.086214 | 4.981402 | 5.08936 | 5.064869 | 5.179152 | 5.313857 | 4.636872 |
| 1385595_at | 7.413389 | 7.203759 | 7.354751 | 7.055188 | 7.336935 | 7.428443 | 6.497058 |
| 1370554_at | 10.96688 | 10.63323 | 11.04918 | 10.86727 | 10.68507 | 10.94256 | 9.973723 |
| 1383009_at | 6.912869 | 6.664708 | 6.737424 | 6.795533 | 6.032179 | 6.359737 | 4.896072 |
| 1398473_at | 9.753551 | 9.703123 | 9.524262 | 9.416597 | 9.388871 | 9.461454 | 8.930461 |
| 1373296_a_at | 9.384805 | 9.125605 | 9.186713 | 8.814689 | 8.853889 | 8.798578 | 7.816156 |
| 1371826_at | 7.386995 | 7.635351 | 7.697299 | 7.448534 | 7.646439 | 7.596388 | 8.253428 |
| 1371860_at | 10.56361 | 10.56474 | 10.03409 | 10.3387 | 10.62167 | 10.26466 | 9.31406 |
| 1389724_at | 7.923676 | 7.97331 | 7.905505 | 7.859257 | 7.659414 | 7.516264 | 8.566793 |
| 1367731_at | 9.359254 | 9.304589 | 9.260637 | 9.422495 | 9.333472 | 9.523438 | 8.230864 |
| 1372098_at | 8.296248 | 7.905817 | 8.032189 | 8.191926 | 8.360776 | 8.035287 | 7.460235 |
| 1375675_at | 6.832403 | 6.715793 | 6.580838 | 6.815643 | 7.195003 | 7.519896 | 5.71638 |
| 1391391_at | 4.143391 | 4.042431 | 3.900949 | 4.018972 | 4.557771 | 3.926286 | 3.34483 |
| 1393203_at | 8.509968 | 8.081169 | 8.156145 | 8.383277 | 8.075688 | 8.017791 | 7.439902 |
| 1389140_at | 11.42882 | 11.54041 | 11.41288 | 11.33641 | 11.77431 | 11.23695 | 10.63705 |
| 1378079_at | 8.705622 | 8.561362 | 8.485119 | 8.859117 | 8.812967 | 8.496126 | 7.767726 |
| 1377414_at | 7.240502 | 6.928997 | 7.119189 | 7.130626 | 7.072199 | 7.13773 | 6.377688 |
| 1376112_a_at | 8.120983 | 7.953225 | 8.275113 | 8.471656 | 8.212972 | 8.521376 | 8.864748 |
| 1380499_at | 7.096902 | 7.555627 | 7.36757 | 7.296937 | 6.971908 | 7.759879 | 8.262432 |
| 1387117_at | 9.239842 | 8.627353 | 9.067328 | 9.07941 | 9.908435 | 9.018607 | 7.679751 |
| 1372265_at | 9.936615 | 9.480063 | 9.263571 | 9.708231 | 9.929595 | 9.455903 | 8.375739 |
| 1372788_at | 6.272475 | 6.507809 | 6.304112 | 6.304166 | 6.228585 | 6.604101 | 7.070823 |
| 1389144_at | 10.52908 | 10.17599 | 10.50651 | 10.18955 | 10.31199 | 9.989998 | 9.457652 |
| 1397556_at | 10.95355 | 10.70988 | 10.44129 | 10.70957 | 10.22468 | 10.42136 | 9.342931 |
| 1371437_at | 10.93905 | 10.76274 | 10.68221 | 10.52552 | 10.22575 | 10.51585 | 9.731583 |
| 1394705_at | 6.854201 | 7.276433 | 7.35696 | 7.054683 | 7.576879 | 7.303773 | 7.946257 |
| 1377959_at | 10.31669 | 10.03976 | 9.963576 | 10.32545 | 9.840776 | 10.19258 | 9.013614 |
| 1387369_at | 8.757439 | 8.614517 | 8.932913 | 8.365343 | 8.682775 | 8.739358 | 7.907229 |
| 1393998_at | 7.287344 | 7.182058 | 6.957263 | 7.223258 | 8.005533 | 7.074296 | 6.157775 |
| 1380833_at | 5.863611 | 5.753466 | 5.826175 | 5.954673 | 5.894869 | 6.025795 | 6.621568 |
| 1389971_at | 11.53048 | 11.05212 | 11.3547 | 11.36379 | 11.62663 | 11.35344 | 10.41034 |
| 1373004_at | 8.407372 | 8.13739 | 7.804182 | 7.877106 | 8.325943 | 8.350611 | 7.263904 |
| 1374312_at | 8.317274 | 8.256462 | 8.159731 | 8.179248 | 7.76911 | 8.54096 | 8.859167 |
| 1371579_at | 10.47745 | 10.48049 | 10.49635 | 10.57486 | 10.38978 | 10.62749 | 9.709853 |
| 1384167_at | 6.830933 | 6.471175 | 6.559634 | 6.597221 | 6.292671 | 6.55103 | 5.863349 |
| 1373028_at | 10.48876 | 10.10403 | 10.01306 | 10.24261 | 10.50268 | 10.05094 | 9.116917 |
| 1368963_at | 9.202991 | 8.674288 | 8.788592 | 8.910909 | 9.510717 | 8.398826 | 7.731451 |
| 1396356_at | 5.884557 | 5.843796 | 5.889292 | 5.635707 | 5.422936 | 5.611467 | 6.406629 |
| 1389569_at | 10.33898 | 10.39449 | 10.04929 | 9.751926 | 9.792833 | 9.885402 | 9.291233 |
| AFFX-r2-Ec-bioB-3_at | 9.104404 | 8.916084 | 9.018956 | 8.602207 | 8.803377 | 9.229762 | 7.500797 |
| 1381894_at | 4.233439 | 4.298526 | 4.229693 | 4.307812 | 4.413648 | 4.362579 | 3.861528 |
| 1375137_at | 11.40949 | 11.16414 | 11.2307 | 11.41449 | 11.50925 | 11.05817 | 9.678642 |
| 1367927_at | 11.05998 | 10.96912 | 10.74842 | 10.83516 | 11.12415 | 10.98489 | 10.12094 |
| 1388983_at | 11.23725 | 11.23175 | 11.10347 | 10.95097 | 10.80165 | 11.02894 | 10.04058 |
| 1378048_at | 5.428737 | 5.380666 | 5.647621 | 5.313228 | 5.44218 | 5.732851 | 5.910779 |
| 1398380_at | 5.077252 | 5.015269 | 5.323194 | 5.201322 | 5.576184 | 5.463766 | 5.928657 |
| 1379320_at | 8.83969 | 8.756363 | 8.362041 | 8.313219 | 8.679595 | 9.191647 | 7.44084 |
| 1389544_at | 11.6342 | 11.85547 | 11.68407 | 11.3681 | 11.08268 | 11.22586 | 10.34572 |
| 1365442_at | 6.637823 | 6.818357 | 7.161564 | 6.759673 | 6.713581 | 6.62821 | 7.577412 |
| 1387954_a_at | 5.934968 | 6.031584 | 6.572301 | 6.092943 | 5.652663 | 5.830332 | 7.384813 |
| 1370933_at | 7.018824 | 7.080786 | 7.042022 | 7.411129 | 6.808394 | 6.699491 | 7.997149 |
| 1388931_at | 9.340996 | 9.704072 | 9.332376 | 8.934497 | 8.709092 | 9.182742 | 7.747403 |
| 1387778_at | 10.91236 | 10.62176 | 11.01682 | 10.99603 | 11.08399 | 11.06963 | 10.13167 |
| 1369179_a_at | 11.21994 | 11.03563 | 11.08728 | 11.36139 | 11.67994 | 11.35442 | 10.52307 |
| 1387668_at | 9.457034 | 8.81163 | 8.933284 | 8.860127 | 9.547914 | 8.462499 | 7.619094 |
| 1372218_at | 8.523125 | 8.360769 | 8.477298 | 8.318522 | 8.054949 | 8.265508 | 7.767317 |
| 1380544_at | 6.823932 | 6.952942 | 7.576106 | 7.296624 | 6.879099 | 7.310688 | 8.143329 |
| 1380542_at | 10.76558 | 10.77796 | 10.13388 | 10.64957 | 10.84431 | 10.4028 | 9.718057 |
| 1381611_at | 8.37599 | 8.068632 | 7.966115 | 8.294607 | 8.339909 | 8.098871 | 7.287355 |
| 1373412_at | 9.626165 | 9.319306 | 9.43173 | 9.45788 | 9.494756 | 9.958502 | 8.870172 |
| 1389659_at | 12.2334 | 12.13639 | 12.22741 | 12.00843 | 11.71399 | 11.78545 | 10.74108 |
| 1395662_at | 5.747917 | 5.708503 | 5.748032 | 6.228952 | 5.510929 | 6.012814 | 6.777294 |
| 1374358_at | 8.693209 | 8.704209 | 8.853696 | 8.71325 | 9.329224 | 8.956513 | 7.786054 |
| 1376086_at | 10.28986 | 10.43061 | 10.53099 | 9.759148 | 9.642587 | 10.00148 | 9.042845 |
| 1367491_at | 9.345176 | 9.579957 | 9.387091 | 9.248561 | 9.488142 | 9.427264 | 9.069241 |
| 1383659_a_at | 4.653612 | 5.331668 | 4.785695 | 5.12682 | 4.2096 | 5.130224 | 5.941054 |
| 1386519_x_at | 6.343099 | 6.847277 | 6.72809 | 6.413871 | 6.374874 | 7.185006 | 7.639416 |
| 1391709_at | 5.02859 | 5.12148 | 4.616199 | 4.766828 | 4.807043 | 4.822637 | 4.244203 |
| 1387280_a_at | 8.618922 | 9.719254 | 9.402267 | 9.693709 | 8.503693 | 9.386348 | 10.61001 |
| 1373574_at | 7.828976 | 7.979729 | 7.752996 | 7.780403 | 7.950851 | 8.067547 | 7.261557 |
| 1387851_at | 5.451756 | 5.218911 | 5.232182 | 5.2913 | 5.269652 | 5.486081 | 4.885362 |
| 1373822_at | 10.55978 | 10.71066 | 10.12106 | 10.33351 | 10.57441 | 10.11521 | 9.581621 |
| 1381802_at | 5.377716 | 5.395032 | 5.4132 | 5.366549 | 5.43052 | 5.297408 | 4.967644 |
| AFFX-BioB-5_at | 7.884688 | 8.076084 | 8.207758 | 7.554983 | 8.313151 | 8.286193 | 6.293382 |
| 1373312_at | 5.83417 | 5.958778 | 5.853042 | 5.745991 | 5.888422 | 5.679851 | 6.472997 |
| 1377267_at | 6.118475 | 6.53454 | 6.438749 | 6.15939 | 6.239287 | 6.140425 | 6.768212 |
| 1383643_at | 10.31661 | 10.57479 | 10.22989 | 9.969485 | 10.14963 | 9.899697 | 9.1559 |
| 1390524_at | 11.5106 | 11.12282 | 11.3799 | 11.40346 | 11.19683 | 11.24161 | 10.39042 |
| 1384007_at | 6.347491 | 6.340735 | 6.818929 | 6.615791 | 6.158216 | 6.631019 | 5.708891 |
| 1388488_at | 10.82017 | 11.00375 | 10.82933 | 10.43464 | 10.28504 | 10.68849 | 9.901916 |
| 1369786_at | 6.182177 | 6.044187 | 6.05767 | 6.19088 | 6.169734 | 5.980992 | 6.821094 |
| 1373943_at | 8.186405 | 8.176809 | 7.988301 | 8.304441 | 8.112037 | 8.127284 | 8.676683 |
| 1374792_at | 10.23933 | 9.998099 | 9.946066 | 9.877693 | 9.980774 | 9.919604 | 8.924216 |
| 1377067_at | 7.792694 | 7.908309 | 8.142457 | 7.876263 | 7.662474 | 7.922558 | 7.143462 |
| 1368587_at | 7.479179 | 7.342326 | 7.696387 | 7.550693 | 6.995942 | 7.072739 | 5.750006 |
| 1372152_at | 7.885238 | 7.897129 | 7.725253 | 7.659561 | 7.79469 | 7.874677 | 7.307988 |
| 1383380_at | 7.549448 | 7.283893 | 7.320321 | 7.456799 | 7.688685 | 7.49807 | 6.581571 |
| 1388777_at | 11.61357 | 11.36316 | 11.50383 | 11.39279 | 11.31538 | 11.62964 | 10.76956 |
| 1372547_at | 10.0671 | 10.11672 | 10.10716 | 9.822688 | 9.621243 | 10.06482 | 9.497121 |
| 1369445_at | 6.564591 | 6.555711 | 6.426586 | 6.616392 | 7.067066 | 6.561103 | 5.951585 |
| 1370745_at | 9.417545 | 8.73559 | 8.849631 | 8.652294 | 9.34045 | 9.281257 | 7.686914 |
| 1379948_at | 5.327545 | 5.154886 | 5.609054 | 5.597887 | 5.265244 | 5.5988 | 6.133078 |
| 1385698_at | 3.574118 | 3.749458 | 3.60862 | 3.742227 | 3.859492 | 3.614214 | 3.359176 |
| 1398781_at | 10.77701 | 10.6136 | 10.4277 | 10.4991 | 10.84694 | 10.35435 | 9.756388 |
| 1387798_a_at | 11.70704 | 11.52625 | 11.36092 | 11.66534 | 11.60245 | 11.59078 | 10.58468 |
| 1375945_at | 8.821124 | 8.814753 | 8.490458 | 8.143376 | 8.456712 | 7.977894 | 6.892831 |
| AFFX-r2-Ec-bioC-5_at | 10.75757 | 10.80698 | 10.91323 | 10.44974 | 10.76316 | 11.09963 | 9.116538 |
| 1374779_at | 6.934084 | 7.459993 | 7.262924 | 7.091138 | 7.000356 | 7.211512 | 6.616411 |
| 1368967_at | 8.299141 | 8.410174 | 8.04482 | 8.023519 | 7.981767 | 8.011829 | 7.433885 |
| 1372555_at | 7.169263 | 7.417301 | 7.272769 | 7.233717 | 6.821348 | 7.334815 | 7.926548 |
| 1382466_at | 8.533596 | 7.603011 | 8.412406 | 7.813268 | 8.033407 | 7.976726 | 7.039717 |
| 1381949_at | 5.975897 | 5.563435 | 5.510064 | 5.353271 | 6.076968 | 5.935246 | 4.611199 |
| 1388076_at | 11.10687 | 10.96948 | 10.49196 | 10.91121 | 10.79407 | 10.77854 | 9.912192 |
| 1374632_at | 8.049818 | 7.974612 | 7.467886 | 7.839303 | 8.120709 | 7.53313 | 6.959545 |
| 1377154_at | 6.545643 | 6.42385 | 6.151914 | 6.524133 | 6.006484 | 6.325225 | 5.313661 |
| 1370132_at | 6.855397 | 7.188383 | 6.850852 | 6.794809 | 6.905098 | 6.833138 | 5.794741 |
| 1373520_at | 8.977977 | 8.563481 | 8.615316 | 8.837165 | 8.780465 | 8.815774 | 9.250291 |
| 1380065_at | 8.135359 | 7.846636 | 7.819177 | 7.411543 | 7.199977 | 7.743512 | 6.316821 |
| 1385874_at | 6.329574 | 7.177705 | 5.910126 | 6.766094 | 6.575103 | 6.847777 | 4.408541 |
| 1390128_at | 9.222077 | 8.693432 | 8.394276 | 8.930095 | 9.180642 | 9.05576 | 7.725773 |
| 1389184_at | 9.046778 | 9.088023 | 8.794416 | 8.383261 | 8.307731 | 8.737334 | 7.681275 |
| 1375116_at | 5.46002 | 4.927421 | 5.434626 | 5.19372 | 5.187635 | 5.337275 | 4.503593 |
| 1368158_at | 10.40462 | 11.03377 | 10.73676 | 9.849494 | 9.554942 | 10.14591 | 8.631675 |
| AFFX-r2-Ec-bioB-5_at | 9.420403 | 9.173057 | 9.34067 | 8.777724 | 8.993713 | 9.567741 | 7.637679 |
| 1380732_at | 5.803142 | 5.729254 | 5.727748 | 5.753556 | 5.73498 | 5.732851 | 5.368619 |
| 1393368_at | 8.27869 | 8.445003 | 8.488706 | 8.599857 | 8.237893 | 8.288356 | 8.955829 |
| 1397680_at | 6.673534 | 6.589098 | 6.605751 | 6.259559 | 6.16055 | 6.273066 | 5.503821 |
| 1367981_at | 8.69738 | 8.576886 | 8.728003 | 8.825631 | 8.841395 | 8.586845 | 8.140733 |
| 1386851_a_at | 7.028297 | 6.953665 | 7.03152 | 7.260696 | 7.082441 | 6.81793 | 7.573606 |
| 1382310_at | 7.943187 | 7.617944 | 7.411962 | 7.448429 | 7.524249 | 7.520273 | 6.989008 |
| 1388313_at | 12.01223 | 12.43483 | 12.12562 | 11.71834 | 12.16061 | 12.2448 | 11.06161 |
| 1392476_at | 4.600697 | 4.952306 | 4.67426 | 4.774767 | 4.528378 | 4.786297 | 5.581648 |
| 1396265_at | 5.51761 | 5.339544 | 5.664069 | 5.59527 | 5.393931 | 5.68039 | 5.982135 |
| 1398316_at | 9.615721 | 9.560049 | 9.641917 | 9.809657 | 9.488379 | 9.885193 | 8.996917 |
| 1378297_at | 5.64003 | 5.581406 | 5.762889 | 5.789231 | 5.557349 | 5.814004 | 5.004532 |
| 1387519_at | 5.433014 | 5.378445 | 5.545016 | 5.428613 | 5.541033 | 5.495983 | 6.123898 |
| 1371718_at | 10.72274 | 10.23296 | 10.24606 | 10.34896 | 10.17323 | 10.48605 | 9.470822 |
| 1370353_at | 8.547415 | 8.174945 | 8.124709 | 8.216242 | 8.344061 | 8.358292 | 7.534117 |
| 1398429_at | 8.539098 | 8.202459 | 7.78695 | 8.275477 | 8.12569 | 8.357184 | 7.077549 |
| 1373707_at | 5.456777 | 5.718204 | 5.395482 | 5.66843 | 5.314901 | 5.313857 | 5.845882 |
| 1369084_a_at | 7.427758 | 7.636771 | 7.812229 | 7.624286 | 7.282453 | 7.816859 | 8.530877 |
| 1398972_at | 11.00841 | 11.07049 | 11.2225 | 10.70053 | 10.77038 | 11.03499 | 10.24555 |
| 1373121_at | 8.928827 | 9.04061 | 8.38164 | 8.313625 | 8.502678 | 8.531994 | 7.803954 |
| 1374390_at | 8.286418 | 8.23935 | 8.014833 | 8.172433 | 8.103821 | 8.141124 | 7.116686 |
| 1374245_at | 6.857086 | 6.484481 | 6.289268 | 6.482033 | 6.653339 | 6.728924 | 5.784886 |
| 1385842_at | 3.664971 | 3.671606 | 3.908613 | 3.624065 | 3.818389 | 3.875155 | 3.404174 |
| 1383797_a_at | 6.96823 | 6.669463 | 6.620841 | 6.925013 | 7.108937 | 6.798645 | 5.98945 |
| 1391161_at | 9.221779 | 9.29588 | 8.789443 | 9.031722 | 8.819901 | 8.529995 | 8.01873 |
| 1398983_at | 8.727155 | 8.497709 | 8.380428 | 8.002253 | 9.275658 | 8.487093 | 7.4554 |
| 1394280_at | 10.3422 | 9.475144 | 9.549217 | 9.701906 | 9.886174 | 9.695198 | 8.320196 |
| 1386129_at | 9.112622 | 8.989488 | 8.704591 | 8.959622 | 9.257718 | 8.51029 | 7.733661 |
| 1393982_at | 5.652437 | 5.548936 | 5.339262 | 5.430089 | 5.463696 | 5.323064 | 4.938421 |
| 1398312_s_at | 7.955425 | 7.59796 | 7.735129 | 7.655637 | 8.244525 | 7.095295 | 6.239227 |
| 1369996_at | 9.437712 | 9.472289 | 9.066947 | 9.239503 | 9.246812 | 9.294278 | 8.562754 |
| 1384327_at | 9.220263 | 9.377869 | 9.237635 | 9.658493 | 9.291579 | 9.399473 | 9.855333 |
| 1372483_at | 7.641772 | 8.168228 | 7.59112 | 7.642479 | 7.908959 | 7.259589 | 8.462928 |
| 1374575_at | 8.672844 | 9.139667 | 9.366762 | 9.259736 | 9.23817 | 9.164956 | 9.781004 |
| 1394383_at | 5.190077 | 5.191393 | 5.170524 | 5.515372 | 5.124596 | 5.118458 | 6.508132 |
| 1373103_at | 7.160123 | 7.224783 | 7.275109 | 7.513835 | 7.006621 | 7.408637 | 7.93256 |
| 1390836_at | 7.030715 | 7.042261 | 7.095564 | 6.484308 | 7.234028 | 7.198633 | 5.823807 |
| 1383017_at | 6.449769 | 6.755461 | 6.302895 | 6.506357 | 6.179847 | 5.87988 | 7.186737 |
| 1385461_at | 3.630642 | 3.442638 | 3.503516 | 3.507606 | 3.678778 | 3.576925 | 3.228609 |
| 1373769_at | 10.78306 | 10.32667 | 10.06404 | 9.954013 | 9.913968 | 9.820518 | 9.095233 |
| 1370073_at | 11.12395 | 10.69568 | 10.90156 | 10.88956 | 11.54969 | 10.95389 | 9.956153 |
| 1388869_at | 8.531097 | 8.701492 | 9.103695 | 8.344569 | 8.086582 | 8.625368 | 7.582649 |
| 1370769_a_at | 7.502889 | 6.426721 | 7.024413 | 6.435966 | 6.722415 | 6.410577 | 5.498569 |
| 1375550_at | 11.27235 | 10.82123 | 11.24794 | 11.09898 | 10.88392 | 10.9153 | 10.32525 |
| 1367501_at | 8.903749 | 9.055351 | 8.601381 | 8.730907 | 8.607119 | 8.443329 | 8.114646 |
| 1394445_at | 5.95503 | 5.974408 | 6.228186 | 6.186632 | 5.678592 | 5.995531 | 6.684009 |
| 1375738_at | 4.903972 | 4.914839 | 5.037582 | 4.780325 | 4.938754 | 5.12578 | 4.338394 |
| 1388853_at | 10.43456 | 10.39714 | 10.25604 | 10.17667 | 9.987344 | 10.27403 | 9.256092 |
| 1388397_at | 9.383407 | 9.239829 | 8.836492 | 9.170058 | 8.856984 | 9.431066 | 8.148727 |
| 1367831_at | 6.503634 | 7.108054 | 6.927438 | 7.085159 | 6.757764 | 6.811264 | 7.605849 |
| 1378569_at | 8.716384 | 8.209134 | 7.528101 | 8.081252 | 7.905942 | 7.536875 | 6.642356 |
| 1370142_at | 12.04635 | 12.08341 | 12.00047 | 11.96728 | 11.6952 | 11.4267 | 10.63202 |
| 1374332_at | 8.249751 | 7.676363 | 7.927869 | 7.953274 | 8.23012 | 7.975229 | 6.862446 |
| 1371971_at | 10.02232 | 9.862104 | 9.92434 | 10.09209 | 10.10368 | 9.763912 | 9.062588 |
| 1385476_x_at | 8.277342 | 8.367667 | 8.71692 | 8.76344 | 8.264143 | 8.353736 | 9.127885 |
| 1395637_at | 5.199141 | 5.32554 | 5.423601 | 5.224072 | 5.45806 | 5.184531 | 5.903754 |
| 1367586_at | 12.49783 | 12.35309 | 12.06841 | 12.43379 | 12.2533 | 12.09806 | 11.49527 |
| 1383068_at | 9.198225 | 8.936998 | 8.500378 | 8.623427 | 8.694119 | 9.144327 | 7.839437 |
| 1369720_at | 9.935782 | 9.462496 | 9.496313 | 9.453101 | 9.402832 | 9.133685 | 8.331377 |
| 1397271_at | 3.909212 | 3.77757 | 3.954154 | 3.943202 | 3.984845 | 3.910987 | 3.721575 |
| 1390190_at | 10.60472 | 10.08459 | 9.290084 | 9.915992 | 9.225897 | 9.365589 | 7.781048 |
| 1372219_at | 11.89317 | 11.81175 | 11.26311 | 11.50214 | 11.55188 | 11.23233 | 10.48791 |
| 1371977_at | 12.43388 | 12.54358 | 12.55431 | 12.34154 | 11.95579 | 12.51187 | 11.31954 |
| 1372119_at | 10.35158 | 10.30154 | 9.650608 | 10.00311 | 9.719291 | 9.542828 | 8.858429 |
| 1375669_at | 11.19044 | 10.71386 | 10.97012 | 11.19939 | 10.51998 | 10.80139 | 9.886157 |
| 1376209_at | 10.80562 | 10.9206 | 10.79093 | 10.64129 | 10.72828 | 10.46391 | 9.599445 |
| 1382266_at | 7.03075 | 7.126907 | 6.962091 | 7.012727 | 7.513528 | 7.113323 | 6.297605 |
| 1390559_at | 7.401403 | 7.339441 | 7.152271 | 7.953094 | 7.581964 | 7.61153 | 8.210349 |
| 1371812_at | 10.24831 | 10.01957 | 9.575026 | 10.14004 | 9.897517 | 10.19473 | 9.149246 |
| 1384428_at | 10.0576 | 9.815859 | 9.644881 | 9.95722 | 9.791205 | 10.17086 | 9.221903 |
| 1370817_at | 10.68306 | 10.55428 | 10.84359 | 10.6598 | 10.03393 | 10.69442 | 9.579288 |
| 1370040_at | 7.824764 | 7.802077 | 7.819936 | 7.978912 | 7.558501 | 7.847503 | 7.044322 |
| 1381492_at | 3.545007 | 3.579936 | 3.54666 | 3.536446 | 3.551384 | 3.338879 | 3.250333 |
| 1391494_at | 8.486105 | 8.289653 | 8.086382 | 8.628593 | 8.393531 | 8.500375 | 7.867493 |
| 1373041_at | 11.86722 | 12.30445 | 12.07367 | 11.75839 | 11.27816 | 11.7232 | 10.68742 |
| 1398777_at | 11.18328 | 11.10758 | 11.03105 | 10.66563 | 10.96733 | 10.89982 | 9.950038 |
| 1376143_at | 6.211125 | 6.251398 | 6.37412 | 6.3155 | 6.011453 | 6.232295 | 6.783417 |
| 1388351_at | 12.17621 | 12.20148 | 12.12514 | 11.93548 | 12.36843 | 11.98376 | 11.15336 |
| 1372786_at | 7.288936 | 7.500145 | 7.407274 | 7.274387 | 7.12005 | 7.434843 | 6.553575 |
| 1374004_at | 9.054887 | 8.87397 | 8.428339 | 8.907309 | 9.011019 | 8.75225 | 7.922688 |
| 1372794_at | 9.786688 | 9.554288 | 9.076588 | 9.240444 | 9.44353 | 9.398894 | 8.567296 |
| 1385754_a_at | 3.759867 | 3.714047 | 3.830468 | 3.622441 | 3.879629 | 3.77747 | 3.550581 |
| 1388363_at | 8.885957 | 9.104819 | 8.774542 | 9.022769 | 9.239762 | 8.427797 | 8.113387 |
| 1371633_at | 8.680475 | 8.60148 | 8.136978 | 8.704852 | 8.129205 | 8.314309 | 7.417277 |
| 1383260_at | 9.11262 | 9.326057 | 9.058267 | 9.362756 | 9.191955 | 9.498131 | 9.732601 |
| 1378400_at | 9.959037 | 9.539484 | 9.808858 | 9.451363 | 9.140839 | 9.072562 | 8.388681 |
| 1388112_at | 12.36728 | 12.36379 | 12.43484 | 12.42303 | 12.43905 | 12.23277 | 11.52311 |
| 1395871_at | 8.6161 | 8.990945 | 8.955609 | 8.862382 | 9.088026 | 8.72175 | 8.208284 |
| 1395488_at | 5.792636 | 6.018052 | 5.560567 | 5.109911 | 5.609942 | 5.843733 | 4.809275 |
| 1378206_a_at | 3.277718 | 3.327161 | 3.330898 | 3.368483 | 3.480531 | 3.34902 | 3.181561 |
| 1385785_at | 7.938763 | 7.732723 | 8.040244 | 7.484419 | 7.660321 | 7.071199 | 6.976742 |
| 1376971_at | 7.561155 | 7.552095 | 7.329573 | 7.531359 | 7.035504 | 7.626478 | 6.395097 |
| 1368116 a_at | 7.691474 | 7.807168 | 7.327687 | 7.698906 | 7.90816 | 7.39755 | 6.260976 |
| 1385755_at | 6.554187 | 6.906546 | 7.046552 | 7.005773 | 7.117844 | 7.001331 | 7.477989 |
| 1386687_at | 5.967246 | 6.070237 | 6.545995 | 6.23398 | 6.088609 | 6.60754 | 5.681151 |
| 1396057_at | 4.827305 | 5.26005 | 5.608439 | 4.917848 | 4.444208 | 4.817074 | 6.125593 |
| 1374711_at | 9.092302 | 8.940625 | 8.672471 | 9.056207 | 9.136088 | 8.926163 | 8.448197 |
| 1373440_at | 9.122579 | 9.569308 | 9.123375 | 8.760114 | 8.566953 | 9.135714 | 7.798163 |
| 1390649_at | 5.999955 | 6.441421 | 6.835051 | 6.616184 | 6.074488 | 6.894853 | 7.604169 |
| 1397504_at | 5.435327 | 5.460876 | 5.204937 | 5.162746 | 5.408879 | 5.284567 | 4.863371 |
| 1370282_at | 10.62357 | 10.77413 | 10.56951 | 10.66926 | 10.61061 | 10.30038 | 9.379386 |
| 1369510_at | 7.225022 | 7.226285 | 7.1753 | 7.017576 | 7.262259 | 6.9486 | 6.477264 |
| 1371869_at | 11.42101 | 11.14096 | 11.09282 | 11.43189 | 11.43011 | 11.27595 | 10.5534 |
| 1377735_at | 8.47047 | 8.832542 | 8.719615 | 8.490958 | 8.196444 | 8.451089 | 9.031784 |
| 1378567_at | 4.911359 | 4.888521 | 4.901132 | 4.798689 | 4.8392 | 4.946497 | 4.567359 |
| 1393938_at | 4.093552 | 4.335675 | 4.227933 | 4.073561 | 4.155841 | 3.841069 | 5.042275 |
| 1388542_at | 10.66939 | 10.33549 | 10.47014 | 10.51966 | 10.8483 | 10.40274 | 9.651672 |
| 1378449_at | 5.150645 | 5.15244 | 5.065539 | 5.061155 | 5.271796 | 5.244663 | 5.490331 |
| 1374268_at | 6.597486 | 6.987534 | 6.772467 | 7.07579 | 6.885779 | 6.827805 | 7.329569 |
| 1396623_at | 4.469416 | 4.299252 | 4.400769 | 4.488716 | 4.238936 | 4.250827 | 5.194306 |
| 1378203_at | 10.09926 | 9.757446 | 9.923665 | 9.704923 | 9.838416 | 9.533185 | 8.988104 |
| 1399158_a_at | 12.28305 | 12.42188 | 12.54719 | 12.00749 | 12.35177 | 12.3606 | 11.5713 |
| 1386125_at | 6.239131 | 6.103746 | 6.059425 | 6.065777 | 6.106269 | 5.823383 | 6.571218 |
| 1388478_at | 8.245284 | 8.507095 | 8.28382 | 8.939038 | 7.229571 | 8.347079 | 9.620268 |
| 1393085_at | 7.612322 | 7.506023 | 7.500458 | 7.152353 | 6.923561 | 7.197563 | 6.451345 |
| 1375521_at | 9.298725 | 9.60217 | 9.704636 | 8.917589 | 9.06814 | 9.602361 | 8.058678 |
| 1372981_at | 6.426917 | 6.397895 | 6.006682 | 6.179578 | 6.364589 | 6.052964 | 5.561908 |
| 1375173_at | 8.228476 | 8.263561 | 8.404688 | 8.667598 | 7.907767 | 8.859213 | 9.281527 |
| 1382318_at | 5.308187 | 5.099993 | 5.037948 | 5.311462 | 5.60017 | 5.331827 | 4.695611 |
| 1375554_at | 9.295298 | 9.00693 | 9.499635 | 9.255392 | 9.309052 | 9.466352 | 8.881835 |
| 1374629_at | 8.461878 | 8.462973 | 7.746929 | 8.189491 | 8.636755 | 8.093095 | 6.642224 |
| 1370944_at | 4.231313 | 4.088357 | 4.125342 | 4.175458 | 4.140088 | 4.167861 | 3.981804 |
| 1367696_at | 9.937185 | 10.01233 | 9.779358 | 9.602958 | 9.843477 | 10.01357 | 9.131795 |
| 1392504_at | 8.792292 | 9.1577 | 9.103804 | 8.373079 | 8.713664 | 9.37037 | 7.864499 |
| 1373840_at | 8.364185 | 8.472244 | 7.707612 | 8.16851 | 8.546883 | 7.898455 | 7.33696 |
| 1398772_at | 10.09132 | 9.877974 | 9.833789 | 10.10506 | 10.61225 | 9.806638 | 9.231795 |
| 1372941_at | 9.07774 | 8.605998 | 9.143803 | 9.227855 | 9.444166 | 9.160492 | 8.44131 |
| 1372891_at | 8.672415 | 8.501344 | 8.210153 | 8.556365 | 9.031954 | 8.427908 | 7.599659 |
| 1368665_at | 9.160761 | 8.838066 | 9.215996 | 9.223356 | 8.934809 | 9.289804 | 8.584223 |
| 1388067_a_at | 7.906626 | 8.318045 | 8.201978 | 7.868929 | 7.773406 | 8.192526 | 8.520595 |
| 1390924_at | 4.06192 | 4.043903 | 3.68541 | 4.279472 | 3.842681 | 4.140545 | 4.875183 |
| 1368123_at | 6.730894 | 7.176786 | 7.002677 | 7.263455 | 7.025015 | 6.732185 | 7.579371 |
| 1372393_at | 9.576884 | 9.749771 | 9.414518 | 9.463698 | 9.63369 | 9.514482 | 9.106114 |
| 1384943_at | 7.959989 | 7.448934 | 7.674723 | 7.924165 | 8.323245 | 7.668086 | 6.802452 |
| 1380768_at | 8.731238 | 8.341727 | 7.846456 | 8.561807 | 8.047375 | 7.758742 | 6.934635 |
| 1388013_at | 5.160067 | 5.20965 | 5.049472 | 5.054162 | 5.358765 | 5.305936 | 4.751945 |
| 1398241_a_at | 4.893755 | 4.750782 | 5.112981 | 4.922326 | 4.518246 | 4.79891 | 5.61564 |
| 1384207_at | 6.217328 | 5.919125 | 6.160232 | 6.461805 | 6.184369 | 6.039839 | 5.619473 |
| 1387617_at | 7.75292 | 7.626209 | 7.853034 | 7.236446 | 7.788922 | 8.130843 | 6.369482 |
| 1388745_at | 5.889647 | 5.706042 | 5.635169 | 5.850333 | 5.457495 | 5.699104 | 6.410927 |
| 1385975_at | 3.796771 | 3.895678 | 3.90319 | 3.765107 | 3.954042 | 3.917138 | 3.622503 |
| 1392745_at | 5.483165 | 5.522551 | 5.107172 | 5.673576 | 5.550157 | 5.409833 | 4.853226 |
| 1378229_at | 7.342366 | 7.286174 | 7.285885 | 7.34427 | 7.431781 | 7.36869 | 8.334038 |
| 1395860_at | 5.572803 | 5.54316 | 5.536056 | 5.524492 | 5.567243 | 5.520279 | 5.072472 |
| 1380257_at | 8.697206 | 8.378319 | 8.071879 | 8.238734 | 8.016195 | 8.362252 | 7.779837 |
| 1383627_a_at | 9.053065 | 8.883289 | 8.585575 | 8.944205 | 8.785793 | 8.957707 | 7.963417 |
| 1371120_a_at | 4.939244 | 4.887632 | 5.022229 | 5.160425 | 5.12247 | 5.134274 | 4.364065 |
| 1373135_at | 7.409322 | 7.174705 | 7.108247 | 7.114267 | 7.72691 | 7.180819 | 6.347497 |
| 1368062_at | 10.17505 | 9.893661 | 9.674087 | 9.659842 | 9.225017 | 9.707014 | 8.60567 |
| AFFX-r2-Ec-bioC-3_at | 10.5331 | 10.51947 | 10.57944 | 10.3609 | 10.26145 | 10.76676 | 9.034145 |
| 1398909_at | 10.90963 | 10.56166 | 10.1675 | 10.58963 | 10.48162 | 10.65575 | 9.581061 |
| 1399074_at | 9.686949 | 9.523367 | 9.441631 | 9.634288 | 9.92879 | 9.619097 | 9.150135 |
| 1384370_at | 6.979182 | 6.563088 | 6.157254 | 6.323197 | 6.504324 | 6.249173 | 5.880126 |
| 1370253_at | 11.77683 | 11.76233 | 11.87246 | 11.81606 | 11.82624 | 11.72085 | 10.84469 |
| 1374357_at | 8.677194 | 8.388554 | 8.638684 | 8.744697 | 9.033426 | 8.60606 | 7.938629 |
| 1384344_at | 7.655308 | 8.028686 | 7.74502 | 7.47159 | 7.784152 | 7.917863 | 7.263554 |
| 1396142_at | 8.540442 | 8.102148 | 8.122258 | 8.080623 | 7.63919 | 8.112175 | 7.414424 |
| 1371944_at | 11.52704 | 11.46512 | 11.47761 | 11.15331 | 11.1786 | 11.18823 | 10.49397 |
| 1378396_at | 8.785335 | 8.783595 | 8.307941 | 8.013725 | 8.754448 | 7.846316 | 7.117145 |
| 1376570_at | 11.9772 | 11.66166 | 11.61629 | 11.86291 | 11.74849 | 12.11282 | 10.85099 |
| 1371810_at | 8.393878 | 8.628044 | 8.31428 | 8.622637 | 8.6163 | 8.49628 | 9.033293 |
| 1367465_at | 11.2355 | 11.12897 | 11.17142 | 10.93655 | 10.86164 | 11.03392 | 10.27496 |
| 1376272_a_at | 11.10578 | 10.95727 | 10.82376 | 10.78794 | 10.70216 | 10.81952 | 9.840194 |
| 1389178_at | 9.340713 | 9.147465 | 9.253503 | 9.431504 | 9.266445 | 9.361841 | 8.659559 |
| 1376745_at | 7.906369 | 7.809776 | 7.778699 | 7.680884 | 7.870697 | 7.599536 | 7.180078 |
| 1391527_at | 5.883582 | 5.990601 | 5.865843 | 5.808785 | 6.080055 | 5.565411 | 6.353182 |
| 1369656_at | 7.258121 | 6.955004 | 6.806345 | 6.854458 | 7.036905 | 6.921142 | 6.396637 |
| 1389045_at | 6.881189 | 7.05104 | 7.021495 | 6.784965 | 7.191061 | 6.930738 | 6.480641 |
| 1384950_at | 7.612529 | 7.27074 | 7.562885 | 7.38059 | 7.106685 | 7.139099 | 6.843331 |
| 1387027_a_at | 9.610315 | 9.51282 | 9.236159 | 9.309089 | 9.237463 | 8.916966 | 8.699309 |
| 1370613_a_at | 8.060574 | 8.258322 | 8.017331 | 8.106267 | 8.131107 | 8.452003 | 8.428892 |
| 1392975_at | 8.021707 | 7.853626 | 7.928344 | 8.067241 | 8.087088 | 8.145476 | 7.0817 |
| 1396387_at | 4.619274 | 4.664927 | 4.652913 | 4.638131 | 4.297272 | 4.805021 | 4.131007 |
| ProbeSet | Cont | Cont | Cont | Cont | Cont | Cont | Cont |
| 1368220_at | 9.653834 | 9.613306 | 9.751509 | 9.802516 | 9.901954 | 9.740394 | 9.816408 |
| 1374716_at | 7.216461 | 7.390954 | 6.950959 | 7.40104 | 7.189865 | 7.265041 | 7.263649 |
| 1389323_at | 9.908174 | 9.758693 | 9.844095 | 9.955789 | 9.909206 | 10.01583 | 9.761389 |
| 1372137_at | 8.332438 | 8.421062 | 8.256026 | 8.366934 | 8.393782 | 8.520492 | 8.403472 |
| 1372829_at | 9.713535 | 9.372494 | 9.741099 | 9.706049 | 9.687592 | 9.590062 | 9.706159 |
| 1382415_at | 7.923295 | 7.934123 | 7.789955 | 8.022652 | 8.255266 | 8.09799 | 8.123367 |
| 1398572_at | 9.356603 | 9.392782 | 9.552094 | 9.379228 | 9.411425 | 9.374829 | 9.347443 |
| 1372269_at | 9.146252 | 8.946271 | 9.007884 | 9.052343 | 9.103104 | 8.963188 | 9.0467 |
| 1373711_at | 6.22161 | 6.240824 | 6.457751 | 6.662432 | 6.441491 | 6.544585 | 6.550978 |
| 1379623_at | 7.572664 | 7.428142 | 7.54863 | 7.607247 | 7.446549 | 7.760735 | 7.764145 |
| 1387663_at | 8.288776 | 8.390786 | 8.581683 | 8.655249 | 8.489414 | 8.314434 | 8.513276 |
| 1371745_at | 9.328014 | 9.089616 | 8.999589 | 9.182398 | 9.433273 | 9.092541 | 9.034973 |
| 1372977_at | 7.450589 | 7.484742 | 7.552006 | 7.848232 | 7.863487 | 7.556791 | 7.815581 |
| 1373490_at | 8.238269 | 8.158054 | 8.247392 | 8.496313 | 8.41424 | 8.37873 | 8.240092 |
| 1372209_at | 10.18446 | 10.00986 | 9.992081 | 10.23668 | 9.930347 | 10.09832 | 10.01536 |
| 1374790_at | 7.231058 | 7.425909 | 7.367049 | 7.58967 | 7.318426 | 7.365015 | 7.506072 |
| 1373632_at | 7.677435 | 7.441455 | 7.610775 | 8.080215 | 8.003776 | 8.043988 | 7.759112 |
| 1377025_at | 7.672326 | 8.044244 | 7.70837 | 7.60281 | 7.764422 | 7.823114 | 8.043589 |
| 1373660_at | 7.797569 | 7.756871 | 7.851719 | 7.837147 | 7.91015 | 7.995807 | 7.917596 |
| 1388171_at | 9.292573 | 9.095868 | 9.330543 | 9.184165 | 9.292742 | 9.20504 | 9.453952 |
| 1375345_at | 9.108234 | 9.134016 | 9.033444 | 9.359395 | 9.413448 | 9.215414 | 9.424971 |
| 1368164_at | 6.364968 | 5.842454 | 6.08368 | 6.289509 | 6.300155 | 6.189445 | 5.943217 |
| 1367929_at | 10.96173 | 11.11078 | 11.14862 | 11.36371 | 10.98079 | 10.90533 | 11.51518 |
| 1367945_at | 9.566811 | 9.592185 | 9.620033 | 9.590715 | 9.768888 | 9.79612 | 9.798014 |
| 1389970_at | 9.380145 | 9.315252 | 9.138343 | 9.579984 | 9.534429 | 9.31315 | 9.409601 |
| 1371484_at | 8.48348 | 8.423552 | 8.297052 | 8.540473 | 8.632938 | 8.43857 | 8.391625 |
| 1371435_at | 10.85905 | 10.81443 | 11.04899 | 10.98103 | 10.92913 | 11.12779 | 11.47571 |
| 1398770_at | 10.94405 | 10.8982 | 10.83529 | 11.18835 | 11.16987 | 11.27335 | 11.20023 |
| 1371421_at | 9.020823 | 8.75823 | 9.101526 | 8.999771 | 9.016944 | 9.203141 | 9.12982 |
| 1386930_at | 8.863005 | 8.776905 | 9.048524 | 8.9402 | 8.861528 | 9.313969 | 9.287128 |
| 1369588_a_at | 10.93149 | 10.39832 | 10.54365 | 10.79463 | 10.92203 | 10.6409 | 10.50991 |
| 1370321_at | 8.667936 | 8.54831 | 8.888157 | 8.621349 | 8.712261 | 8.852681 | 8.747769 |
| 1382825_at | 6.531674 | 6.719819 | 6.432039 | 6.564026 | 6.517236 | 6.536675 | 6.355054 |
| 1397956_at | 5.794244 | 5.841886 | 5.981651 | 5.729635 | 5.508516 | 5.764364 | 5.89736 |
| 1373982_at | 7.025288 | 7.002463 | 7.159019 | 7.133105 | 7.160775 | 6.950082 | 6.98517 |
| 1372436_at | 8.405846 | 8.311549 | 8.383372 | 8.439813 | 8.39598 | 8.549395 | 8.28441 |
| 1389009_at | 7.625302 | 7.622777 | 7.643129 | 7.588602 | 7.760825 | 7.547423 | 7.659211 |
| 1388328_at | 9.465118 | 9.482946 | 9.413395 | 9.658354 | 9.483672 | 9.691829 | 9.477855 |
| 1389053_at | 7.736656 | 7.582637 | 7.546453 | 7.766703 | 7.777574 | 7.829125 | 7.784148 |
| 1373184_at | 8.191098 | 8.365914 | 8.457245 | 8.393928 | 8.103611 | 8.350732 | 8.47948 |
| 1389409_at | 7.673193 | 8.075026 | 7.951605 | 8.488424 | 7.897603 | 7.999054 | 8.393088 |
| 1374291_at | 6.127086 | 6.362576 | 6.46708 | 6.47337 | 6.30078 | 6.197043 | 6.402778 |
| 1392517_at | 7.063267 | 7.464668 | 7.305467 | 7.329443 | 7.255915 | 7.234276 | 7.152618 |
| 1370931_at | 6.56399 | 6.560593 | 6.428021 | 6.546635 | 6.954216 | 6.553834 | 6.920546 |
| 1367454_at | 9.760477 | 9.981148 | 10.06223 | 10.09209 | 10.11029 | 10.23443 | 10.27881 |
| 1375329_at | 3.071477 | 3.037557 | 3.065659 | 3.109987 | 3.104286 | 3.040702 | 3.151466 |
| 1375788_at | 5.853605 | 5.613848 | 5.913141 | 5.511293 | 6.049114 | 5.745571 | 5.519636 |
| 1367492_at | 10.13283 | 9.927115 | 10.06942 | 10.12666 | 9.994521 | 10.29347 | 10.2648 |
| 1373346_at | 10.64224 | 10.57696 | 10.74608 | 10.57693 | 10.54422 | 10.79802 | 10.82191 |
| 1392613_at | 6.643804 | 6.151914 | 6.579364 | 6.391224 | 6.282915 | 6.609612 | 6.559769 |
| 1373868_at | 6.927279 | 7.034356 | 7.116095 | 6.924716 | 7.171428 | 7.073265 | 7.581429 |
| 1388974_at | 10.13807 | 10.14938 | 10.2876 | 10.17747 | 10.21081 | 9.924702 | 10.05949 |
| 1373155_at | 7.460041 | 7.639794 | 7.482996 | 7.661073 | 7.65725 | 7.573771 | 7.495351 |
| 1375423_at | 9.247397 | 9.159463 | 8.525143 | 9.50023 | 9.514849 | 9.331387 | 9.207059 |
| 1377983_at | 5.338988 | 5.039887 | 5.018583 | 5.187556 | 5.317287 | 5.296383 | 5.316521 |
| 1374703_at | 7.157669 | 6.999934 | 7.295538 | 7.235737 | 7.212398 | 6.715472 | 6.588358 |
| 1382288_at | 7.547851 | 7.170655 | 7.248748 | 7.258271 | 7.3556 | 7.558654 | 7.306933 |
| 1374411_at | 8.118375 | 8.04415 | 7.845144 | 8.277099 | 8.149607 | 8.203087 | 7.94924 |
| 1377748_at | 5.039089 | 5.047354 | 5.013054 | 5.234766 | 5.009313 | 5.242242 | 5.187063 |
| 1377257_at | 5.540815 | 5.854701 | 5.613673 | 5.353572 | 5.382229 | 5.494573 | 5.764019 |
| 1396280_at | 6.222816 | 6.136918 | 6.177345 | 6.220489 | 6.18222 | 6.478603 | 6.377348 |
| 1395832_at | 4.319589 | 4.234608 | 4.281347 | 4.209023 | 4.291632 | 4.365289 | 4.327183 |
| 1379759_at | 8.303636 | 8.375446 | 8.386139 | 8.679773 | 8.470053 | 8.333462 | 8.382845 |
| 1387002_at | 9.838814 | 9.499998 | 9.800198 | 9.950808 | 9.953632 | 9.570551 | 9.694058 |
| 1373824_at | 10.22365 | 10.05346 | 10.27845 | 10.17465 | 10.23006 | 9.923338 | 10.0084 |
| 1392258_at | 5.223374 | 4.936544 | 4.96905 | 4.969653 | 5.073034 | 5.262274 | 5.206817 |
| 1392514_at | 7.266832 | 7.31672 | 7.106388 | 7.17561 | 7.197302 | 7.158357 | 7.224119 |
| 1390789_at | 7.805398 | 8.235014 | 8.179543 | 8.351143 | 8.179184 | 8.351237 | 8.519111 |
| 1387258_a_at | 9.602888 | 9.542198 | 9.764402 | 9.948383 | 9.656637 | 9.85221 | 9.973836 |
| 1398107_at | 4.049959 | 3.967135 | 4.091614 | 4.026759 | 4.000037 | 3.988107 | 4.034144 |
| 1398919_at | 10.20135 | 10.00517 | 10.13321 | 10.27811 | 10.3298 | 10.37206 | 10.10729 |
| 1369975_at | 9.113125 | 8.833654 | 9.144399 | 9.248898 | 8.912069 | 9.071859 | 9.076613 |
| 1388798_at | 8.923525 | 8.757726 | 8.564628 | 9.377526 | 9.167551 | 9.223845 | 9.165259 |
| 1373475_at | 8.577434 | 8.26893 | 8.430146 | 8.774542 | 8.440304 | 8.603792 | 8.577267 |
| 1372653_at | 8.24248 | 8.376603 | 7.73434 | 8.080044 | 8.328574 | 8.724909 | 8.540927 |
| 1376144_at | 7.69435 | 7.73614 | 7.691013 | 7.987716 | 7.929875 | 7.782972 | 8.019711 |
| 1375416_at | 8.89349 | 8.674303 | 8.612014 | 8.859321 | 8.866606 | 8.960429 | 8.730339 |
| 1371480_at | 9.25519 | 9.059479 | 9.048466 | 9.194829 | 9.232182 | 9.034594 | 8.93823 |
| 1368266_at | 5.207003 | 5.478505 | 5.98107 | 5.533941 | 5.764177 | 5.278213 | 5.511802 |
| 1371783_at | 9.820777 | 9.667189 | 9.607785 | 9.849394 | 9.983331 | 10.01587 | 10.0779 |
| 1387151_at | 7.891303 | 7.766522 | 7.849548 | 7.868702 | 7.826623 | 7.803401 | 7.71099 |
| 1386818_at | 5.134036 | 5.479484 | 5.111098 | 5.134336 | 5.240009 | 5.212592 | 5.314944 |
| 1374502_at | 7.944046 | 7.810094 | 8.090097 | 8.649345 | 8.463163 | 8.222263 | 8.284877 |
| 1388217_a_at | 10.28459 | 10.15025 | 10.30606 | 10.41095 | 10.15075 | 10.32432 | 10.69384 |
| 1376576_at | 8.850427 | 8.758749 | 8.645326 | 8.760429 | 8.947084 | 8.746395 | 8.591645 |
| 1370000_at | 9.796261 | 9.761008 | 9.71484 | 9.769645 | 9.804143 | 10.19102 | 9.948545 |
| 1372141_at | 9.066067 | 9.118846 | 9.163499 | 9.256716 | 9.178907 | 9.187034 | 9.558824 |
| 1370393_at | 7.005012 | 6.833603 | 7.049584 | 7.194719 | 6.979463 | 6.975569 | 7.015726 |
| 1389576_at | 9.430444 | 9.377893 | 9.248937 | 9.406268 | 9.507169 | 9.043209 | 9.429165 |
| 1382830_at | 5.667489 | 5.565345 | 5.562776 | 5.478086 | 5.479949 | 5.610762 | 5.466196 |
| 1374033_at | 7.11412 | 7.084229 | 6.845779 | 7.051444 | 6.984657 | 6.716771 | 6.842309 |
| 1376671_at | 10.50605 | 10.78179 | 10.76676 | 11.09455 | 10.8939 | 10.94725 | 10.93852 |
| 1387358_at | 10.45246 | 10.37732 | 10.5768 | 10.50808 | 10.65674 | 10.66108 | 10.67683 |
| 1388770_at | 9.784046 | 9.995755 | 9.640942 | 10.19142 | 10.07757 | 10.07966 | 9.799496 |
| 1371423_at | 8.740699 | 8.829713 | 8.649535 | 9.009971 | 8.984172 | 8.742437 | 8.739554 |
| 1368271_a_at | 4.966151 | 4.808825 | 5.007246 | 4.721571 | 6.260734 | 5.352101 | 5.014153 |
| 1395460_at | 9.492133 | 9.39874 | 9.233975 | 9.671084 | 9.379137 | 9.366003 | 9.507433 |
| 1390825_at | 7.974075 | 8.05409 | 8.139648 | 8.15082 | 8.204437 | 8.0638 | 8.012046 |
| 1372445_at | 8.20502 | 8.16901 | 8.211179 | 8.456086 | 8.342743 | 8.399089 | 8.29435 |
| 1372697_at | 7.518571 | 7.399024 | 7.382786 | 7.621987 | 7.801486 | 7.444319 | 7.336089 |
| 1374327_at | 4.318602 | 4.24072 | 4.38115 | 4.268277 | 4.289088 | 4.482657 | 4.644195 |
| 1385595_at | 6.085176 | 6.373179 | 6.534627 | 6.479467 | 6.51718 | 6.623829 | 6.68187 |
| 1370554_at | 9.315952 | 9.361318 | 9.428295 | 9.681884 | 9.359376 | 9.866765 | 10.0216 |
| 1383009_at | 4.464336 | 4.239068 | 4.452961 | 4.931569 | 4.712102 | 5.283319 | 5.300596 |
| 1398473_at | 8.984519 | 9.035425 | 8.761812 | 8.92563 | 8.873497 | 9.052939 | 8.973254 |
| 1373296_a_at | 7.669535 | 7.690542 | 7.561096 | 8.091705 | 7.788816 | 8.159108 | 8.145614 |
| 1371826_at | 8.220765 | 8.299376 | 8.150677 | 8.610986 | 8.413523 | 8.10266 | 8.223716 |
| 1371860_at | 9.387385 | 9.311294 | 9.15528 | 9.661137 | 9.564964 | 9.515983 | 9.581458 |
| 1389724_at | 8.693326 | 8.301776 | 8.491013 | 8.58323 | 8.490989 | 8.542394 | 8.436262 |
| 1367731_at | 8.590666 | 8.67017 | 8.54421 | 8.716679 | 8.320948 | 8.027091 | 8.440193 |
| 1372098_at | 7.353977 | 7.496302 | 7.257407 | 7.562267 | 7.56665 | 7.348907 | 7.250609 |
| 1375675_at | 5.571161 | 5.706857 | 5.935755 | 5.868945 | 5.553661 | 5.847495 | 5.687046 |
| 1391391_at | 3.254302 | 3.295368 | 3.176084 | 3.332497 | 3.194105 | 3.25479 | 3.418926 |
| 1393203_at | 7.143849 | 7.373181 | 7.564006 | 7.492225 | 7.169206 | 7.408211 | 7.441881 |
| 1389140_at | 10.55324 | 10.49167 | 10.52294 | 10.64962 | 10.67737 | 10.96543 | 10.72884 |
| 1378079_at | 7.257383 | 7.403049 | 7.375767 | 7.452319 | 7.370411 | 7.707995 | 8.049484 |
| 1377414_at | 6.317311 | 6.045799 | 6.398293 | 6.437756 | 6.154128 | 6.461192 | 6.641882 |
| 1376112_a_at | 9.157607 | 9.146404 | 9.048216 | 9.3596 | 9.066428 | 9.124907 | 9.028397 |
| 1380499_at | 8.494152 | 8.483988 | 8.836062 | 8.628059 | 8.293904 | 8.479304 | 8.344125 |
| 1387117_at | 7.49646 | 7.26906 | 7.655663 | 7.715697 | 7.569023 | 7.883457 | 7.897103 |
| 1372265_at | 8.280532 | 8.103065 | 8.183192 | 8.697397 | 8.538316 | 8.706206 | 8.565607 |
| 1372788_at | 6.913111 | 6.784491 | 7.006146 | 6.960325 | 6.922715 | 7.06605 | 6.890393 |
| 1389144_at | 9.119197 | 9.026225 | 8.92469 | 9.057368 | 8.728955 | 9.370588 | 9.42371 |
| 1397556_at | 9.261701 | 9.084929 | 9.17943 | 9.513192 | 9.229159 | 9.710992 | 9.718362 |
| 1371437_at | 9.702702 | 9.836713 | 9.762023 | 9.848816 | 9.857547 | 9.900241 | 9.647962 |
| 1394705_at | 8.035372 | 8.179636 | 8.386381 | 8.204637 | 8.17121 | 8.192342 | 8.069509 |
| 1377959_at | 9.217312 | 9.164285 | 8.719402 | 9.273007 | 9.367498 | 9.298777 | 9.178847 |
| 1387369_at | 8.082835 | 7.955214 | 7.842472 | 8.069653 | 8.075706 | 7.84323 | 8.089583 |
| 1393998_at | 6.220091 | 6.07813 | 6.053283 | 6.190981 | 6.11879 | 6.125797 | 6.146988 |
| 1380833_at | 6.726801 | 6.491247 | 6.43567 | 6.528461 | 6.423774 | 6.694573 | 6.256765 |
| 1389971_at | 10.53671 | 10.45684 | 10.41455 | 10.7635 | 10.68086 | 10.73994 | 10.66517 |
| 1373004_at | 7.340038 | 7.347483 | 7.136682 | 6.944059 | 7.341681 | 7.214345 | 7.103259 |
| 1374312_at | 9.197937 | 9.026879 | 9.169408 | 9.236282 | 9.093836 | 9.039792 | 9.058059 |
| 1371579_at | 9.840194 | 9.890448 | 10.2319 | 9.884999 | 9.920561 | 9.93445 | 9.965834 |
| 1384167_at | 5.648656 | 5.55073 | 5.824802 | 6.047078 | 5.691822 | 5.732187 | 5.881933 |
| 1373028_at | 9.23652 | 9.435814 | 9.367577 | 9.433669 | 9.241678 | 9.598161 | 9.499128 |
| 1368963_at | 7.206068 | 7.360908 | 7.198754 | 7.613415 | 7.694034 | 7.251171 | 7.647539 |
| 1396356_at | 6.446798 | 6.352549 | 6.438778 | 6.238132 | 6.31352 | 6.241376 | 6.401422 |
| 1389569_at | 9.147256 | 9.153133 | 9.060609 | 8.982943 | 9.17923 | 8.894731 | 9.036023 |
| AFFX-r2-Ec-bioB-3_at | 7.180867 | 7.117483 | 7.150611 | 7.474551 | 7.584297 | 8.006147 | 8.03028 |
| 1381894_at | 4.012989 | 3.59395 | 3.775328 | 3.765985 | 3.691247 | 3.904732 | 3.818887 |
| 1375137_at | 9.702332 | 9.67407 | 9.811091 | 9.723944 | 9.6669 | 10.43034 | 10.55382 |
| 1367927_at | 10.29063 | 10.05677 | 10.14234 | 10.54564 | 10.3614 | 10.30239 | 10.28941 |
| 1388983_at | 10.14473 | 10.04552 | 10.1421 | 10.28077 | 10.34838 | 10.45797 | 10.50706 |
| 1378048_at | 6.238276 | 6.160493 | 6.27368 | 6.332724 | 6.173286 | 6.431126 | 6.338632 |
| 1398380_at | 6.484364 | 5.990335 | 6.317457 | 6.189673 | 6.358057 | 6.434771 | 6.202494 |
| 1379320_at | 7.336512 | 7.337212 | 7.915862 | 7.409374 | 7.586854 | 7.300678 | 7.190615 |
| 1389544_at | 10.46287 | 10.1982 | 10.20514 | 10.45537 | 10.64661 | 10.58916 | 10.21326 |
| 1365442_at | 7.574966 | 7.395331 | 7.680526 | 7.505959 | 7.596168 | 7.465275 | 7.302924 |
| 1387954_a_at | 7.598039 | 7.559635 | 7.839852 | 7.576037 | 7.42256 | 6.785334 | 7.347007 |
| 1370933_at | 8.173625 | 8.080293 | 8.221579 | 7.80936 | 7.96666 | 7.711278 | 7.844831 |
| 1388931_at | 7.94429 | 7.744826 | 7.882884 | 8.066405 | 8.139902 | 8.251143 | 8.08972 |
| 1387778_at | 10.06858 | 10.11961 | 9.919712 | 10.44711 | 10.35668 | 10.20412 | 10.19834 |
| 1369179_a_at | 10.37059 | 10.3969 | 10.45293 | 10.43878 | 10.40244 | 10.59847 | 10.71145 |
| 1387668_at | 6.506948 | 6.842064 | 6.730364 | 7.529765 | 7.133032 | 7.473687 | 7.26146 |
| 1372218_at | 7.760357 | 7.379802 | 7.65412 | 7.631416 | 7.710772 | 7.537081 | 7.391128 |
| 1380544_at | 8.314316 | 8.19687 | 8.931784 | 8.307644 | 8.399071 | 8.358795 | 8.694089 |
| 1380542_at | 9.367772 | 9.525627 | 9.284472 | 9.657058 | 9.2417 | 9.664293 | 9.735141 |
| 1381611_at | 7.149797 | 7.120713 | 7.469576 | 7.11515 | 7.052081 | 7.531915 | 7.608519 |
| 1373412_at | 8.704065 | 8.535094 | 8.616941 | 8.939843 | 8.824119 | 8.591645 | 8.698549 |
| 1389659_at | 10.25132 | 9.926691 | 9.653977 | 10.69682 | 10.70259 | 10.65138 | 10.69349 |
| 1395662_at | 6.803611 | 6.805203 | 7.368845 | 6.910296 | 6.627417 | 7.06249 | 7.115875 |
| 1374358_at | 8.125397 | 7.771655 | 8.018233 | 8.108162 | 8.062086 | 8.106332 | 8.0421 |
| 1376086_at | 8.94085 | 9.021965 | 8.933149 | 9.013849 | 9.179259 | 8.915017 | 8.944574 |
| 1367491_at | 8.946981 | 9.00809 | 9.065927 | 8.92641 | 8.865164 | 8.981322 | 9.057647 |
| 1383659_a_at | 6.225572 | 6.434002 | 6.664831 | 6.693204 | 6.224589 | 6.213375 | 6.419965 |
| 1386519_x_at | 7.697284 | 7.744801 | 7.988437 | 8.201759 | 7.855421 | 7.706035 | 7.735644 |
| 1391709_at | 4.364638 | 4.175316 | 4.333243 | 4.204283 | 4.177999 | 4.170717 | 4.349233 |
| 1387280_a_at | 11.62104 | 11.1 | 11.56877 | 11.62003 | 11.65946 | 10.84028 | 11.35313 |
| 1373574_at | 6.776128 | 7.07613 | 7.232019 | 7.262739 | 6.718328 | 6.995441 | 7.189131 |
| 1387851_at | 4.834195 | 4.953541 | 4.683093 | 4.827549 | 4.860307 | 4.878332 | 4.994257 |
| 1373822_at | 9.610756 | 9.56302 | 9.519638 | 9.761939 | 9.390009 | 9.685369 | 9.558301 |
| 1381802_at | 5.133389 | 5.021544 | 4.939602 | 4.800999 | 5.029379 | 5.107063 | 4.921184 |
| AFFX-BioB-5_at | 6.191643 | 6.099997 | 5.866418 | 6.164723 | 6.493296 | 6.895724 | 7.109796 |
| 1373312_at | 6.974079 | 6.585195 | 6.856666 | 7.195152 | 7.093823 | 6.58407 | 6.602163 |
| 1377267_at | 7.030366 | 7.083777 | 7.000356 | 6.786624 | 7.091193 | 6.857144 | 6.897397 |
| 1383643_at | 9.414651 | 9.195636 | 9.078208 | 9.296962 | 9.549539 | 9.390623 | 9.062807 |
| 1390524_at | 10.34741 | 10.10698 | 10.47689 | 10.65227 | 10.44658 | 10.5547 | 10.81836 |
| 1384007_at | 5.402787 | 5.608722 | 5.340987 | 5.537179 | 5.637549 | 5.579712 | 5.868322 |
| 1388488_at | 9.918415 | 9.84744 | 9.792371 | 9.833111 | 9.948385 | 9.667702 | 9.669216 |
| 1369786_at | 6.925641 | 6.640537 | 6.772097 | 6.697978 | 6.72894 | 6.492473 | 6.466996 |
| 1373943_at | 8.971493 | 8.878458 | 9.118229 | 8.942941 | 8.611125 | 8.631861 | 8.810626 |
| 1374792_at | 8.97371 | 8.832425 | 9.315143 | 9.223822 | 8.929621 | 9.329097 | 9.455198 |
| 1377067_at | 6.815986 | 6.472088 | 6.131349 | 6.701503 | 6.719077 | 7.035752 | 6.773645 |
| 1368587_at | 5.61992 | 6.203566 | 5.10047 | 5.702196 | 6.392133 | 5.779497 | 5.997662 |
| 1372152_at | 7.306013 | 7.184793 | 7.331543 | 7.555883 | 7.296914 | 7.288823 | 7.124092 |
| 1383380_at | 6.787443 | 6.679663 | 6.691958 | 6.985708 | 6.895662 | 6.98444 | 6.862489 |
| 1388777_at | 10.91106 | 10.75676 | 10.87209 | 10.82748 | 11.00405 | 11.13415 | 10.9396 |
| 1372547_at | 9.187429 | 9.269425 | 9.239551 | 9.271586 | 9.363323 | 9.153975 | 9.11682 |
| 1369445_at | 5.745999 | 5.997809 | 5.858027 | 5.961865 | 5.835804 | 6.063878 | 5.911927 |
| 1370745_at | 7.855499 | 7.569003 | 8.183798 | 7.920496 | 8.098453 | 7.775945 | 7.646872 |
| 1379948_at | 5.947673 | 6.105553 | 6.340087 | 6.109147 | 6.072914 | 6.046058 | 6.091871 |
| 1385698_at | 3.298256 | 3.364521 | 3.314536 | 3.393035 | 3.338055 | 3.342325 | 3.414804 |
| 1398781_at | 9.821768 | 9.716559 | 9.675101 | 10.14793 | 9.838288 | 9.854432 | 9.930738 |
| 1387798_a_at | 10.76014 | 10.65338 | 10.92651 | 10.95048 | 11.02438 | 11.04875 | 11.03559 |
| 1375945_at | 6.092357 | 6.579615 | 6.019518 | 6.753838 | 6.140772 | 7.260384 | 7.052927 |
| AFFX-r2-Ec-bioC-5_at | 9.146365 | 9.075491 | 8.946104 | 9.247122 | 9.566802 | 9.892755 | 9.963453 |
| 1374779_at | 6.325698 | 6.082248 | 6.537306 | 6.138392 | 6.426555 | 6.2728 | 6.345401 |
| 1368967_at | 7.413096 | 7.438864 | 7.413151 | 7.560074 | 7.525405 | 7.194025 | 7.075911 |
| 1372555_at | 8.110697 | 8.048412 | 7.812677 | 7.969236 | 8.196956 | 7.784623 | 7.881314 |
| 1382466_at | 6.35215 | 6.752973 | 6.604515 | 6.718194 | 6.723047 | 6.903969 | 7.113119 |
| 1381949_at | 4.783321 | 4.68354 | 4.826719 | 4.952799 | 4.883776 | 4.936281 | 4.814208 |
| 1388076_at | 9.991928 | 10.21376 | 10.00341 | 10.27569 | 9.91507 | 10.10664 | 10.22521 |
| 1374632_at | 6.99028 | 7.16095 | 6.970782 | 7.010869 | 7.006634 | 7.110734 | 6.897876 |
| 1377154_at | 5.407544 | 5.465662 | 5.484999 | 5.632782 | 5.787936 | 5.59281 | 5.655322 |
| 1370132_at | 6.026494 | 6.151731 | 5.783816 | 6.315001 | 6.40515 | 6.063878 | 5.979944 |
| 1373520_at | 9.797819 | 9.480656 | 9.862158 | 9.833582 | 9.660839 | 9.39614 | 9.627033 |
| 1380065_at | 6.147534 | 5.513744 | 6.210199 | 6.384391 | 6.166094 | 6.621542 | 6.516608 |
| 1385874_at | 4.788051 | 4.746153 | 5.499339 | 4.843012 | 4.518437 | 5.051292 | 5.217419 |
| 1390128_at | 7.698957 | 7.711194 | 7.607988 | 7.882625 | 8.131594 | 8.00687 | 7.952855 |
| 1389184_at | 7.938302 | 7.66815 | 7.771063 | 7.730461 | 7.796179 | 7.583377 | 7.493476 |
| 1375116_at | 4.706483 | 4.678171 | 4.732123 | 4.649198 | 4.614635 | 4.706483 | 4.609699 |
| 1368158_at | 8.708552 | 8.591588 | 8.227174 | 8.810564 | 8.869976 | 8.484019 | 8.517939 |
| AFFX-r2-Ec-bioB-5_at | 7.353342 | 7.574685 | 7.341096 | 7.605825 | 7.820091 | 8.318869 | 8.253989 |
| 1380732_at | 5.431774 | 5.356537 | 5.472805 | 5.482876 | 5.544972 | 5.526105 | 5.577282 |
| 1393368_at | 9.505332 | 9.226868 | 9.452172 | 9.968472 | 9.397741 | 9.408219 | 9.339896 |
| 1397680_at | 5.656297 | 5.434696 | 5.29624 | 5.673769 | 5.517495 | 5.753782 | 5.830889 |
| 1367981_at | 7.760294 | 8.336312 | 7.981452 | 7.91106 | 7.825218 | 7.699373 | 7.763871 |
| 1386851_a_at | 7.760932 | 7.961813 | 7.495712 | 7.827499 | 7.889379 | 7.53896 | 7.725725 |
| 1382310_at | 6.797861 | 6.999245 | 6.961889 | 7.000743 | 6.927368 | 6.918478 | 7.121128 |
| 1388313_at | 10.84844 | 11.01878 | 10.87013 | 11.22353 | 11.27085 | 11.34498 | 11.44579 |
| 1392476_at | 5.512431 | 5.506363 | 5.464288 | 5.951507 | 5.695284 | 5.279111 | 5.311628 |
| 1396265_at | 6.087611 | 5.996187 | 6.106408 | 5.876395 | 6.010482 | 6.144059 | 6.204385 |
| 1398316_at | 9.012061 | 8.561774 | 8.97785 | 9.087252 | 8.97474 | 8.584836 | 8.704014 |
| 1378297_at | 5.175138 | 4.798178 | 5.0128 | 4.94877 | 4.95673 | 5.260167 | 5.290027 |
| 1387519_at | 6.030607 | 6.240345 | 5.997602 | 5.828335 | 5.919945 | 5.968116 | 5.791177 |
| 1371718_at | 9.739085 | 9.658461 | 9.509062 | 9.764748 | 9.851166 | 9.637623 | 9.692875 |
| 1370353_at | 7.728174 | 7.695456 | 7.491503 | 7.753443 | 7.742999 | 7.556106 | 7.260227 |
| 1398429_at | 7.256687 | 7.223021 | 7.363524 | 7.443006 | 7.452301 | 7.572463 | 7.416961 |
| 1373707_at | 6.158298 | 6.166102 | 6.29911 | 6.099049 | 6.080423 | 6.239648 | 6.163975 |
| 1369084_a_at | 8.750741 | 8.236399 | 8.713287 | 8.599022 | 8.369874 | 8.33876 | 8.261815 |
| 1398972_at | 10.3482 | 10.21579 | 10.10672 | 10.43203 | 10.49573 | 10.24591 | 10.21388 |
| 1373121_at | 7.779757 | 7.722471 | 7.642762 | 7.779388 | 7.771772 | 7.503198 | 7.435442 |
| 1374390_at | 7.546961 | 7.329734 | 7.171851 | 7.743763 | 7.538235 | 7.198017 | 7.491702 |
| 1374245_at | 5.379223 | 5.429461 | 5.60097 | 5.797645 | 5.742579 | 6.00915 | 5.823725 |
| 1385842_at | 3.350558 | 3.157126 | 3.270699 | 3.194356 | 3.223982 | 3.420383 | 3.354307 |
| 1383797_a_at | 5.681534 | 5.894448 | 5.874273 | 6.202177 | 6.085068 | 6.291706 | 6.155209 |
| 1391161_at | 7.583184 | 7.485386 | 7.731925 | 7.837504 | 7.524826 | 8.085382 | 8.123955 |
| 1398983_at | 7.304515 | 7.302743 | 6.846152 | 7.416484 | 7.208806 | 7.101109 | 7.316611 |
| 1394280_at | 7.62929 | 8.067375 | 8.090227 | 8.402874 | 8.382814 | 8.830627 | 8.657991 |
| 1386129_at | 7.498188 | 7.893244 | 8.130031 | 7.663454 | 7.376398 | 7.928327 | 8.060381 |
| 1393982_at | 4.882573 | 4.942199 | 4.852007 | 4.864439 | 5.025438 | 5.036091 | 5.178242 |
| 1398312_s_at | 6.129481 | 6.497782 | 6.032401 | 6.716337 | 6.619162 | 6.573782 | 6.376829 |
| 1369996_at | 8.420409 | 8.675221 | 8.288344 | 8.894635 | 8.654631 | 8.62881 | 8.478255 |
| 1384327_at | 10.19544 | 10.02916 | 10.04503 | 10.05827 | 9.926531 | 9.946923 | 9.768086 |
| 1372483_at | 8.64353 | 8.562799 | 8.623293 | 8.760144 | 8.618125 | 8.592191 | 8.484938 |
| 1374575_at | 9.974064 | 10.11951 | 9.755545 | 9.826487 | 9.934232 | 10.01621 | 9.881983 |
| 1394383_at | 6.083598 | 6.310787 | 6.088823 | 5.755994 | 5.951376 | 5.915808 | 6.003865 |
| 1373103_at | 8.334484 | 8.410893 | 8.405907 | 8.43169 | 8.198832 | 7.899356 | 7.885699 |
| 1390836_at | 5.758431 | 5.941285 | 5.965545 | 6.044759 | 6.152101 | 6.339301 | 6.242937 |
| 1383017_at | 7.210814 | 7.475091 | 7.66777 | 7.395356 | 7.123632 | 7.297249 | 7.338557 |
| 1385461_at | 3.304497 | 3.26206 | 3.305945 | 3.262951 | 3.311728 | 3.298642 | 3.345778 |
| 1373769_at | 8.196541 | 8.523536 | 8.207773 | 8.666415 | 8.385473 | 8.846042 | 9.105451 |
| 1370073_at | 9.596717 | 9.587639 | 9.608055 | 9.508361 | 9.757028 | 10.34565 | 10.03016 |
| 1388869_at | 7.657102 | 7.542555 | 7.380516 | 7.521751 | 7.783771 | 7.428494 | 7.419054 |
| 1370769_a_at | 5.354796 | 5.394914 | 5.201245 | 5.755596 | 5.502894 | 5.473419 | 5.528637 |
| 1375550_at | 10.21916 | 10.32873 | 10.14313 | 10.442 | 10.39579 | 10.58203 | 10.4249 |
| 1367501_at | 7.838297 | 8.120957 | 8.000561 | 7.813478 | 7.693701 | 8.00265 | 8.008291 |
| 1394445_at | 6.770849 | 6.497587 | 6.570086 | 6.878644 | 6.924572 | 6.745798 | 6.621969 |
| 1375738_at | 4.54013 | 4.367381 | 4.327658 | 4.228379 | 4.348922 | 4.571517 | 4.59348 |
| 1388853_at | 9.609569 | 9.418027 | 9.425038 | 9.745141 | 9.745005 | 9.658217 | 9.409431 |
| 1388397_at | 8.143987 | 8.110743 | 8.428367 | 8.378087 | 8.183792 | 8.371388 | 8.555252 |
| 1367831_at | 7.744997 | 7.635018 | 7.468572 | 7.86319 | 7.921725 | 7.493097 | 7.602243 |
| 1378569_at | 6.720359 | 6.61528 | 6.473998 | 6.944225 | 6.613194 | 6.879834 | 6.684017 |
| 1370142_at | 10.853 | 10.84834 | 11.06932 | 11.03877 | 10.74723 | 11.02003 | 11.25514 |
| 1374332_at | 7.202475 | 7.134932 | 6.870358 | 7.371813 | 7.276047 | 7.304017 | 7.263626 |
| 1371971_at | 9.405391 | 9.173049 | 9.078253 | 9.543587 | 9.460941 | 9.324656 | 9.131877 |
| 1385476_x_at | 9.310768 | 9.250461 | 9.379681 | 9.734942 | 9.52475 | 9.900593 | 9.4644 |
| 1395637_at | 5.829581 | 5.65854 | 5.869216 | 5.635714 | 5.834362 | 5.814878 | 6.045101 |
| 1367586_at | 11.61197 | 11.30852 | 11.4998 | 11.89117 | 11.69537 | 11.52663 | 11.48752 |
| 1383068_at | 7.644511 | 7.764109 | 8.224168 | 7.947136 | 7.8514 | 7.891338 | 7.52891 |
| 1369720_at | 8.476457 | 8.724201 | 8.775707 | 8.62996 | 8.431769 | 8.678622 | 8.784221 |
| 1397271_at | 3.682842 | 3.580478 | 3.672846 | 3.664035 | 3.664885 | 3.730402 | 3.640981 |
| 1390190_at | 7.744449 | 7.880334 | 7.402099 | 8.216039 | 8.088161 | 8.507533 | 7.890114 |
| 1372219_at | 10.77539 | 10.46397 | 10.64533 | 10.6656 | 10.76588 | 10.90214 | 10.71358 |
| 1371977_at | 11.31848 | 11.20233 | 11.19717 | 11.52406 | 11.36541 | 11.78542 | 11.76626 |
| 1372119_at | 8.71293 | 8.744668 | 8.981912 | 8.930306 | 8.604262 | 9.019948 | 9.089139 |
| 1375669_at | 9.986041 | 9.900512 | 9.719238 | 10.07503 | 10.07643 | 10.16479 | 10.23423 |
| 1376209_at | 10.06254 | 9.755191 | 9.666472 | 10.07573 | 10.15286 | 10.13078 | 9.810066 |
| 1382266_at | 6.157537 | 6.26822 | 5.92479 | 6.485441 | 6.558173 | 6.113988 | 6.423705 |
| 1390559_at | 8.637663 | 8.539927 | 8.389252 | 8.684861 | 8.518872 | 8.208703 | 8.332016 |
| 1371812_at | 9.089815 | 9.14666 | 9.120569 | 9.319473 | 9.18651 | 9.466983 | 9.350153 |
| 1384428_at | 8.997867 | 9.064058 | 8.878613 | 9.47123 | 9.245315 | 8.964126 | 8.937311 |
| 1370817_at | 9.393647 | 9.076357 | 8.964216 | 9.317365 | 9.6338 | 9.794505 | 9.549815 |
| 1370040_at | 7.27302 | 7.035847 | 6.830653 | 7.411757 | 7.174282 | 7.214452 | 6.956239 |
| 1381492_at | 3.188588 | 3.121968 | 3.068946 | 3.118881 | 3.191294 | 3.315838 | 3.135624 |
| 1391494_at | 7.785763 | 7.656651 | 7.771972 | 7.923371 | 7.883842 | 7.692001 | 7.8863 |
| 1373041_at | 10.92977 | 10.70835 | 10.80864 | 10.93429 | 11.02897 | 10.72582 | 10.61024 |
| 1398777_at | 10.16239 | 9.863448 | 9.786986 | 10.3974 | 10.39054 | 9.850589 | 9.738627 |
| 1376143_at | 7.002313 | 6.904608 | 6.650731 | 7.231468 | 7.123673 | 6.937599 | 6.749701 |
| 1388351_at | 11.51858 | 11.00566 | 11.13582 | 11.52284 | 11.58884 | 11.43893 | 11.144 |
| 1372786_at | 6.707432 | 6.529415 | 6.606182 | 6.943399 | 6.915553 | 6.73007 | 6.818028 |
| 1374004_at | 7.969102 | 8.110002 | 8.011206 | 7.818446 | 7.956683 | 8.234099 | 8.319813 |
| 1372794_at | 8.575266 | 8.799251 | 8.347034 | 8.684987 | 8.659691 | 8.697904 | 8.74523 |
| 1385754_a_at | 3.444933 | 3.517922 | 3.481559 | 3.446812 | 3.444933 | 3.513775 | 3.535115 |
| 1388363_at | 8.085413 | 7.940204 | 8.193235 | 8.235421 | 7.98099 | 7.97968 | 8.14914 |
| 1371633_at | 7.577723 | 7.528029 | 7.706263 | 7.713312 | 7.631667 | 7.76314 | 7.736306 |
| 1383260_at | 9.865527 | 9.687894 | 9.890183 | 9.941066 | 9.985714 | 9.790576 | 9.683569 |
| 1378400_at | 7.854706 | 8.057327 | 7.668309 | 8.467475 | 8.251181 | 8.114069 | 8.562287 |
| 1388112_at | 11.83893 | 11.70304 | 12.13269 | 11.76528 | 11.68697 | 11.60979 | 11.86582 |
| 1395871_at | 8.260702 | 8.271152 | 8.313653 | 8.106482 | 8.168685 | 8.299195 | 8.529323 |
| 1395488_at | 4.873863 | 4.842373 | 4.584238 | 4.869415 | 4.681618 | 4.816625 | 4.631674 |
| 1378206_a_at | 3.15205 | 3.130031 | 3.148878 | 3.096235 | 3.179428 | 3.19287 | 3.123777 |
| 1385785_at | 6.474794 | 6.649713 | 6.383188 | 6.642885 | 6.542426 | 6.515189 | 6.376028 |
| 1376971_at | 6.74616 | 6.317346 | 6.587028 | 6.622517 | 6.941548 | 6.563199 | 6.672203 |
| 1368116 a_at | 6.620825 | 6.732027 | 7.060667 | 6.551448 | 6.408426 | 6.685943 | 6.899316 |
| 1385755_at | 7.716029 | 7.645071 | 8.128826 | 7.870825 | 7.569261 | 7.896907 | 7.922242 |
| 1386687_at | 5.209302 | 5.380964 | 5.227657 | 5.392466 | 5.491504 | 5.040777 | 5.223584 |
| 1396057_at | 6.466001 | 6.284811 | 6.439636 | 6.271437 | 6.300475 | 6.057393 | 5.808118 |
| 1374711_at | 8.327207 | 8.519948 | 8.40107 | 8.594931 | 8.393531 | 8.535542 | 8.444115 |
| 1373440_at | 8.15467 | 7.986503 | 7.93414 | 8.058511 | 8.228353 | 8.153623 | 8.077321 |
| 1390649_at | 7.866861 | 7.466253 | 7.673847 | 7.520572 | 7.858971 | 7.387393 | 7.456762 |
| 1397504_at | 4.914408 | 4.922794 | 4.684775 | 4.96054 | 4.851512 | 5.013417 | 4.823121 |
| 1370282_at | 9.824078 | 9.746749 | 9.130013 | 9.743674 | 9.84059 | 9.970792 | 9.530264 |
| 1369510_at | 6.763927 | 6.435619 | 6.298524 | 6.63333 | 6.690112 | 6.676548 | 6.46321 |
| 1371869_at | 10.75698 | 10.63691 | 10.59599 | 10.93867 | 10.89993 | 10.60385 | 10.65626 |
| 1377735_at | 9.361932 | 9.188498 | 9.439323 | 9.396021 | 9.284534 | 9.191507 | 9.113175 |
| 1378567_at | 4.45454 | 4.569303 | 4.371049 | 4.372755 | 4.550153 | 4.249538 | 4.612275 |
| 1393938_at | 4.711079 | 4.882573 | 4.818353 | 4.889962 | 5.038077 | 5.515855 | 4.921388 |
| 1388542_at | 9.687893 | 9.561521 | 9.338706 | 9.943886 | 9.923754 | 9.971334 | 9.665778 |
| 1378449_at | 5.630717 | 5.449786 | 5.505526 | 5.348569 | 5.527883 | 5.559005 | 5.461809 |
| 1374268_at | 7.682587 | 7.384005 | 7.653967 | 7.465068 | 7.454798 | 7.392089 | 7.322999 |
| 1396623_at | 4.916139 | 5.133227 | 4.938465 | 4.729353 | 4.808834 | 4.812075 | 4.865741 |
| 1378203_at | 8.650156 | 8.526446 | 8.943865 | 8.852407 | 8.625692 | 9.055259 | 9.317636 |
| 1399158_a_at | 11.36625 | 11.41586 | 11.33652 | 11.72634 | 11.66369 | 11.73656 | 11.8518 |
| 1386125_at | 6.55785 | 6.709559 | 6.338986 | 6.532785 | 6.746082 | 6.590108 | 6.566563 |
| 1388478_at | 10.11596 | 9.911651 | 10.35117 | 9.930923 | 9.690721 | 10.50298 | 10.63114 |
| 1393085_at | 6.595999 | 6.534269 | 6.129105 | 6.516882 | 6.686597 | 6.431635 | 6.410039 |
| 1375521_at | 8.207909 | 8.509426 | 8.327851 | 8.439077 | 8.582423 | 8.121633 | 8.51449 |
| 1372981_at | 5.723002 | 5.792251 | 5.487913 | 5.716107 | 5.724011 | 5.739695 | 5.580934 |
| 1375173_at | 9.501624 | 9.361864 | 9.344087 | 9.730982 | 9.661443 | 9.201346 | 9.33112 |
| 1382318_at | 4.670235 | 4.680718 | 4.606665 | 4.563787 | 4.794625 | 4.80293 | 4.7969 |
| 1375554_at | 8.88296 | 8.823002 | 8.798919 | 8.827708 | 8.867399 | 8.846522 | 8.767938 |
| 1374629_at | 6.900791 | 6.885689 | 7.069158 | 7.582315 | 7.121951 | 7.310341 | 7.060941 |
| 1370944_at | 3.822805 | 3.623016 | 3.748974 | 3.766901 | 3.771253 | 3.887607 | 3.860286 |
| 1367696_at | 9.106851 | 8.642553 | 8.728564 | 9.305039 | 9.295057 | 9.047237 | 8.864368 |
| 1392504_at | 7.933318 | 7.543484 | 7.820675 | 8.082911 | 8.065387 | 7.786117 | 7.697777 |
| 1373840_at | 7.08288 | 7.271265 | 7.117343 | 7.462853 | 7.318676 | 7.303766 | 7.248509 |
| 1398772_at | 9.253729 | 9.178471 | 9.206046 | 9.355841 | 9.223731 | 9.340595 | 9.302826 |
| 1372941_at | 7.792659 | 7.854876 | 7.507321 | 8.308551 | 7.974964 | 7.770276 | 8.036412 |
| 1372891_at | 7.638174 | 7.758204 | 7.293063 | 7.817409 | 7.933449 | 7.59012 | 7.437592 |
| 1368665_at | 8.367155 | 8.427347 | 8.312457 | 8.634957 | 8.573368 | 8.627 | 8.591522 |
| 1388067_a_at | 8.796525 | 8.772141 | 8.861678 | 8.627837 | 8.698711 | 8.875414 | 8.620972 |
| 1390924_at | 4.42203 | 4.827431 | 5.172974 | 4.99579 | 4.739177 | 4.999355 | 5.017769 |
| 1368123_at | 7.904951 | 7.636417 | 8.057934 | 7.928757 | 7.763832 | 7.584241 | 8.068762 |
| 1372393_at | 8.992121 | 9.00477 | 8.802998 | 9.196878 | 8.849224 | 9.182446 | 9.085267 |
| 1384943_at | 6.100576 | 6.412126 | 6.437269 | 7.188635 | 6.669894 | 6.461192 | 6.692244 |
| 1380768_at | 6.209552 | 6.235426 | 6.728205 | 6.988368 | 6.367614 | 6.878134 | 7.231529 |
| 1388013_at | 4.71294 | 4.794352 | 4.690289 | 4.654907 | 4.782202 | 4.94372 | 4.821069 |
| 1398241_a_at | 5.370259 | 5.409038 | 5.330118 | 5.461689 | 5.282215 | 5.543443 | 5.539157 |
| 1384207_at | 5.664329 | 5.803087 | 5.644646 | 5.70154 | 5.590847 | 5.646159 | 5.632695 |
| 1387617_at | 6.774814 | 5.916818 | 6.981282 | 6.275169 | 6.749048 | 6.402013 | 6.35743 |
| 1388745_at | 6.360753 | 6.429884 | 6.35904 | 6.222398 | 6.690005 | 6.16909 | 6.172013 |
| 1385975_at | 3.655451 | 3.639901 | 3.687412 | 3.51673 | 3.546149 | 3.660697 | 3.594977 |
| 1392745_at | 4.832234 | 4.963644 | 4.874342 | 4.984741 | 5.003458 | 4.859814 | 4.681391 |
| 1378229_at | 8.179664 | 8.01499 | 7.806255 | 7.806977 | 7.963169 | 7.841969 | 7.765816 |
| 1395860_at | 5.018488 | 4.634436 | 4.740092 | 4.977493 | 5.101506 | 5.04169 | 5.202587 |
| 1380257_at | 7.37725 | 7.462511 | 7.578229 | 7.455303 | 7.616852 | 7.684983 | 7.511676 |
| 1383627_a_at | 7.411349 | 7.706995 | 8.290465 | 7.639293 | 7.480893 | 7.991505 | 8.165251 |
| 1371120_a_at | 4.816896 | 4.590357 | 4.56291 | 4.515408 | 4.621802 | 4.575861 | 4.476161 |
| 1373135_at | 6.538211 | 6.087747 | 6.38477 | 6.703653 | 6.634717 | 6.46332 | 6.487785 |
| 1368062_at | 8.801505 | 8.852804 | 8.826192 | 8.960214 | 8.947813 | 8.980855 | 8.601901 |
| AFFX-r2-Ec-bioC-3_at | 9.18812 | 8.887714 | 8.903624 | 9.12256 | 9.526751 | 9.814525 | 9.817275 |
| 1398909_at | 9.716631 | 9.684194 | 9.495219 | 10.01092 | 9.886352 | 9.904391 | 9.82862 |
| 1399074_at | 9.086419 | 9.135042 | 9.203997 | 9.210174 | 8.976006 | 9.272859 | 9.095237 |
| 1384370_at | 5.498746 | 5.586697 | 5.537012 | 5.696068 | 5.638748 | 5.571183 | 5.357013 |
| 1370253_at | 11.15965 | 10.85646 | 11.02103 | 11.26431 | 11.31469 | 11.33746 | 11.37567 |
| 1374357_at | 7.858552 | 7.852273 | 7.808009 | 8.27856 | 8.063117 | 8.079088 | 7.990019 |
| 1384344_at | 6.80036 | 6.544967 | 6.758 | 6.943233 | 7.119518 | 7.102233 | 6.875752 |
| 1396142_at | 7.277484 | 6.904796 | 7.287323 | 7.303102 | 7.318411 | 7.274101 | 7.296395 |
| 1371944_at | 10.72233 | 10.49927 | 10.44866 | 10.87346 | 10.88892 | 10.63346 | 10.4073 |
| 1378396_at | 7.031593 | 7.176079 | 7.288923 | 6.983694 | 7.010744 | 7.196433 | 7.658381 |
| 1376570_at | 10.82502 | 10.80457 | 10.96014 | 11.13391 | 10.96831 | 11.35412 | 11.32161 |
| 1371810_at | 9.332541 | 9.056085 | 9.310855 | 9.045788 | 9.236917 | 9.011199 | 8.847815 |
| 1367465_at | 10.57096 | 10.19517 | 9.895014 | 10.4014 | 10.56095 | 10.31431 | 10.0588 |
| 1376272_a_at | 10.28879 | 9.932648 | 9.954964 | 10.36673 | 10.43656 | 9.988782 | 9.913219 |
| 1389178_at | 8.721315 | 8.85939 | 8.843272 | 8.97333 | 8.569128 | 8.967769 | 8.869086 |
| 1376745_at | 7.18613 | 7.52318 | 7.382136 | 7.224264 | 7.018712 | 7.200412 | 7.263262 |
| 1391527_at | 6.846371 | 6.36652 | 6.613354 | 6.779794 | 6.902837 | 6.574278 | 6.518157 |
| 1369656_at | 6.176432 | 6.229931 | 6.233386 | 6.47454 | 6.119669 | 6.529218 | 6.540164 |
| 1389045_at | 6.481584 | 6.412969 | 6.254184 | 6.297129 | 6.534825 | 6.641746 | 6.545324 |
| 1384950_at | 6.5579 | 6.735608 | 6.679444 | 6.818978 | 6.539319 | 6.526219 | 6.811573 |
| 1387027_a_at | 8.509784 | 8.588066 | 8.48455 | 8.79372 | 8.451693 | 8.639001 | 8.678782 |
| 1370613_a_at | 9.169934 | 8.957994 | 9.050581 | 9.025818 | 9.028429 | 9.195828 | 8.945095 |
| 1392975_at | 7.412611 | 7.151055 | 7.388451 | 7.521015 | 7.50785 | 7.56331 | 7.583048 |
| 1396387_at | 4.211046 | 4.291444 | 4.070767 | 4.075371 | 4.090993 | 4.052181 | 4.015742 |

TABLE 7

| | Probeset | cr50 > cont | cr70 > cr50 | upup | cr50 < cont | cr70 < cr50 | downdown |
|---|---|---|---|---|---|---|---|
| 1 | 1395846_at | true | true | TRUE | false | false | |
| 2 | 1390943_at | true | true | TRUE | false | false | |
| 3 | 1389844_at | true | true | TRUE | false | false | |
| 4 | 1368247_at | true | true | TRUE | false | false | |
| 5 | 1374857_at | true | true | TRUE | false | false | |
| 6 | 1370811_at | true | true | TRUE | false | false | |
| 7 | 1397918_at | true | true | TRUE | false | false | |
| 8 | 1371294_at | true | true | TRUE | false | false | |
| 9 | 1387307_at | true | true | TRUE | false | false | |
| 10 | 1373177_x_at | true | true | TRUE | false | false | |
| 11 | 1396398_at | true | true | TRUE | false | false | |
| 12 | 1374699_at | true | true | TRUE | false | false | |
| 13 | 1385606_at | true | true | TRUE | false | false | |
| 14 | 1385365_at | true | true | TRUE | false | false | |
| 15 | 1385133_at | true | true | TRUE | false | false | |
| 16 | 1375014_at | true | true | TRUE | false | false | |
| 17 | 1390982_at | true | true | TRUE | false | false | |
| 18 | 1367904_at | true | true | TRUE | false | false | |
| 19 | 1387048_at | true | true | TRUE | false | false | |
| 20 | 1379592_at | true | true | TRUE | false | false | |
| 21 | 1389765_at | true | true | TRUE | false | false | |
| 22 | 1395731_at | true | true | TRUE | false | false | |
| 23 | 1393963_at | true | true | TRUE | false | false | |
| 24 | 1396468_at | true | true | TRUE | false | false | |
| 25 | 1398877_at | true | true | TRUE | false | false | |
| 26 | 1371188_a_at | true | true | TRUE | false | false | |
| 27 | 1392356_at | true | true | TRUE | false | false | |
| 28 | 1369021_at | true | true | TRUE | false | false | |
| 29 | 1383967_at | true | true | TRUE | false | false | |
| 30 | 1367741_at | true | true | TRUE | false | false | |
| 31 | 1378552_at | true | true | TRUE | false | false | |
| 32 | 1389472_at | true | true | TRUE | false | false | |
| 33 | 1388270_at | true | true | TRUE | false | false | |
| 34 | 1383346_at | true | true | TRUE | false | false | |
| 35 | 1391321_at | true | true | TRUE | false | false | |
| 36 | 1385699_at | true | true | TRUE | false | false | |
| 37 | 1377627_at | true | true | TRUE | false | false | |
| 38 | 1394507_at | true | true | TRUE | false | false | |
| 39 | 1393961_at | true | true | TRUE | false | false | |
| 40 | 1378247_at | true | true | TRUE | false | false | |
| 41 | 1386669_at | true | true | TRUE | false | false | |
| 42 | 1377129_at | true | true | TRUE | false | false | |
| 43 | 1379021_a_at | true | true | TRUE | false | false | |
| 44 | 1375206_at | true | true | TRUE | false | false | |
| 45 | 1398636_at | true | true | TRUE | false | false | |
| 46 | 1385808_at | true | true | TRUE | false | false | |
| 47 | 1393873_s_at | true | true | TRUE | false | false | |
| 48 | 1385620_at | true | true | TRUE | false | false | |
| 49 | 1387779_at | true | true | TRUE | false | false | |
| 50 | 1381510_at | true | true | TRUE | false | false | |
| 51 | 1389203_at | true | true | TRUE | false | false | |
| 52 | 1386186_s_at | true | true | TRUE | false | false | |
| 53 | 1388438_at | true | true | TRUE | false | false | |
| 54 | 1372191_at | true | true | TRUE | false | false | |
| 55 | 1396301_x_at | true | true | TRUE | false | false | |
| 56 | 1395364_at | true | true | TRUE | false | false | |
| 57 | 1368338_at | true | true | TRUE | false | false | |
| 58 | 1388971_s_at | true | true | TRUE | false | false | |
| 59 | 1389565_at | true | true | TRUE | false | false | |
| 60 | 1393988_at | true | true | TRUE | false | false | |
| 61 | 1399032_at | true | true | TRUE | false | false | |
| 62 | 1385303_at | true | true | TRUE | false | false | |
| 63 | 1384064_at | true | true | TRUE | false | false | |
| 64 | 1387511_at | true | true | TRUE | false | false | |
| 65 | 1372646_at | false | false | | true | true | TRUE |
| 66 | 1368474_at | false | false | | true | true | TRUE |
| 67 | 1376624_at | false | false | | true | true | TRUE |
| 68 | 1388879_at | false | false | | true | true | TRUE |
| 69 | 1391563_at | false | false | | true | true | TRUE |
| 70 | 1387029_at | false | false | | true | true | TRUE |
| 71 | 1387854_at | false | false | | true | true | TRUE |
| 72 | 1372585_at | false | false | | true | true | TRUE |
| 73 | 1397173_at | false | false | | true | true | TRUE |
| 74 | 1368558_s_at | false | false | | true | true | TRUE |
| 75 | 1383589_at | false | false | | true | true | TRUE |
| 76 | 1380962_at | false | false | | true | true | TRUE |
| 77 | 1395519_at | false | false | | true | true | TRUE |
| 78 | 1381993_at | false | false | | true | true | TRUE |
| 79 | 1388996_at | false | false | | true | true | TRUE |
| 80 | 1368751_at | false | false | | true | true | TRUE |
| 81 | 1389553_at | false | false | | true | true | TRUE |
| 82 | 1387796_at | false | false | | true | true | TRUE |
| 83 | 1369132_at | false | false | | true | true | TRUE |
| 84 | 1382146_at | false | false | | true | true | TRUE |
| 85 | 1393008_at | false | false | | true | true | TRUE |
| 86 | 1368064_a_at | false | false | | true | true | TRUE |
| 87 | 1397670_at | false | false | | true | true | TRUE |
| 88 | 1388116_at | false | false | | true | true | TRUE |
| 89 | 1370959_at | false | false | | true | true | TRUE |
| 90 | 1371677_at | false | false | | true | true | TRUE |
| 91 | 1381556_at | false | false | | true | true | TRUE |
| 92 | 1379314_at | false | false | | true | true | TRUE |
| 93 | 1371015_at | false | false | | true | true | TRUE |
| 94 | 1380822_at | false | false | | true | true | TRUE |
| 95 | 1377086_at | false | false | | true | true | TRUE |
| 96 | 1375350_at | false | false | | true | true | TRUE |
| 97 | 1368829_at | false | false | | true | true | TRUE |
| 98 | 1370155_at | false | false | | true | true | TRUE |
| 99 | 1391916_at | false | false | | true | true | TRUE |
| 100 | 1387893_at | false | false | | true | true | TRUE |
| 101 | 1371614_at | false | false | | true | true | TRUE |
| 102 | 1390510_at | false | false | | true | true | TRUE |
| 103 | 1384310_at | false | false | | true | true | TRUE |
| 104 | 1379357_at | false | false | | true | true | TRUE |
| 105 | 1373410_at | false | false | | true | true | TRUE |
| 106 | 1389164_at | false | false | | true | true | TRUE |
| 107 | 1377751_at | false | false | | true | true | TRUE |
| 108 | 1388054_a_at | false | false | | true | true | TRUE |
| 109 | 1387570_at | false | false | | true | true | TRUE |
| 110 | 1368399_a_at | false | false | | true | true | TRUE |
| 111 | 1382960_at | false | false | | true | true | TRUE |
| 112 | 1384558_at | false | false | | true | true | TRUE |
| 113 | 1377353_a_at | false | false | | true | true | TRUE |
| 114 | 1384311_at | false | false | | true | true | TRUE |
| 115 | 1382028_at | false | false | | true | true | TRUE |
| 116 | 1389718_at | false | false | | true | true | TRUE |
| 117 | 1371483_at | false | false | | true | true | TRUE |
| 118 | 1372146_at | false | false | | true | true | TRUE |
| 119 | 1379932_at | false | false | | true | true | TRUE |
| 120 | 1368332_at | false | false | | true | true | TRUE |
| 121 | 1389413_at | false | false | | true | true | TRUE |
| 122 | 1381452_at | false | false | | true | true | TRUE |
| 123 | 1375982_at | false | false | | true | true | TRUE |
| 124 | 1387795_at | false | false | | true | true | TRUE |
| 125 | 1382711_at | false | false | | true | true | TRUE |
| 126 | 1373882_at | false | false | | true | true | TRUE |
| 127 | 1375729_at | false | false | | true | true | TRUE |
| 128 | 1393217_at | false | false | | true | true | TRUE |
| 129 | 1391610_at | false | false | | true | true | TRUE |
| 130 | 1376071_at | false | false | | true | true | TRUE |
| 131 | 1370827_at | false | false | | true | true | TRUE |
| 132 | 1383453_at | false | false | | true | true | TRUE |
| 133 | 1378474_at | false | false | | true | true | TRUE |
| 134 | 1372922_at | false | false | | true | true | TRUE |
| 135 | 1373891_at | false | false | | true | true | TRUE |
| 136 | 1376749_at | false | false | | true | true | TRUE |
| 137 | 1376575_at | false | false | | true | true | TRUE |
| 138 | 1379055_x_at | false | false | | true | true | TRUE |
| 139 | 1377171_at | false | false | | true | true | TRUE |
| 140 | 1391462_at | false | false | | true | true | TRUE |
| 141 | 1377640_at | false | false | | true | true | TRUE |
| 142 | 1382659_at | false | false | | true | true | TRUE |
| 143 | 1373944_at | false | false | | true | true | TRUE |
| 144 | 1369186_at | false | false | | true | true | TRUE |
| 145 | 1393866_at | false | false | | true | true | TRUE |
| 146 | 1388936_at | false | false | | true | true | TRUE |
| 147 | 1390914_at | false | false | | true | true | TRUE |
| 148 | 1391428_at | false | false | | true | true | TRUE |
| 149 | 1373577_at | false | false | | true | true | TRUE |
| 150 | 1370280_at | false | false | | true | true | TRUE |
| 151 | 1394101_at | false | false | | true | true | TRUE |
| 152 | 1372013_at | false | false | | true | true | TRUE |
| 153 | 1391030_at | false | false | | true | true | TRUE |
| 154 | 1390440_at | false | false | | true | true | TRUE |
| 155 | 1391211_at | false | false | | true | true | TRUE |
| 156 | 1368156_at | false | false | | true | true | TRUE |
| 157 | 1390638_at | false | false | | true | true | TRUE |
| 158 | 1375966_at | false | false | | true | true | TRUE |
| 159 | 1397536_at | false | false | | true | true | TRUE |
| 160 | 1375378_at | false | false | | true | true | TRUE |
| 161 | 1384180_at | false | false | | true | true | TRUE |
| 162 | 1381504_at | false | false | | true | true | TRUE |
| 163 | 1381577_at | false | false | | true | true | TRUE |
| 164 | 1392705_at | false | false | | true | true | TRUE |
| 165 | 1397221_s_at | false | false | | true | true | TRUE |
| 166 | 1372947_at | false | false | | true | true | TRUE |
| 167 | 1375523_at | false | false | | true | true | TRUE |
| 168 | 1370583_s_at | false | false | | true | true | TRUE |
| 169 | 1369648_at | false | false | | true | true | TRUE |
| 170 | 1391106_at | false | false | | true | true | TRUE |
| 171 | 1378269_at | false | false | | true | true | TRUE |
| 172 | 1371241_x_at | false | false | | true | true | TRUE |
| 173 | 1376660_at | false | false | | true | true | TRUE |
| 174 | 1379274_at | false | false | | true | true | TRUE |
| 175 | 1388789_at | false | false | | true | true | TRUE |
| 176 | 1371202_a_at | false | false | | true | true | TRUE |
| 177 | 1383529_at | false | false | | true | true | TRUE |
| 178 | 1390427_at | false | false | | true | true | TRUE |
| 179 | 1377950_at | false | false | | true | true | TRUE |
| 180 | 1379683_at | false | false | | true | true | TRUE |
| 181 | 1368822_at | false | false | | true | true | TRUE |
| 182 | 1389305_at | false | false | | true | true | TRUE |
| 183 | 1382660_at | false | false | | true | true | TRUE |
| 184 | 1387690_at | false | false | | true | true | TRUE |
| 185 | 1372587_at | false | false | | true | true | TRUE |
| 186 | 1369633_at | false | false | | true | true | TRUE |
| 187 | 1370333_a_at | false | false | | true | true | TRUE |
| 188 | 1392856_at | false | false | | true | true | TRUE |
| 189 | 1391551_at | false | false | | true | true | TRUE |
| 190 | 1388903_at | false | false | | true | true | TRUE |
| 191 | 1371875_at | false | false | | true | true | TRUE |
| 192 | 1372332_at | false | false | | true | true | TRUE |
| 193 | 1374224_at | false | false | | true | true | TRUE |
| 194 | 1371040_at | false | false | | true | true | TRUE |
| 195 | 1379677_at | false | false | | true | true | TRUE |
| 196 | 1394059_s_at | false | false | | true | true | TRUE |
| 197 | 1368658_at | false | false | | true | true | TRUE |
| 198 | 1389690_at | false | false | | true | true | TRUE |
| 199 | 1379652_at | false | false | | true | true | TRUE |
| 200 | 1374700_at | false | false | | true | true | TRUE |
| 201 | 1379482_at | false | false | | true | true | TRUE |
| 202 | 1394012_at | false | false | | true | true | TRUE |
| 203 | 1396957_at | false | false | | true | true | TRUE |
| 204 | 1397167_at | false | false | | true | true | TRUE |
| 205 | 1376747_at | false | false | | true | true | TRUE |
| 206 | 1389006_at | false | false | | true | true | TRUE |
| 207 | 1388544_at | false | false | | true | true | TRUE |
| 208 | 1378243_at | false | false | | true | true | TRUE |

TABLE 9
Group Predication Table from Systat Discrim. Analysis
using 10 genes listed in text of update.

| Animal | Actual GRP | Predicted GRP |
|---|---|---|
| 1 | 1.000 | 1.000 |
| 2 | 1.000 | 1.000 |
| 3 | 1.000 | 1.000 |
| 4 | 1.000 | 1.000 |
| 5 | 1.000 | 1.000 |
| 6 | 1.000 | 1.000 |
| 7 | 1.000 | 1.000 |
| 8 | 1.000 | 1.000 |
| 9 | 2.000 | 2.000 |
| 10 | 2.000 | 2.000 |
| 11 | 2.000 | 2.000 |
| 12 | 2.000 | 2.000 |
| 13 | 2.000 | 2.000 |
| 14 | 3.000 | 3.000 |
| 15 | 3.000 | 3.000 |
| 16 | 3.000 | 3.000 |
| 17 | 3.000 | 3.000 |
| 18 | 3.000 | 3.000 |
| 19 | 4.000 | 4.000 |
| 20 | 4.000 | 4.000 |
| 21 | 4.000 | 4.000 |
| 22 | 4.000 | 4.000 |
| 23 | 4.000 | 4.000 |
| 24 | 4.000 | 4.000 |

Group (GRP) 1 = control
GRP2 = 50% caloric restriction
GRP3 = 70% caloric restriction
GRP4 = High Fat diet

TABLE 10

| Probeset | SSRI | SSRI | SSRI | SSRI | SSRI | SSRI | C |
|---|---|---|---|---|---|---|---|
| 1382608_at | 5.492892 | 5.536467 | 5.440813 | 5.641229 | 5.553487 | 5.572907 | 5.323105 |
| 1376218_a_at | 4.961073 | 5.159801 | 4.667357 | 4.873284 | 4.561898 | 4.552214 | 5.749471 |
| 1386519_x_at | 7.187885 | 7.294603 | 7.082242 | 7.037942 | 7.202921 | 7.126005 | 7.601578 |
| 1385442_at | 7.159886 | 7.404339 | 6.996945 | 6.879263 | 6.919968 | 6.943116 | 7.831252 |
| 1384923_at | 6.976946 | 7.200037 | 6.817507 | 6.938916 | 6.836614 | 6.982019 | 7.388793 |
| 1385163_at | 6.063322 | 5.993012 | 6.045161 | 5.954846 | 5.884809 | 6.124244 | 5.653232 |
| 1383659_a_at | 5.774419 | 5.519035 | 5.386271 | 5.431161 | 4.833011 | 6.495673 | 6.266045 |
| 1393517_at | 6.631046 | 6.746358 | 6.287015 | 6.482644 | 6.515204 | 6.633106 | 7.091107 |
| 1376289_at | 3.746401 | 3.74974 | 3.659435 | 3.77225 | 3.709668 | 3.697027 | 3.470574 |
| 1390090_at | 7.476481 | 7.464459 | 7.498371 | 7.392979 | 7.300129 | 7.578367 | 6.855826 |
| 1379140_at | 4.025837 | 4.280388 | 4.108266 | 3.886397 | 4.070737 | 4.078684 | 4.796397 |
| 1381966_at | 8.360305 | 8.854557 | 8.773542 | 8.518282 | 8.759615 | 8.756927 | 9.646637 |
| 1378355_a_at | 5.806788 | 6.412462 | 5.853144 | 5.535617 | 5.776693 | 6.036624 | 7.608109 |
| 1377123_at | 8.957729 | 9.068757 | 8.728816 | 8.818872 | 8.853459 | 8.789082 | 9.154175 |
| 1398571_x_at | 5.476362 | 5.783264 | 5.338547 | 5.581326 | 5.637951 | 5.597321 | 6.85281 |
| 1383472_at | 3.95501 | 3.951148 | 3.984368 | 4.032821 | 3.886509 | 3.898694 | 3.699795 |
| 1383900_at | 4.742212 | 4.616846 | 4.606327 | 4.705032 | 4.74939 | 4.764865 | 4.517066 |
| 1382015_at | 7.649175 | 7.911227 | 7.375985 | 7.558502 | 7.728075 | 7.549806 | 8.304304 |
| 1384467_at | 8.339893 | 8.506768 | 8.417856 | 8.05848 | 8.062486 | 8.234164 | 8.586584 |
| 1382825_at | 5.701211 | 6.303403 | 6.179596 | 5.770345 | 6.003744 | 5.788324 | 6.412377 |
| 1395990_at | 7.226975 | 7.567221 | 7.454375 | 7.384037 | 7.119715 | 7.239466 | 7.906828 |
| 1376844_at | 3.958214 | 3.91987 | 4.036005 | 4.210816 | 4.082233 | 4.21383 | 3.793962 |
| 1367978_at | 5.913543 | 6.324466 | 5.573653 | 5.778349 | 5.944067 | 5.841989 | 6.445117 |
| 1387406_at | 6.009577 | 6.243269 | 5.981099 | 5.981192 | 5.9156698 | 6.086563 | 6.552047 |
| 1379110_at | 3.96473 | 4.036266 | 3.739473 | 3.917379 | 3.885783 | 3.803709 | 4.3956561 |
| 1381122_at | 5.405765 | 5.218806 | 5.498901 | 5.147761 | 5.433531 | 5.211904 | 4.704723 |
| 1381872_at | 6.498182 | 7.027084 | 7.041754 | 6.737296 | 6.910882 | 6.996371 | 7.684974 |
| 1393266_at | 4.99029 | 5.173955 | 4.982205 | 4.609065 | 4.823981 | 4.636297 | 5.661941 |
| 1372462_at | 7.91245 | 7.819118 | 7.863875 | 7.873258 | 6.183853 | 8.214012 | 8.489119 |
| 1394498_at | 5.625738 | 5.476476 | 5.77752 | 5.780313 | 5.589403 | 5.687828 | 5.354906 |
| 1381941_at | 7.9719 | 8.218287 | 7.48277 | 7.553401 | 7.612148 | 7.329846 | 8.887315 |
| 1397738_at | 4.15853 | 4.153792 | 4.198277 | 4.196809 | 4.130645 | 4.270782 | 4.042414 |
| 1397834_at | 6.222785 | 6.440389 | 5.751763 | 5.565274 | 5.809979 | 5.977959 | 6.931394 |
| 1373925_at | 6.767744 | 6.886409 | 6.335062 | 6.194636 | 6.159356 | 6.382243 | 7.138185 |
| 1384374_at | 6.358261 | 6.642625 | 6.364911 | 5.886558 | 6.42059 | 6.36247 | 7.089011 |
| 1397187_at | 5.846689 | 6.088335 | 6.023635 | 5.914559 | 5.910328 | 5.885615 | 6.251108 |
| 1368985_at | 4.724642 | 4.454151 | 4.645631 | 4.715157 | 4.58814 | 4.472289 | 4.27078 |
| 1386414_at | 6.861174 | 7.195748 | 6.931775 | 7.199033 | 7.249204 | 7.033052 | 7.604695 |
| 1377750_at | 8.621961 | 9.024142 | 8.834947 | 8.889638 | 9.137156 | 8.948241 | 7.862024 |
| 1372514_s_at | 8.136352 | 8.210145 | 8.071195 | 8.128063 | 7.74062 | 8.133797 | 8.385963 |
| 1387459_at | 3.483577 | 3.638799 | 3.932181 | 3.860213 | 3.992271 | 3.799193 | 4.335378 |
| 1379235_x_at | 7.486198 | 7.694947 | 7.128343 | 7.25222 | 7.527172 | 7.243722 | 8.10408 |
| 1396326_at | 5.927748 | 5.926368 | 5.856986 | 6.008159 | 5.925758 | 5.801999 | 6.386887 |
| 1389835_at | 4.06394 | 3.784412 | 4.093119 | 4.04471 | 4.044924 | 4.18013 | 3.634727 |
| 1394414_at | 9.565232 | 9.66703 | 9.428135 | 9.393557 | 9.769193 | 9.655295 | 9.827319 |
| 1374656_at | 8.728528 | 9.034337 | 8.507261 | 8.742939 | 9.004444 | 8.9546 | 9.446713 |
| 1387083_at | 5.565117 | 6.278194 | 5.686051 | 5.780761 | 5.789342 | 5.938091 | 6.659477 |
| 1383984_at | 3.997443 | 4.264316 | 4.188617 | 4.265712 | 4.140383 | 4.315107 | 4.698688 |
| 1385760_at | 7.137235 | 7.353692 | 6.64775 | 7.089898 | 7.315844 | 7.320543 | 8.131391 |
| 1369005_at | 5.188243 | 5.545873 | 5.29123 | 5.305288 | 5.241218 | 5.222472 | 5.769169 |
| 1384527_at | 5.000136 | 5.159761 | 4.805931 | 4.891068 | 4.943786 | 4.995309 | 5.334889 |
| 1390464_at | 5.625381 | 5.847429 | 5.540981 | 5.806628 | 5.982722 | 5.830163 | 6.125056 |
| 1377414_at | 6.705963 | 7.101224 | 7.192647 | 7.164024 | 7.592751 | 7.073248 | 6.356363 |
| 1376278_at | 6.191189 | 6.405209 | 5.934088 | 6.175075 | 6.204845 | 6.006336 | 6.643587 |
| 1368646_at | 5.643903 | 5.881878 | 5.514679 | 5.483266 | 5.351527 | 5.292263 | 6.209936 |
| 1374245_at | 6.521767 | 6.226722 | 6.451731 | 6.338844 | 6.201398 | 6.356633 | 5.899628 |
| 1379499_at | 4.577697 | 4.495448 | 4.850826 | 4.557536 | 4.564405 | 4.699985 | 4.311151 |
| 1374807_at | 6.504529 | 6.90416 | 6.69714 | 6.907318 | 6.749177 | 6.774888 | 7.102589 |
| 1370115_at | 4.966575 | 4.917132 | 4.823981 | 4.621892 | 4.817953 | 4.719415 | 5.344088 |
| 1387912_at | 8.618418 | 8.518387 | 8.718407 | 9.049765 | 9.009861 | 8.757163 | 8.309068 |
| 1385652_at | 7.796885 | 7.898147 | 7.543269 | 7.484183 | 7.574632 | 7.378301 | 8.118309 |
| 1370688_at | 7.903932 | 7.963357 | 8.095767 | 7.914974 | 8.157005 | 7.989948 | 7.20193 |
| 1397911_at | 5.777085 | 6.102984 | 5.668644 | 5.522263 | 5.663644 | 5.596978 | 6.920386 |
| 1393853_at | 5.345946 | 5.932592 | 5.864851 | 5.835369 | 5.820583 | 5.912021 | 6.816743 |
| 1395951_at | 4.460255 | 4.582372 | 4.341001 | 4.395351 | 4.51615 | 4.568306 | 4.851354 |
| 1385775_at | 4.144712 | 4.37534 | 3.895951 | 3.993257 | 4.078934 | 4.161077 | 4.494862 |
| 1397500_x_at | 6.720934 | 7.079243 | 6.81847 | 6.587375 | 6.983111 | 6.477375 | 7.440144 |
| 1380152_at | 5.469969 | 5.228252 | 5.460724 | 5.179281 | 5.390314 | 5.269045 | 5.066724 |
| 1371813_at | 6.757321 | 6.607921 | 7.253639 | 7.022433 | 6.663198 | 6.909071 | 6.161254 |
| 1384377_at | 7.232772 | 7.134292 | 7.08513 | 7.149567 | 7.214216 | 7.15771 | 7.092002 |
| 1388486_at | 7.701491 | 7.565311 | 7.722823 | 7.751002 | 7.920888 | 7.749108 | 7.30164 |
| 1383667_at | 7.999644 | 8.041242 | 8.381136 | 8.125775 | 8.243658 | 8.175399 | 7.499266 |
| 1373423_at | 5.592849 | 5.787906 | 5.428351 | 5.355132 | 5.522468 | 5.51037 | 6.174582 |
| 1379408_at | 8.687982 | 8.66201 | 8.769811 | 8.887081 | 8.853017 | 8.911685 | 7.994738 |
| 1389551_at | 8.862161 | 8.606281 | 8.908632 | 8.801096 | 8.621364 | 8.842152 | 8.245416 |
| 1390259_at | 4.303999 | 4.545589 | 4.676909 | 4.255006 | 4.357835 | 4.411383 | 4.754853 |
| 1395303_at | 3.283546 | 3.242265 | 3.289337 | 3.299909 | 3.260577 | 3.27396 | 3.159386 |
| 1393580_at | 5.687082 | 6.029898 | 5.819162 | 5.753099 | 6.108495 | 5.966885 | 6.260583 |
| 1378275_at | 8.581232 | 8.429552 | 8.772929 | 8.828356 | 8.514891 | 8.554967 | 8.033699 |
| 1388938_at | 9.060702 | 9.267915 | 8.804801 | 8.994285 | 8.727635 | 9.107246 | 9.37707 |
| 1385518_at | 6.93739 | 6.773344 | 6.989627 | 6.986454 | 7.075521 | 6.759866 | 6.75783 |
| 1368397_at | 3.806273 | 3.896596 | 3.869808 | 3.843412 | 3.779624 | 3.762497 | 3.98816 |
| 1379980_at | 7.864229 | 7.81658 | 8.195439 | 8.083096 | 7.784396 | 8.002908 | 7.359861 |
| 1388781_at | 5.825339 | 6.004404 | 5.674498 | 5.734784 | 5.979118 | 5.672688 | 6.195365 |
| 1367866_at | 5.591173 | 5.570562 | 5.174466 | 5.047476 | 5.189581 | 5.136266 | 5.74946 |
| 1383132_at | 5.886752 | 6.390654 | 5.864872 | 5.893019 | 5.977328 | 5.95143 | 6.371194 |
| 1391709_at | 4.661094 | 4.68357 | 4.722356 | 4.806143 | 4.712504 | 4.469939 | 4.385907 |
| 1369051_at | 5.58844 | 5.206642 | 4.834847 | 5.01122 | 5.143924 | 5.186464 | 5.854885 |
| 1388037_at | 4.856483 | 4.679861 | 4.647 | 4.894296 | 4.952721 | 4.682282 | 4.490424 |
| 1386158_at | 4.00427 | 4.128207 | 4.062764 | 4.152343 | 3.987931 | 3.931191 | 3.849937 |
| 1378705_at | 8.278514 | 8.33992 | 7.828146 | 7.560535 | 7.946619 | 7.980371 | 8.487012 |
| 1375738_at | 4.993498 | 4.842595 | 5.387981 | 5.322236 | 5.141327 | 4.993316 | 4.552753 |
| 1387899_at | 5.335379 | 5.407877 | 5.338836 | 5.025946 | 4.957844 | 5.214891 | 5.666053 |
| 1369014_at | 4.756823 | 5.003892 | 4.708004 | 4.543487 | 4.616409 | 4.99275 | 5.247274 |
| 1398196_at | 5.274579 | 5.258664 | 5.614139 | 5.100729 | 5.598682 | 5.56614 | 4.758058 |
| 1380392_at | 6.093443 | 5.621599 | 5.424734 | 5.398348 | 5.78052 | 5.645733 | 5.096378 |
| 1388447_at | 7.564371 | 7.841207 | 7.682079 | 7.537375 | 7.380012 | 7.531893 | 7.887908 |
| 1372593_at | 7.225093 | 7.132906 | 7.723599 | 8.014863 | 7.571018 | 7.718979 | 6.813001 |
| 1395875_at | 7.410532 | 7.21151 | 7.607199 | 7.66723 | 7.917539 | 7.659763 | 6.876513 |
| 1378642_at | 6.33055 | 6.196697 | 6.546369 | 6.860656 | 6.597517 | 6.634403 | 6.045077 |
| 1370260_at | 7.313409 | 7.598517 | 7.248552 | 7.038278 | 7.707215 | 6.879237 | 6.558557 |
| 1376136_at | 5.073951 | 4.867253 | 4.556924 | 4.873284 | 4.804809 | 4.931059 | 5.171854 |
| 1369894_at | 3.61584 | 3.758347 | 3.896797 | 3.839078 | 4.008275 | 3.982282 | 3.466153 |
| 1391555_at | 7.012294 | 7.728313 | 6.643505 | 6.541149 | 6.178025 | 6.968483 | 7.63804 |
| 1382932_at | 5.366568 | 5.210585 | 5.239073 | 5.25206 | 5.387036 | 5.359002 | 4.880667 |
| 1389431_at | 6.55798 | 6.300196 | 6.381237 | 6.490001 | 6.951419 | 6.470954 | 5.42671 |
| 1378872_at | 4.781621 | 4.711402 | 4.621241 | 4.86313 | 4.867011 | 4.774975 | 4.629041 |
| 1382110_at | 7.205521 | 7.183247 | 6.692034 | 6.852022 | 6.784075 | 6.86181 | 7.424706 |
| 1379206_at | 6.485998 | 6.611079 | 6.213463 | 6.404673 | 6.472055 | 6.395471 | 6.831665 |
| 1394361_a_at | 4.203955 | 4.506677 | 4.246594 | 4.096835 | 4.402447 | 4.275132 | 5.803643 |
| 1390534_at | 7.499708 | 7.48788 | 7.224098 | 6.47796 | 7.350713 | 7.293165 | 7.8105 |
| 1379342_at | 6.296636 | 6.300452 | 6.275341 | 6.188146 | 6.25933 | 6.187146 | 6.4575 |
| 1393798_at | 3.547485 | 3.620464 | 3.802003 | 3.629104 | 3.836384 | 3.782214 | 3.399 |
| 1396104_at | 5.464411 | 5.416275 | 5.46843 | 5.563807 | 5.566789 | 5.601152 | 5.82413 |
| 1376852_at | 6.779731 | 6.536632 | 6.455416 | 6.69941 | 6.862407 | 6.681653 | 6.371801 |
| 1385292_at | 4.663043 | 4.709036 | 5.003794 | 4.749097 | 4.726512 | 4.656687 | 4.168593 |
| 1367489_at | 7.838786 | 8.052143 | 7.770734 | 7.613518 | 7.860252 | 7.737932 | 7.95258 |
| 1385336_at | 4.378747 | 4.498968 | 4.425919 | 4.398978 | 4.645115 | 4.456929 | 5.326985 |
| 1379796_at | 5.200578 | 4.920435 | 5.205128 | 5.071747 | 5.170982 | 5.031005 | 4.989594 |
| 1392936_at | 8.882013 | 8.406152 | 9.262261 | 8.975443 | 9.393243 | 8.806762 | 7.959987 |
| 1395970_at | 4.781092 | 4.846477 | 4.596515 | 4.342038 | 4.706959 | 4.732536 | 5.091663 |
| 1369192_at | 5.561184 | 5.36412 | 5.201538 | 5.355132 | 5.326577 | 5.659776 | 4.955503 |
| 1368542_at | 6.355367 | 7.586223 | 6.711754 | 6.346843 | 6.56316 | 7.000522 | 7.277155 |
| 1383430_at | 6.21334 | 5.851398 | 6.75433 | 6.641164 | 6.445778 | 6.50764 | 5.806739 |
| 1385535_at | 4.019736 | 3.841945 | 4.062764 | 4.089753 | 4.042077 | 4.165624 | 3.831006 |
| 1377476_at | 3.935243 | 3.898896 | 3.840274 | 3.865414 | 3.998356 | 4.107018 | 3.720902 |
| 1384135_at | 7.574115 | 7.592611 | 7.557376 | 7.726628 | 7.711299 | 7.721932 | 7.560397 |
| 1392231_at | 6.878903 | 7.147771 | 7.61474 | 7.766599 | 7.91292 | 7.35021 | 5.771132 |
| 1385698_at | 3.877547 | 3.859262 | 3.777443 | 3.605165 | 3.7424 | 3.745963 | 3.587921 |
| 1382056_at | 8.204262 | 7.69305 | 8.301708 | 8.683247 | 9.183301 | 8.357295 | 7.16518 |
| 1387764_at | 5.733395 | 5.267832 | 5.536713 | 5.548715 | 5.493569 | 5.602545 | 5.13591 |
| 1389664_at | 7.741098 | 8.146133 | 7.528683 | 7.949046 | 7.981937 | 7.952165 | 8.531566 |
| 1381874_at | 6.733795 | 6.452854 | 6.456884 | 6.588163 | 6.284997 | 6.523057 | 6.100073 |
| 1394762_at | 3.747766 | 3.836961 | 3.726768 | 3.741149 | 3.941334 | 3.852886 | 3.618362 |
| 1383376_at | 6.631789 | 7.028277 | 7.303449 | 6.986742 | 6.760352 | 6.954577 | 7.371024 |
| 1397116_at | 3.722633 | 3.555765 | 3.710999 | 3.773147 | 3.705339 | 3.588653 | 3.874232 |
| 1392028_at | 6.161642 | 6.747499 | 6.346406 | 6.241838 | 6.323136 | 6.15851 | 7.27295 |
| 1393752_at | 6.207948 | 6.547756 | 6.148272 | 6.455214 | 6.651246 | 6.417392 | 5.840618 |
| 1391042_at | 7.196761 | 7.167479 | 6.843896 | 6.389245 | 6.757482 | 6.834679 | 7.544293 |
| 368199_at | 8.585706 | 8.944472 | 9.045243 | 9.265143 | 9.276926 | 9.377389 | 8.110664 |
| 1374231_at | 8.493602 | 8.997742 | 8.11689 | 8.315554 | 7.948455 | 8.465599 | 9.330748 |
| 1384641_at | 5.867063 | 5.877272 | 5.784143 | 5.513184 | 5.517425 | 5.626088 | 6.624306 |
| 1384195_at | 5.325397 | 5.825996 | 5.609386 | 5.355132 | 5.715931 | 5.367555 | 6.123647 |
| 1381483_at | 3.995783 | 4.010133 | 3.983216 | 3.971882 | 3.969843 | 4.01653 | 4.079169 |
| 1390603_at | 6.467269 | 6.867608 | 6.526795 | 6.621128 | 6.24163 | 6.673742 | 6.998137 |
| 1381950_at | 3.397326 | 3.369785 | 3.469694 | 3.379281 | 3.403623 | 3.397326 | 3.267779 |
| 1383443_at | 5.894824 | 5.907375 | 6.024725 | 5.853701 | 5.85921 | 6.136313 | 5.652074 |
| 1378318_at | 7.571926 | 7.800492 | 8.112397 | 7.987475 | 7.863851 | 8.146883 | 7.37359 |
| 1370521_at | 8.479857 | 8.580032 | 8.736684 | 8.764304 | 8.623886 | 8.798383 | 8.197548 |
| 1376294_at | 7.49468 | 7.950701 | 7.425112 | 7.689459 | 7.578406 | 7.479639 | 7.969805 |
| 1398025_at | 6.981408 | 7.04521 | 6.560595 | 6.889435 | 6.772504 | 6.868271 | 7.236035 |
| 1393939_at | 4.710334 | 4.865161 | 4.741891 | 4.586409 | 4.59178 | 4.506588 | 4.492715 |
| 1390649_at | 7.280403 | 7.574769 | 6.820701 | 7.03359 | 7.132005 | 6.938208 | 7.766439 |
| 1383309_at | 7.874241 | 8.135468 | 8.528866 | 8.112229 | 8.518259 | 7.954912 | 8.694724 |
| 1373319_at | 9.594949 | 9.441479 | 10.04752 | 10.11119 | 10.06627 | 9.901417 | 9.307665 |
| 1372141_at | 9.643188 | 9.597338 | 10.33766 | 10.39224 | 9.956746 | 10.18856 | 9.317099 |
| 1368083_at | 8.501241 | 8.375055 | 9.053139 | 8.928284 | 9.102935 | 8.728133 | 7.970649 |
| 1371435_at | 11.52837 | 11.47006 | 12.47228 | 12.43289 | 12.2189 | 12.11999 | 11.05863 |
| 1389917_at | 5.997911 | 5.507105 | 6.011787 | 5.98703 | 5.90818 | 5.888303 | 5.515914 |
| 1375407_at | 4.487324 | 4.330094 | 4.343735 | 4.488657 | 4.605165 | 4.326224 | 4.284785 |
| 1374112_at | 7.049012 | 6.725406 | 6.891224 | 7.196357 | 7.137989 | 6.729652 | 6.554617 |
| 1374739_at | 8.348062 | 8.649788 | 7.424175 | 7.760232 | 7.662029 | 7.815292 | 8.75921 |
| 1389095_at | 5.48851 | 5.217066 | 5.112291 | 5.397198 | 5.477914 | 5.072796 | 4.712782 |
| 1393104_at | 5.321407 | 5.704145 | 5.504231 | 5.332799 | 5.921095 | 5.580055 | 5.9151 |
| 1397676_at | 6.024075 | 6.128123 | 5.934997 | 6.174332 | 6.310561 | 5.89814 | 4.877137 |
| 1381286_at | 5.260874 | 5.373715 | 5.29669 | 5.03364 | 5.316363 | 5.388673 | 5.722119 |
| 1377028_at | 5.527717 | 5.845925 | 5.205825 | 5.427227 | 5.577792 | 5.374572 | 6.135862 |
| 1391078_at | 9.616963 | 9.613574 | 9.751381 | 9.433867 | 10.05209 | 9.263934 | 8.452422 |
| 1388396_at | 8.176018 | 7.835593 | 8.032324 | 8.018258 | 7.773587 | 8.066992 | 8.331665 |
| 1381074_at | 4.343351 | 4.301245 | 4.059032 | 4.183486 | 4.169704 | 4.085939 | 4.794154 |
| 1377210_at | 5.830016 | 5.616854 | 6.031799 | 5.853978 | 6.197914 | 5.930427 | 5.139862 |
| 1370180_at | 10.45966 | 10.83245 | 10.78238 | 10.88764 | 10.97763 | 10.78669 | 9.891602 |
| 1370766_at | 4.632916 | 4.678613 | 4.503179 | 4.577649 | 4.207348 | 4.398448 | 5.115596 |
| 1383670_at | 7.783752 | 7.593217 | 7.739582 | 7.592363 | 7.856423 | 7.209327 | 6.603798 |
| 1379827_at | 7.897097 | 7.849569 | 7.7663 | 7.924552 | 7.631788 | 7.663447 | 7.090357 |
| 1377746_at | 6.432148 | 6.590917 | 6.424811 | 6.508534 | 6.483669 | 6.596686 | 6.651214 |
| 1393989_at | 5.98976 | 6.474916 | 6.070125 | 6.001044 | 6.425105 | 6.268224 | 6.401078 |
| 1373804_at | 7.893696 | 7.963357 | 7.857588 | 8.120415 | 7.699296 | 8.068101 | 6.845052 |
| 1380454_at | 5.809904 | 5.870655 | 5.693093 | 5.71665 | 5.721161 | 5.87459 | 6.036958 |
| 1373702_at | 7.559095 | 7.845701 | 7.181182 | 7.331758 | 7.377075 | 7.495515 | 7.857952 |
| 1381130_at | 4.241232 | 4.333855 | 4.403803 | 4.14683 | 4.25664 | 4.293638 | 3.916558 |
| 1385113_at | 6.664915 | 7.042515 | 6.533225 | 6.690134 | 6.864402 | 6.917223 | 7.576541 |
| 1387136_at | 6.690675 | 6.697399 | 6.592852 | 6.718138 | 6.669893 | 6.53701 | 6.797323 |
| 1383300_at | 8.422153 | 8.731652 | 8.860932 | 8.982295 | 9.070573 | 8.854202 | 8.294344 |
| 1367877_at | 9.4471 | 9.92287 | 9.62197 | 8.882809 | 9.902495 | 8.730134 | 10.35139 |
| 1369754_a_at | 8.583344 | 8.276827 | 8.264377 | 8.283313 | 8.553526 | 8.482807 | 7.911593 |
| 1392950_at | 3.949199 | 4.09359 | 3.99867 | 4.08534 | 4.163895 | 3.918969 | 3.742798 |
| 1388091_at | 4.362346 | 4.793774 | 4.301891 | 4.13253 | 4.448635 | 4.329039 | 4.858257 |
| 1385676_at | 8.114047 | 8.06074 | 8.462139 | 8.533084 | 8.03906 | 8.343921 | 7.607347 |
| 1370682_at | 5.578813 | 5.507271 | 5.510365 | 5.944159 | 5.573662 | 5.843012 | 5.231738 |
| 1394790_at | 6.185159 | 6.363023 | 6.208196 | 6.068894 | 6.016272 | 6.007775 | 6.317944 |
| 1399108_at | 7.807585 | 7.455511 | 7.953623 | 7.823088 | 8.257681 | 7.911227 | 7.197025 |
| 1367856_at | 9.744949 | 9.796989 | 10.22231 | 10.50507 | 10.38059 | 10.14276 | 9.652992 |
| 1384849_at | 4.659446 | 4.628871 | 5.174466 | 5.222754 | 4.879312 | 4.919623 | 4.364507 |
| 1389938_at | 3.637811 | 3.579232 | 3.677572 | 3.837286 | 3.753255 | 3.592532 | 3.54826 |
| 1395316_at | 6.785841 | 6.744271 | 6.399231 | 5.525145 | 5.977116 | 6.479509 | 7.247047 |
| 1388390_at | 10.86305 | 10.86714 | 11.34452 | 11.8393 | 11.4165 | 11.4451 | 10.42769 |
| 1393539_at | 6.156527 | 5.168607 | 4.952404 | 5.172468 | 5.083432 | 5.052701 | 5.433546 |
| 1373396_at | 8.164183 | 8.316997 | 8.210297 | 8.055544 | 8.146979 | 8.247369 | 8.606754 |
| 1384528_at | 7.099858 | 7.424157 | 7.730496 | 8.039531 | 7.505124 | 7.682238 | 6.597373 |
| 1379963_at | 6.924796 | 7.095268 | 7.164582 | 7.115317 | 7.306993 | 7.145928 | 6.809468 |
| 1378992_at | 3.831981 | 3.701334 | 3.850427 | 3.85933 | 3.911037 | 3.948604 | 3.678719 |
| 1391376_at | 7.297353 | 7.473486 | 6.881233 | 6.738031 | 6.837921 | 6.654336 | 7.951333 |
| 1385137_at | 6.326237 | 6.421135 | 6.153827 | 5.911823 | 6.019728 | 6.247922 | 6.734987 |
| 1390892_at | 5.111681 | 5.3382 | 4.890708 | 5.20997 | 4.943911 | 5.357849 | 5.67563 |
| 1397956_at | 5.496901 | 5.765396 | 4.57526 | 4.745237 | 5.255328 | 5.146637 | 6.23139 |
| 1398208_s_at | 6.67011 | 6.821534 | 6.248422 | 6.320022 | 5.982574 | 6.358707 | 6.87605 |
| 1377621_at | 8.676155 | 8.675185 | 8.549399 | 8.432255 | 8.895957 | 8.592707 | 8.878285 |
| 1370293_at | 4.666933 | 4.609996 | 4.657549 | 4.806802 | 4.576158 | 4.727404 | 5.242464 |
| 1382513_at | 6.93924 | 6.903926 | 6.917757 | 6.843559 | 6.990688 | 6.991736 | 5.898944 |
| 1386125_at | 6.311382 | 6.381109 | 6.133764 | 6.260611 | 6.054617 | 6.325394 | 6.632 |
| 1383599_at | 9.633858 | 10.03068 | 10.32757 | 9.89942 | 10.08312 | 10.06208 | 8.2919 |
| 1369775_at | 9.454192 | 9.493169 | 9.497181 | 9.710562 | 9.436253 | 9.608369 | 8.090458 |
| 1387693_a_at | 4.774831 | 5.712843 | 5.222303 | 5.078581 | 4.94382 | 5.128631 | 5.751641 |
| 1389213_at | 7.553784 | 7.673512 | 7.17997 | 7.082279 | 7.28493 | 7.531156 | 7.664021 |
| 1391509_at | 7.810495 | 8.329447 | 7.825097 | 7.91756 | 8.117024 | 8.184319 | 8.45448 |
| 1398136_at | 4.138744 | 4.20485 | 4.177933 | 4.174646 | 4.278477 | 4.21161 | 4.050902 |
| AFFX-r2-Ec-bioC-5_at | 10.01607 | 10.05255 | 11.29769 | 11.00998 | 10.96151 | 11.01275 | 9.226769 |
| 1385386_at | 4.050452 | 4.199871 | 4.367786 | 4.404437 | 4.111017 | 4.351472 | 4.072888 |
| 1373647_at | 8.537195 | 8.662428 | 9.067445 | 8.963148 | 8.81195 | 8.987866 | 8.350924 |
| 1390477_at | 5.087529 | 5.212648 | 4.637308 | 4.863596 | 4.989594 | 4.71236 | 5.589063 |
| 1388131_at | 5.481963 | 5.72433 | 5.627964 | 5.313138 | 5.378316 | 5.320352 | 5.897792 |
| 1382670_at | 8.210095 | 8.049077 | 7.136542 | 7.314101 | 7.642927 | 7.445864 | 8.363667 |
| 1384137_at | 3.991965 | 3.996326 | 3.889869 | 4.067517 | 3.97958 | 3.898925 | 4.17252 |
| 1374707_at | 7.351236 | 7.672796 | 7.488287 | 7.697405 | 7.515403 | 7.602865 | 7.977337 |
| 1381492_at | 3.535668 | 3.463724 | 3.556002 | 3.687325 | 3.747469 | 3.650054 | 3.469432 |
| 1389599_at | 6.535401 | 6.775697 | 5.917576 | 6.014632 | 6.326524 | 6.204528 | 6.976052 |
| 1380175_at | 4.934431 | 4.975092 | 4.849851 | 4.929599 | 4.966442 | 4.859071 | 5.19297 |
| 1370983_at | 5.357496 | 5.442921 | 5.315738 | 5.143299 | 5.248557 | 5.359862 | 5.760543 |
| 1396130_at | 3.756449 | 3.764036 | 3.744044 | 3.847158 | 3.745616 | 3.741162 | 3.978887 |
| 1387914_at | 7.455623 | 7.375048 | 7.640913 | 7.405158 | 7.419675 | 7.231261 | 6.820446 |
| 1368824_at | 10.44398 | 10.30705 | 10.74571 | 10.8577 | 10.39224 | 10.80334 | 9.256274 |
| 1381456_at | 5.821987 | 5.673469 | 5.760094 | 5.587039 | 5.629439 | 5.737215 | 5.593373 |
| 1375178_at | 5.30388 | 5.523211 | 5.583334 | 5.437246 | 5.443804 | 5.620169 | 5.738722 |
| 1377441_at | 5.025587 | 5.070331 | 5.302622 | 5.118077 | 5.047172 | 5.16626 | 5.045742 |
| 1385290_at | 5.664179 | 5.663567 | 5.339044 | 5.510906 | 5.598518 | 5.437871 | 5.788163 |
| 1368198_at | 5.472901 | 5.92179 | 5.313882 | 5.480196 | 5.569974 | 5.485599 | 6.415973 |
| 1370253_at | 11.46042 | 11.55801 | 12.13153 | 12.02011 | 11.88533 | 11.72252 | 10.83548 |
| 1378906_at | 3.9529 | 3.996871 | 3.869548 | 3.959773 | 3.996177 | 3.762815 | 4.094419 |
| 1393438_at | 5.067597 | 5.185457 | 5.0534 | 5.206258 | 4.959296 | 5.153154 | 5.25502 |
| 1376661_at | 8.407186 | 8.641049 | 7.860358 | 7.939628 | 7.632013 | 8.114194 | 8.559167 |
| 1387822_at | 5.838364 | 6.31866 | 5.873867 | 5.790026 | 5.5082 | 5.909333 | 6.475088 |
| 1391283_at | 5.168301 | 5.670731 | 5.215901 | 5.355132 | 5.486444 | 5.460731 | 5.714625 |
| 1379691_at | 5.812119 | 5.90977 | 6.656428 | 6.738031 | 5.925239 | 6.572693 | 5.491544 |
| 1378200_at | 3.755446 | 3.651398 | 3.848461 | 3.847158 | 4.03614 | 3.881774 | 3.591021 |
| 1391962_at | 4.079075 | 4.011254 | 3.937441 | 4.017668 | 4.023766 | 3.917669 | 3.906001 |
| 1374608_at | 9.785419 | 9.985785 | 10.28848 | 10.32674 | 10.25331 | 10.05825 | 9.557885 |
| 1390406_at | 10.61951 | 10.62303 | 11.01077 | 10.92891 | 11.29526 | 11.09831 | 10.38777 |
| 1394343_s_at | 3.66591 | 3.629114 | 3.872273 | 3.785153 | 3.852298 | 3.840573 | 3.48113 |
| 1370317_at | 8.581536 | 8.538325 | 9.288004 | 9.273765 | 9.399475 | 9.017291 | 8.225464 |
| 1385391_at | 4.583987 | 4.423231 | 5.217826 | 4.527849 | 4.756794 | 4.618392 | 4.141161 |
| 1382156_at | 4.177727 | 4.599799 | 4.280314 | 4.23184 | 4.706416 | 4.383562 | 4.830796 |
| 1377365_at | 5.846111 | 5.083179 | 5.531814 | 6.126892 | 5.642908 | 5.686954 | 4.948696 |
| 1397587_at | 6.561215 | 6.659465 | 6.326444 | 6.196304 | 6.180505 | 6.178594 | 7.275823 |
| 1383054_at | 9.671877 | 9.604753 | 9.478561 | 9.484444 | 9.459532 | 9.528337 | 8.831633 |
| 1398949_at | 9.031281 | 8.873869 | 9.095255 | 9.297021 | 9.003186 | 9.17015 | 8.560669 |
| 1379822_at | 4.961073 | 4.830668 | 5.298664 | 4.847941 | 5.227081 | 5.084299 | 4.536292 |
| 1373572_at | 8.281455 | 8.071155 | 8.725759 | 8.553317 | 8.625631 | 8.451843 | 7.925965 |
| 1395817_at | 6.936618 | 7.226047 | 6.714064 | 6.489645 | 6.649266 | 6.782707 | 7.206994 |
| 1387694_at | 5.038728 | 4.873559 | 4.91933 | 4.932759 | 5.07253 | 4.690848 | 4.759871 |
| 1384573_at | 9.396305 | 9.335087 | 9.668519 | 9.820306 | 10.29503 | 9.807943 | 9.0429 |
| 1385967_at | 7.374976 | 7.407112 | 6.997503 | 7.073656 | 6.439714 | 7.158605 | 7.699587 |
| 1397934_at | 8.072479 | 8.117771 | 7.76032 | 7.912047 | 7.672647 | 7.855582 | 8.212134 |
| 1380887_at | 4.704517 | 4.566671 | 4.558343 | 4.498599 | 4.469823 | 4.532469 | 4.988036 |
| 1390592_at | 9.083445 | 9.08483 | 9.027625 | 9.632865 | 10.27661 | 9.301206 | 7.974309 |
| 1394383_at | 5.712843 | 6.062 | 5.32417 | 5.199135 | 5.600375 | 5.246925 | 6.689685 |
| 1376622_at | 8.515894 | 8.814472 | 8.398168 | 8.068337 | 8.49399 | 8.378894 | 9.057895 |
| 1385074_at | 6.094208 | 5.703184 | 6.068776 | 5.94621 | 6.214383 | 6.403681 | 5.559301 |
| 1385790_at | 8.262242 | 8.645411 | 8.216791 | 8.092521 | 8.253879 | 8.458125 | 8.520391 |
| 1371174_s_at | 6.130192 | 6.189456 | 5.805434 | 6.222562 | 5.865165 | 5.867801 | 6.548081 |
| 1387836_at | 6.664029 | 6.514591 | 7.320945 | 7.119733 | 6.632225 | 6.906481 | 6.310191 |
| 1370918_a_at | 11.47515 | 11.56737 | 11.98988 | 11.81814 | 11.71168 | 11.76732 | 11.13211 |
| 1398486_at | 5.620235 | 5.738726 | 6.477335 | 7.050119 | 7.67263 | 6.596901 | 4.542619 |
| 1397268_at | 4.564852 | 5.071743 | 4.48244 | 4.729605 | 4.645403 | 4.566671 | 5.231618 |
| 1393428_at | 6.698202 | 6.376886 | 7.113966 | 7.290344 | 7.298931 | 6.75517 | 6.068753 |
| 1391678_at | 6.727646 | 7.030317 | 6.037111 | 5.683982 | 5.954492 | 6.263617 | 6.870096 |
| 1388363_at | 8.480147 | 8.557988 | 9.033092 | 9.198227 | 8.642246 | 9.018138 | 8.167374 |
| 1388669_at | 7.942719 | 7.729223 | 8.560117 | 8.667614 | 8.518848 | 8.408285 | 7.724199 |
| 1394887_x_at | 4.062696 | 4.143489 | 3.655798 | 3.535668 | 3.475193 | 3.918852 | 4.205032 |
| 1384367_at | 5.151534 | 5.5769 | 5.098234 | 5.103271 | 5.136866 | 5.448825 | 5.648899 |
| 1383329_at | 5.298821 | 5.567031 | 5.281227 | 5.280507 | 5.561147 | 5.338702 | 5.60479 |
| Probeset | C | C | C | C | C | C | C |
| 1382608_at | 5.237744 | 5.272204 | 5.236143 | 5.289465 | 5.216091 | 5.254174 | 5.25778 |
| 1376218_a_at | 5.875681 | 6.087847 | 6.374832 | 5.645509 | 6.013108 | 6.381223 | 6.093614 |
| 1386519_x_at | 7.830681 | 7.749506 | 7.903098 | 8.288516 | 7.962189 | 7.933318 | 7.815044 |
| 1385442_at | 7.82583 | 7.64524 | 7.92864 | 7.733391 | 7.858502 | 7.715819 | 7.545482 |
| 1384923_at | 7.364752 | 7.365495 | 7.465344 | 7.415414 | 7.445385 | 7.473401 | 7.313717 |
| 1385163_at | 5.631046 | 5.722119 | 5.606002 | 5.509671 | 5.462721 | 5.761826 | 5.544381 |
| 1383659_a_at | 6.517474 | 6.730487 | 7.065184 | 6.930894 | 6.538912 | 6.294332 | 6.717881 |
| 1393517_at | 7.123498 | 7.190511 | 7.201717 | 6.995643 | 7.291952 | 6.938515 | 7.099921 |
| 1376289_at | 3.529993 | 3.443991 | 3.474249 | 3.519759 | 3.573975 | 3.619594 | 3.533455 |
| 1390090_at | 6.99303 | 6.866285 | 6.674141 | 7.093017 | 7.107792 | 7.016472 | 7.090415 |
| 1379140_at | 4.95892 | 4.96916 | 4.965008 | 4.695874 | 4.613397 | 4.478106 | 4.482764 |
| 1381966_at | 9.675863 | 9.646928 | 9.435287 | 9.197302 | 9.488352 | 9.284593 | 9.163014 |
| 1378355_a_at | 7.286371 | 7.448578 | 7.42337 | 6.6439 | 7.065871 | 6.970562 | 6.66817 |
| 1377123_at | 9.45978 | 9.347403 | 9.322108 | 9.462491 | 9.447508 | 9.420515 | 9.197153 |
| 1398571_x_at | 6.678639 | 6.99583 | 6.752649 | 6.590249 | 6.586327 | 6.127059 | 6.031502 |
| 1383472_at | 3.74166 | 3.79001 | 3.703913 | 3.652824 | 3.802661 | 3.750435 | 3.817995 |
| 1383900_at | 4.527613 | 4.364276 | 4.440317 | 4.5013 | 4.451543 | 4.433715 | 4.445819 |
| 1382015_at | 8.354576 | 8.1445 | 8.409086 | 8.170871 | 8.230312 | 8.158051 | 7.955302 |
| 1384467_at | 8.975141 | 8.884631 | 8.788698 | 8.918757 | 8.86129 | 8.915307 | 8.846082 |
| 1382825_at | 6.690117 | 6.815207 | 6.605777 | 6.815207 | 6.785439 | 6.712093 | 6.528925 |
| 1395990_at | 7.938661 | 7.80234 | 7.774826 | 7.781617 | 7.863002 | 7.788823 | 7.671122 |
| 1376844_at | 3.656266 | 3.74887 | 3.757629 | 3.714402 | 3.77409 | 3.746111 | 3.635083 |
| 1367978_at | 6.546412 | 6.5001 | 6.505364 | 6.571766 | 6.655691 | 6.603533 | 6.490598 |
| 1387406_at | 6.540777 | 6.538825 | 6.67007 | 6.315783 | 6.397716 | 6.325319 | 6.413598 |
| 1379110_at | 4.37422 | 4.263525 | 4.197585 | 4.104217 | 4.265249 | 4.181891 | 4.350189 |
| 1381122_at | 4.973314 | 4.797912 | 4.800685 | 4.895353 | 5.062703 | 4.85751 | 4.931449 |
| 1381872_at | 7.586651 | 7.512342 | 7.598962 | 7.285015 | 7.624096 | 7.473142 | 7.274368 |
| 1393266_at | 5.743452 | 5.544381 | 5.486993 | 5.306598 | 5.554374 | 5.42669 | 5.400927 |
| 1372462_at | 8.364795 | 8.603433 | 8.551539 | 8.397251 | 8.5568264 | 8.568455 | 8.298081 |
| 1394498_at | 5.419531 | 5.198261 | 5.224532 | 5.319775 | 5.323577 | 5.33164 | 5.397302 |
| 1381941_at | 8.694863 | 8.905404 | 8.752977 | 8.572081 | 8.49661 | 8.340797 | 8.472677 |
| 1397738_at | 4.019499 | 4.100877 | 4.030312 | 4.010981 | 4.030298 | 4.082993 | 4.044111 |
| 1397834_at | 6.963689 | 7.008077 | 6.887756 | 6.859623 | 6.82511 | 6.664778 | 6.50166 |
| 1373925_at | 7.369652 | 7.424104 | 7.286968 | 7.241315 | 7.417064 | 7.121076 | 7.009828 |
| 1384374_at | 6.997938 | 7.00376 | 7.116975 | 6.819456 | 6.939509 | 6.880446 | 6.859112 |
| 1397187_at | 6.410882 | 6.122884 | 6.307554 | 6.16956 | 6.29398 | 6.208331 | 6.301008 |
| 1368985_at | 4.344123 | 4.192491 | 4.231533 | 4.221849 | 4.296797 | 4.412011 | 4.247998 |
| 1386414_at | 7.501521 | 7.575122 | 7.399769 | 7.423739 | 7.443794 | 7.64695 | 7.462433 |
| 1377750_at | 7.768057 | 7.973285 | 8.40535 | 7.903111 | 8.123004 | 8.358795 | 8.45301 |
| 1372514_s_at | 8.795483 | 8.624616 | 8.782141 | 8.918743 | 8.801713 | 8.718723 | 8.431679 |
| 1387459_at | 4.300175 | 4.301082 | 4.720045 | 4.518814 | 4.459496 | 4.521545 | 4.123799 |
| 1379235_x_at | 8.050582 | 8.025153 | 7.932474 | 7.722358 | 8.030917 | 7.947163 | 7.82587 |
| 1396326_at | 6.161165 | 6.561937 | 6.302463 | 6.203722 | 6.208262 | 6.257762 | 6.17307 |
| 1389835_at | 3.812722 | 3.598725 | 3.669667 | 3.602518 | 3.773353 | 3.730474 | 3.69211 |
| 1394414_at | 9.969117 | 9.897804 | 10.14609 | 10.14356 | 10.12581 | 10.24219 | 10.23343 |
| 1374656_at | 9.629452 | 9.576869 | 9.816997 | 9.559234 | 9.546773 | 9.32456 | 9.128706 |
| 1387083_at | 6.883273 | 6.759039 | 6.441383 | 6.794412 | 6.999748 | 6.270687 | 6.422242 |
| 1383984_at | 4.623921 | 4.633568 | 4.564507 | 4.539056 | 4.527172 | 4.607618 | 4.325744 |
| 1385760_at | 8.081191 | 8.049202 | 8.247117 | 7.892154 | 7.965312 | 7.556368 | 7.6695 |
| 1369005_at | 5.687619 | 5.544381 | 5.661943 | 5.741251 | 5.537835 | 5.566714 | 5.739558 |
| 1384527_at | 5.141262 | 5.368642 | 5.319001 | 5.185683 | 5.322784 | 5.315419 | 5.342408 |
| 1390464_at | 6.099983 | 6.372503 | 6.266014 | 6.241456 | 6.184791 | 6.169358 | 6.063681 |
| 1377414_at | 6.482402 | 6.149155 | 6.464091 | 6.53701 | 6.273317 | 6.558447 | 6.615859 |
| 1376278_at | 6.543892 | 6.502883 | 6.995767 | 6.573368 | 6.753383 | 6.617303 | 6.714534 |
| 1368646_at | 6.069268 | 6.076303 | 6.160336 | 5.946667 | 6.029633 | 6.046796 | 5.854462 |
| 1374245_at | 5.545787 | 5.603729 | 5.62681 | 5.90412 | 5.887429 | 6.114687 | 5.899921 |
| 1379499_at | 4.173024 | 3.949435 | 4.001183 | 4.162551 | 4.307143 | 4.18339 | 4.355149 |
| 1374807_at | 7.327211 | 7.253629 | 7.110172 | 7.192801 | 7.372729 | 7.088235 | 7.028255 |
| 1370115_at | 5.218799 | 5.147045 | 5.078444 | 5.012252 | 5.13361 | 5.193181 | 5.223045 |
| 1387912_at | 7.91239 | 8.316286 | 8.232833 | 8.199003 | 8.09284 | 7.998546 | 8.392527 |
| 1385652_at | 8.222899 | 8.248344 | 8.234552 | 7.968577 | 8.051608 | 8.045238 | 7.960335 |
| 1370688_at | 7.36588 | 7.518903 | 7.701268 | 7.555538 | 7.578135 | 7.759163 | 7.58329 |
| 1397911_at | 6.543011 | 6.562404 | 6.375987 | 6.363199 | 6.536549 | 6.190737 | 6.119697 |
| 1393853_at | 6.731545 | 6.783132 | 6.644372 | 6.372678 | 6.611951 | 6.214813 | 6.162276 |
| 1395951_at | 4.695693 | 4.758304 | 4.780238 | 4.643683 | 4.749183 | 4.836736 | 4.652167 |
| 1385775_at | 4.674896 | 4.580182 | 4.704516 | 4.51669 | 4.481854 | 4.618286 | 4.326023 |
| 1397500_x_at | 7.382126 | 7.45492 | 7.417569 | 7.013223 | 7.519891 | 7.493919 | 7.317633 |
| 1380152_at | 4.953241 | 5.05765 | 5.086932 | 4.992159 | 5.018866 | 4.923829 | 5.132639 |
| 1371813_at | 6.396741 | 6.364824 | 6.474042 | 6.258213 | 6.110988 | 6.283867 | 6.343924 |
| 1384377_at | 6.882679 | 6.918349 | 7.010346 | 6.908804 | 6.894845 | 6.923368 | 7.012494 |
| 1388486_at | 7.318186 | 7.182537 | 7.520851 | 7.276855 | 7.074702 | 7.406231 | 7.470965 |
| 1383667_at | 7.527523 | 7.385785 | 7.473843 | 7.993592 | 7.674573 | 7.730061 | 7.746763 |
| 1373423_at | 6.304946 | 6.164446 | 6.700276 | 5.946891 | 5.95794 | 6.015735 | 6.097276 |
| 1379408_at | 8.124533 | 8.287372 | 8.288827 | 8.177428 | 7.97983 | 8.316875 | 8.694256 |
| 1389551_at | 8.349044 | 8.145229 | 8.2726 | 8.60557 | 8.460846 | 8.113041 | 8.400359 |
| 1390259_at | 4.66775 | 5.000864 | 5.153662 | 5.220275 | 5.160327 | 4.853355 | 5.016075 |
| 1395303_at | 3.247565 | 3.214602 | 3.138627 | 3.153899 | 3.121769 | 3.139992 | 3.111204 |
| 1393580_at | 6.518933 | 6.478083 | 6.254367 | 6.4354 | 6.830166 | 6.516244 | 6.258315 |
| 1378275_at | 8.040595 | 7.942403 | 8.202556 | 8.150152 | 7.935424 | 8.206129 | 8.455176 |
| 1388938_at | 9.778912 | 9.530297 | 9.680033 | 9.901099 | 9.810025 | 9.494555 | 9.334763 |
| 1385518_at | 6.536772 | 6.546027 | 6.618618 | 6.620546 | 6.385759 | 6.383429 | 6.483809 |
| 1368397_at | 3.965976 | 4.088714 | 4.006536 | 3.927212 | 3.96137 | 3.981893 | 3.928241 |
| 1379980_at | 7.472297 | 7.420474 | 7.562075 | 7.537294 | 7.328248 | 7.494855 | 7.78891 |
| 1388781_at | 6.270394 | 6.245816 | 6.501316 | 6.234612 | 6.186758 | 6.077479 | 6.119421 |
| 1367866_at | 6.277639 | 5.863702 | 6.152303 | 6.008075 | 5.894587 | 5.756599 | 5.809089 |
| 1383132_at | 6.862921 | 6.633653 | 6.563514 | 6.634268 | 6.409718 | 6.520276 | 6.355964 |
| 1391709_at | 4.482328 | 4.228935 | 4.368574 | 4.293601 | 4.381164 | 4.314025 | 4.489163 |
| 1369051_at | 5.894917 | 5.761939 | 6.265352 | 5.65474 | 5.875081 | 5.563537 | 5.851093 |
| 1388037_at | 4.448134 | 4.387766 | 4.347017 | 4.401957 | 4.49354 | 4.324617 | 4.633814 |
| 1386158_at | 3.862222 | 3.742301 | 3.814765 | 3.765849 | 3.829825 | 3.812779 | 3.938524 |
| 1378705_at | 8.914849 | 8.882903 | 8.979451 | 8.693889 | 8.453753 | 8.589615 | 8.683902 |
| 1375738_at | 4.655386 | 4.407726 | 4.595206 | 4.440245 | 4.527604 | 4.779067 | 4.796527 |
| 1387899_at | 5.697924 | 5.876212 | 5.757785 | 5.646864 | 5.63307 | 5.501503 | 5.517219 |
| 1369014_at | 5.160412 | 5.263623 | 5.386469 | 5.057213 | 5.110736 | 5.397346 | 5.217783 |
| 1398196_at | 4.953522 | 4.911203 | 4.869875 | 4.83505 | 5.128952 | 4.741245 | 4.884861 |
| 1380392_at | 4.830327 | 4.865947 | 5.107021 | 5.056031 | 5.136112 | 5.193119 | 5.244334 |
| 1388447_at | 7.947177 | 7.909805 | 8.07842 | 8.022986 | 7.872428 | 7.97634 | 7.878162 |
| 1372593_at | 6.840674 | 6.902462 | 6.911244 | 6.728593 | 6.597356 | 6.633972 | 7.08344 |
| 1395875_at | 7.14728 | 7.111489 | 6.958799 | 6.898352 | 7.028319 | 7.10653 | 7.161164 |
| 1378642_at | 6.1585 | 6.11586 | 5.94312 | 5.989403 | 6.101192 | 5.888353 | 5.896107 |
| 1370260_at | 6.392463 | 6.686918 | 6.693615 | 6.692952 | 6.485528 | 6.795548 | 6.63388 |
| 1376136_at | 5.320349 | 5.25436 | 5.183097 | 5.116965 | 5.184211 | 5.239656 | 5.280122 |
| 1369894_at | 3.501081 | 3.602518 | 3.495698 | 3.511197 | 3.535245 | 3.636022 | 3.526432 |
| 1391555_at | 8.083806 | 7.999867 | 8.307345 | 8.0932 | 7.908228 | 7.945851 | 7.849791 |
| 1382932_at | 4.965125 | 4.927613 | 4.653916 | 4.844157 | 4.831355 | 5.184245 | 5.051153 |
| 1389431_at | 5.838387 | 5.736865 | 5.959344 | 5.662533 | 5.689092 | 6.019244 | 6.220915 |
| 1378872_at | 4.390531 | 4.487633 | 4.425033 | 4.530049 | 4.526018 | 4.598366 | 4.539138 |
| 1382110_at | 7.527317 | 7.644603 | 7.275493 | 7.590899 | 7.560787 | 7.254669 | 7.366466 |
| 1379206_at | 6.751652 | 6.62528 | 6.713236 | 6.669916 | 6.732019 | 6.675263 | 6.759973 |
| 1394361_a_at | 5.220908 | 5.271112 | 5.301559 | 5.092201 | 5.308119 | 4.644793 | 4.690529 |
| 1390534_at | 8.042464 | 8.129693 | 8.583872 | 7.886003 | 8.173897 | 8.12737 | 8.130038 |
| 1379342_at | 6.494045 | 6.62753 | 6.667481 | 6.473165 | 6.521787 | 6.390028 | 6.378516 |
| 1393798_at | 3.300071 | 3.401115 | 3.486278 | 3.435249 | 3.324261 | 3.352595 | 3.562655 |
| 1396104_at | 5.822264 | 5.89363 | 5.866673 | 5.733808 | 6.027052 | 5.645224 | 5.699922 |
| 1376852_at | 5.764757 | 6.209448 | 5.845733 | 6.007231 | 5.838274 | 6.153732 | 6.366836 |
| 1385292_at | 4.278333 | 4.0349 | 4.434404 | 4.369305 | 4.430857 | 4.43546 | 4.483979 |
| 1367489_at | 8.514793 | 8.389283 | 8.394011 | 8.367865 | 8.380845 | 8.185003 | 8.162172 |
| 1385336_at | 5.110112 | 4.722005 | 5.410576 | 4.829183 | 5.072895 | 5.069473 | 4.798046 |
| 1379796_at | 4.703331 | 4.666933 | 4.609902 | 4.641956 | 4.730378 | 4.791742 | 4.858253 |
| 1392936_at | 7.668291 | 8.084027 | 8.168558 | 7.72082 | 7.378934 | 8.020758 | 8.517279 |
| 1395970_at | 5.399796 | 5.037643 | 5.316555 | 4.966114 | 5.024739 | 5.416674 | 5.240194 |
| 1369192_at | 5.147122 | 4.780859 | 5.06547 | 4.907973 | 4.74804 | 5.07179 | 5.05255 |
| 1368542_at | 8.206008 | 7.775654 | 8.171132 | 8.004963 | 8.135756 | 7.735831 | 7.747696 |
| 1383430_at | 5.478167 | 5.466583 | 5.653591 | 5.813197 | 5.449554 | 5.836753 | 5.873596 |
| 1385535_at | 3.794345 | 3.811859 | 3.906691 | 3.735849 | 3.732864 | 3.817411 | 3.761728 |
| 1377476_at | 3.711524 | 3.670412 | 3.716365 | 3.711606 | 3.727182 | 3.823032 | 3.771248 |
| 1384135_at | 7.085586 | 7.186797 | 7.319472 | 7.261056 | 7.17095 | 7.386418 | 7.346428 |
| 1392231_at | 5.758788 | 6.233316 | 6.820614 | 6.333507 | 5.895723 | 6.562334 | 6.745554 |
| 1385698_at | 3.517968 | 3.50435 | 3.495625 | 3.60559 | 3.497468 | 3.561142 | 3.636274 |
| 1382056_at | 7.083643 | 7.252334 | 7.361255 | 7.384962 | 7.009626 | 7.499321 | 7.822615 |
| 1387764_at | 5.322744 | 5.107956 | 5.133345 | 5.273161 | 5.226465 | 5.159104 | 5.249519 |
| 1389664_at | 8.336394 | 8.705423 | 8.534367 | 8.692155 | 8.569625 | 8.289446 | 8.136463 |
| 1381874_at | 6.099969 | 6.00581 | 6.144595 | 6.210897 | 6.28222 | 6.127144 | 6.290771 |
| 1394762_at | 3.615049 | 3.521752 | 3.658248 | 3.639301 | 3.594899 | 3.693107 | 3.610157 |
| 1383376_at | 7.568335 | 7.458378 | 7.290963 | 7.567987 | 7.600682 | 7.440106 | 7.290963 |
| 1397116_at | 3.799208 | 3.861322 | 3.884911 | 3.820761 | 3.90878 | 3.88995 | 3.807507 |
| 1392028_at | 7.377159 | 7.071283 | 7.095986 | 6.540505 | 6.936805 | 6.96648 | 6.871274 |
| 1393752_at | 5.659136 | 5.438947 | 5.811106 | 5.841673 | 5.839612 | 6.050303 | 6.119885 |
| 1391042_at | 7.478658 | 7.445333 | 7.430156 | 7.440064 | 7.613535 | 7.965356 | 8.029414 |
| 368199_at | 8.204284 | 8.34858 | 8.33714 | 8.335964 | 8.198246 | 8.61636 | 8.767744 |
| 1374231_at | 9.508273 | 9.252295 | 9.300917 | 9.419778 | 9.431831 | 8.811543 | 8.918308 |
| 1384641_at | 6.336515 | 6.107025 | 6.345958 | 5.95118 | 6.332307 | 6.069465 | 6.149665 |
| 1384195_at | 6.221077 | 5.764872 | 6.51754 | 6.276005 | 6.330413 | 6.399849 | 5.915063 |
| 1381483_at | 4.08954 | 4.213387 | 4.215619 | 4.121345 | 4.197003 | 4.054011 | 4.108611 |
| 1390603_at | 7.170917 | 7.008214 | 7.59984 | 6.975703 | 7.243353 | 7.285737 | 7.020418 |
| 1381950_at | 3.272313 | 3.247869 | 3.335784 | 3.303979 | 3.31902 | 3.327817 | 3.348928 |
| 1383443_at | 5.683651 | 5.567135 | 5.754669 | 5.467944 | 5.751992 | 5.677403 | 5.701218 |
| 1378318_at | 7.344914 | 7.180416 | 7.153283 | 7.37251 | 7.28878 | 7.671009 | 7.572664 |
| 1370521_at | 8.445812 | 8.335108 | 8.321042 | 8.443235 | 8.48602 | 8.351527 | 8.365915 |
| 1376294_at | 8.240748 | 8.136396 | 8.121804 | 8.206327 | 8.031389 | 7.939353 | 7.843639 |
| 1398025_at | 7.378956 | 7.276747 | 7.364328 | 7.10214 | 7.171768 | 7.094738 | 7.304103 |
| 1393939_at | 4.455979 | 4.321684 | 4.388084 | 4.384665 | 4.239433 | 4.385835 | 4.367218 |
| 1390649_at | 7.938102 | 7.687889 | 7.673324 | 7.791348 | 7.858171 | 7.491383 | 7.527008 |
| 1383309_at | 8.849448 | 8.689932 | 8.969284 | 8.891145 | 8.933948 | 8.617682 | 8.604857 |
| 1373319_at | 9.054832 | 9.237388 | 9.183479 | 9.331507 | 9.182054 | 9.351879 | 9.526916 |
| 1372141_at | 9.138563 | 9.133975 | 9.291849 | 9.34714 | 9.239232 | 9.295156 | 9.670249 |
| 1368083_at | 8.131899 | 7.967377 | 8.205659 | 8.272071 | 8.390661 | 8.145126 | 8.221627 |
| 1371435_at | 11.00867 | 10.90612 | 11.23199 | 11.07677 | 10.98521 | 11.27023 | 11.55239 |
| 1389917_at | 5.655532 | 5.424886 | 5.451375 | 5.570937 | 5.415385 | 5.4535 | 5.45669 |
| 1375407_at | 4.146777 | 4.198401 | 4.162417 | 4.179388 | 4.179573 | 4.210067 | 4.246474 |
| 1374112_at | 6.447901 | 6.478707 | 6.686114 | 6.529024 | 6.500093 | 6.600799 | 6.615228 |
| 1374739_at | 8.910156 | 8.84001 | 9.070197 | 9.07402 | 8.858293 | 8.669296 | 8.599008 |
| 1389095_at | 4.79639 | 4.404411 | 4.732173 | 4.906512 | 5.026744 | 4.47297 | 4.326963 |
| 1393104_at | 5.975915 | 6.598068 | 6.661273 | 6.589076 | 6.111802 | 6.522552 | 6.20425 |
| 1397676_at | 4.932901 | 5.266384 | 5.715198 | 5.111778 | 5.274206 | 5.345541 | 5.855012 |
| 1381286_at | 5.782027 | 5.606054 | 5.685497 | 5.498501 | 5.475041 | 5.605097 | 5.508228 |
| 1377028_at | 6.24998 | 5.820455 | 6.063891 | 5.970941 | 6.061626 | 5.739891 | 6.085501 |
| 1391078_at | 8.106426 | 8.271168 | 8.740164 | 8.604317 | 8.221337 | 9.078712 | 9.316119 |
| 1388396_at | 8.582576 | 8.467639 | 8.632741 | 8.876118 | 8.642394 | 8.157713 | 8.432301 |
| 1381074_at | 4.776857 | 4.698159 | 4.460426 | 4.412136 | 4.875408 | 4.545246 | 4.428375 |
| 1377210_at | 5.248109 | 4.862723 | 5.700934 | 5.485466 | 5.014901 | 5.171668 | 5.372469 |
| 1370180_at | 10.05871 | 9.922619 | 10.49934 | 10.19777 | 9.827023 | 10.40441 | 10.42711 |
| 1370766_at | 4.942443 | 4.938172 | 5.166151 | 5.488656 | 5.322127 | 4.71238 | 5.042534 |
| 1383670_at | 6.710051 | 6.607325 | 6.621336 | 6.709491 | 6.712093 | 7.383305 | 7.343236 |
| 1379827_at | 7.363442 | 6.822804 | 7.052309 | 7.07748 | 7.079997 | 7.625611 | 7.462587 |
| 1377746_at | 6.748035 | 6.562857 | 6.854945 | 6.829264 | 6.771986 | 6.746288 | 6.820429 |
| 1393989_at | 7.171054 | 6.991178 | 7.09134 | 6.963071 | 7.056965 | 6.799059 | 6.583873 |
| 1373804_at | 6.825274 | 6.702404 | 7.208495 | 7.237738 | 6.824612 | 7.527008 | 7.716825 |
| 1380454_at | 6.241324 | 5.983879 | 6.00935 | 5.947613 | 5.973758 | 6.055284 | 5.955546 |
| 1373702_at | 8.33185 | 8.305246 | 8.161594 | 8.513912 | 8.321965 | 7.81442 | 7.873549 |
| 1381130_at | 3.90718 | 4.131394 | 4.062093 | 4.00716 | 3.997464 | 4.159053 | 4.070978 |
| 1385113_at | 7.347283 | 7.428331 | 7.30104 | 7.161173 | 7.416947 | 7.087856 | 7.051209 |
| 1387136_at | 6.857859 | 6.879307 | 6.715853 | 6.835796 | 6.916941 | 6.910151 | 6.800685 |
| 1383300_at | 8.006082 | 8.006505 | 8.361511 | 8.2749 | 8.022177 | 8.359105 | 8.558606 |
| 1367877_at | 10.90156 | 11.00311 | 11.20721 | 11.24657 | 10.7225 | 10.35455 | 9.967765 |
| 1369754_a_at | 8.0392 | 8.245252 | 7.920427 | 7.959489 | 8.000222 | 8.032917 | 8.209331 |
| 1392950_at | 3.887129 | 3.883732 | 3.808656 | 3.812032 | 3.820393 | 3.88542 | 3.706304 |
| 1388091_at | 4.883372 | 4.895909 | 4.860044 | 4.659865 | 4.763117 | 5.100578 | 5.081373 |
| 1385676_at | 7.767475 | 7.783984 | 7.600493 | 7.923105 | 7.825277 | 7.957292 | 7.848675 |
| 1370682_at | 5.233868 | 5.32624 | 5.095332 | 5.170874 | 5.242305 | 5.48155 | 5.263955 |
| 1394790_at | 6.517335 | 6.383183 | 6.546073 | 6.377263 | 6.520926 | 6.432519 | 6.411025 |
| 1399108_at | 7.197649 | 7.339689 | 7.478999 | 7.320491 | 7.149115 | 7.20594 | 7.58427 |
| 1367856_at | 9.22695 | 9.529068 | 9.365102 | 9.385048 | 9.389596 | 9.76226 | 9.547979 |
| 1384849_at | 4.388693 | 4.320512 | 4.282258 | 4.323105 | 4.50827 | 4.489202 | 4.609522 |
| 1389938_at | 3.470526 | 3.407639 | 3.488442 | 3.499909 | 3.515915 | 3.490367 | 3.425181 |
| 1395316_at | 7.32684 | 7.191957 | 7.672393 | 7.075363 | 7.224963 | 7.175964 | 7.116155 |
| 1388390_at | 10.41323 | 10.51712 | 10.485 | 10.62885 | 10.5942 | 10.75473 | 10.7773 |
| 1393539_at | 5.341786 | 5.529504 | 5.246557 | 5.254529 | 5.362529 | 5.602461 | 5.540961 |
| 1373396_at | 9.154677 | 8.864159 | 9.202926 | 9.102311 | 8.932804 | 8.366235 | 8.549971 |
| 1384528_at | 6.294633 | 6.639533 | 6.851434 | 6.655983 | 6.499612 | 7.042111 | 7.278227 |
| 1379963_at | 6.946089 | 6.646229 | 6.730348 | 6.720061 | 6.709743 | 6.949842 | 6.90215 |
| 1378992_at | 3.715271 | 3.630485 | 3.611331 | 3.743885 | 3.56719 | 3.654911 | 3.629206 |
| 1391376_at | 7.649773 | 7.981893 | 7.683304 | 7.517512 | 7.49073 | 7.601751 | 7.527206 |
| 1385137_at | 6.486932 | 6.690671 | 6.524926 | 6.506544 | 6.656693 | 6.51492 | 6.473502 |
| 1390892_at | 5.596472 | 5.604225 | 5.50656 | 5.408264 | 5.570845 | 5.5082 | 5.409615 |
| 1397956_at | 6.013105 | 6.030972 | 6.260479 | 5.900233 | 5.673819 | 6.03905 | 6.140571 |
| 1398208_s_at | 6.851001 | 6.896828 | 7.111651 | 6.972396 | 6.89232 | 7.05601 | 7.049933 |
| 1377621_at | 9.057845 | 9.147932 | 9.271816 | 8.936416 | 8.844168 | 9.113891 | 9.058689 |
| 1370293_at | 5.002656 | 5.145953 | 4.947144 | 4.866258 | 4.888348 | 5.155453 | 4.853477 |
| 1382513_at | 6.674163 | 6.665315 | 6.480641 | 6.607116 | 6.736527 | 6.658752 | 6.65185 |
| 1386125_at | 6.722093 | 6.815417 | 6.393768 | 6.646826 | 6.915203 | 6.55516 | 6.508228 |
| 1383599_at | 8.080652 | 7.625851 | 7.843413 | 7.828882 | 8.126902 | 9.642324 | 9.524186 |
| 1369775_at | 8.114099 | 8.46892 | 8.776746 | 8.329269 | 7.897293 | 9.18486 | 9.188358 |
| 1387693_a_at | 6.34591 | 5.564146 | 5.898449 | 6.298315 | 6.189505 | 6.092007 | 5.62898 |
| 1389213_at | 8.130876 | 8.027784 | 7.744935 | 7.914217 | 8.246469 | 8.134782 | 7.863013 |
| 1391509_at | 8.435513 | 8.439677 | 8.928973 | 8.686683 | 8.507594 | 8.573678 | 8.37123 |
| 1398136_at | 4.108807 | 4.099462 | 4.034506 | 4.046964 | 4.062351 | 4.154086 | 4.099306 |
| AFFX-r2-Ec-bioC-5_at | 9.336654 | 9.16583 | 9.068818 | 9.341125 | 9.591659 | 10.03402 | 10.11081 |
| 1385386_at | 3.879526 | 3.886862 | 3.72369 | 3.939486 | 3.964475 | 3.997111 | 3.855288 |
| 1373647_at | 8.371392 | 8.229571 | 8.671911 | 8.447359 | 8.268229 | 8.235445 | 8.429099 |
| 1390477_at | 5.454602 | 5.297205 | 5.407532 | 5.152992 | 5.57538 | 5.332575 | 5.384387 |
| 1388131_at | 5.882669 | 5.853117 | 5.898871 | 5.700392 | 5.652823 | 5.852608 | 5.941806 |
| 1382670_at | 8.607625 | 8.306811 | 8.398667 | 8.687534 | 8.345867 | 8.2197 | 8.552141 |
| 1384137_at | 4.248103 | 4.469068 | 4.06801 | 4.20896 | 4.162241 | 4.320374 | 4.276134 |
| 1374707_at | 8.157394 | 7.84171 | 8.125784 | 8.108103 | 8.19244 | 7.698878 | 7.818295 |
| 1381492_at | 3.39466 | 3.35917 | 3.314776 | 3.410916 | 3.370553 | 3.46529 | 3.38042 |
| 1389599_at | 7.149853 | 6.872076 | 6.833596 | 7.063181 | 7.294202 | 6.816706 | 6.740096 |
| 1380175_at | 5.160696 | 4.961601 | 5.291116 | 5.161053 | 5.160221 | 5.030026 | 5.212806 |
| 1370983_at | 5.629613 | 5.842618 | 5.666426 | 5.47773 | 5.572271 | 5.443443 | 5.688576 |
| 1396130_at | 3.996715 | 4.120051 | 3.889792 | 3.814712 | 3.974254 | 3.921034 | 3.958764 |
| 1387914_at | 7.113919 | 7.094792 | 6.970047 | 7.202853 | 7.171078 | 6.742761 | 7.040104 |
| 1368824_at | 8.825603 | 8.855378 | 9.531276 | 9.130224 | 8.806001 | 9.975893 | 10.39037 |
| 1381456_at | 5.510472 | 5.294116 | 5.39214 | 5.361957 | 5.478666 | 5.537828 | 5.46222 |
| 1375178_at | 5.860735 | 5.693961 | 5.836406 | 5.573762 | 5.872586 | 5.941907 | 5.899785 |
| 1377441_at | 4.926312 | 4.73964 | 4.842323 | 4.89651 | 4.794061 | 4.800346 | 4.851957 |
| 1385290_at | 5.912105 | 5.666417 | 5.934266 | 5.764757 | 5.85546 | 6.049654 | 5.921483 |
| 1368198_at | 6.200761 | 5.96258 | 5.963996 | 5.90697 | 5.996109 | 5.98626 | 5.902249 |
| 1370253_at | 11.14977 | 10.85983 | 11.06 | 11.28014 | 11.35627 | 11.36815 | 11.38424 |
| 1378906_at | 4.061325 | 4.197327 | 4.19609 | 4.263281 | 4.048104 | 4.09945 | 4.154332 |
| 1393438_at | 5.321704 | 5.380563 | 5.359406 | 5.376186 | 5.366238 | 5.286912 | 5.185457 |
| 1376661_at | 9.242853 | 8.930118 | 9.16586 | 9.212681 | 9.040385 | 8.674148 | 8.646203 |
| 1387822_at | 6.542111 | 6.445634 | 6.117231 | 6.626474 | 6.633556 | 6.308574 | 6.36465 |
| 1391283_at | 5.833841 | 5.874396 | 5.96024 | 5.702392 | 5.598074 | 5.89814 | 5.762315 |
| 1379691_at | 5.194612 | 5.150488 | 5.649781 | 5.355132 | 5.020573 | 5.42467 | 5.74783 |
| 1378200_at | 3.49684 | 3.655582 | 3.453484 | 3.611351 | 3.53718 | 3.652948 | 3.589423 |
| 1391962_at | 3.905926 | 3.859192 | 3.835026 | 3.837576 | 3.857248 | 3.917669 | 3.892958 |
| 1374608_at | 9.770124 | 9.608656 | 9.788637 | 9.721411 | 9.556286 | 9.834954 | 9.733287 |
| 1390406_at | 10.34409 | 10.3557 | 10.50253 | 10.18781 | 10.45781 | 10.31091 | 10.63144 |
| 1394343_s_at | 3.577921 | 3.611894 | 3.498068 | 3.586376 | 3.587633 | 3.604372 | 3.615012 |
| 1370317_at | 8.087234 | 8.10199 | 8.255473 | 8.307677 | 8.149681 | 8.490723 | 8.609283 |
| 1385391_at | 3.906025 | 4.128003 | 4.117534 | 4.065756 | 4.119367 | 4.247722 | 4.378803 |
| 1382156_at | 4.656724 | 4.812004 | 4.82626 | 4.711744 | 4.874337 | 4.95205 | 4.751615 |
| 1377365_at | 4.943028 | 5.013649 | 4.818194 | 5.177219 | 5.083958 | 4.986952 | 5.169062 |
| 1397587_at | 7.541917 | 7.10395 | 7.071707 | 7.170173 | 7.225319 | 6.495967 | 6.76673 |
| 1383054_at | 8.807657 | 9.017428 | 9.070439 | 8.990953 | 8.711281 | 9.325601 | 9.41996 |
| 1398949_at | 8.606254 | 8.576337 | 8.66358 | 8.940726 | 8.595456 | 8.818478 | 8.839183 |
| 1379822_at | 4.426261 | 4.487098 | 4.337195 | 4.482956 | 4.602365 | 4.653999 | 4.927613 |
| 1373572_at | 7.868107 | 7.821535 | 7.630051 | 7.99082 | 8.104034 | 8.16109 | 7.916932 |
| 1395817_at | 7.359672 | 7.160284 | 7.247123 | 7.595406 | 7.497324 | 7.412344 | 7.191531 |
| 1387694_at | 4.568289 | 4.709052 | 4.716824 | 4.567781 | 4.66874 | 4.59178 | 4.597032 |
| 1384573_at | 8.900946 | 8.97017 | 9.056015 | 9.080516 | 8.76583 | 9.187965 | 9.330243 |
| 1385967_at | 7.8281 | 7.603817 | 7.783287 | 7.852722 | 7.73947 | 7.586877 | 7.564309 |
| 1397934_at | 8.298753 | 8.220983 | 8.121526 | 8.187293 | 8.222358 | 8.186979 | 8.200005 |
| 1380887_at | 4.806778 | 4.819667 | 4.710192 | 4.69149 | 4.822587 | 4.923829 | 4.702949 |
| 1390592_at | 7.922063 | 8.041783 | 8.387599 | 8.099686 | 7.908114 | 8.782791 | 8.955466 |
| 1394383_at | 6.225513 | 6.542422 | 6.277278 | 5.892306 | 6.154807 | 6.123525 | 6.229771 |
| 1376622_at | 9.17885 | 9.105556 | 9.15757 | 9.007202 | 8.827936 | 8.849989 | 8.682105 |
| 1385074_at | 5.671337 | 5.562711 | 5.767921 | 5.52647 | 5.194523 | 5.636218 | 5.513816 |
| 1385790_at | 8.873689 | 8.702837 | 8.93601 | 8.711004 | 8.796927 | 8.832008 | 8.617366 |
| 1371174_s_at | 6.567701 | 6.371281 | 6.498948 | 6.175262 | 6.345222 | 6.398374 | 6.404599 |
| 1387836_at | 6.084263 | 6.194557 | 5.759553 | 6.332689 | 6.223032 | 6.426774 | 6.244026 |
| 1370918_a_at | 11.30863 | 11.2792 | 11.24724 | 11.36628 | 11.45237 | 11.48919 | 11.41651 |
| 1398486_at | 4.355109 | 4.744269 | 5.03229 | 4.74111 | 4.489842 | 5.709248 | 5.543765 |
| 1397268_at | 5.156685 | 5.261241 | 5.068187 | 4.989594 | 5.172585 | 4.920382 | 5.027522 |
| 1393428_at | 6.022873 | 6.1025 | 6.339632 | 6.136967 | 6.165123 | 5.934912 | 6.608032 |
| 1391678_at | 7.246252 | 7.316186 | 7.365494 | 7.474511 | 7.386948 | 7.42633 | 6.897458 |
| 1388363_at | 8.334143 | 8.136501 | 8.387347 | 8.484121 | 8.155221 | 8.23048 | 8.22643 |
| 1388669_at | 7.592953 | 7.652917 | 7.716242 | 7.739342 | 7.557408 | 7.505974 | 7.68724 |
| 1394887_x_at | 4.368967 | 4.298035 | 4.252718 | 4.257702 | 4.269459 | 4.251022 | 4.360876 |
| 1384367_at | 5.722115 | 5.628084 | 5.603972 | 6.024885 | 5.734842 | 5.669305 | 5.559763 |
| 1383329_at | 5.894521 | 5.614171 | 5.919978 | 5.669108 | 5.593446 | 5.825996 | 5.854211 |

TABLE 11

| | | | T-val | df |
|---|---|---|---|---|
| Probe set | Gene Title | Gene Symbol | contr.vs.5 | contr.vs.5 |
| 1367901_at | glucuronidase, beta | Gusb | 2.34973 | 8 |
| 1368264_at | peroxisomal biogenesis factor 6 | Pex6 | 4.697176 | 8 |
| 1368356_a_at | type 1 tumor necrosis factor receptor shedding | Arts1 | 2.94295 | 7 |
| | aminopeptidase regulator | | | |
| 1369725_at | centaurin, alpha 2 | Centa2 | 2.385189 | 11 |
| 1370237_at | L-3-hydroxyacyl-Coenzyme A dehydrogenase, short chain | Hadhsc | 2.587076 | 7 |
| 1372806_at | vacuolar protein sorting 35 (mapped) | Vps35_mapped | 3.396093 | 10 |
| 1373392_at | TPA regulated locus | Tparl | 2.297901 | 10 |
| 1374076_at | similar to hypothetical protein FLJ34389 (predicted) | RGD1305243_predicted | 2.488868 | 8 |
| 1374540_at | cell division cycle associated 7 | Cdca7 | 8.391325 | 11 |
| 1375297_at | similar to RIKEN cDNA 0610008C08 (predicted) | RGD1565289_predicted | 4.693393 | 11 |
| 1377021_at | Transcribed locus | — | 4.466365 | 10 |
| 1377263_at | cofactor required for Sp1 transcriptional activation, subunit 9 | Crsp9_predicted | 2.847021 | 6 |
| | (predicted) | | | |
| 1377866_a_at | geranylgeranyl diphosphate synthase 1 | Ggps1 | 2.359719 | 10 |
| 1378127_at | cullin 2 (predicted) | Cul2_predicted | 3.329496 | 6 |
| 1379488_at | TP53 regulating kinase (predicted) | Trp53rk_predicted | 3.065082 | 6 |
| 1381229_at | PR domain containing 2, with ZNF domain (mapped) | Prdm2_mapped | 2.490891 | 10 |
| 1383635_at | DEAD (Asp-Glu-Ala-Asp) box polypeptide 59 | Ddx59 | 2.268593 | 9 |
| 1387334_at | mast cell protease 6 | Mcpt6 | 2.814419 | 9 |
| 1388304_at | NADH dehydrogenase (ubiquinone) 1 beta subcomplex, 5 | Ndufb5_predicted | 3.089763 | 9 |
| | (predicted) | | | |
| 1388465_at | Transcribed locus | — | 2.22618 | 11 |
| 1388660_at | malignant T cell amplified sequence 1 | Mcts1 | 2.683183 | 11 |
| 1388926_at | Ectonucleotide pyrophosphatase/phosphodiesterase 5 | Enpp5 | 2.520732 | 11 |
| 1389111_at | Transcribed locus | — | 5.97356 | 11 |
| 1389325_at | similar to programmed cell death 10 | MGC72992 | 2.713215 | 10 |
| 1389833_at | Sulfatase modifying factor 1 (predicted) | Sumf1_predicted | 2.260779 | 10 |
| 1390687_at | pleckstrin | Plek | 4.190275 | 8 |
| 1391714_at | pleiomorphic adenoma gene 1 | Plag1 | 3.379754 | 9 |
| 1392286_at | Transcribed locus | — | 2.766072 | 11 |
| 1393226_at | Transcribed locus | — | 3.139333 | 9 |
| 1393310_at | Transcribed locus | — | 4.556303 | 7 |
| 1393980_at | Transcribed locus | — | 5.043674 | 10 |
| 1394077_at | Rho family GTPase 3 | Rnd3 | 2.445492 | 10 |
| 1394340_at | inositol polyphosphate-1-phosphatase | Inpp1 | 3.193406 | 8 |
| 1394591_at | zinc finger protein 207 | Zfp207 | 4.155383 | 10 |
| 1367843_at | aldo-keto reductase family 7, member A2 (aflatoxin aldehyde | Akr7a2 | 3.493363 | 9 |
| | reductase) | | | |
| 1368418_a_at | ceruloplasmin | Cp | 4.225434 | 11 |
| 1373607_at | ST3 beta-galactoside alpha-2,3-sialyltransferase 3 | St3gal3 | 2.830407 | 11 |
| 1373627_at | Similar to putative phosphoinositide 5-phosphatase type II; | LOC287533 | 2.45387 | 11 |
| | C62 | | | |
| 1374366_at | solute carrier family 39 (zinc transporter), member 4 | Slc39a4_predicted | 2.449188 | 11 |
| | (predicted) | | | |
| 1374415_at | polymerase (RNA) III (DNA directed) polypeptide E | Polr3e_predicted | 3.423041 | 7 |
| | (predicted) | | | |
| 1374454_at | Protein-L-isoaspartate (D-aspartate) O-methyltransferase | Pcmtd2_predicted | 2.510643 | 10 |
| | domain containing 2 (predicted) | | | |
| 1374614_at | kelch repeat and BTB (POZ) domain containing 4 (predicted) | Kbtbd4_predicted | 2.369813 | 8 |
| 1374770_at | N-acylsphingosine amidohydrolase 1 | Asah1 | 2.644989 | 11 |
| 1375191_at | RGD1564011 (predicted) | RGD1564011_predicted | 2.201641 | 11 |
| 1376745_at | Mss4 protein | Mss4 | 2.45043 | 7 |
| 1379314_at | — | — | 3.922643 | 11 |
| 1379496_at | RT1 class lb, locus Aw2 | RT1-Aw2 | 3.905773 | 11 |
| 1383571_at | hypothetical protein LOC303515 | LOC303515 | 2.731924 | 11 |
| 1383789_at | Transcribed locus | — | 2.475547 | 9 |
| 1385639_at | — | — | 2.411824 | 10 |
| 1387021_at | wild-type p53-induced gene 1 | Wig1 | 2.593846 | 7 |
| 1389582_at | — | — | 3.019152 | 11 |
| 1389718_at | Transcribed locus | — | 3.754451 | 11 |
| 1392953_at | — | — | 3.00961 | 8 |
| 1393037_at | — | — | 2.272651 | 11 |
| 1393245_at | Phytanoyl-CoA hydroxylase | Phyh | 4.432412 | 9 |
| 1399108_at | similar to expressed sequence AV340375 | RGD1308959 | 2.219593 | 11 |

| | p-val | T-val | df_contr.vs. | p-val | T-val 5 vs. | df | p-val 5 vs. | Mean | |
|---|---|---|---|---|---|---|---|---|---|
| Probe set | contr.vs.5 | Contr.vs_10 | 10 | contr.cs.10 | 10 | 5 vs. 10 | 10 | Contr | Mean 5% |
| 1367901_at | 0.046701 | 5.512363 | 7 | 8.95E−04 | 2.692259 | 6 | 0.035944 | 7.420203 | 6.974745 |
| 1368264_at | 0.001547 | 7.560118 | 6 | 2.78E−04 | 2.52082 | 6 | 0.045237 | 6.259265 | 5.664209 |
| 1368356_a_at | 0.021622 | 5.28988 | 4 | 0.006129 | 2.588352 | 5 | 0.048934 | 9.55972 | 9.181831 |
| 1369725_at | 0.036168 | 4.52113 | 9 | 0.0014445 | 2.45629 | 6 | 0.049367 | 5.497342 | 5.089061 |
| 1370237_at | 0.036098 | 4.990147 | 4 | 0.0075429 | 2.671601 | 5 | 0.044263 | 8.793343 | 8.480746 |
| 1372806_at | 0.006816 | 4.811583 | 10 | 7.11E−04 | 2.466162 | 6 | 0.048711 | 10.46183 | 10.13292 |
| 1373392_at | 0.044413 | 5.144197 | 4 | 0.0067716 | 4.448717 | 3 | 0.021131 | 8.308305 | 8.164291 |
| 1374076_at | 0.037589 | 5.51807 | 4 | 0.0052654 | 3.751059 | 4 | 0.019929 | 7.300542 | 6.97963 |
| 1374540_at | 4.14E−06 | 13.50243 | 10 | 9.56E−08 | 4.273706 | 6 | 0.005242 | 6.195992 | 5.287047 |
| 1375297_at | 6.57E−04 | 6.666884 | 10 | 5.59E−05 | 2.722782 | 6 | 0.034515 | 7.731478 | 6.937547 |
| 1377021_at | 0.001204 | 7.68836 | 9 | 3.04E−05 | 2.839884 | 6 | 0.029571 | 5.80011 | 5.148572 |
| 1377263_at | 0.029296 | 5.91322 | 5 | 0.0019703 | 2.475036 | 6 | 0.048128 | 7.471023 | 6.737194 |
| 1377866_a_at | 0.039972 | 4.71138 | 10 | 8.27E−04 | 4.026064 | 4 | 0.015785 | 5.646626 | 5.273044 |
| 1378127_at | 0.015817 | 6.525445 | 4 | 0.0028484 | 2.54369 | 6 | 0.043862 | 6.258107 | 5.628944 |
| 1379488_at | 0.022081 | 8.042026 | 5 | 4.81E−04 | 3.750402 | 6 | 0.009505 | 5.228085 | 4.922934 |
| 1381229_at | 0.031941 | 3.802602 | 8 | 0.0052173 | 2.537826 | 6 | 0.04421 | 5.346211 | 4.872466 |
| 1383635_at | 0.049477 | 5.90793 | 6 | 0.0010459 | 3.473082 | 6 | 0.013254 | 6.981191 | 6.626937 |
| 1387334_at | 0.020231 | 7.931182 | 8 | 4.65E−05 | 3.391524 | 4 | 0.027491 | 5.601581 | 5.206432 |
| 1388304_at | 0.012934 | 5.031626 | 7 | 0.0015106 | 3.612408 | 3 | 0.036442 | 10.12286 | 9.746815 |
| 1388465_at | 0.047845 | 4.609294 | 10 | 9.66E−04 | 3.38009 | 6 | 0.014856 | 6.549543 | 6.133349 |
| 1388660_at | 0.021287 | 4.670909 | 9 | 0.0011671 | 2.929763 | 5 | 0.032642 | 8.623529 | 8.191288 |
| 1388926_at | 0.028438 | 5.335912 | 10 | 3.30E−04 | 2.980559 | 6 | 0.024619 | 5.277582 | 4.893534 |
| 1389111_at | 9.27E−05 | 5.94563 | 3 | 0.0095132 | 3.541052 | 3 | 0.038335 | 5.875325 | 5.420104 |
| 1389325_at | 0.021813 | 5.021052 | 10 | 5.21E−04 | 3.723483 | 5 | 0.013663 | 10.24563 | 9.919337 |
| 1389833_at | 0.047306 | 5.854823 | 7 | 6.28E−04 | 3.815016 | 6 | 0.008812 | 6.839621 | 6.464176 |
| 1390687_at | 0.003037 | 8.053889 | 5 | 4.78E−04 | 3.659266 | 6 | 0.010589 | 6.105485 | 5.535644 |
| 1391714_at | 0.00813 | 4.655716 | 9 | 0.0011924 | 3.156521 | 6 | 0.019652 | 6.670053 | 5.858906 |
| 1392286_at | 0.018355 | 4.578884 | 6 | 0.003775 | 2.85526 | 4 | 0.04615 | 5.585624 | 5.242275 |
| 1393226_at | 0.011941 | 6.362825 | 9 | 1.31E−04 | 2.46958 | 6 | 0.048485 | 7.705027 | 6.778772 |
| 1393310_at | 0.002616 | 7.140214 | 4 | 0.0020349 | 3.107414 | 5 | 0.026629 | 7.364799 | 6.579394 |
| 1393980_at | 5.04E−04 | 7.18862 | 10 | 2.97E−05 | 3.874667 | 5 | 0.011705 | 5.991591 | 5.335233 |
| 1394077_at | 0.034523 | 5.820561 | 10 | 1.68E−04 | 2.778249 | 5 | 0.038983 | 7.018906 | 6.516009 |
| 1394340_at | 0.012738 | 5.333216 | 4 | 0.0059524 | 2.730803 | 5 | 0.041241 | 5.05511 | 4.704473 |
| 1394591_at | 0.001963 | 6.584037 | 6 | 5.89E−04 | 3.252108 | 5 | 0.022643 | 8.125942 | 7.429881 |
| 1367843_at | 0.006794 | 6.510363 | 7 | 3.31E−04 | 3.009154 | 6 | 0.023726 | 8.276972 | 7.547364 |
| 1368418_a_at | 0.001423 | 6.119508 | 8 | 2.83E−04 | 2.476306 | 6 | 0.048045 | 5.816824 | 5.33169 |
| 1373607_at | 0.01636 | 4.710264 | 9 | 0.0011041 | 3.115436 | 4 | 0.035684 | 6.298338 | 5.956115 |
| 1373627_at | 0.032025 | 4.955513 | 8 | 0.0011132 | 3.032061 | 4 | 0.038702 | 6.526312 | 5.829701 |
| 1374366_at | 0.032292 | 4.133168 | 7 | 0.0043865 | 2.822719 | 4 | 0.047696 | 7.252498 | 6.553385 |
| 1374415_at | 0.01109 | 5.935718 | 4 | 0.0040387 | 2.860821 | 5 | 0.035372 | 6.849002 | 6.356022 |
| 1374454_at | 0.030879 | 4.646292 | 5 | 0.0056008 | 2.724148 | 5 | 0.041569 | 6.083112 | 5.463531 |
| 1374614_at | 0.04526 | 5.824873 | 8 | 3.94E−04 | 2.818489 | 6 | 0.030415 | 6.314496 | 5.867744 |
| 1374770_at | 0.022789 | 5.234397 | 7 | 0.0012066 | 3.375706 | 5 | 0.019766 | 9.074708 | 8.620237 |
| 1375191_at | 0.049943 | 4.16391 | 10 | 0.0019366 | 2.759861 | 6 | 0.03286 | 5.471081 | 5.007805 |
| 1376745_at | 0.044079 | 6.322446 | 8 | 2.27E−04 | 2.592822 | 6 | 0.041056 | 6.629539 | 6.082428 |
| 1379314_at | 0.002382 | 5.301394 | 6 | 0.0018274 | 2.787818 | 4 | 0.049422 | 6.970231 | 6.194515 |
| 1379496_at | 0.002452 | 6.909599 | 10 | 4.15E−05 | 2.467657 | 6 | 0.048612 | 5.19844 | 4.649908 |
| 1383571_at | 0.019511 | 4.451128 | 10 | 0.001233 | 2.779463 | 6 | 0.03202 | 5.867879 | 5.339246 |
| 1383789_at | 0.035248 | 4.783315 | 5 | 0.0049561 | 2.77602 | 5 | 0.039086 | 5.574571 | 5.342271 |
| 1385639_at | 0.036569 | 4.022082 | 8 | 0.00383 | 2.936126 | 4 | 0.042553 | 4.981165 | 4.503729 |
| 1387021_at | 0.035744 | 6.194133 | 7 | 4.48E−04 | 2.582999 | 6 | 0.041601 | 6.895168 | 6.437024 |
| 1389582_at | 0.011673 | 5.212483 | 8 | 8.10E−04 | 2.852321 | 5 | 0.035726 | 7.588214 | 6.741736 |
| 1389718_at | 0.003185 | 6.329516 | 8 | 2.26E−04 | 4.108077 | 4 | 0.014755 | 5.751838 | 5.089968 |
| 1392953_at | 0.016824 | 6.345901 | 6 | 7.17E−04 | 2.831348 | 6 | 0.029905 | 7.435411 | 6.788869 |
| 1393037_at | 0.044102 | 4.865021 | 10 | 6.56E−04 | 3.829033 | 6 | 0.008669 | 7.020197 | 6.710681 |
| 1393245_at | 0.001641 | 8.437993 | 10 | 7.36E−06 | 2.536255 | 6 | 0.044304 | 6.483711 | 5.646391 |
| 1399108_at | 0.0484 | 4.479248 | 8 | 0.0020578 | 3.270939 | 4 | 0.030762 | 6.859582 | 6.436919 |

| Probe set | Mean 10% | Contr < 5 | 5 < 10 | Contr < 5 | c < 5 < 10 | Contr > 5 | 5 > 10 | Contr > 10 | c > 5 > 10 |
|---|---|---|---|---|---|---|---|---|---|
| 1367901_at | 6.431816 | NO | NO | NO | NO | YES | YES | YES | YES |
| 1368264_at | 5.303002 | NO | NO | NO | NO | YES | YES | YES | YES |
| 1368356_a_at | 8.724713 | NO | NO | NO | NO | YES | YES | YES | YES |
| 1369725_at | 4.691663 | NO | NO | NO | NO | YES | YES | YES | YES |
| 1370237_at | 8.014916 | NO | NO | NO | NO | YES | YES | YES | YES |
| 1372806_at | 9.969095 | NO | NO | NO | NO | YES | YES | YES | YES |
| 1373392_at | 7.589092 | NO | NO | NO | NO | YES | YES | YES | YES |
| 1374076_at | 6.175178 | NO | NO | NO | NO | YES | YES | YES | YES |
| 1374540_at | 4.94827 | NO | NO | NO | NO | YES | YES | YES | YES |
| 1375297_at | 6.614794 | NO | NO | NO | NO | YES | YES | YES | YES |
| 1377021_at | 4.752157 | NO | NO | NO | NO | YES | YES | YES | YES |
| 1377263_at | 5.97359 | NO | NO | NO | NO | YES | YES | YES | YES |
| 1377866_a_at | 4.808311 | NO | NO | NO | NO | YES | YES | YES | YES |
| 1378127_at | 5.031834 | NO | NO | NO | NO | YES | YES | YES | YES |
| 1379488_at | 4.494457 | NO | NO | NO | NO | YES | YES | YES | YES |
| 1381229_at | 4.661432 | NO | NO | NO | NO | YES | YES | YES | YES |
| 1383635_at | 6.017881 | NO | NO | NO | NO | YES | YES | YES | YES |
| 1387334_at | 4.848615 | NO | NO | NO | NO | YES | YES | YES | YES |
| 1388304_at | 9.267339 | NO | NO | NO | NO | YES | YES | YES | YES |
| 1388465_at | 5.667039 | NO | NO | NO | NO | YES | YES | YES | YES |
| 1388660_at | 7.770533 | NO | NO | NO | NO | YES | YES | YES | YES |
| 1388926_at | 4.550642 | NO | NO | NO | NO | YES | YES | YES | YES |
| 1389111_at | 4.793567 | NO | NO | NO | NO | YES | YES | YES | YES |
| 1389325_at | 9.571108 | NO | NO | NO | NO | YES | YES | YES | YES |
| 1389833_at | 5.77432 | NO | NO | NO | NO | YES | YES | YES | YES |
| 1390687_at | 4.932622 | NO | NO | NO | NO | YES | YES | YES | YES |
| 1391714_at | 5.553782 | NO | NO | NO | NO | YES | YES | YES | YES |
| 1392286_at | 4.791402 | NO | NO | NO | NO | YES | YES | YES | YES |
| 1393226_at | 6.077027 | NO | NO | NO | NO | YES | YES | YES | YES |
| 1393310_at | 5.833555 | NO | NO | NO | NO | YES | YES | YES | YES |
| 1393980_at | 4.943026 | NO | NO | NO | NO | YES | YES | YES | YES |
| 1394077_at | 6.068581 | NO | NO | NO | NO | YES | YES | YES | YES |
| 1394340_at | 4.268504 | NO | NO | NO | NO | YES | YES | YES | YES |
| 1394591_at | 6.769234 | NO | NO | NO | NO | YES | YES | YES | YES |
| 1367843_at | 6.866282 | NO | NO | NO | NO | YES | YES | YES | YES |
| 1368418_a_at | 5.049638 | NO | NO | NO | NO | YES | YES | YES | YES |
| 1373607_at | 5.590178 | NO | NO | NO | NO | YES | YES | YES | YES |
| 1373627_at | 5.350663 | NO | NO | NO | NO | YES | YES | YES | YES |
| 1374366_at | 5.652028 | NO | NO | NO | NO | YES | YES | YES | YES |
| 1374415_at | 5.780101 | NO | NO | NO | NO | YES | YES | YES | YES |
| 1374454_at | 4.611595 | NO | NO | NO | NO | YES | YES | YES | YES |
| 1374614_at | 5.325021 | NO | NO | NO | NO | YES | YES | YES | YES |
| 1374770_at | 7.95164 | NO | NO | NO | NO | YES | YES | YES | YES |
| 1375191_at | 4.638899 | NO | NO | NO | NO | YES | YES | YES | YES |
| 1376745_at | 5.489871 | NO | NO | NO | NO | YES | YES | YES | YES |
| 1379314_at | 5.514398 | NO | NO | NO | NO | YES | YES | YES | YES |
| 1379496_at | 4.376474 | NO | NO | NO | NO | YES | YES | YES | YES |
| 1383571_at | 5.001663 | NO | NO | NO | NO | YES | YES | YES | YES |
| 1383789_at | 4.98583 | NO | NO | NO | NO | YES | YES | YES | YES |
| 1385639_at | 4.258831 | NO | NO | NO | NO | YES | YES | YES | YES |
| 1387021_at | 5.949945 | NO | NO | NO | NO | YES | YES | YES | YES |
| 1389582_at | 6.297198 | NO | NO | NO | NO | YES | YES | YES | YES |
| 1389718_at | 4.744662 | NO | NO | NO | NO | YES | YES | YES | YES |
| 1392953_at | 6.103489 | NO | NO | NO | NO | YES | YES | YES | YES |
| 1393037_at | 6.383948 | NO | NO | NO | NO | YES | YES | YES | YES |
| 1393245_at | 5.227239 | NO | NO | NO | NO | YES | YES | YES | YES |
| 1399108_at | 5.828719 | NO | NO | NO | NO | YES | YES | YES | YES |

| | | | | | | | sig down | |
|---|---|---|---|---|---|---|---|---|
| | | | Sig | | | sig up and | and spec | |
| Probe set | sig SSRI | Sig HF | MMMCR | sig up | sig down | spec alc | alc | |
| 1367901_at | NO | NO | NO | No | Yes | NO | YES | 0.001679 |
| 1368264_at | NO | NO | NO | No | Yes | NO | YES | 7E−05 |
| 1368356_a_at | NO | NO | NO | No | Yes | NO | YES | 0.001058 |
| 1369725_at | NO | NO | NO | No | Yes | NO | YES | 0.001786 |
| 1370237_at | NO | NO | NO | No | Yes | NO | YES | 0.001598 |
| 1372806_at | NO | NO | NO | No | Yes | NO | YES | 0.000332 |
| 1373392_at | NO | NO | NO | No | Yes | NO | YES | 0.000938 |
| 1374076_at | NO | NO | NO | No | Yes | NO | YES | 0.000749 |
| 1374540_at | NO | NO | NO | No | Yes | NO | YES | 2.17E−08 |
| 1375297_at | NO | NO | NO | No | Yes | NO | YES | 2.27E−05 |
| 1377021_at | NO | NO | NO | No | Yes | NO | YES | 3.56E−05 |
| 1377263_at | NO | NO | NO | No | Yes | NO | YES | 0.00141 |
| 1377866_a_at | NO | NO | NO | No | Yes | NO | YES | 0.000631 |
| 1378127_at | NO | NO | NO | No | Yes | NO | YES | 0.000694 |
| 1379488_at | NO | NO | NO | No | Yes | NO | YES | 0.00021 |
| 1381229_at | NO | NO | NO | No | Yes | NO | YES | 0.001412 |
| 1383635_at | NO | NO | NO | No | Yes | NO | YES | 0.000656 |
| 1387334_at | NO | NO | NO | No | Yes | NO | YES | 0.000556 |
| 1388304_at | NO | NO | NO | No | Yes | NO | YES | 0.000471 |
| 1388465_at | NO | NO | NO | No | Yes | NO | YES | 0.000711 |
| 1388660_at | NO | NO | NO | No | Yes | NO | YES | 0.000695 |
| 1388926_at | NO | NO | NO | No | Yes | NO | YES | 0.0007 |
| 1389111_at | NO | NO | NO | No | Yes | NO | YES | 3.55E−06 |
| 1389325_at | NO | NO | NO | No | Yes | NO | YES | 0.000298 |
| 1389833_at | NO | NO | NO | No | Yes | NO | YES | 0.000417 |
| 1390687_at | NO | NO | NO | No | Yes | NO | YES | 3.22E−05 |
| 1391714_at | NO | NO | NO | No | Yes | NO | YES | 0.00016 |
| 1392286_at | NO | NO | NO | No | Yes | NO | YES | 0.000847 |
| 1393226_at | NO | NO | NO | No | Yes | NO | YES | 0.000579 |
| 1393310_at | NO | NO | NO | No | Yes | NO | YES | 6.97E−05 |
| 1393980_at | NO | NO | NO | No | Yes | NO | YES | 5.9E−06 |
| 1394077_at | NO | NO | NO | No | Yes | NO | YES | 0.001346 |
| 1394340_at | NO | NO | NO | No | Yes | NO | YES | 0.000525 |
| 1394591_at | NO | NO | NO | No | Yes | NO | YES | 4.45E−05 |
| 1367843_at | NO | NO | NO | No | Yes | NO | YES | 0.000161 |
| 1368418_a_at | NO | NO | YES | No | Yes | NO | NO | 6.84E−05 |
| 1373607_at | NO | NO | YES | No | Yes | NO | NO | 0.000584 |
| 1373627_at | NO | NO | YES | No | Yes | NO | NO | 0.001239 |
| 1374366_at | NO | YES | YES | No | Yes | NO | NO | 0.00154 |
| 1374415_at | NO | YES | YES | No | Yes | NO | NO | 0.000392 |
| 1374454_at | NO | YES | NO | No | Yes | NO | NO | 0.001284 |
| 1374614_at | NO | YES | NO | No | Yes | NO | NO | 0.001377 |
| 1374770_at | NO | YES | NO | No | Yes | NO | NO | 0.00045 |
| 1375191_at | NO | YES | YES | No | Yes | NO | NO | 0.001641 |
| 1376745_at | NO | YES | YES | No | Yes | NO | NO | 0.00181 |
| 1379314_at | NO | NO | YES | No | Yes | NO | NO | 0.000118 |
| 1379496_at | NO | YES | YES | No | Yes | NO | NO | 0.000119 |
| 1383571_at | NO | YES | YES | No | Yes | NO | NO | 0.000625 |
| 1383789_at | NO | YES | NO | No | Yes | NO | NO | 0.001378 |
| 1385639_at | NO | YES | NO | No | Yes | NO | NO | 0.001556 |
| 1387021_at | NO | YES | YES | No | Yes | NO | NO | 0.001487 |
| 1389582_at | NO | YES | YES | No | Yes | NO | NO | 0.000417 |
| 1389718_at | NO | NO | YES | No | Yes | NO | NO | 4.7E−05 |
| 1392953_at | NO | YES | NO | No | Yes | NO | NO | 0.000503 |
| 1393037_at | NO | YES | NO | No | Yes | NO | NO | 0.000382 |
| 1393245_at | NO | YES | NO | No | Yes | NO | NO | 7.27E−05 |
| 1399108_at | YES | NO | YES | No | Yes | NO | NO | 0.001489 |

TABLE 12

| | | Gene | T-val | df | p-val | T-val |
|---|---|---|---|---|---|---|
| Probe set | Gene Title | Symbol | contr.vs.5 | contr.vs.5 | contr.vs.5 | Contr.vs_10 |
| 1370527_a_at | casein kinase 1, delta | Csnk1d | 2.986261 | 8 | 0.017433 | 3.973956 |
| 1380351_at | — | — | 4.308549 | 11 | 0.001238 | 4.307602 |
| 1381613_at | Transcribed locus | — | 5.680522 | 11 | 1.42E−04 | 5.946172 |
| 1384272_at | Zinc finger protein 365 | Zfp365 | 2.911772 | 5 | 0.033331 | 5.77205 |
| 1384691_at | Transcribed locus, weakly similar | — | 4.243576 | 5 | 0.008142 | 6.725371 |
| | to XP_577161.1 PREDICTED: | | | | | |
| | similar to ORF2 consensus | | | | | |
| | sequence encoding | | | | | |
| | endonuclease and reverse | | | | | |
| | transcriptase minus RNaseH | | | | | |
| | [Rattus norvegicus] | | | | | |
| 1384793_at | — | — | 2.895417 | 6 | 0.027498 | 9.938201 |
| 1388932_at | laminin, alpha 5 | Lama5 | 2.484535 | 9 | 0.034732 | 3.873799 |
| 1391879_at | Transcribed locus | — | 2.524571 | 6 | 0.045008 | 6.73664 |

| | df_contr. | p-val | T-val | df 5 | p-val | Mean | | |
|---|---|---|---|---|---|---|---|---|
| Probe set | vs.10 | contr.cs.10 | 5 vs. 10 | vs. 10 | 5 vs. 10 | Contr | Mean 5% | Mean 10% |
| 1370527_a_at | 8 | 0.004096 | 3.327782 | 6 | 0.01585 | 9.270172 | 9.839312 | 10.02957 |
| 1380351_at | 3 | 0.023032 | 3.287598 | 3 | 0.046151 | 4.084842 | 4.281886 | 4.907576 |
| 1381613_at | 3 | 0.009511 | 3.55257 | 3 | 0.038021 | 4.040707 | 4.29003 | 4.634067 |
| 1384272_at | 3 | 0.010312 | 2.800243 | 6 | 0.040646 | 4.004522 | 4.22733 | 4.516728 |
| 1384691_at | 3 | 0.006711 | 2.545145 | 6 | 0.043776 | 4.1106.3 | 4.649739 | 5.112041 |
| 1384793_at | 9 | 3.77E−06 | 3.045839 | 5 | 0.02856 | 4.215733 | 4.484639 | 4.760827 |
| 1388932_at | 10 | 0.00309 | 2.860363 | 6 | 0.028788 | 9.205238 | 9.683496 | 9.896758 |
| 1391879_at | 6 | 5.21E−04 | 2.617131 | 6 | 0.039739 | 4.20034 | 4.546958 | 4.936622 |

| Probe set | Contr < 5 | 5 < 10 | Contr < 5 | c < 5 < 10 | Contr > 5 | 5 > 10 | Contr > 10 | c > 5 > 10 |
|---|---|---|---|---|---|---|---|---|
| 1370527_a_at | YES | YES | YES | YES | NO | NO | NO | NO |
| 1380351_at | YES | YES | YES | YES | NO | NO | NO | NO |
| 1381613_at | YES | YES | YES | YES | NO | NO | NO | NO |
| 1384272_at | YES | YES | YES | YES | NO | NO | NO | NO |
| 1384691_at | YES | YES | YES | YES | NO | NO | NO | NO |
| 1384793_at | YES | YES | YES | YES | NO | NO | NO | NO |
| 1388932_at | YES | YES | YES | YES | NO | NO | NO | NO |
| 1391879_at | YES | YES | YES | YES | NO | NO | NO | NO |

| | | | | | | | sig down | |
|---|---|---|---|---|---|---|---|---|
| | | | Sig | | | sig up and | and | |
| Probe set | sig SSRI | Sig HF | MMMCR | sig up | sig down | spec alc | spec alc | |
| 1370527_a_at | NO | NO | NO | Yes | No | YES | NO | 0.000276 |
| 1380351_at | NO | NO | NO | Yes | No | YES | NO | 5.72E−05 |
| 1381613_at | NO | NO | NO | Yes | No | YES | NO | 5.41E−06 |
| 1384272_at | NO | NO | NO | Yes | No | YES | NO | 0.001355 |
| 1384691_at | NO | NO | NO | Yes | No | YES | NO | 0.000356 |
| 1384793_at | NO | NO | NO | Yes | No | YES | NO | 0.000785 |
| 1388932_at | NO | NO | NO | Yes | No | YES | NO | 0.001 |
| 1391879_at | NO | YES | YES | Yes | No | NO | NO | 0.001789 |

We claim:

1. A method of diagnosing true low birth weight in an infant subject comprising
(a) measuring the level of gene expression of a true low birth weight related gene in a subject sample;
(b) comparing measured expression of the true low birth weight related gene in a control sample;
wherein a variation in gene expression characterized by a p-value of at least 0.05 between the subject sample and the control sample indicates a diagnosis of true low birth weight.

2. The method of claim 1, wherein the subject sample is derived from the placenta.

3. The method of claim 2, wherein the subject sample is derived from the fetus.

4. The method of claim 1, wherein measuring the level of gene expression is performed by the group consisting of DNA chip and real-time polymerase chain reaction.

5. The method of claim 1, wherein the infant subject is at risk of true low birth weight.

6. The method of claim 5, wherein the infant subject at risk of true low birth weight exhibits a condition selected from the group consisting of poor maternal nutrition, maternal alcohol abuse, maternal tobacco abuse, maternal drug abuse, bacterial or viral infections, illness, and genetic factors.

7. The method of claim 1, wherein the true low birth weight related gene is selected from the group consisting of genes encoding IGF, genes encoding IGF binding proteins, and genes encoding IGF receptors.

8. The method of claim 7, wherein the genes are selected from the group consisting of ALS, CTGF/CCN2, Endocan, IGFBP-1, IGFBP-2, IGFBP-3, IGFBP-4, IGFBP-5, IGFBP-6, IGFBP-7, and Nov/CCN3.

9. The method of claim 7, wherein the true low birth weight related gene is IGF1.

10. The method of claim 1, wherein the true low birth weight related gene is selected from the group consisting of solute carrier 18 (member 2) probe set 1369132), vascular adhesion molecule 1 (probe set 1368474), collagen type 1 alpha (probe set 1388116), allograft inflammatory factor 1 (1368558), arachidonate 12 lipoxygenase (probe set 1387796).

11. A kit for diagnosing true low birth weight comprising at least one oligonucleotide probe directed to a true low birth weight related gene; and a control selected from the group consisting of a standard value, a control sample, or a reference standard.

12. A method of predicting the dietary conditions during pregnancy in an infant comprising
(a) measuring the level of gene expression of one or more genes related to a dietary condition in a subject sample;
(b) comparing measured expression of the genes related to a dietary condition in a control sample;
wherein a variation in gene expression characterized by a p-value of at least 0.05 between the subject sample and the control sample indicates the presence of a dietary condition.

13. The method of claim 12, wherein the gene related to a dietary condition is selected from the group consisting of solute carrier 18 (member 2) (probe set 1369132), vascular adhesion molecule 1 (probe set 1368474), collagen type 1 alpha (probe set 1388116), allograft inflammatory factor 1 (1368558), arachidonate 12 lipoxygenase (probe set 1387796).

14. The method of claim 12, wherein the gene related to a dietary condition is selected from the group consisting of peroxisome proliferation activated receptor (gamma) (probe set 1369179), fatty acid binding protein 4 (probe set 1368271), apolipoprotein C-1 (probe set 1368587), lectin (galactose binding, soluble 9) (probe set 1387027), and glia maturation factor beta (probe set 1387663).

15. The method of claim 1, wherein the true low birth weight gene is a fetal alcohol syndrome related gene.

16. The method of claim 15, wherein the fetal alcohol syndrome related gene is selected from the group consisting of Gusb, Pex6, Arts1, Centa2, Hadhsc, Vps35 (mapped), Tpar1, RGD1305243 (predicted), Cdca7, RGD1565289 (predicted), Crsp9 (predicted), Ggps1, Cul2 (predicted), Trp53rk (predicted), Prdm2 (mapped), Ddx59, Mcpt6, Ndufb5 (predicted), Mcts1, Enpp5, MGC72992, Sumf1 (predicted), Plek, Plag1, Rnd3, Inpp1, Zfp207, Akr7a2, Cp, St3gal3, LOC287533, Slc39a4 (predicted), Polr3e (predicted), Pcmtd2 (predicted), Kbtbd4 (predicted), Asah1, RGD1564011 (predicted), Mss4, RT1-Aw2, LOC303515, Wig1, Phyh, RGD1308959, Csnk1d, Zfp365, and Lama5.
